(19) United States
(12) Patent Application Publication
(10) Pub. No.: US 2026/0117200 A1
(43) Pub. Date: Apr. 30, 2026

(54) Recombinant rhabdovirus encoding for a gasdermin

(71) Applicant: Boehringer Ingelheim International GmbH, Ingelheim am Rhein (DE)

(72) Inventors: Tobias NOLDEN, Birgitz (AT); Krishna DAS, Innsbruck (AT); Sabrina SCHNEIDER, Schwaz (AT); Theresa SCHWAIGER, Innsbruck (AT); Bart Gerrit SPIESSCHAERT, Angerberg (AT)

(21) Appl. No.: 18/985,108

(22) Filed: Dec. 18, 2024

(30) Foreign Application Priority Data
Dec. 19, 2023 (EP) 23218131.3

(51) Int. Cl. C12N 7/00; A61K 35/766; A61P 35/00; C07K 16/28

(52) U.S. Cl. CPC C12N 7/00; A61K 35/766; A61P 35/00; C07K 16/2896; C12N 2760/20221; C12N 2760/20232

(57) ABSTRACT

The present invention relates to the field of oncolytic viruses and in particular to a recombinant rhabdovirus, such as vesicular stomatitis virus encoding in its genome for a gasdermin. The invention is further directed to the use of the recombinant virus in the treatment of cancer and also to methods for producing such viruses.

### SEQUENCE LISTING

The instant application contains a Sequence Listing which has been submitted electronically in XML format and is hereby incorporated by reference in its entirety. Said XML copy, created on Dec. 12, 2024, is named 01-3577-US-1_SL.xml and is 144,214 bytes in size.

### FIELD OF THE INVENTION

The present invention relates to the field of oncolytic viruses and in particular to a recombinant rhabdovirus encoding in its genome for a gasdermin (GSDM). The invention is further directed to the use of the recombinant rhabdovirus in the treatment of cancer and to methods for producing such viruses.

### BACKGROUND OF THE INVENTION

Oncolytic viruses are an emerging class of biologicals, which selectively replicate in and kill cancer cells and can spread within tumors. Efforts to further improve oncolytic viruses, to increase their therapeutic potential, led to the development of so-called armed viruses, which encode in their genome tumor antigens or immune modulatory transgenes, to improve their efficacy in tumor treatment. A particular field of interest concentrates on identifying suitable and effective immune modulating cargos, that can be expressed from a viral backbone, and which act together with the oncolytic virus to potentiate anti-tumor efficacy.

Recently, the family of gasdermin (GSDM) proteins was proposed to play a key role in the progression of cancer. In various cancers, both epigenetic silencing and loss-of-function mutations of GSDMs were observed. GSDMs possess a C-terminal repressor domain (GSDM-CT), a cytotoxic N-terminal domain (GSDM-CT) and a flexible linker domain. GSDMs need to be activated in the cells, e.g. via caspase cleavage. The cleaved GSDM-NT domain, forms large oligomeric pores in the cell membrane, resulting in pyroptosis—a highly inflammatory form of lytic programmed cell death.

It was proposed to deliver only the active N-terminal domain of GSDMs to avoid the additional activation step. However, in the context of replicative viruses, overcoming pyroptosis induced toxicity of active GSDM-NT, remains a challenge. Once the GSDM-NT is expressed without the inhibitory GSDM-CT domain, active pores are formed in the production cell line leading to rapid cell death.

Several strategies were proposed to overcome the high toxicity of active GSDMD-NT during production of viruses. In one approach, for AAV particles, a promoter was chosen that could drive GSDMD-NT expression in tumorigenic mammalian cells, while remaining inactive in Sf9 insect cells. In another approach, an AAV was used containing a double floxed inverted GSDMD-NT, which required reversion through co-infection with an additional AAV-Cre. In another approach, a non-replication competent AAV vector was used for delivering active GSDMD-NT to glioblastoma (Lu, Y., He, W., Huang, X. et al. Strategies to package recombinant Adeno-Associated Virus expressing the N-terminal gasdermin domain for tumor treatment. Nat Commun 12, 7155 (2021). Yet others, utilized a bio-orthogonal chemical system, using the cancer-imaging probe phenylalanine trifluoroborate, to selectively release GSDMA3-NT from a nanoparticle conjugate in 4T1 cells, leading to tumor regression and enhanced anti-tumor immune responses (Wang Q, Wang Y, Ding J, Wang C, Zhou X, Gao W, Huang H, Shao F, Liu Z. A bioorthogonal system reveals antitumour immune function of pyroptosis. Nature. 2020 March; 579(7799):421-426).

All these different approaches for delivering active N-terminal GSDM into cancer cells suffer from one or more disadvantages: (i) the difficulty of delivering two components into the same cell limiting the effectiveness of the treatment, (ii) use of replication-incompetent rAAVs, meaning there is no virus spread within the tumor, limiting the therapeutic effect, and (iii) the risk of unintended consequences due to incomplete promoter specificity.

Hence, there is an ongoing need in the art for further improved viruses that can be used in effective cancer treatments.

### SUMMARY OF THE INVENTION

The present invention addresses the above needs by providing a recombinant rhabdovirus, such as a vesicular stomatitis virus, which encodes in its genome at least one GSDM or a functional variant thereof, preferably a human GSDM.

It is to be understood that any embodiment relating to a specific aspect might also be combined with another embodiment also relating to that specific aspect, even in multiple tiers and combinations comprising several embodiments to that specific aspect.

In a first aspect, the present invention relates to a recombinant rhabdovirus encoding in its genome at least one gasdermin (GSDM) or a functional variant thereof, preferably a human GSDM.

In an embodiment, relating to the first aspect or any of its embodiments, the GSDM is selected from the group consisting of: gasdermin A (GSDMA), gasdermin B (GSDMB), gasdermin C (GSDMC), gasdermin D (GSDMD), gasdermin E (GSDME or DFNA5) or DFNB59 (Pejvakin).

In a further embodiment, relating to the first aspect or any of its embodiments, the GSDM or functional variant thereof comprises (i) the N-terminal domain (GSDM-NT) of a GSDM, and (ii) the C-terminal domain (GSDM-CT) of a GSDM. In a related embodiment, the GSDM-NT comprises or consists of any one of SEQ ID NOs: 51-55. In a further related embodiment, the GSDM-CT comprises or consists of any one of SEQ ID NOs: 56-60. In a further related embodiment, the GSDM-NT comprises or consists of any one of SEQ ID NOs: 51-55, and the GSDM-CT comprises or consists of any one of SEQ ID NOs: 56-60.

In a further embodiment, relating to the first aspect or any of its embodiments, the GSDM further comprises a cleavable peptide sequence not naturally occurring in said GSDM. In a related embodiment, the cleavable peptide sequence is protease cleavable. In a related embodiment, the protease cleavable peptide sequence is specifically cleavable by caspases, preferably caspase-3. In a related embodiment, the cleavable peptide sequence comprises the consensus sequence DxxD (SEQ ID NO:62). In a related embodiment, the cleavable peptide sequence comprises the sequence DMPD (SEQ ID NO:63), DEVD (SEQ ID NO:64) or DLPD (SEQ ID NO:65).

In a further embodiment, relating to the first aspect or any of its embodiments, the GSDM comprises or consists of any one of SEQ ID NOs: 45-50.

In a further embodiment, relating to the first aspect or any of its embodiments, the recombinant rhabdovirus is a vesiculovirus. In a related embodiment, the vesiculovirus is selected from the group consisting of: Vesicular stomatitis alagoas virus (VSAV), Carajás virus (CJSV), Chandipura virus (CHPV), Cocal virus (COCV), Vesicular stomatitis Indiana virus (VSIV), Isfahan virus (ISFV), Maraba virus (MARAV), Vesicular stomatitis New Jersey virus (VSNJV), or Piry virus (PIRYV).

In a further embodiment, relating to the first aspect or any of its embodiments, the rhabdovirus is a vesicular stomatitis virus, preferably a Vesicular stomatitis Indiana virus (VSIV) or Vesicular stomatitis New Jersey virus (VSNJV). In a related embodiment, the rhabdovirus is replication-competent.

In a further embodiment, relating to the first aspect or any of its embodiments, the rhabdovirus
(i) lacks a functional gene coding for glycoprotein G, and/or
(ii) lacks a functional glycoprotein G.

In a further embodiment, relating to the first aspect or any of its embodiments, the rhabdovirus
(i) gene coding for the glycoprotein G is replaced by the gene coding for the glycoprotein GP of another virus, and/or
(ii) glycoprotein G is replaced by the glycoprotein GP of another virus.

In a further embodiment, relating to the first aspect or any of its embodiments, the rhabdovirus
(i) gene coding for the glycoprotein G is replaced by the gene coding for the glycoprotein GP of an arenavirus, and/or
(ii) the glycoprotein G is replaced by the glycoprotein GP of an arenavirus.

In a further embodiment, relating to the first aspect or any of its embodiments, the rhabdovirus
(i) gene coding for the glycoprotein G is replaced by the gene coding for the glycoprotein GP of Dandenong virus or Mopeia virus, and/or
(ii) the glycoprotein G is replaced by the glycoprotein GP of Dandenong virus or Mopeia virus.

In a further embodiment, relating to the first aspect or any of its embodiments, the rhabdovirus
(i) gene coding for the glycoprotein G is replaced by the gene coding for the glycoprotein GP of Lymphocyte choriomeningitis virus (LCMV), and/or
(ii) the glycoprotein G is replaced by the glycoprotein GP of LCMV.

In a further embodiment, relating to the first aspect or any of its embodiments, the recombinant rhabdovirus further encodes for at least one cytokine, preferably an interleukin or an interferon. In a related embodiment, the cytokine is interleukin18 (IL18), interleukin12 (IL12), and/or interleukin1 (IL1). In a related embodiment, the interferon is an interferon-type-I (IFN-type-I), preferably IFN-alpha.

In a further embodiment, relating to the first aspect or any of its embodiments, the recombinant rhabdovirus further encodes for (i) IL18 and IL12, (ii) IL18 and IL1, or (iii) IL18 and IL1 and IFN-alpha-2.

In a further embodiment, relating to the first aspect or any of its embodiments, the recombinant rhabdovirus further encodes for an IL12p35 and an IL12p40 subunit of IL12. In a related embodiment, the IL12p35 subunit and the IL12p40 subunit are human. In a related embodiment, the IL12p35 subunit comprises a polypeptide having at least 95% identity to SEQ ID NO:1 and the IL12p40 subunit comprises a polypeptide having at least 95% identity to SEQ ID NO:2, preferably the IL12p35 subunit comprises the polypeptide of SEQ ID NO:1 and the IL12p40 subunit comprises the polypeptide of SEQ ID NO:2. In a related embodiment, the IL12p40 subunit and the IL12p35 subunit are linked in a single-chain having the configuration IL12p40-IL12p35 or IL12p35-IL12p40. In a related embodiment, the IL12p40 subunit and the IL12p35 subunit are linked to each other via a linker that is rich in amino acid residues glycine and serine, preferably having a length of 5 to 20 amino acids and only including the amino acids glycine and serine, more preferably a glycine and serine linker having the amino acid sequence of SEQ ID NO:22. In a related embodiment, the single-chain IL12p40-IL12p35 comprises a polypeptide having at least 95% identity to SEQ ID NO:3 or SEQ ID NO:5; or the single-chain IL12p35-IL12p40 comprises a polypeptide having at least 95% identity to SEQ ID NO:4 or SEQ ID NO: 6. In a related embodiment, the recombinant rhabdovirus further comprises a signal peptide sequence linked to the single-chain IL12p40-IL12p35 or IL12p35-IL12p40. In a related embodiment, the signal peptide sequence comprises an amino acid sequence having at least 90% identity to SEQ ID NO:68, preferably being identical to SEQ ID NO:68. In a related embodiment, the single-chain IL12p40-IL12p35 comprises a polypeptide having at least 95% identity to SEQ ID NO:66 or SEQ ID NO: 67, preferably being identical to SEQ ID NO:66 or SEQ ID NO:67.

In a further embodiment, relating to the first aspect or any of its embodiments, the recombinant rhabdovirus further comprises a 2A-peptide, preferably selected from the group consisting of: T2A, P2A, E2A, or F2A peptide. In a related embodiment, the 2A-peptide is located between the GSDM and the IL12 protein. In a related embodiment, the 2A-peptide comprises the consensus sequence DxExNPGP (SEQ ID NO:69). In a further related embodiment, the 2A-peptide comprises an amino acid sequence having at least 90% identity to SEQ ID NOs: 70-71 and 73-75, preferably being identical to SEQ ID NOs: 70-71 and 73-75.

In a second aspect, the present invention relates to a recombinant vesicular stomatitis virus encoding in its genome at least one GSDM or a functional variant thereof, preferably a human GSDM, wherein the gene coding for the glycoprotein G of the recombinant vesicular stomatitis virus is replaced by the gene coding for the glycoprotein GP of Lymphocyte choriomeningitis virus (LCMV), and/or the glycoprotein G is replaced by the glycoprotein GP of LCMV.

In a further embodiment, relating to the second aspect or any of its embodiments, the GSDM is selected from the group consisting of: Gasdermin A (GSDMA), Gasdermin B (GSDMB), Gasdermin C (GSDMC), Gasdermin D (GSDMD), Gasdermin E (GSDME or DFNA5) or DFNB59 (Pejvakin). In a related embodiment, the GSDM or functional variant thereof comprises (i) the N-terminal domain (GSDM-NT) of a GSDM, and (ii) the C-terminal domain (GSDM-CT) of a GSDM. In a related embodiment, the GSDM-NT comprises or consists of any one of SEQ ID NOs: 51-55. In a related embodiment, the GSDM-CT comprises or consists of any one of SEQ ID NOs: 56-60. In a related embodiment, the GSDM-NT comprises or consists of any one of SEQ ID NOs: 51-55, and the GSDM-CT comprises or consists of any one of SEQ ID NOs: 56-60.

In a further embodiment, relating to the second aspect or any of its embodiments, the GSDM further comprises a cleavable peptide sequence not naturally occurring in said GSDM. In a related embodiment, the cleavable peptide sequence is protease cleavable. In a related embodiment, the protease cleavable peptide sequence is specifically cleavable by caspases, preferably caspase-3. In a related embodiment, the cleavable peptide sequence comprises the consensus sequence DxxD (SEQ ID NO:62). In a related embodiment, the cleavable peptide sequence comprises the sequence DMPD (SEQ ID NO:63), DEVD (SEQ ID NO:64) or DLPD (SEQ ID NO:65).

In a further embodiment, relating to the second aspect or any of its embodiments, the GSDM comprises or consists of any one of SEQ ID NOs: 45-50.

In a further embodiment, relating to the second aspect or any of its embodiments, the recombinant vesicular stomatitis virus further encodes for at least one cytokine, preferably an interleukin or an interferon. In a related embodiment, the cytokine is interleukin18 (IL18), interleukin12 (IL12), and/or interleukin1 (IL1). In a related embodiment, the interferon is an interferon-type-I (IFN-type-I), preferably IFN-alpha.

In a further embodiment, relating to the second aspect or any of its embodiments, the recombinant vesicular stomatitis virus further encodes for (i) IL18 and IL12, (ii) IL18 and IL1, or (iii) IL18 and IL1 and IFN-alpha-2.

In a further embodiment, relating to the second aspect or any of its embodiments, the recombinant vesicular stomatitis further encodes for an IL12p35 and an IL12p40 subunit of IL12. In a related embodiment, the IL12p35 subunit and the IL12p40 subunit are human. In a related embodiment, the IL12p35 subunit comprises a polypeptide having at least 95% identity to SEQ ID NO:1 and the IL12p40 subunit comprises a polypeptide having at least 95% identity to SEQ ID NO:2, preferably the IL12p35 subunit comprises the polypeptide of SEQ ID NO:1 and the IL12p40 subunit comprises the polypeptide of SEQ ID NO:2. In a related embodiment, the IL12p40 subunit and the IL12p35 subunit are linked in a single-chain having the configuration IL12p40-IL12p35 or IL12p35-IL12p40. In a related embodiment, the IL12p40 subunit and the IL12p35 subunit are linked to each other via a linker that is rich in amino acid residues glycine and serine, preferably having a length of 5 to 20 amino acids and only including the amino acids glycine and serine, more preferably a glycine and serine linker having the amino acid sequence of SEQ ID NO:22. In a related embodiment, the single-chain IL12p40-IL12p35 comprises a polypeptide having at least 95% identity to SEQ ID NO:3 or SEQ ID NO:5; or the single-chain IL12p35-IL12p40 comprises a polypeptide having at least 95% identity to SEQ ID NO:4 or SEQ ID NO: 6. In a related embodiment, the recombinant vesicular stomatitis virus further comprises a signal peptide sequence linked to the single-chain IL12p40-IL12p35 or IL12p35-IL12p40. In a related embodiment, the signal peptide sequence comprises an amino acid sequence having at least 90% identity to SEQ ID NO:68, preferably being identical to SEQ ID NO:68. In a related embodiment, the single-chain IL12p40-IL12p35 comprises a polypeptide having at least 95% identity to SEQ ID NO:66 or SEQ ID NO:67, preferably being identical to SEQ ID NO:66 or SEQ ID NO:67.

In a further embodiment, relating to the second aspect or any of its embodiments, the recombinant vesicular stomatitis virus further comprises a 2A-peptide, preferably selected from the group consisting of: T2A, P2A, E2A, or F2A peptide. In a related embodiment, the 2A-peptide is located between the GSDM and the IL12 protein. In a related embodiment, the 2A-peptide comprises the consensus sequence DxExNPGP (SEQ ID NO:69). In a related embodiment, the 2A-peptide comprises an amino acid sequence having at least 90% identity to SEQ ID NOs: 70-71 and 73-75, preferably being identical to SEQ ID NOs: 70-71 and 73-75.

In a third aspect, the present invention relates to a recombinant vesicular stomatitis virus encoding in its genome at least one GSDM comprising the amino acid of sequence of SEQ ID NO:49, and an IL12p35 and an IL12p40 subunit of IL12 linked in a single-chain having the configuration IL12p40-IL12p35 and comprising the amino acid sequence of SEQ ID NO:5 or SEQ ID NO:66, wherein the gene coding for the glycoprotein G of the recombinant vesicular stomatitis virus is replaced by the gene coding for the glycoprotein GP of Lymphocyte choriomeningitis virus (LCMV), and/or the glycoprotein G is replaced by the glycoprotein GP of LCMV.

In a fourth aspect, the present invention relates to a recombinant vesicular stomatitis virus encoding in its genome an amino acid sequence with at least 90% identity to SEQ ID NO:72, preferably an amino acid sequence identical to SEQ ID NO: 72, wherein the gene coding for the glycoprotein G of the recombinant vesicular stomatitis virus is replaced by the gene coding for the glycoprotein GP of Lymphocyte choriomeningitis virus (LCMV), and/or the glycoprotein G is replaced by the glycoprotein GP of LCMV.

In a fifth aspect, the present invention relates to a recombinant vesicular stomatitis virus, encoding in its genome at least for a vesicular stomatitis virus nucleoprotein (N), large protein (L), phosphoprotein (P), matrix protein (M), glycoprotein (G) and at least one GSDM or a functional variant thereof, preferably a human GSDM.

In a further embodiment, relating to the fifth aspect or any of its embodiments, the GSDM is selected from the group consisting of: Gasdermin A (GSDMA), Gasdermin B (GSDMB), Gasdermin C (GSDMC), Gasdermin D (GSDMD), Gasdermin E (GSDME or DFNA5) or DFNB59 (Pejvakin). In a related embodiment, the GSDM or functional variant thereof comprises (i) the N-terminal domain (GSDM-NT) of a GSDM, and (ii) the C-terminal domain (GSDM-CT) of a GSDM. In a related embodiment, the GSDM-NT comprises or consists of any one of SEQ ID NOs: 51-55. In a related embodiment, the GSDM-CT comprises or consists of any one of SEQ ID NOs: 56-60. In a related embodiment, the GSDM-NT comprises or consists of any one of SEQ ID NOs: 51-55, and the GSDM-CT comprises or consists of any one of SEQ ID NOs: 56-60.

In a further embodiment, relating to the fifth aspect or any of its embodiments, the GSDM further comprises a cleavable peptide sequence not naturally occurring in said GSDM. In a related embodiment, the cleavable peptide sequence is protease cleavable. In a related embodiment, the protease cleavable peptide sequence is specifically cleavable by caspases, preferably caspase-3. In a related embodiment, the cleavable peptide sequence comprises the consensus sequence DxxD (SEQ ID NO:62). In a related embodiment, the cleavable peptide sequence comprises the sequence DMPD (SEQ ID NO:63), DEVD (SEQ ID NO:64), or DLPD (SEQ ID NO:65).

In a further embodiment, relating to the fifth aspect or any of its embodiments, the GSDM comprises or consists of any one of SEQ ID NOs: 45-50. In a related embodiment, the recombinant vesicular stomatitis virus comprises a nucleoprotein (N) comprising an amino acid sequence as set forth in SEQ ID NO:28 or a functional variant at least 80%, 85%, 90%, 92%, 94%, 96%, 98% identical to SEQ ID NO: 28. In a related embodiment, the recombinant vesicular stomatitis virus comprises a phosphoprotein (P) comprising an amino acid sequence as set forth in SEQ ID NO:29 or a functional variant at least 80%, 85%, 90%, 92%, 94%, 96%, 98% identical to SEQ ID NO:29. In a related embodiment, the recombinant vesicular stomatitis virus comprises a large protein (L) comprising an amino acid sequence as set forth in SEQ ID NO:30 or a functional variant at least 80%, 85%, 90%, 92%, 94%, 96%, 98% identical to SEQ ID NO:30. In a related embodiment, the recombinant vesicular stomatitis virus comprises a matrix protein (M) comprising an amino acid sequence as set forth in SEQ ID NO:31 or a functional variant at least 80%, 85%, 90%, 92%, 94%, 96%, 98% identical to SEQ ID NO:31.

In a further embodiment, relating to the fifth aspect or any of its embodiments, the recombinant vesicular stomatitis virus comprises
a nucleoprotein (N) comprising an amino acid sequence as set forth in SEQ ID NO: 28 or a functional variant at least 80%, 85%, 90%, 92%, 94%, 96%, 98% identical to SEQ ID NO:28,
a phosphoprotein (P) comprising an amino acid sequence as set forth in SEQ ID NO: 29 or a functional variant at least 80%, 85%, 90%, 92%, 94%, 96%, 98% identical to SEQ ID NO:29,
a large protein (L) comprising an amino acid sequence as set forth in SEQ ID NO: 30 or a functional variant at least 80%, 85%, 90%, 92%, 94%, 96%, 98% identical to SEQ ID NO:30, and
a matrix protein (M) comprising an amino acid sequence as set forth in SEQ ID NO: 31 or a functional variant at least 80%, 85%, 90%, 92%, 94%, 96%, 98% identical to SEQ ID NO:31.

In a further embodiment, relating to the fifth aspect or any of its embodiments, the recombinant vesicular stomatitis virus is replication-competent.

In a further embodiment, relating to the fifth aspect or any of its embodiments, the recombinant vesicular stomatitis virus
(i) lacks a functional gene coding for glycoprotein G, and/or
(ii) lacks a functional glycoprotein G.

In a further embodiment, relating to the fifth aspect or any of its embodiments, the vesicular stomatitis virus
(i) gene coding for the glycoprotein G is replaced by the gene coding for the glycoprotein GP of another virus, and/or
(ii) glycoprotein G is replaced by the glycoprotein GP of another virus.

In a further embodiment, relating to the fifth aspect or any of its embodiments, the vesicular stomatitis virus
(i) gene coding for the glycoprotein G is replaced by the gene coding for the glycoprotein GP of an arenavirus, and/or
(ii) the glycoprotein G is replaced by the glycoprotein GP of an arenavirus.

In a further embodiment, relating to the fifth aspect or any of its embodiments, the vesicular stomatitis virus
(i) gene coding for the glycoprotein G is replaced by the gene coding for the glycoprotein GP of Dandenong virus or Mopeia virus, and/or
(ii) the glycoprotein G is replaced by the glycoprotein GP of Dandenong virus or Mopeia virus.

In a further embodiment, relating to the fifth aspect or any of its embodiments, the vesicular stomatitis virus
(i) gene coding for the glycoprotein G is replaced by the gene coding for the glycoprotein GP of Lymphocyte choriomeningitis virus (LCMV), and/or
(ii) the glycoprotein G is replaced by the glycoprotein GP of LCMV.

In a sixth aspect, the present invention relates to a recombinant vesicular stomatitis virus encoding in its genome a vesicular stomatitis virus nucleoprotein (N), large protein (L), phosphoprotein (P), matrix protein (M), glycoprotein (G) and at least one GSDM or a functional variant thereof, preferably human GSDM, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, and wherein
the nucleoprotein (N) comprises an amino acid as set forth in SEQ ID NO:28 or a functional variant at least 80%, 85%, 90%, 92%, 94%, 96%, 98% identical to SEQ ID NO:28,
wherein the phosphoprotein (P) comprises an amino acid as set forth in SEQ ID NO: 29 or a functional variant at least 80%, 85%, 90%, 92%, 94%, 96%, 98% identical to SEQ ID NO:29,
wherein the large protein (L) comprises an amino acid as set forth in SEQ ID NO: 30 or a functional variant at least 80%, 85%, 90%, 92%, 94%, 96%, 98% identical to SEQ ID NO:30, and
the matrix protein (M) comprises an amino acid as set forth in SEQ ID NO:31 or a functional variant at least 80%, 85%, 90%, 92%, 94%, 96%, 98% identical to SEQ ID NO:31.

In a further embodiment, relating to the sixth aspect or any of its embodiments, the GSDM is selected from the group consisting of: Gasdermin A (GSDMA), Gasdermin B (GSDMB), Gasdermin C (GSDMC), Gasdermin D (GSDMD), Gasdermin E (GSDME or DFNA5) or DFNB59 (Pejvakin). In a related embodiment, the GSDM or functional variant thereof comprises (i) the N-terminal domain (GSDM-NT) of a GSDM, and (ii) the C-terminal domain (GSDM-CT) of a GSDM. In a related embodiment, the GSDM-NT comprises or consists of any one of SEQ ID NOs: 51-55. In a related embodiment, the GSDM-CT comprises or consists of any one of SEQ ID NOs: 56-60. In a related embodiment, the GSDM-NT comprises or consists of any one of SEQ ID NOs: 51-55, and the GSDM-CT comprises or consists of any one of SEQ ID NOs: 56-60.

In a further embodiment, relating to the sixth aspect or any of its embodiments, the GSDM further comprises a cleavable peptide sequence not naturally occurring in said GSDM. In a related embodiment, the cleavable peptide sequence is protease cleavable. In a related embodiment, the protease cleavable peptide sequence is specifically cleavable by caspases, preferably caspase-3. In a related embodiment, the cleavable peptide sequence comprises the consensus sequence DxxD (SEQ ID NO:62). In a related embodiment, the cleavable peptide sequence comprises the sequence DMPD (SEQ ID NO:63), DEVD (SEQ ID NO:64) or DLPD (SEQ ID NO:65).

In a further embodiment, relating to the sixth aspect or any of its embodiments, the GSDM comprises or consists of any one of SEQ ID NOs: 45-50.

In a further embodiment, relating to the sixth aspect or any of its embodiments, the recombinant vesicular stomatitis virus further encodes for at least one cytokine, preferably an interleukin or an interferon. In a relate embodiment, the cytokine is interleukin18 (IL18), interleukin12 (IL12), and/or interleukin1 (IL1). In a related embodiment, the interferon is an interferon-type-I (IFN-type-I), preferably IFN-alpha.

In a further embodiment, relating to the sixth aspect or any of its embodiments, the recombinant vesicular stomatitis virus further encodes for (i) IL18 and IL12, (ii) IL18 and IL1, or (iii) IL18 and IL1 and IFN-alpha-2.

In a further embodiment, relating to the sixth aspect or any of its embodiments, the recombinant vesicular stomatitis virus further encodes for an IL12p35 and an IL12p40 subunit of IL12. In a related embodiment, the IL12p35 subunit and the IL12p40 subunit are human. In a related embodiment, the IL12p35 subunit comprises a polypeptide having at least 95% identity to SEQ ID NO:1 and the IL12p40 subunit comprises a polypeptide having at least 95% identity to SEQ ID NO:2, preferably the IL12p35 subunit comprises the polypeptide of SEQ ID NO:1 and the IL12p40 subunit comprises the polypeptide of SEQ ID NO:2. In a related embodiment, the IL12p40 subunit and the IL12p35 subunit are linked in a single-chain having the configuration IL12p40-IL12p35 or IL12p35-IL12p40. In a related embodiment, the IL12p40 subunit and the IL12p35 subunit are linked to each other via a linker that is rich in amino acid residues glycine and serine, preferably having a length of 5 to 20 amino acids and only including the amino acids glycine and serine, more preferably a glycine and serine linker having the amino acid sequence of SEQ ID NO:22. In a related embodiment, the single-chain IL12p40-IL12p35 comprises a polypeptide having at least 95% identity to SEQ ID NO:3 or SEQ ID NO:5; or the single-chain IL12p35-IL12p40 comprises a polypeptide having at least 95% identity to SEQ ID NO: 4 or SEQ ID NO:6. In a related embodiment, the recombinant vesicular stomatitis virus further comprises a signal peptide sequence linked to the single-chain IL12p40-IL12p35 or IL12p35-IL12p40. In a related embodiment, the signal peptide sequence comprises an amino acid sequence having at least 90% identity to SEQ ID NO:68, preferably being identical to SEQ ID NO:68. In a related embodiment, the single-chain IL12p40-IL12p35 comprises a polypeptide having at least 95% identity to SEQ ID NO: 66 or SEQ ID NO:67, preferably being identical to SEQ ID NO:66 or SEQ ID NO:67.

In a further embodiment, relating to the sixth aspect or any of its embodiments, the recombinant vesicular stomatitis virus further comprises a 2A-peptide, preferably selected from the group consisting of: T2A, P2A, E2A, or F2A peptide. In a related embodiment, the 2A-peptide is located between the GSDM and the IL12 protein. In a related embodiment, the 2A-peptide comprises the consensus sequence DxExNPGP (SEQ ID NO:69). In a related embodiment, the 2A-peptide comprises an amino acid sequence having at least 90% identity to SEQ ID NOs: 70-71 and 73-75, preferably being identical to SEQ ID NOs: 70-71 and 73-75.

In a seventh aspect, the invention relates to a recombinant vesicular stomatitis virus encoding in its genome a vesicular stomatitis virus nucleoprotein (N), large protein (L), phosphoprotein (P), matrix protein (M), glycoprotein (G) and at least one GSDM comprising the amino acid of sequence of SEQ ID NO:49, and an IL12p35 and an IL12p40 subunit of IL12 linked in a single-chain having the configuration IL12p40-IL12p35 and comprising the amino acid sequence of SEQ ID NO:5 or SEQ ID NO: 66, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, and wherein
the nucleoprotein (N) comprises an amino acid as set forth in SEQ ID NO:28 or a functional variant at least 80%, 85%, 90%, 92%, 94%, 96%, 98% identical to SEQ ID NO:28
wherein the phosphoprotein (P) comprises an amino acid as set forth in SEQ ID NO: 29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO:31 or a functional variant at least 80%, 85%, 90%, 92%, 94%, 96%, 98% identical to SEQ ID NO:31.

In an eight aspect, the invention relates to a recombinant vesicular stomatitis virus encoding in its genome a vesicular stomatitis virus nucleoprotein (N), large protein (L), phosphoprotein (P), matrix protein (M), glycoprotein (G) and an amino acid sequence with at least 90% identity to SEQ ID NO:72, preferably an amino acid sequence identical to SEQ ID NO:72, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, and wherein
the nucleoprotein (N) comprises an amino acid as set forth in SEQ ID NO:28 or a functional variant at least 80%, 85%, 90%, 92%, 94%, 96%, 98% identical to SEQ ID NO:28
wherein the phosphoprotein (P) comprises an amino acid as set forth in SEQ ID NO: 29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO:31 or a functional variant at least 80%, 85%, 90%, 92%, 94%, 96%, 98% identical to SEQ ID NO:31.

In a ninth aspect, the invention relates to a pharmaceutical composition, characterized in that the composition comprises a recombinant rhabdovirus according to the first aspect and/or any of its embodiments, or a recombinant vesicular stomatitis virus according to the second to eight aspect and/or any of their embodiments.

In a tenth aspect, the invention relates to a recombinant rhabdovirus according to the first aspect and/or any of its embodiments, a recombinant vesicular stomatitis virus according to according to the second to eight aspect and/or any of their embodiments, or a pharmaceutical composition according to the ninth aspect for use as a medicament.

In an eleventh aspect, the invention relates to a recombinant rhabdovirus according to the first aspect and/or any of its embodiments, a recombinant vesicular stomatitis virus according to according to the second to eight aspect and/or any of their embodiments, or a pharmaceutical composition according to the ninth aspect for use in the treatment of cancer, preferably solid cancers. In a related embodiment, the recombinant rhabdovirus, the recombinant vesicular stomatitis virus or the pharmaceutical composition for use in the treatment of solid cancer, wherein the solid cancer is selected from the list comprising: reproductive cancer, ovarian cancer, testicular cancer, endocrine cancer, gastrointestinal cancer, pancreatic cancer, pancreatic adenocarcinoma, liver cancer, kidney cancer, colon cancer, colorectal cancer, bladder cancer, bladder urothelial carcinoma, muscle invasive bladder cancer (MIBC), non-muscle invasive bladder cancer (NMIBC), prostate cancer or carcinoma, skin cancer, (metastatic) melanoma, respiratory cancer, lung cancer, non-small cell lung cancer, small cell lung cancer, (metastatic) breast cancer or carcinoma, (metastatic) triple negative breast cancer (TNBC), head & neck cancer, head and neck squamous-cell carcinoma (HNSCC), bone cancer, gastric cancer, brain cancer, endometrial cancer, vaginal cancer, anal cancer, oropharyngeal squamous cell carcinoma, gastroesophageal junction adenocarcinoma, esophageal carcinoma, gastro esophageal junction (GEJ) cancer, oesophageal and gastroesophageal junction cancer, adenocarcinoma of the GEJ, hepatocellular carcinoma, cholangiocarcinoma, squamous cell carcinoma, and glioblastoma.

In a twelfth aspect, the invention relates to the recombinant rhabdovirus, the recombinant vesicular stomatitis virus or the pharmaceutical composition for use according to the eleventh aspect and/or any of its embodiments, wherein the recombinant rhabdovirus, the recombinant vesicular stomatitis virus, or the pharmaceutical composition is to be administered intratumorally or intravenously.

In a thirteenth aspect, the invention relates to the recombinant rhabdovirus, the recombinant vesicular stomatitis virus or the pharmaceutical composition for use according to the eleventh aspect and/or any of its embodiments, wherein the recombinant rhabdovirus, the recombinant vesicular stomatitis virus or the pharmaceutical composition is to be administered at least once intratumorally and subsequently intravenously. In a related embodiment, the subsequent intravenous administration is given 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or 31 days after the initial intratumoral administration.

In a fourteenth aspect, the invention relates to a composition comprising a recombinant rhabdovirus or a recombinant vesicular stomatitis virus according to any of the preceding aspects and/or their embodiments and further a PD-1 pathway inhibitor. In a related embodiment, the PD-1 pathway inhibitor is an antagonistic antibody, which is directed against PD-1 or PD-L1. In a related embodiment, the PD-1 pathway inhibitor is an antagonist selected from the group consisting of pembrolizumab, nivolumab, pidilizumab, atezolizumab, avelumab, durvalumab, PDR-001, PD1-1, PD1-2, PD1-3, PD1-4 and PD1-5.

In a fifteenth aspect, the invention relates to a kit of parts comprising:
a) a recombinant rhabdovirus, a recombinant vesicular stomatitis virus or a pharmaceutical composition as defined in any one of the preceding aspects and/or their embodiments, and
b) a PD-1 pathway inhibitor as defined in the fourteenth aspects and/or any of its embodiments.

In a sixteenth aspect, the invention relates to a recombinant rhabdovirus, a recombinant vesicular stomatitis virus, or a pharmaceutical composition for use according to the tenth to eleventh aspect and/or any of their embodiments in combination with a PD-1 pathway inhibitor. In a related embodiment, the recombinant rhabdovirus, the recombinant vesicular stomatitis virus, or the pharmaceutical composition is administered concomitantly, sequentially or alternately with the PD-1 pathway inhibitor. In a related embodiment, the PD-1 pathway inhibitor is selected from the group consisting of pembrolizumab, nivolumab, pidilizumab, atezolizumab, avelumab, durvalumab, PDR-001, PD1-1, PD1-2, PD1-3, PD1-4 and PD1-5. In a related embodiment, the recombinant rhabdovirus, the recombinant vesicular stomatitis virus, or the pharmaceutical composition is administered via a different administration route then the PD-1 pathway inhibitor. In a related embodiment, the recombinant rhabdovirus, the recombinant vesicular stomatitis virus, or the pharmaceutical composition are administered at least once intratumorally and the PD-1 pathway inhibitor is administered intravenously.

In a seventeenth aspect, the invention relates to a virus producing cell, characterized in that the cell produces a recombinant rhabdovirus or recombinant vesicular stomatitis virus according to any of the preceding aspects and or their embodiments. In a related embodiment, the cell is a Vero cell, a HEK cell, a HEK293 cell, a Chinese hamster ovary cell (CHO), or a baby hamster kidney (BHK) cell.

### BRIEF DESCRIPTION OF THE DRAWINGS

FIGS. 1A-B Gasdermin expression and activation by caspase-3 can be separated and attributed to the early and late-stage stage of the VSV-GP lifecycle. (A) Following the initial stages of the VSV life cycle, which involve virion entry (1) and viral genome uncoating (2), the GSDM recombinant virus expresses its viral proteins and a recombinant caspase-3 (Cas3)-cleavable gasdermin (GSDMCas3), like gasdermin E in the cytoplasm of the infected cell (3). Within the first 1-8 hours, the C-terminal gasdermin domain inhibits gasdermin pore formation by the N-terminal domain. At later stages of the life cycle, viral genomes and proteins accumulate in the infected cells. (B) Expression of the VSV-M protein results in a blockage of nuclear mRNA and cessation of cellular protein translation (4). Consequently, cellular stress is detected by the mitochondrial system, which activates effector caspase-9 and subsequently leads to Cas3 cleavage. Active cleaved Cas3 then activates gasdermin by cleaving the inhibitory C-terminal domain and the pore-forming active N-terminal GSDM domain (5). Accumulation and subsequent pore formation of the GSDM-NT domain at the plasma membrane release danger-associated molecular patterns (DAMPs). Concurrently, progeny virions bud from the plasma membrane (6). Ultimately, cell swelling caused by water influx through the GSDM pore results in membrane rupture and pyroptosis-like cell death.

FIGS. 2A-C VSV-GP infection leads to caspase-3/7 activation in murine colorectal tumor cells. CT26CI.25-IFNAR1−/− cells were infected at an MOI=0.001. Caspase-3/7 activity (A) and Annexin V surface exposure (B) were measured in triplicate using an image-based analysis with an automated microscopic system (Incucyte S3, Sartorius). Cytotoxicity (C) was monitored using a cell viability stain that is incorporated into the DNA of dying cells. For positive control, apoptosis was induced by Staurosporin (Stauro) or tumor necrosis factor-α (TNF-α) and cycloheximide (CHX). For negative control, cells were treated with DMSO or apoptosis induction was blocked by adding Z-VAD-fmk (ZVAD) to TNF-α/CHX treated cells. Caspase 3/7+ and relative Annexin V signals were normalized by cell confluency. Cytotoxicity is represented as a percentage of maximal cytotoxicity induced by TNF-α/CHX after 48 hours post-infection (hpi).

FIGS. 3A-B GSDME is cleaved after Raptinal and VSV-GP infection in a murine breast cancer cell line. GSDME knock out (GSDME−/−), GSDME overexpressing (GSDME oe) or Caspase3 knock out (CAS3−/−) EMT6 cells were treated with 10 μM Raptinal (+) for 2 hours or left untreated (−). Cellular extracts were analyzed for GSDME (A, upper panel) and Caspase3 GSDME (A, middle panel) cleavage by western blotting using an automated protein separation and immunodetection system (Jess ProteinSimple, Biotechne). As loading control, β-actin (ACTB) was detected (A, lower panel). GSDME−/− and GSDME oe EMT6 cells were infected with VSV-GP (empty vector) at an MOI=3 or left uninfected (−). Cells were harvested 24 h post-infection and cellular extracts were analyzed for GSDME cleavage (B, upper panel). As controls, β-actin (ACTB) and viral proteins (VSV-N, -P, and -M) were detected (B, lower panels).

FIGS. 4A-E Mouse tumor cell lines express low levels of GSDME. Expression of GSDME and GSDMD were analyzed at the transcript and protein level. mRNA transcripts for GSDME and GSDMD were detected by next-generation sequencing. Relative expression (Log 2) of gasdermin transcripts is plotted in counts per million (CPM) relative to the expression of housekeeping genes (A). GSDME and GSDMD protein in cellular extracts derived from various murine tumor cell lines were analyzed using an automated protein separation and immunodetection system (Jess ProteinSimple, Biotechne). For control, GSDME over-expressing B16-F10 cells (B) or GSDMD-expressing THP-1 cells were used (D). Quantification of GSDMD and GSDME signals normalized to β-actin relative to GSDME overexpressing B16.F10 (C) or endogenous GSDMD expression in THP-1 (E).

FIGS. 5A-C VSV-GP-GSDME and VSV-GP-ΔM51-GSDME display distinct GSDME cleavage kinetics. (A) Full-length GSDME, which can be cleaved and activated by either caspase-3 or Granzyme B, is positioned between the viral glycoprotein LCMV-GP and the VSV polymerase L. (B) VSV-GP-ΔM51-GSDME contains a single methionine deletion at position 51 of the VSV matrix protein (M). This mutation disrupts the interaction between the matrix protein M and the nuclear pore complex. Consequently, VSV-GP-ΔM51-GSDME is unable to shut off the host cell's nuclear mRNA transport. (C) Analysis of GSDME and cleaved GSDME (p30). 4T1 cells were infected at an MOI=10 with either VSV-GP-GSDME or VSV-GP-ΔM51-GSDME. At specified timepoints post-infection (hpi), cell extracts were analyzed by western blotting for GSDME cleavage (upper panel). For control, cells were either left untreated (mock) or infected with the parental VSV-GP (empty vector). Detection of VSV proteins confirmed virus infection (middle panel), while actin (ACTB) served as a loading control (lower panel).

FIGS. 6A-D Replication of VSV-GP-GSDME and VSV-GP-DM51-GSDME viruses in HEK293T cells. HEK293F cells were infected at an MOI of 0.0005, and supernatants were harvested at the indicated timepoints (n=2 for each virus). Mock (untreated) or VSV-GP (empty vector) infected cells were used as controls. Infectious titers in cell culture supernatants were determined by the standard TCID50 assay (A) on BHK21 cells (n=2 per timepoint), and genomic copies were measured by VSV-N specific qPCR (B). Total cell numbers were counted automatically in duplicates (C) using cell counting cassettes, and dead cell counts were determined using acridine orange stain (D).

FIGS. 7A-B Genetic engineered caspase-3 cleavable gasdermins. Structure-based sequence alignment of human (hs) and murine (mm) GSDMD and GSDME (A). The secondary structures of GSDME are marked above the sequences. Identical residues in GSDMD and GSDME are shown in white within black shaded boxes. The N-terminal domain (NTD) and the C-terminal domain (CTD) are separated by a flexible LINKER region. Cas1 and Cas3 cleavage sites are indicated by black arrowheads. The minimal conserved caspase cleavage signals within GSDMD and GSDME are boxed. Cas1 exosite binding residues are marked by black dots. Genomic organization of a VSV-GP variant that incorporates a caspase-3-cleavable Gasdermin. Caspase-3-cleavable GSDMD or GSDME is inserted between the LCMV glycoprotein GP and the viral polymerase L. GSDMD wildtype is engineered to become a caspase-3 substrate (GSDMD_{DEVD}) by substituting the caspase-1-specific tetrapeptide FLTD with DEVD, which can be cleaved by caspase 3 (B).

FIGS. 8A-B Human GSDMD_{DEVD} but not GSDMD is cleaved in 4T1 mouse breast cancer cells or human CRC cell lines HT-29 and HCT-116 after infection with VSV-GP-hsGSDMD or VSV-GP-hsGSDMD_{DEVD}. 4T1 cells were infected at an MOI=10. GSDMD and GSDMD_{DEVD} expression and cleavage in cell extracts at indicated timepoints post infection (hpi) were analyzed by Western blotting (A, upper panel). HT-29 and HCT-116 were infected at an MOI=10. GSDMD and GSDMD_{DEVD} expression and cleavage in cell extracts at 20 h post infection (hpi) was analyzed (B, upper panel). For control, cells were left untreated (mock) or were infected with the parental VSV-GP (empty vector). Actin (ACTB) serves as a loading control (A and B, lower panels).

FIGS. 9A-D Replication of VSV-GP-GSDMD and VSV-GP-GSDMD_{DEVD} viruses in HEK293F cells. HEK293F cells were infected at an MOI of 0.0005, and supernatants were harvested at indicated timepoints (n=2 for each virus). Mock (untreated) or VSV-GP (empty vector) infected cells were used as controls. Infectious titers in cell culture supernatants were determined by the standard TCID50 assay (A) on BHK21 cells (n=2 per timepoint), and genomic copies were measured by VSV-N specific qPCR (B). Total cell numbers were counted automatically in duplicates (C) using cell counting cassettes, and dead cell counts were determined using Acridine orange stain (D).

FIGS. 10A-D Activated GSDME-NT or GSDMD-NT is not incorporated into the viral envelope and impacts virus particle stability. Short term stability TCID50 data of sucrose cushion purified virus particles at 20° C. RT were calculated as a percent of Log 10 TCID50 relative to t=0, either VSV-GP (empty vector) or VSV-GP expressing the named cargo. Data are represented as the mean of n=3 replicates±SEM (A). TCID50 titers of the master seed virus stock (MVSS) were compared after approximately 1 (Mar21) year and after 3.5 years (Nov23) of storage at −80° C. (B). Western blot analysis of viral preparations probed with αGSDME, aGSDMD mAB or αVSV polyclonal rabbit serum (C, D). Total amount of approximately 1×10¹⁰ TCID₅₀ sucrose cushion (sucrose) or AEC/SEC-purified GSDME virions were loaded per lane. For GSDMD and GSDMD_{DEVD} only sucrose cushion purified stocks were used. As positive control (Ctrl), 0.4 mg protein extract from VSV-GP-GSDME (C), VSV- or VSV-GP-GSDMD_{DEVD} (GSDMD_{DEVD}) infected cells was used (D).

FIGS. 11A-D Characterization of mouse VSV-GP-GSDME virus particles by multi-angle light scattering (MALS) and CryoEM. Example CryoEM images of VSV-GP (A) and VSV-GP-mmGSDME (B) at the indicated magnification. Tails of particles are indicated by arrow heads. LCMV-GP trimeric spikes are indicated by black arrows (B). Particle length was determined from CryoEM images (C) and radius of gyration (RMS) by MALS (D) from several genetically modified VSV-GPs of different genomic sizes Values are plotted as mean against genomic length (kb)-VSV-GP (empty vector) or VSV-GP expressing the named cargo. The dotted line represents the 95% confidence interval of the linear regression.

FIGS. 12A-C Pyroptotic phenotype in VSV-GP-GSDME and VSV-GP-GSDMD_{DEVD} infected 4T1 cells. 4T1 cells infected with GSDME (A) or GSDMD_{DEVD} and GSDMD (B) at MOI of 1 in a 96 well cell culture plate. Control cells treated with empty vector (VSV-GP) or left untreated (mock). CytoToxGreen uptake (1 μM) was monitored using an Incucyte S3 live cell imaging system every 10 min at 10× magnification. Green Object count/Phase area of the cell monolayer displayed hourly. Increased CytoToxGreen uptake in GSDME and GSDMD_{DEVD} compared to control, indicating gasdermin pore formation and membrane integrity loss. Representative images (C) show phenotypic change from apoptosis to pyroptotic cell death, characterized by cell swelling and membrane rupture at indicated timepoints.

FIGS. 13A-D 4T1 breast cancer cells undergoing VSV-GP-Gasdermin-induced pyroptosis do not exhibit early apoptotic characteristics. Pyroptotic tumor cells release various DAMPs, such as Annexins, ATP, and HMGB1. Additionally, dying cells can expose ER-resident Calreticulin on the cell surface (ectoCRT). DAMPs serve as “Eat Me” or “Find Me” signals and trigger receptor signaling on antigen-presenting dendritic cells, as depicted in (A). The schematic workflow of image-based cell-by-cell analysis is used to detect viable, early apoptotic, and dying cells. Uptake of Annexin-Red or Cytotox-Green dyes was monitored by an Incucyte live-cell-imaging system. For image analysis, recorded microscopic pictures were masked and classified according to their mean fluorescent intensity (B). 4T1 breast cancer cells were infected at an MOI=10 TCID50 with VSV-GP-hsGSDME, -hsGSDMD, or -hsGSDMD_{DEVD} (C and D). For control, cells were infected with VSV-GP (empty vector) or left untreated (mock). Data were analyzed using PRISM software (Vers.9.5.0). Data are represented as the percent of total cells per analyzed image, calculated from the mean of n=3 replicates.

FIGS. 14A-C ATP is released from cells after VSV-GP-hsGSDME infection. 4T1 breast cancer cells were infected with either VSV-GP-hsGSDME or VSV-GP (empty vector) at an MOI of 10 or left untreated as a control (mock). Extracellular ATP released by pyroptotic cells was measured by the ATP-dependent bioluminescence of the Luciferase present in the cell culture assay. The bar graph in (A) represents single timepoints, as indicated by the arrows from the time course analysis on the left. For the dose-response, cells were infected at different MOIs as indicated in (B). To confirm the Cas3-dependency of the pyroptotic cell death induced by the indicated viruses, cells were treated with the Cas3-specific inhibitor zDEVDfmk or the pan-caspase inhibitor zVADfmk at 50 and 100 μM. The counts per second of the Luciferase signal at 16 hpi (B and C) are represented as the mean of n=3 replicates±SEM.

FIGS. 15A-C ATP is released from cells infected with VSV-GP-hsGSDMD_{DEVD} but not with -GSDMD or empty vector. 4T1 breast cancer cells were infected with VSV-GP-hsGSDMD, VSV-GP-hsGSDMD_{DEVD} and VSV-GP (empty vector) at an MOI of 10 or left untreated (mock). Extracellular ATP released by pyroptotic cells was measured by the ATP-dependent bioluminescence of the Luciferase present in the cell culture assay. The bar graph in (A) represents single timepoints, as indicated by the arrows from the time course analysis on the left. For the dose-response, cells were infected at different MOIs as indicated in (B). To confirm the Cas3-dependency of the pyroptotic cell death induced by the indicated viruses, cells were treated with the Cas3-specific inhibitor zDEVDfmk or the pan-caspase inhibitor zVADfmk at 50 and 100 μM. The counts per second of the Luciferase signal at 16 hpi (B and C) are represented as the mean of n=3 replicates±SEM.

FIGS. 16A-E Improved tumor control by low dose treatment with VSV-GP-hsGSDME. C57BL/6J mice were subcutaneously injected with TC-1 tumor cells (10⁵ cells), deficient for the interferon alpha 1 receptor (TC-1-IFNAR1^{-/-}), into the right flank (ipsilateral). Following the same procedure, mice were injected subcutaneously in the left flank with 1×10⁵ TC-1 cells on Day 6 (CT=contralateral tumor). A virus treatment (10² TCID₅₀) was given intratumorally either with VSV-GP (empty vector) (B) or VSV-GP (A) expressing the named cargo and animals were evaluated for tumor growth over time. The x-axis shows the time (in days after tumor implantation) and the y-axis the tumor volume (in mm³). Individual tumor graphs are depicted. Following the same procedure but without the implantation of a contralateral tumor, on day 7 post-treatment, tumors were collected and a single-cell suspension was prepared for flow cytometric analysis. A virus treatment (10² TCID₅₀) was given intratumorally either with VSV-GP (empty vector) or VSV-GP expressing the named cargo. Tumors were analyzed by flow cytometry harvested on day seven post virus treatment. Total count of (C) tumor-infiltrating CD8 T cells, (D) VSV-N-specific and (E) E7-specific CD8 T cells per gram tumor are shown as means±SEM (n=5).

FIGS. 17A-E In vivo efficacy in B16-F10 melanoma model. (A) Schematic representation of the study design. Mice were implanted with 10⁶ B16F10 cells and treated on day 10 and 13 post tumor implantation with a viral dose of 1×10⁸ TCID50 intratumorally (bold black dotted line) and on day 14, 17 and 20 with 10 mg/kg of the checkpoint inhibitor anti-PD1 intraperitoneally (thin black dotted line). The figures depict (B, D) tumor growth curves, (C, E) and tumor volumes at day 21 post tumor implantation of mice, bearing B16-F10 tumors, treated with either hsGSDME (B, C) or empty vector (D, E), with or without anti-PD-1 co-treatment. (B, D) The x-axis shows the time (in days after tumor implantation) and the y-axis the tumor volume (in mm³). The figures depict the group mean with standard error of the mean with last observation carried forward until 70% of the group size was reached. (C, E) The bar graphs depict the mean with standard error of the mean of tumor volumes at day 21 of the same animals as figure B and D. The x-axis shows whether (+) or not (−) there was co-treatment with anti-PD1 and the y-axis the tumor volume (in mm³). A one-way ANOVA was performed (*p<0.05).

FIGS. 18A-B Flow cytometric analysis of migratory and costimulatory capacity of different dendritic cell populations obtained from mice from the study in FIG. 17 but without PD-1 treatment. Tumor-draining lymph nodes were harvested on day seven post viral treatment and analyzed by flow cytometry. Frequency of (A) migratory (CCR7-positive) and (B) costimulatory (CD86-positive) cells among pDCs (plasmacytoid dendritic cells), cDCs (conventional dendritic cells) and inflammatory moDCs (monocyte-derived dendritic cells) are depicted as means±SEM (n=5).

FIGS. 19A-B In vivo efficacy. (A) A schematic representation of the study design. Mice were engrafted with 10⁶ EMT-6 cells deficient for the interferon type I receptor complex Ifnar1 gene and treated on day 7 and 10 post tumor implantation with a viral dose of 1×10⁸ TCID₅₀ intratumorally. (B) The x-axis shows the time (in days) and the y-axis the percentage of mice that survived. The legend depicts which virus was used as treatment, the number of complete responders relative to the treatment group size (CR) and the mean survival (ms) as number of days post engraftment.

FIGS. 20A-B Gasdermin (GSDM) expressing VSV-GP virus can be engineered to express up to three additional immunomodulatory proteins. Cargos in VSV-GP can be placed between the viral glycoprotein LCMV-GP and the viral polymerase VSV-L. Transcription of the cargo mRNA is regulated by adding additional start and stop sequences in the intergenic region upstream and downstream of the cargo gene (A). VSV-GP-GSDM can be armed with additional immunomodulatory proteins such as IL1, IL12, IL18, or IFN type I, separated by 2A peptides derived from several members of the Picornaviridae family. TSS=transcriptional start site (B).

FIGS. 21A-G Expression of multiple cargoes by VSV-GP has little impact on viral fitness and shows no strong positioning effects. GSDMD_{DEVD} was combined with IL-1, IL18, and IFNα in different combinations (A). The integration of up to four proteins has only a minimal effect on the infectious (B) or genome titers (C). Viability of cells remains high when HEK293F cells are infected at a low MOI (0.0005). However, between 36 h and 48 h, the viability of the GSDMD_{DEVD} variants exhibits a significantly stronger decrease compared to the empty vector (D). This decrease can be explained by the gasdermin function. The expression of IL1 and IL18 at indicated timepoints (E, F) is not influenced by their position within the fusion protein. IFNα positioning at the end of both variants is used as an internal control for cargo expression (G).

FIGS. 22A-B Upregulation of IL12 receptor post VSV-GP treatment. LLC1-IFNAR1^{-/-} bearing mice treated with a single intravenous dose of 1×10⁸ TCID50 of empty vector and tumors were collected 3- and 7-days post treatment (A). RNA from tumor homogenates was used for transcriptome analysis, using the nCounter analysis system from NanoString Technologies. The fold change in gene expression of both receptor subunits is visualized (x-axis) relative to its p-value (y-axis) for both collection timepoints (B).

FIGS. 23A-F Combining VSV-GP-GSDME with a cytokine cargo that improves functionality of T cells. VSV-GP expressing GSDME and IL12 impacts the immunogenicity of oncolysis and the subsequent anti-tumor immune response (A). Genomic organization of the VSV-GP-GSDME-IL12 oncolytic virus vector (B). Replication kinetics of single cargo VSV-GP-IL12, -GSDME, and dual-cargo -GSDME-IL12 viruses (C), as well as a comparison of human (hs) GSDME-IL12 and mouse (mm) GSDME-IL12 viruses (D, E) HEK293F cells were infected at an MOI=0.0005. Infectious titers were measured by TCID50 assay on BHK21 cells (C, D), while genomic titers (E) were determined by VSV-N-specific qPCR from supernatants. For control, parental VSV-GP was used. Data points are displayed as the mean value of two biological replicates. TCID50 was performed with two technical replicates, and VSV-N qPCR with three technical replicates. Expression of hs- and mmIL12 in supernatants of infected HEK293F cells at indicated timepoints was measured by ELISA in duplicates (F).

FIGS. 24A-C Performance of VSV-GP-GSDME-IL12 in the downstream manufacturing process to generate clinical grade drug substance. Host cell protein of in-process control samples during the VSV-GP hsGSDME-IL12 manufacturing process were analyzed by an ELISA-based method and compared to the parameters of the parental virus (empty vector) (A). Infectious titers were determined by TCID50 assay on BHK21 cells from infected cell culture supernatants (harvest) and drug substances. The data are representative of two manufacturing runs each of VSV-GP (empty vector) and VSV-GP-hsGSDME-IL12 (B). IL12 in the harvest and drug substance was measured in duplicates by an IL12 ELISA to confirm low levels of free IL12 protein in the drug substance after downstream processing (C).

FIGS. 25A-C Characterization of mouse and human VSV-GP-GSDME-IL12 virus particles by multi-angle light scattering (MALS) and CryoEM. Particle length was determined from CryoEM images and radius of gyration (RMS) by MALS from several genetically modified VSV-GPs of different genomic sizes, and plotted as mean against genomic length (kb). The dotted line represents the 95% confidence interval of the linear regression (A). Example CryoEM images of VSV-GP-mmGSDME-IL12 at the indicated magnification. Tails of particles are indicated by arrow heads. LCMV-GP trimeric spikes are indicated by black arrows (B). Stability data at 20° C. (RT) were calculated as a percent of Log 10 relative t=0. Data are represented as the mean of n=3 replicates±SEM (C).

FIGS. 26A-B Survival and immune modulation in preclinical TC-1 model. (A) A schematic representation of the study design. Tumors were implanted in C57BL/6J mice by subcutaneously injecting mixture of tumor cells (10⁵ cells) into the right flank. The mixture of tumor cells consisted of 80% wildtype TC-1 and 20% TC-1-IFNAR1^{-/-} (deficient for interferon alpha 1 receptor expression). (B) The x-axis shows the time (in days) and the y-axis the percentage of mice that survived. The legend depicts which virus was used as treatment, the number of complete responders relative to the treatment group size (CR) and the mean survival (ms) as number of days post engraftment.

FIGS. 27A-B Flow cytometric analysis of splenocytes. Spleens harvested on day three post virus treatment were analyzed by flow cytometry. Total count of CD8 T cells that express (A) activation markers (CD69 and CD25) and (B) cytotoxic molecules are depicted. Data are displayed as individual points superimposed on the mean value bar (n=6).

FIGS. 28A-D Flow cytometric analysis of tumor-infiltrating leukocytes. Tumors harvested on day seven post virus treatment were analyzed by flow cytometry. Total count of tumor-infiltrating CD8 T cells that (A) express activation markers (CD69 and CD25) and (B) showing effector-memory phenotype are depicted. Count of (C) E7-specific CD8 T cells and (D) the expression of cytotoxic molecules within this tumor-specific T cell population are shown. Data are displayed as individual points superimposed on the mean value bar (n=6).

FIGS. 29A-D In vivo dose response. (A) A schematic representation of the study design. Mice were subcutaneously implanted with 10⁶ CT26.CI25-IFNaR1^{-/-} cells into the right flank. Different doses of virus treatment were given intravenously (black dotted line). (B) The x-axis shows the time (in days after tumor implantation) and the y-axis the tumor volume (in mm³). The figure depicts the group mean with standard error of the mean with last observation carried forward until 70% of the group size was reached. The vehicle control in this study did not grow properly and was therefore omitted. (C) A schematic representation of the study design. Mice were subcutaneously implanted with 106 CT26.CI25-IFNaR1^{-/-} cells into the right flank. Different doses of virus treatment were given intratumorally (black dotted line). (D) The x-axis shows the time (in days after tumor implantation) and the y-axis the tumor volume (in mm³). The figure depicts the group mean with standard error of the mean with last observation carried forward until 70% of the group size was reached. One-way ANOVA test was performed at day 32 (***p<0.001) and day 46 (** P<0.01).

FIGS. 30A-C In vivo abscopal effect. (A) A schematic representation of the study design. Tumors were implanted in C57BL/6J mice by subcutaneously injecting TC1-IFNAR1^{-/-} tumor cells (10⁵ cells) into the right flank, followed by the implantation of TC1 wildtype cells on the left flank after a 7-day interval. Virus treatments (10⁶ TCID₅₀) were given intratumorally 12 days after ipsilateral tumor implantation. (B) The figure depicts the tumor volumes of the ipsilateral tumor (group mean with standard error of the mean with last observation carried forward until 70% of the group size was reached). The x-axis shows the time (in days after tumor implantation) and the y-axis the tumor volume (in mm³). Time of virus treatment is marked by the black dotted line. (C) The figure depicts the tumor volumes of the contralateral tumor (group mean with standard error of the mean with last observation carried forward until 70% of the group size was reached). The x-axis shows the time (in days after tumor implantation) and the y-axis the tumor volume (in mm³). Time of virus treatment is marked by the black dotted line.

FIGS. 31A-E Colorectal cancer (CRC) tissue infected with VSV-GP-hsGSDME-IL12 triggers GSDME activation, IL12 expression, and subsequent IFNγ release. Overview of the experimental setup (A). Patients-derived human CRCs were cut by a vibratome into 200 μm slices. Slices from different layers were allocated to different treatment groups and the respective baseline samples were preserved. All treatment arms comprised 4 to 6 replicates from different layers of the biospecimen. Freshly untreated slices were fixed, embedded and stained by H&E for base-line characterization after preparing 4 μm ultrathin sections. Tumor cells (Tumor, □), cells of the tumor microenvironment (TME, Δ) and dead cells (Dead, ◯) were quantified using digital pathology (B/C). CRC slices were infected with VSV-GP-Katushka, VSV-GP-Katushka-hsGSDME or VSV-GP-hsGSDME-IL12. At 72 h post-infection, cell lysates were generated and analyzed via Western blotting with antibodies for GSDME and β-actin. Western blots were quantitated using the software Compass for Simple Western (Version.6.1.0). Levels of GSDME and GSDME-NT (p30) protein were normalized to levels of actin for each sample (D). Results represent the averages of three slices derived from CRC-43 experiments±standard errors of the means (SEM). Quantification of IL12 and IFNγ secretion in the supernatants of three different CRC specimen (n=4-6/CRC) by Legendplex assay 72 hpi (E). Replicates from the same specimen were labeled by the same corresponding symbols.

FIGS. 32A-B In vivo efficacy. Mice were orthotopically (OT) engrafted with 2.5×10⁵ EMT-6 cells and treated on day 7, 10, 13, 16 (A) or 6, 9, 12, 15 (B) post tumor implantation with a viral dose of 1×10⁸ TCID₅₀ intratumorally, respectively. The x-axis shows the time (in days) and the y-axis the percentage of mice that survived. The legend depicts which virus was used as treatment; the number of complete responders relative to the treatment group size (CR). Log-rank test (*p<0.05).

FIGS. 33A-B Upregulation of antigen processing and presentation gene cluster. EMT-6-IFNAR1 \-bearing mice were treated with a single intra-tumoral dose of 1×10⁸ TCID₅₀ of VSV-GP-mmIL12 or VSV-GP-mmGSDME-IL12 and tumors were collected 1 day post treatment (A). RNA from tumor homogenates was used for transcriptome analysis, using the nCounter analysis system from NanoString Technologies. The fold change in gene expression of both receptor subunits is visualized (x-axis) relative to its p-value (y-axis) (B).

FIGS. 34A-C The depletion of CD8⁺ cells negatively impacts the anti-tumor effects of oncolytic virus treatment on MC-38 tumors. MC-38 cells (5×10⁵) were implanted subcutaneously (s.c.) in the flanks of C57BL/6 mice (n=10/group). On day 12 post tumor implantation, mice were given the virus treatment (10⁶ TCID₅₀). Either 50 μg Anti-CD8 monoclonal antibody (InvivoPlus anti-mouse CD8 (Clone 2.43), ref.: BX-BP0061-25 MG, 25 mg, from BioXcell) or isotype control (InvivoPlus rat IgG2 anti-KLH, ref.: BX-BP0090-25 MG, 25 mg, from BioXcell) were injected on days 6, 9, 12, 15, 18, 21 post implantation (A). The successful depletion of CD45⁺CD90.2⁺CD8⁺ cells was confirmed on day 12 post tumor implantation by flow cytometry (B) Changes in tumor volume across groups were measured over time (C). Bars indicate means±SEM. * p<0.05, **p<0.01.

FIGS. 35A-B VSV-GP-GSDME infection results in a decrease of apoptotic pathway activation upstream and downstream of Caspase3 and less prominent nuclear DNA fragmentation. EMT6 IFNaR^{-/-}GSDME^{-/-} murine breast cancer cells were infected with VSV-GP (empty vector), VSV-GP-mmGSDME, VSV-GP-mmGSDME_{F2A}, and VSV-GP-mmGSDME_{D270A} for 8 and 16 hours or were left untreated (mock) for control. Cellular extracts were examined for GSDME (S1A, upper panel) and Caspase9-, Caspase3-, and PARP-cleavage (A, middle panels) by western blotting using an automated protein separation and immunodetection system (Jess ProteinSimple, Biotechne). For loading control, β-actin and VSV-N and -M/P proteins (ACTB) were detected (A, lower panels). Fragmentation of nuclear DNA appears less significant in VSV-GP-mmGSDME infected EMT6 IFNaR^{-/-} GSDME^{-/-} murine breast cancer cells (arrows). Cells stained with CytotoxGreen and AnnexinVRed dyes (EssenBioSciences) were infected with either VSV-GP or VSV-GP-mmGSDME at an MOI as specified. Images were taken with an Incucyte live-imaging system every two hours for 48 h. Representative images for VSV-GP and VSV-GP-mmGSDME infected EMT6 IFNaR^{-/-} GSDME^{-/-} murine breast cancer cells are displayed (B).

### DETAILED DESCRIPTION OF THE INVENTION

The inventors set out to design a new and effective oncolytic immunotherapy by harnessing the power of the immune system and thereby inducing a more potent anti-tumor immune response. In the past, it was proposed that oncolytic viruses can induce tumor cell lysis combined with immunogenic cell death and stimulation of innate immune cells in the tumor microenvironment. However, although oncolytic viruses can specifically infect and lyse tumor cells, the accompanying cell death resembles an apoptotic phenotype which can be less immunogenic. The inventors hypothesized, that to improve oncolytic viral therapy, “dying of the tumor cells in the right way” may be a key factor on the path of inducing an optimal and potent anti-tumor immune response.

GSDMs belong to the family of pore-forming effector proteins, that can cause membrane permeabilization and pyroptosis. Pyroptosis is a highly inflammatory form of lytic programmed cell death. GSDMs contain a cytotoxic N-terminal domain (GSDM-NT) and a C-terminal repressor domain (GSDM-CT). Proteolytic cleavage between these two domains releases the intramolecular inhibition on the cytotoxic domain allowing it to insert into cell membranes and form large oligomeric pores, which disrupt ion homeostasis and induces cell death. However, oncolytic viruses encoding or delivering only the active GSDM-NT (GSDM-NT without the inhibitory GSDM-CT domain) cannot be produced viably in production cell lines, because expression of the active GSDM-NT is toxic for the production cell line, before sufficient viral progeny can be obtained. On the other hand, encoding or delivering the full-length non-active GSDM, leaves the question on how to activate the GSDM once it has been expressed within the tumor cells. Hence, optimal delivery, expression and activation of GSDM are key factors to address.

It was found, that a recombinant rhabdovirus according to the invention encoding for at least one GSDM is effective for the treatment of cancer and for eliciting a potent anti-tumor immune response. Moreover, a recombinant rhabdovirus encoding for at least one GSDM and encoding additionally interleukin12 (IL12) was effective in further improving the quality of the immune response to the tumor.

Until today, there was no successful approach to specifically deliver and activate GSDMs via a replication-competent oncolytic virus without impacting the viral fitness, the ability to produce high-quality oncolytic virus at high titers for clinical applications and the ability of progeny virus to spread in the tumor.

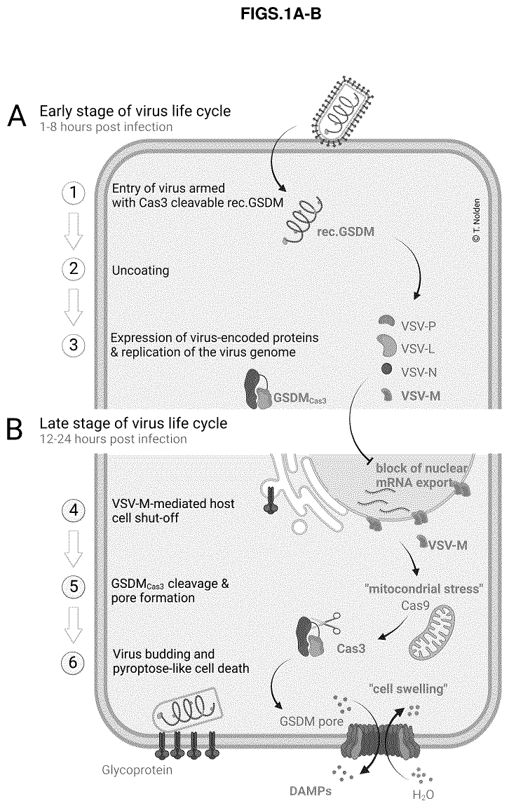

Without wishing to be bound by theory, it is believed that the strong anti-tumoral and immune stimulating effects obtained by the recombinant rhabdovirus according to the invention encoding for a GSDM is based at least on a two-fold mechanism, including, first the optimal time point of expression of the GSDM from the viral genome and second, a viral induced caspase-3 induction. It was found, that during the viral lifecycle (FIG. 1A) GSDM expression occurs in the early stages and was separated from the late-stage proteolytic activation by caspase-3. Initially, the viral proteins and the GSDM are expressed from the viral backbone in the infected cell's cytoplasm. At this stage pore formation by the N-terminal domain is still prevented by the C-terminal GSDM domain. In the later stages of the virus life cycle (FIG. 1B) viral genomes and proteins accumulate in the infected cells leading to a block of nuclear mRNA and cellular protein translation. This triggers cellular stress, activating effector caspase-9 and subsequently activation of caspase-3. Caspase-3 then activates GSDM by cleaving the inhibitory C-terminal domain, allowing pore formation and the induction of pyroptotic cell death. Hence, GSDM expression and activation by caspase-3 can be separated and attributed to the early and late-stage of the viral lifecycle, respectively.

Surprisingly, both the effects of virus induced tumor cell lysis, including replication and subsequent expression of the GSDM, correlated well with viral induced caspase-3 upregulation and lead to optimal cleavage and activation of the cytotoxic GSDM-NT within the infected tumors. Such a virus not only preserved its properties as an oncolytic virus but also showed increased immunogenicity by inducing a pyroptotic cell death phenotype through delivery and activation of GSDM.

The viral immune stimulating effect can be further boosted by additionally encoding IL12 into the virus genome. The infection of tumor cells with viruses leads to cell lysis and inflammation. This induces an influx of tumor infiltrating immune cells, which recognize tumor-derived (neo) antigens or viral proteins. The expression of GSDMs transforms the induced cell death of the infected tumor cells to a more immunogenic mode, namely pyroptosis. The local inflammation and immune cell influx leads to a substantial increase of IL12 receptor. Independent of the baseline presence of IL12 receptor, the viral expressed IL12 can activate the IL12 signaling cascade. This in turn can induce immune cell activation, Th1 (re) polarization, IFN-γ release and improve T-cell function. In conclusion, both GSDMs and IL12 support the oncolytic virus in its ability to turn the tumor microenvironment more immunogenic, thereby supporting immune-mediated tumor shrinkage.

This new therapeutic concept augments the functionality of oncolytic viruses by endowing them with transgenes that promote immune responses and modulate cell death, thereby enhancing their therapeutic potential and facilitating improved clinical dosing. The initiation of cell death pathways, distinct from apoptosis provides an option to treat the inherent resistance of many tumors to immunogenic cell death.

In one aspect, a recombinant rhabdovirus encoding for at least one GSDM is useful to induce pyroptosis in tumor cells, including e.g. membrane rupture and/or release of DAMPS.

In one aspect, a recombinant rhabdovirus encoding for at least one GSDM can be obtained from a (suspension) cell culture in sufficient quantities and quality for clinical applications in man.

In one aspect, a recombinant rhabdovirus encoding for at least one GSDM is useful to treat tumors that do not express GSDM. In another aspect, a recombinant rhabdovirus encoding for at least one GSDM is useful to treat tumors that only have a low expression level of GSDM. In another aspect, a recombinant rhabdovirus encoding for at least one GSDM is useful to treat tumors that express GSDM at baseline levels or higher.

In one aspect, a recombinant rhabdovirus encoding for at least one GSDM is useful for an improved activation of dendritic cells. In a related aspect, a recombinant rhabdovirus encoding for at least one GSDM is useful to increase the numbers of DC in tumor-draining lymph nodes as measured by flow cytometry. In a further related aspect, a recombinant rhabdovirus encoding for at least one GSDM is useful for enhancing the migratory capacity of dendritic cells, as indicated by increased CCR7. In a further related aspect, a recombinant rhabdovirus encoding for at least one GSDM is useful for improving the co-stimulatory capacity of dendritic cells, as indicated by increased CD86.

In one aspect, a recombinant rhabdovirus encoding for at least one GSDM is useful to sensitize tumors, previously resistant to anti-PD1 treatment, to be eligible again for anti-PD1 treatment.

In one aspect, a recombinant rhabdovirus encoding for at least one GSDM and IL12 is useful to improve the quality of the T-cell response. In a related aspect, a recombinant rhabdovirus encoding for at least one GSDM and IL12 is useful to increase CD8+ T-cell infiltration and tumor cell death. In a further related aspect, a recombinant rhabdovirus encoding for at least one GSDM and IL12 is useful for the activation of CD8 T-cells. In a further related aspect, a recombinant rhabdovirus encoding for at least one GSDM and IL12 is useful for increasing the number of splenic CD8 T cells expressing the cytotoxic molecules Granzyme B and Perforin. In a further related aspect, a recombinant rhabdovirus encoding for at least one GSDM and IL12 is useful for increasing the cell counts of active CD8 T-cells in tumor tissues. In a related aspect, a recombinant rhabdovirus encoding for at least one GSDM and IL12 is useful to increase effector memory T cell count, to induce a long-lasting and recallable immune response.

In one aspect, a recombinant VSV-GP encoding for at least one GSDM and IL12 showed a more pronounced amelioration in therapeutic efficacy and prolonged survival. For example, analogous levels of efficacy were attainable, in comparison to VSV-GP, upon administering a 10-fold reduced systemic dose. This augmentation in efficacy became even more prominent when the viral agents were delivered intratumorally. In this context, a recombinant VSV-GP encoding for at least one GSDM and IL12 led to a significant improvement over a 100-fold higher dose of VSV-GP treatment.

In one aspect, a recombinant VSV-GP encoding for at least one GSDM and IL12 when given intratumorally is useful to induce a systemic immune response resulting in an abscopal therapeutic effect.

In the following detailed description, numerous specific details are set forth to provide a full understanding of the present invention. It will be apparent, however, to one ordinarily skilled in the art that the subject technology may be practiced without some of these specific details. In other instances, well-known structures and techniques have not been shown in detail so as not to obscure the present invention. The headings are included merely for convenience to assist in reading and shall not be understood to limit the invention to specific aspects or embodiments.

### Gasdermin (GSDM) or Gasdermins (GSDMs)

The gasdermin (GSDM) protein family comprises a group of structure-related proteins, such as gasdermin A (GSDMA), gasdermin B (GSDMB), gasdermin C (GSDMC), gasdermin D (GSDMD), gasdermin E (GSDME or DFNA5) or DFNB59 (Pejvakin). The common structure of GSDMs include a cytotoxic N-terminal domain (GSDM-NT), a C-terminal repressor domain (GSDM-CT) and an interdomain linker harboring a cleavable peptide sequence (with notable differences for Pejvakin). Structural features of the different GSDM were reviewed in the past, see for example, Liu Z, Wang C, Yang J, Chen Y, Zhou B, Abbott D W, Xiao T S. Caspase-1 Engages Full-Length Gasdermin D through Two Distinct Interfaces That Mediate Caspase Recruitment and Substrate Cleavage. Immunity. 2020 Jul. 14; 53 (1):106-114; Liu Z, Wang C. Yang J, Zhou B, Yang R, Ramachandran R, Abbott D W, Xiao T S. Crystal Structures of the Full-Length Murine and Human Gasdermin D Reveal Mechanisms of Autoinhibition, Lipid Binding, and Oligomerization. Immunity. 2019 Jul. 16; 51(1):43-49.

Activation of the cytotoxic GSDM-NT is achieved by cleavage of the cleavable peptide sequence. Upon cleavage of this cleavable peptide sequence the GSDM-NT domain is released from the GSDM-CT domain allowing it to form large oligomeric pores in the cell membrane.

The GSDM can be of any origin including from mouse and rat. Preferably, the GSDM is from human origin. In some embodiments, the GSDM has a wild-type sequence. In other embodiments, the GSDM is a natural or engineered variant. In some embodiments, the GSDM comprises one or more mutations, substitutions and/or deletions compared to its wild-type sequence.

In one embodiment, the GSDM comprises (i) the N-terminal domain (GSDM-NT) of a GSDM, and (ii) the C-terminal domain (GSDM-CT) of a GSDM.

In another embodiment, the GSDM comprises (i) the N-terminal domain (GSDM-NT) of a GSDM, and (ii) the C-terminal domain (GSDM-CT) of a GSDM; wherein the GSDM-NT comprises or consists of any one of SEQ ID NOs: 51-55 or the GSDM-NT comprises or consists of amino acids 1-232 of SEQ ID NO:45 or the GSDM-NT comprises or consists of amino acids 1-224 of SEQ ID NO:46 or the GSDM-NT comprises or consists of amino acids 1-242 of SEQ ID NO:47 or the GSDM-NT comprises or consists of amino acids 1-241 of SEQ ID NO:48 or the GSDM-NT comprises or consists of amino acids 1-246 of SEQ ID NO:49.

In another embodiment, the GSDM comprises (i) the N-terminal domain (GSDM-NT) of a GSDM, and (ii) the C-terminal domain (GSDM-CT) of a GSDM; wherein the GSDM-CT comprises or consists of any one of SEQ ID NOs: 56-60.

In another embodiment, the GSDM comprises (i) the N-terminal domain (GSDM-NT) of a GSDM, and (ii) the C-terminal domain (GSDM-CT) of a GSDM; wherein the GSDM-NT comprises or consists of any one of SEQ ID NOs: 51-55, and the GSDM-CT comprises or consists of any one of SEQ ID NOs: 56-60.

In another embodiment, the GSDM comprises (i) the N-terminal domain (GSDM-NT) of a GSDM, and (ii) the C-terminal domain (GSDM-CT) of a GSDM; wherein the GSDM-NT comprises or consists of SEQ ID NOs: 51 or the GSDM-NT comprises or consists of amino acids 1-232 of SEQ ID NO:45, and the GSDM-CT comprises or consists of any one of SEQ ID NOs: 56-60.

In another embodiment, the GSDM comprises (i) the N-terminal domain (GSDM-NT) of a GSDM, and (ii) the C-terminal domain (GSDM-CT) of a; wherein the GSDM-NT comprises or consists of SEQ ID NOs: 52 or the GSDM-NT comprises or consists of amino acids 1-232 of SEQ ID NO:46, and the GSDM-CT comprises or consists of any one of SEQ ID NOs: 56-60.

In another embodiment, the GSDM comprises (i) the N-terminal domain (GSDM-NT) of a GSDM, and (ii) the C-terminal domain (GSDM-CT) of a GSDM; wherein the GSDM-NT comprises or consists of SEQ ID NOs: 53 or the GSDM-NT comprises or consists of amino acids 1-232 of SEQ ID NO:47 and the GSDM-CT comprises or consists of any one of SEQ ID NOs: 56-60.

In another embodiment, the GSDM comprises (i) the N-terminal domain (GSDM-NT) of a GSDM, and (ii) the C-terminal domain (GSDM-CT) of a GSDM; wherein the GSDM-NT comprises or consists of SEQ ID NOs: 54 or the GSDM-NT comprises or consists of amino acids 1-232 of SEQ ID NO:48, and the GSDM-CT comprises or consists of any one of SEQ ID NOs: 56-60.

In another embodiment, the GSDM comprises (i) the N-terminal domain (GSDM-NT) of a GSDM, and (ii) the C-terminal domain (GSDM-CT) of a GSDM; wherein the GSDM-NT comprises or consists of SEQ ID NOs: 55 or the GSDM-NT comprises or consists of amino acids 1-232 of SEQ ID NO:49, and the GSDM-CT comprises or consists of any one of SEQ ID NOs: 56-60.

In another embodiment, the GSDM comprises (i) the N-terminal domain (GSDM-NT) of a GSDM, and (ii) the C-terminal domain (GSDM-CT) of a GSDM; wherein the GSDM-NT comprises or consists of any one of SEQ ID NOs: 51-55, and the GSDM-CT comprises or consists of SEQ ID NOs: 56.

In another embodiment, the GSDM comprises (i) the N-terminal domain (GSDM-NT) of a GSDM, and (ii) the C-terminal domain (GSDM-CT) of a GSDM; wherein the GSDM-NT comprises or consists of any one of SEQ ID NOs: 51-55, and the GSDM-CT comprises or consists of SEQ ID NOs: 57.

In another embodiment, the GSDM comprises (i) the N-terminal domain (GSDM-NT) of a GSDM, and (ii) the C-terminal domain (GSDM-CT) of a GSDM; wherein the GSDM-NT comprises or consists of any one of SEQ ID NOs: 51-55, and the GSDM-CT comprises or consists of SEQ ID NOs: 58.

In another embodiment, the GSDM comprises (i) the N-terminal domain (GSDM-NT) of a GSDM, and (ii) the C-terminal domain (GSDM-CT) of a GSDM; wherein the GSDM-NT comprises or consists of any one of SEQ ID NOs: 51-55, and the GSDM-CT comprises or consists of SEQ ID NOs: 59.

In another embodiment, the GSDM comprises (i) the N-terminal domain (GSDM-NT) of a GSDM, and (ii) the C-terminal domain (GSDM-CT) of a GSDM; wherein the GSDM-NT comprises or consists of any one of SEQ ID NOs: 51-55, and the GSDM-CT comprises or consists of SEQ ID NOs: 60.

In one embodiment, the GSDM comprises or consists of any one of SEQ ID NOs: 45-50.

Based on the finding, that VSV induced caspase-3 induction correlates optimally with expression of GSDMs from the viral genome, the invention also includes, any of the disclosed GSDMs and further comprising a cleavable peptide sequence not naturally occurring in the respective GSDM.

A “cleavable peptide sequence” as used herein refers to an amino acid sequence that is specifically cleavable by a protease. Proteases may include caspases, such as caspase-1, 3, 4, 5, 6, 7, 8, 9, 11; granzyme A, granzyme B, neutrophil elastase, cathepsin G, 3C protease from EV71 and/or caspase-B/caspy2.

A “cleavable peptide sequence not naturally occurring” as used herein may mean any one of the following alternatives: (i) an amino acid sequence of a cleavable peptide sequence that cannot be found in the respective wild-type GSDM; OR (ii) an amino acid sequence of a cleavable peptide sequence that can be found in the respective wild-type GSDM but which is placed at another position within the respective GSDM, either (iia) additionally to the naturally occurring sequence, i.e. the naturally occurring sequence remains in the GSDM, or (iib) instead of the naturally occurring sequence, i.e. the naturally occurring sequence is deleted or changed to be inactive.

In an embodiment, the wild-type GSDM comprises or consists of any one of SEQ ID NOs: 45-50.

In one embodiment, the cleavable peptide sequence is a protease cleavable peptide sequence. In another embodiment, the protease cleavable peptide sequence is specifically cleavable by a caspase. In a preferred embodiment, the protease cleavable peptide sequence is specifically cleavable by caspase-3.

It is known, that different GSDM may harbor more than one cleavable peptide sequence. It is also known, that some cleavable peptide sequence(s) is/are positioned at locations within GSDM that do not result in an active GSDM-NT domain after cleavage. It will be understood, that in relation to any of the embodiments described herein, that cleavage of the cleavable peptide sequence not naturally occurring will result in an active GSDM-NT domain. Hence, the cleavable peptide sequence not naturally occurring is positioned or will be positioned in a region of the GSDM, which after cleavage, will result in an active GSDM-NT domain. Preferably, the cleavable peptide sequence not naturally occurring can be positioned between the GSDM-NT and GSDM-CT domain. Related hereto, the cleavable peptide sequence not naturally occurring can be positioned in the linker region. Related hereto, the cleavable peptide sequence not naturally occurring can be placed at a position of a naturally occurring cleavable peptide sequence, i.e., replacing the naturally occurring cleavable peptide sequence. Likewise, the cleavable peptide sequence not naturally occurring can be placed additionally at or nearby a position of a naturally occurring cleavable peptide sequence, i.e., the naturally occurring cleavable peptide sequence is not replaced.

Preferably, the cleavable peptide sequence is positioned in a region of the GSDM that after cleavage will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NOs: 51-55, respectively.

In a particular embodiment, the cleavable peptide sequence comprises the consensus sequence DxxD (SEQ ID NO:62), wherein X can be any amino acid. In a related embodiment, the protease cleavable peptide sequence is specifically cleavable by caspase-3 and the cleavable peptide sequence comprises the consensus sequence DxxD (SEQ ID NO:62), wherein X can be any amino acid.

In a further embodiment, the cleavable peptide sequence comprises the sequence DMPD (SEQ ID NO:63), DEVD (SEQ ID NO:64), or DLPD (SEQ ID NO:65). In a related embodiment, the protease cleavable peptide sequence is specifically cleavable by caspase-3 and the cleavable peptide sequence comprises the sequence DMPD (SEQ ID NO:63), DEVD (SEQ ID NO:64), or DLPD (SEQ ID NO:65).

In one embodiment, the GSDM is a GSDMA. In a related embodiment, the GSDM is a GSDMA comprising or consisting of an amino acid sequence as shown in SEQ ID NO:45. In another embodiment, the GSDMA comprises a cleavable peptide sequence not naturally occurring. In another embodiment, the GSDMA comprises a cleavable peptide sequence not naturally occurring, wherein the cleavable peptide sequence is specifically cleavable by caspase-3. In another embodiment, the GSDMA comprises a cleavable peptide sequence not naturally occurring, wherein the cleavable peptide sequence comprises the consensus sequence DxxD (SEQ ID NO:62), wherein X can be any amino acid. In a related embodiment, the protease cleavable peptide sequence not naturally occurring is specifically cleavable by caspase-3 and the cleavable peptide sequence comprises the consensus sequence DxxD (SEQ ID NO: 62), wherein X can be any amino acid. In another embodiment, the GSDMA comprises cleavable peptide sequence not naturally occurring, wherein the cleavable peptide sequence is specifically cleavable by caspase-3, and wherein cleavage by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:51.

In one embodiment, the GSDM is a GSDMB. In a related embodiment, the GSDM is a GSDMB comprising or consisting of an amino acid sequence as shown in SEQ ID NO:46 In another embodiment, the GSDMB comprises a cleavable peptide sequence not naturally occurring. In another embodiment, the GSDMB comprises a cleavable peptide sequence not naturally occurring, wherein the cleavable peptide sequence is specifically cleavable by caspase-3. In another embodiment, the GSDMB comprises a cleavable peptide sequence not naturally occurring, wherein the cleavable peptide sequence comprises the consensus sequence DxxD (SEQ ID NO:62), wherein X can be any amino acid. In a related embodiment, the protease cleavable peptide sequence not naturally occurring is specifically cleavable by caspase-3 and the cleavable peptide sequence comprises the consensus sequence DxxD (SEQ ID NO: 62), wherein X can be any amino acid. In another embodiment, the GSDMB comprises a cleavable peptide sequence not naturally occurring, wherein the cleavable peptide sequence is specifically cleavable by caspase-3, and wherein cleavage by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:52.

In one embodiment, the GSDM is a GSDMC. In a related embodiment, the GSDM protein is a GSDMC comprising or consisting of an amino acid sequence as shown in SEQ ID NO:47. In another embodiment, the GSDMC comprises a cleavable peptide sequence not naturally occurring. In another embodiment, the GSDMC comprises a cleavable peptide sequence not naturally occurring, wherein the cleavable peptide sequence is specifically cleavable by caspase-3. In another embodiment, the GSDMC comprises a cleavable peptide sequence, wherein the cleavable peptide sequence comprises the consensus sequence DxxD (SEQ ID NO: 62), wherein X can be any amino acid. In a related embodiment, the protease cleavable peptide sequence is specifically cleavable by caspase-3 and the cleavable peptide sequence comprises the consensus sequence DxxD (SEQ ID NO:62), wherein X can be any amino acid. In another embodiment, the GSDMC comprises a cleavable peptide sequence, wherein the cleavable peptide sequence is specifically cleavable by caspase-3, and wherein cleavage by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:53.

In one embodiment, the GSDM is a GSDMD. In a related embodiment, the GSDM is a GSDMD comprising or consisting of an amino acid sequence as shown in SEQ ID NO:48. In another embodiment, the GSDMD comprises a cleavable peptide sequence not naturally occurring. In another embodiment, the GSDMD comprises a cleavable peptide sequence not naturally occurring, wherein the cleavable peptide sequence is specifically cleavable by caspase-3. In another embodiment, the GSDMD comprises a cleavable peptide sequence, wherein the cleavable peptide sequence comprises the consensus sequence DxxD (SEQ ID NO:62), wherein X can be any amino acid. In a related embodiment, the protease cleavable peptide sequence is specifically cleavable by caspase-3 and the cleavable peptide sequence comprises the consensus sequence DxxD (SEQ ID NO:62), wherein X can be any amino acid. In another embodiment, the GSDMD comprises a cleavable peptide sequence, wherein the cleavable peptide sequence is specifically cleavable by caspase-3, and wherein cleavage by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:54.

In one embodiment, the GSDMD comprising a cleavable peptide sequence not naturally occurring comprises or consists of an amino acid sequence as shown in SEQ ID NO:107

In one embodiment, the GSDM is a GSDME. In a related embodiment, the GSDM is a GSDME comprising or consisting of an amino acid sequence as shown in SEQ ID NO:49.

In one embodiment, the GSDM is a Pejvakin protein. In a related embodiment, the GSDM is a Pejvakin protein comprising or consisting of an amino acid sequence as shown in SEQ ID NO:50. In another embodiment, the Pejvakin protein comprises a cleavable peptide sequence not naturally occurring. In another embodiment, the Pejvakin protein comprises a cleavable peptide sequence not naturally occurring, wherein the cleavable peptide sequence is specifically cleavable by caspase-3. In another embodiment, the Pejvakin protein comprises a cleavable peptide sequence, wherein the cleavable peptide sequence comprises the consensus sequence DxxD (SEQ ID NO:62), wherein X can be any amino acid. In a related embodiment, the protease cleavable peptide sequence is specifically cleavable by caspase-3 and the cleavable peptide sequence comprises the consensus sequence DxxD (SEQ ID NO:62), wherein X can be any amino acid.

The present invention includes functional variants of the disclosed GSDMs.

In one embodiment, the functional variant of the GSDMA comprises or consists of an amino acid sequence having at least 99%, 98%, 97%, 96%, 95%, 94%, 93%, 92%, 91%, 90%, 89%, 88%, 87%, 86%, 85%, 84%, 83%, 82%, 81%, or 80% identity with SEQ ID NO:45 or amino acids 1-232 of SEQ ID NO:45, wherein cleavage of said GSDMA will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:51. In another embodiment, the functional variant of the GSDMA comprises or consists of an amino acid sequence having 1, 2, 3, 4, 5, 6, 7, 8, 9, 10, 11, 12, 13, 14, 15, 16, 17, 18, 19, or 20 amino acid deletions/additions and/or mutations compared to SEQ ID NO:45 or amino acids 1-232 of SEQ ID NO:45, wherein cleavage of said cleavable peptide sequence protein will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:51.

In one embodiment, the functional variant of the GSDMA comprises or consists of an amino acid sequence having at least 99%, 98%, 97%, 96%, 95%, 94%, 93%, 92%, 91%, 90%, 89%, 88%, 87%, 86%, 85%, 84%, 83%, 82%, 81%, or 80% identity with SEQ ID NO:45 or amino acids 1-232 of SEQ ID NO:45, and further comprises a cleavable peptide sequence not naturally occurring and specifically cleavable by caspase-3, wherein cleavage of said GSDMA by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:51. In another embodiment, the functional variant of the GSDMA comprises or consists of an amino acid sequence having 1, 2, 3, 4, 5, 6, 7, 8, 9, 10, 11, 12, 13, 14, 15, 16, 17, 18, 19, or 20 amino acid deletions/additions and/or mutations compared to SEQ ID NO:45 or amino acids 1-232 of SEQ ID NO:45, and further comprises a cleavable peptide sequence not naturally occurring and specifically cleavable by caspase-3, wherein cleavage of said cleavable peptide sequence protein by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:51.

In one embodiment, the functional variant of the GSDMB comprises or consists of an amino acid sequence having at least 99%, 98%, 97%, 96%, 95%, 94%, 93%, 92%, 91%, 90%, 89%, 88%, 87%, 86%, 85%, 84%, 83%, 82%, 81%, or 80% identity with SEQ ID NO:46 or amino acids 1-224 of SEQ ID NO:46, wherein cleavage of said GSDMB by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:52. In another embodiment, the functional variant of the GSDMB comprises or consists of an amino acid sequence having 1, 2, 3, 4, 5, 6, 7, 8, 9, 10, 11, 12, 13, 14, 15, 16, 17, 18, 19, or 20 amino acid deletions/additions and/or mutations compared to SEQ ID NO:46 or amino acids 1-224 of SEQ ID NO:46, wherein cleavage of said cleavable peptide sequence protein by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:52.

In one embodiment, the functional variant of the GSDMB comprises or consists of an amino acid sequence having at least 99%, 98%, 97%, 96%, 95%, 94%, 93%, 92%, 91%, 90%, 89%, 88%, 87%, 86%, 85%, 84%, 83%, 82%, 81%, or 80% identity with SEQ ID NO:46 or amino acids 1-224 of SEQ ID NO:46, and further comprises a cleavable peptide sequence not naturally occurring and specifically cleavable by caspase-3, wherein cleavage of said GSDMB by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:52. In another embodiment, the functional variant of the GSDMB comprises or consists of an amino acid sequence having 1, 2, 3, 4, 5, 6, 7, 8, 9, 10, 11, 12, 13, 14, 15, 16, 17, 18, 19, or 20 amino acid deletions/additions and/or mutations compared to SEQ ID NO:46 or amino acids 1-224 of SEQ ID NO:46, and further comprises a cleavable peptide sequence not naturally occurring and specifically cleavable by caspase-3, wherein cleavage of said cleavable peptide sequence protein by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:52.

In one embodiment, the functional variant of the GSDMC comprises or consists of an amino acid sequence having at least 99%, 98%, 97%, 96%, 95%, 94%, 93%, 92%, 91%, 90%, 89%, 88%, 87%, 86%, 85%, 84%, 83%, 82%, 81%, or 80% identity with SEQ ID NO:47 or amino acids 1-242 of SEQ ID NO:47, wherein cleavage of said GSDMC by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:53. In another embodiment, the functional variant of the GSDMC comprises or consists of an amino acid sequence having 1, 2, 3, 4, 5, 6, 7, 8, 9, 10, 11, 12, 13, 14, 15, 16, 17, 18, 19, or 20 amino acid deletions/additions and/or mutations compared to SEQ ID NO:47 or amino acids 1-242 of SEQ ID NO:47, wherein cleavage of said cleavable peptide sequence protein by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:53.

In one embodiment, the functional variant of the GSDMC comprises or consists of an amino acid sequence having at least 99%, 98%, 97%, 96%, 95%, 94%, 93%, 92%, 91%, 90%, 89%, 88%, 87%, 86%, 85%, 84%, 83%, 82%, 81%, or 80% identity with SEQ ID NO:47 or amino acids 1-242 of SEQ ID NO:47, and further comprises a cleavable peptide sequence not naturally occurring and specifically cleavable by caspase-3, wherein cleavage of said GSDMC by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:53. In another embodiment, the functional variant of the GSDMC comprises or consists of an amino acid sequence having 1, 2, 3, 4, 5, 6, 7, 8, 9, 10, 11, 12, 13, 14, 15, 16, 17, 18, 19, or 20 amino acid deletions/additions and/or mutations compared to SEQ ID NO:47 or amino acids 1-242 of SEQ ID NO:47, and further comprises a cleavable peptide sequence not naturally occurring and specifically cleavable by caspase-3, wherein cleavage of said cleavable peptide sequence protein by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:53.

In one embodiment, the functional variant of the GSDMD comprises or consists of an amino acid sequence having at least 99%, 98%, 97%, 96%, 95%, 94%, 93%, 92%, 91%, 90%, 89%, 88%, 87%, 86%, 85%, 84%, 83%, 82%, 81%, or 80% identity with SEQ ID NO:48 or amino acids 1-241 of SEQ ID NO:48, wherein cleavage of said GSDMD by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:54. In another embodiment, the functional variant of the GSDMD comprises or consists of an amino acid sequence having 1, 2, 3, 4, 5, 6, 7, 8, 9, 10, 11, 12, 13, 14, 15, 16, 17, 18, 19, or 20 amino acid deletions/additions and/or mutations compared to SEQ ID NO:48 or amino acids 1-241 of SEQ ID NO:48, wherein cleavage of said cleavable peptide sequence protein by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:54.

In one embodiment, the functional variant of the GSDMD comprises or consists of an amino acid sequence having at least 99%, 98%, 97%, 96%, 95%, 94%, 93%, 92%, 91%, 90%, 89%, 88%, 87%, 86%, 85%, 84%, 83%, 82%, 81%, or 80% identity with SEQ ID NO:48 or amino acids 1-241 of SEQ ID NO:48, and further comprises a cleavable peptide sequence not naturally occurring and specifically cleavable by caspase-3, wherein cleavage of said GSDMD by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:54. In another embodiment, the functional variant of the GSDMD comprises or consists of an amino acid sequence having 1, 2, 3, 4, 5, 6, 7, 8, 9, 10, 11, 12, 13, 14, 15, 16, 17, 18, 19, or 20 amino acid deletions/additions and/or mutations compared to SEQ ID NO:48 or amino acids 1-241 of SEQ ID NO:48, and further comprises a cleavable peptide sequence not naturally occurring and specifically cleavable by caspase-3, wherein cleavage of said cleavable peptide sequence protein by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:54.

In one embodiment, the functional variant of the GSDME comprises or consists of an amino acid sequence having at least 99%, 98%, 97%, 96%, 95%, 94%, 93%, 92%, 91%, 90%, 89%, 88%, 87%, 86%, 85%, 84%, 83%, 82%, 81%, or 80% identity with SEQ ID NO:49 or amino acids 1-246 of SEQ ID NO:49, and wherein cleavage of said GSDME by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:55. In another embodiment, the functional variant of the GSDME comprises or consists of an amino acid sequence having 1, 2, 3, 4, 5, 6, 7, 8, 9, 10, 11, 12, 13, 14, 15, 16, 17, 18, 19, or 20 amino acid deletions/additions and/or mutations compared to SEQ ID NO:49 or amino acids 1-246 of SEQ ID NO:49, and wherein cleavage of said cleavable peptide sequence protein by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:55.

Activity of a GSDM or GSDM-NT domain can be tested indirectly by looking at the cell death phenotype which is characteristic for pyroptosis by live-cell imaging. An example of such an assay is shown in the Example section (live-cell imaging) and applied in Example 7. Hence, activity of any given GSDM or GSDM-NT domain can be tested in a Live cell imaging assay, e.g. with 4T1 mouse breast cancer cells or CT26CL.25 IFNAR−/− colorectal cancer cells. Specifically, live-cell imaging of the cell death phenotype, wherein cell swelling and loss of membrane integrity is indicative of functional pore forming.

In a preferred embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM comprising the amino acid of sequence of SEQ ID NO:49, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV. In a related preferred embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

TABLE 1

| Identifier | Sequence | SEQ ID NO: |
|---|---|---|
| Gasdermin A | MTMFENVTRALARQLNPRGDLTPLDSLIDFKRFHPFCLVLR | 45 |
| Q96QA5 | KRKSTLFWGARYVRTDYTLLDVLEPGSSPSDPTDTGNFGF | |
| | KNMLDTRVEGDVDVPKTVKVKGTAGLSQNSTLEVQTLSVA | |
| | PKALETVQERKLAADHPFLKEMQDQGENLYVVMEVVETVQ | |
| | EVTLERAGKAEACFSLPFFAPLGLQGSINHKEAVTIPKGCVL | |
| | AFRVRQLMVKGKDEWDIPHICNDNMQTFPPGEKSGEEKVI | |
| | LIQASDVGDVHEGFRTLKEEVQRETQQVEKLSRVGQSSLL | |
| | SSLSKLLGKKKELQDLELALEGALDKGHEVTLEALPKDVLL | |
| | SKEAVGAILYFVGALTELSEAQQKLLVKSMEKKILPVQLKLV | |
| | ESTMEQNFLLDKEGVFPLQPELLSSLGDEELTLTEALVGLS | |
| | GLEVQRSGPQYMWDPDTLPRLCALYAGLSLLQQLTKAS | |
| Gasdermin B | MFSVFEEITRIVVKEMDAGGDMIAVRSLVDADRFRCFHLVG | 46 |
| Q8TAX9 | EKRTFFGCRHYTTGLTLMDILDTDGDKWLDELDSGLQGQK | |
| | AEFQILDNVDSTGELIVRLPKEITISGSFQGFHHQKIKISENRI | |
| | SQQYLATLENRKLKRELPFSFRSINTRENLYLVTETLETVKE | |
| | ETLKSDRQYKFWSQISQGHLSYKHKGQREVTIPPNRVLSY | |
| | RVKQLVFPNKETMNIHFRGKTKSFPEEKDGASSCLGKSLG | |
| | SEDSRNMKEKLEDMESVLKDLTEEKRKDVLNSLAKCLGKE | |
| | DIRQDLEQRVSEVLISGELHMEDPDKPLLSSLFNAAGVLVE | |
| | ARAKAILDFLDALLELSEEQQFVAEALEKGTLPLLKDQVKSV | |
| | MEQNWDELASSPPDMDYDPEARILCALYVVVSILLELAEGP | |
| | TSVSS | |
| Gasdermin C | MPSMLERISKNLVKEIGSKDLTPVKYLLSATKLRQFVILRKK | 47 |
| Q9BYG8 | KDSRSSFWEQSDYVPVEFSLNDILEPSSSVLETVVTGPFHF | |
| | SDIMIQKHKADMGVNVGIEVSVSGEASVDHGCSLEFQIVTIP | |
| | SPNLEDFQKRKLLDPEPSFLKECRRRGDNLYVVTEAVELIN | |
| | NTVLYDSSSVNILGKIALWITYGKGQGQGESLRVKKKALTL | |
| | QKGMVMAYKRKQLVIKEKAILISDDDEQRTFQDEYEISEMV | |
| | GYCAARSEGLLPSFHTISPTLFNASSNDMKLKPELFLTQQF | |
| | LSGHLPKYEQVHILPVGRIEEPFWQNFKHLQEEVFQKIKTL | |
| | AQLSKDVQDVMFYSILAMLRDRGALQDLMNMLELDSSGHL | |
| | DGPGGAILKKLQQDSNHAWFNPKDPILYLLEAIMVLSDFQH | |
| | DLLACSMEKRILLQQQELVRSILEPNFRYPWSIPFTLKPELL | |
| | APLQSEGLAITYGLLEECGLRMELDNPRSTWDVEAKMPLS | |
| | ALYGTLSLLQQLAEA | |
| Gasdermin D | MGSAFERVVRRVVQELDHGGEFIPVTSLQSSTGFQPYCLV | 48 |
| P57764 | VRKPSSSWFWKPRYKCVNLSIKDILEPDAAEPDVQRGRSF | |
| | HFYDAMDGQIQGSVELAAPGQAKIAGGAAVSDSSSTSMNV | |
| | YSLSVDPNTWQTLLHERHLRQPEHKVLQQLRSRGDNVYV | |
| | VTEVLQTQKEVEVTRTHKREGSGRFSLPGATCLQGEGQG | |
| | HLSQKKTVTIPSGSTLAFRVAQLVIDSDLDVLLFPDKKQRTF | |
| | QPPATGHKRSTSEGAWPQLPSGLSMMRCLHNFLTDGVPA | |
| | EGAFTEDFQGLRAEVETISKELELLDRELCQLLLEGLEGVL | |
| | RDQLALRALEEALEQGQSLGPVEPLDGPAGAVLECLVLSS | |
| | GMLVPELAIPVVYLLGALTMLSETQHKLLAEALESQTLLGPL | |
| | ELVGSLLEQSAPWQERSTMSLPPGLLGNSWGEGAPAWVL | |
| | LDECGLELGEDTPHVCWEPQAQGRMCALYASLALLSGLS | |
| | QEPH | |
| Gasdermin E | MFAKATRNFLREVDADGDLIAVSNLNDSDKLQLLSLVTKKK | 49 |
| O60443 | RFWCWQRPKYQFLSLTLGDVLIEDQFPSPVVVESDFVKYE | |
| | GKFANHVSGTLETALGKVKLNLGGSSRVESQSSFGTLRKQ | |
| | EVDLQQLIRDSAERTINLRNPVLQQVLEGRNEVLCVLTQKIT | |
| | TMQKCVISEHMQVEEKCGGIVGIQTKTVQVSATEDGNVTK | |
| | DSNVVLEIPAATTIAYGVIELYVKLDGQFEFCLLRGKQGGFE | |
| | NKKRIDSVYLDPLVFREFAFIDMPDAAHGISSQDGPLSVLK | |
| | QATLLLERNFHPFAELPEPQQTALSDIFQAVLFDDELLMVLE | |
| | PVCDDLVSGLSPTVAVLGELKPRQQQDLVAFLQLVGCSLQ | |
| | GGCPGPEDAGSKQLFMTAYFLVSALAEMPDSAAALLGTCC | |
| | KLQIIPTLCHLLRALSDDGVSDLEDPTLTPLKDTERFGIVQRL | |
| | FASADISLERLKSSVKAVILKDSKVFPLLLCITLNGLCALGRE | |
| | HS | |
| Pejvakin | MFAAATKSFVKQVGDGGRLVPVPSLSEADKYQPLSLVVKK | 50 |
| Q0ZLH3 | KRCFLFPRYKFTSTPFTLKDILLGDREISAGISSYQLLNYEDE | |
| | SDVSLYGRRGNHIVNDVGINVAGSDSIAVKASFGIVTKHEV | |
| | EVSTLLKEITTRKINFDHSLIRQSRSSRKAVLCVVMESIRTTR | |
| | QCSLSVHAGIRGEAMRFHFMDEQNPKGRDKAIVFPAHTTIA | |
| | FSVFELFIYLDGAFDLCVTSVSKGGFEREETATFALLYRLRN | |
| | ILFERNRRVMDVISRSQLYLDDLFSDYYDKPLSMTDISLKEG | |
| | THIRVNLLNHNIPKGPCILCGMGNFKRETVYGCFQCSVDG | |
| | QKYVRLHAVPCFDIWHKRMK | |
| Gasdermin A | MTMFENVTRALARQLNPRGDLTPLDSLIDFKRFHPFCLVLR | 51 |
| GSDM-NT | KRKSTLFWGARYVRTDYTLLDVLEPGSSPSDPTDTGNFGF | |
| Q96QA5 | KNMLDTRVEGDVDVPKTVKVKGTAGLSQNSTLEVQTLSVA | |
| | PKALETVQERKLAADHPFLKEMQDQGENLYVVMEVVETVQ | |
| | EVTLERAGKAEACFSLPFFAPLGLQGSINHKEAVTIPKGCVL | |
| | AFRVRQLMVKGKDEWDIPHICNDNMQTFPPGEKSGEEKVI | |
| | LIQ | |
| Gasdermin B | MFSVFEEITRIVVKEMDAGGDMIAVRSLVDADRFRCFHLVG | 52 |
| GSDM-NT | EKRTFFGCRHYTTGLTLMDILDTDGDKWLDELDSGLQGQK | |
| Q8TAX9 | AEFQILDNVDSTGELIVRLPKEITISGSFQGFHHQKIKISENRI | |
| | SQQYLATLENRKLKRELPFSFRSINTRENLYLVTETLETVKE | |
| | ETLKSDRQYKFWSQISQGHLSYKHKGQREVTIPPNRVLSY | |
| | RVKQLVFPNKETMNIHFRGKTKSFPEEKDGASSCLGK | |
| Gasdermin C | MPSMLERISKNLVKEIGSKDLTPVKYLLSATKLRQFVILRKK | 53 |
| GSDM-NT | KDSRSSFWEQSDYVPVEFSLNDILEPSSSVLETVVTGPFHF | |
| Q9BYG8 | SDIMIQKHKADMGVNVGIEVSVSGEASVDHGCSLEFQIVTIP | |
| | SPNLEDFQKRKLLDPEPSFLKECRRRGDNLYVVTEAVELIN | |
| | NTVLYDSSSVNILGKIALWITYGKGQGQGESLRVKKKALTL | |
| | QKGMVMAYKRKQLVIKEKAILISDDDEQRTFQDEYEISEMV | |
| | GYCAARSEGLLPSFHTISPTLFNASSNDMKLKPELFLTQQF | |
| | LSGHLPKYEQVHILPVGRIEEPFWQNFKHLQEEVFQKIKTL | |
| | AQLSKDVQDVMFYSILAMLRDRGALQDLMNMLELD | |
| Gasdermin D | MGSAFERVVRRVVQELDHGGEFIPVTSLQSSTGFQPYCLV | 54 |
| GSDM-NT | VRKPSSSWFWKPRYKCVNLSIKDILEPDAAEPDVQRGRSF | |
| P57764 | HFYDAMDGQIQGSVELAAPGQAKIAGGAAVSDSSSTSMNV | |
| | YSLSVDPNTWQTLLHERHLRQPEHKVLQQLRSRGDNVYV | |
| | VTEVLQTQKEVEVTRTHKREGSGRFSLPGATCLQGEGQG | |
| | HLSQKKTVTIPSGSTLAFRVAQLVIDSDLDVLLFPDKKQRTF | |
| | QPPATGHKRSTSEGAWPQLPSGLSMMRCLHNFLTD | |
| Gasdermin E | MFAKATRNFLREVDADGDLIAVSNLNDSDKLQLLSLVTKKK | 55 |
| GSDM-NT | RFWCWQRPKYQFLSLTLGDVLIEDQFPSPVVVESDFVKYE | |
| O60443 | GKFANHVSGTLETALGKVKLNLGGSSRVESQSSFGTLRKQ | |
| | EVDLQQLIRDSAERTINLRNPVLQQVLEGRNEVLCVLTQKIT | |
| | TMQKCVISEHMQVEEKCGGIVGIQTKTVQVSATEDGNVTK | |
| | DSNVVLEIPAATTIAYGVIELYVKLDGQFEFCLLRGKQGGFE | |
| | NKKRIDSVYLDPLVFREFAFIDMPD | |
| Gasdermin A | ASDVGDVHEGFRTLKEEVQRETQQVEKLSRVGQSSLLSSL | 56 |
| GSDM-CT | SKLLGKKKELQDLELALEGALDKGHEVTLEALPKDVLLSKE | |
| Q96QA5 | AVGAILYFVGALTELSEAQQKLLVKSMEKKILPVQLKLVEST | |
| | MEQNFLLDKEGVFPLQPELLSSLGDEELTLTEALVGLSGLE | |
| | VQRSGPQYMWDPDTLPRLCALYAGLSLLQQLTKAS | |
| Gasdermin B | SLGSEDSRNMKEKLEDMESVLKDLTEEKRKDVLNSLAKCL | 57 |
| GSDM-CT | GKEDIRQDLEQRVSEVLISGELHMEDPDKPLLSSLFNAAGV | |
| Q8TAX9 | LVEARAKAILDFLDALLELSEEQQFVAEALEKGTLPLLKDQV | |
| | KSVMEQNWDELASSPPDMDYDPEARILCALYVVVSILLELA | |
| | EGPTSVSS | |
| Gasdermin C | SSGHLDGPGGAILKKLQQDSNHAWFNPKDPILYLLEAIMVL | 58 |
| GSDM-CT | SDFQHDLLACSMEKRILLQQQELVRSILEPNFRYPWSIPFTL | |
| Q9BYG8 | KPELLAPLQSEGLAITYGLLEECGLRMELDNPRSTWDVEAK | |
| | MPLSALYGTLSLLQQLAEA | |
| Gasdermin D | GVPAEGAFTEDFQGLRAEVETISKELELLDRELCQLLLEGL | 59 |
| GSDM-CT | EGVLRDQLALRALEEALEQGQSLGPVEPLDGPAGAVLECL | |
| P57764 | VLSSGMLVPELAIPVVYLLGALTMLSETQHKLLAEALESQTL | |
| | LGPLELVGSLLEQSAPWQERSTMSLPPGLLGNSWGEGAP | |
| | AWVLLDECGLELGEDTPHVCWEPQAQGRMCALYASLALL | |
| | SGLSQEPH | |
| Gasdermin E | AAHGISSQDGPLSVLKQATLLLERNFHPFAELPEPQQTALS | 60 |
| GSDM-CT | DIFQAVLFDDELLMVLEPVCDDLVSGLSPTVAVLGELKPRQ | |
| O60443 | QQDLVAFLQLVGCSLQGGCPGPEDAGSKQLFMTAYFLVS | |
| | ALAEMPDSAAALLGTCCKLQIIPTLCHLLRALSDDGVSDLED | |
| | PTLTPLKDTERFGIVQRLFASADISLERLKSSVKAVILKDSKV | |
| | FPLLLCITLNGLCALGREHS | |
| Caspase-3 | MENTENSVDSKSIKNLEPKIIHGSESMDSGISLDNSYKMDY | 61 |
| P42574 | PEMGLCIIINNKNFHKSTGMTSRSGTDVDAANLRETFRNLK | |
| | YEVRNKNDLTREEIVELMRDVSKEDHSKRSSFVCVLLSHG | |
| | EEGIIFGTNGPVDLKKITNFFRGDRCRSLTGKPKLFIIQACR | |
| | GTELDCGIETDSGVDDDMACHKIPVEADFLYAYSTAPGYYS | |
| | WRNSKDGSWFIQSLCAMLKQYADKLEFMHILTRVNRKVAT | |
| | EFESFSFDATFHAKKQIPCIVSMLTKELYFYH | |
| Cleavable peptide | DxxD | 62 |
| sequence | | |
| Cleavable peptide | DMPD | 63 |
| sequece | | |
| Cleavable peptide | DEVD | 64 |
| sequence | | |
| Cleavable peptide | DLPD | 65 |
| sequence | | |
| SS-IL12p40- | MCHQQLVISWFSLVFLASPLVAIWELKKDVYVVELDWYPD | 66 |
| GGGGSGGGGSGGGGS- | APGEMVVLTCDTPEEDGITWTLDQSSEVLGSGKTLTIQVKE | |
| IL12p35 | FGDAGQYTCHKGGEVLSHSLLLLHKKEDGIWSTDILKDQKE | |
| (human) | PKNKTFLRCEAKNYSGRFTCWWLTTISTDLTFSVKSSRGS | |
| | SDPQGVTCGAATLSAERVRGDNKEYEYSVECQEDSACPA | |
| | AEESLPIEVMVDAVHKLKYENYTSSFFIRDIIKPDPPKNLQLK | |
| | PLKNSRQVEVSWEYPDTWSTPHSYFSLTFCVQVQGKSKR | |
| | EKKDRVFTDKTSATVICRKNASISVRAQDRYYSSSWSEWA | |
| | SVPCSGGGGSGGGGSGGGGSRNLPVATPDPGMFPCLHH | |
| | SQNLLRAVSNMLQKARQTLEFYPCTSEEIDHEDITKDKTST | |
| | VEACLPLELTKNESCLNSRETSFITNGSCLASRKTSFMMAL | |
| | CLSSIYEDLKMYQVEFKTMNAKLLMDPKRQIFLDQNMLAVI | |
| | DELMQALNFNSETVPQKSSLEEPDFYKTKIKLCILLHAFRIR | |
| | AVTIDRVMSYLNAS | |
| SS-IL12p35- | MCHQQLVISWFSLVFLASPLVARNLPVATPDPGMFPCLHH | 67 |
| GGGGSGGGGSGGGGS- | SQNLLRAVSNMLQKARQTLEFYPCTSEEIDHEDITKDKTST | |
| IL12p40 | VEACLPLELTKNESCLNSRETSFITNGSCLASRKTSFMMAL | |
| (human) | CLSSIYEDLKMYQVEFKTMNAKLLMDPKRQIFLDQNMLAVI | |
| | DELMQALNFNSETVPQKSSLEEPDFYKTKIKLCILLHAFRIR | |
| | AVTIDRVMSYLNASGGGGSGGGGSGGGGSIWELKKDVYV | |
| | VELDWYPDAPGEMVVLTCDTPEEDGITWTLDQSSEVLGSG | |
| | KTLTIQVKEFGDAGQYTCHKGGEVLSHSLLLLHKKEDGIWS | |
| | TDILKDQKEPKNKTFLRCEAKNYSGRFTCWWLTTISTDLTF | |
| | SVKSSRGSSDPQGVTCGAATLSAERVRGDNKEYEYSVEC | |
| | QEDSACPAAEESLPIEVMVDAVHKLKYENYTSSFFIRDIIKP | |
| | DPPKNLQLKPLKNSRQVEVSWEYPDTWSTPHSYFSLTFCV | |
| | QVQGKSKREKKDRVFTDKTSATVICRKNASISVRAQDRYY | |
| | SSSWSEWASVPCS | |
| Signal sequence | MCHQQLVISWFSLVFLASPLVA | 68 |
| Consensus 2A | DxExNPGP | 69 |
| T2A | EGRGSLLTCGDVEENPGP | 70 |
| GS-T2A | GSGEGRGSLLTCGDVEENPGP | 71 |
| GSDME-IL12 | MFAKATRNFLREVDADGDLIAVSNLNDSDKLQLLSLVTKKK | 72 |
| (human) | RFWCWQRPKYQFLSLTLGDVLIEDQFPSPVVVESDFVKYE | |
| | GKFANHVSGTLETALGKVKLNLGGSSRVESQSSFGTLRKQ | |
| | EVDLQQLIRDSAERTINLRNPVLQQVLEGRNEVLCVLTQKIT | |
| | TMQKCVISEHMQVEEKCGGIVGIQTKTVQVSATEDGNVTK | |
| | DSNVVLEIPAATTIAYGVIELYVKLDGQFEFCLLRGKQGGFE | |
| | NKKRIDSVYLDPLVFREFAFIDMPDAAHGISSQDGPLSVLK | |
| | QATLLLERNFHPFAELPEPQQTALSDIFQAVLFDDELLMVLE | |
| | PVCDDLVSGLSPTVAVLGELKPRQQQDLVAFLQLVGCSLQ | |
| | GGCPGPEDAGSKQLFMTAYFLVSALAEMPDSAAALLGTCC | |
| | KLQIIPTLCHLLRALSDDGVSDLEDPTLTPLKDTERFGIVQRL | |
| | FASADISLERLKSSVKAVILKDSKVFPLLLCITLNGLCALGRE | |
| | HSGSGEGRGSLLTCGDVEENPGPMCHQQLVISWFSLVFL | |
| | ASPLVAIWELKKDVYVVELDWYPDAPGEMVVLTCDTPEED | |
| | GITWTLDQSSEVLGSGKTLTIQVKEFGDAGQYTCHKGGEV | |
| | LSHSLLLLHKKEDGIWSTDILKDQKEPKNKTFLRCEAKNYS | |
| | GRFTCWWLTTISTDLTFSVKSSRGSSDPQGVTCGAATLSA | |
| | ERVRGDNKEYEYSVECQEDSACPAAEESLPIEVMVDAVHK | |
| | LKYENYTSSFFIRDIIKPDPPKNLQLKPLKNSRQVEVSWEYP | |
| | DTWSTPHSYFSLTFCVQVQGKSKREKKDRVFTDKTSATVI | |
| | CRKNASISVRAQDRYYSSSWSEWASVPCSGGGGSGGGG | |
| | SGGGGSRNLPVATPDPGMFPCLHHSQNLLRAVSNMLQKA | |
| | RQTLEFYPCTSEEIDHEDITKDKTSTVEACLPLELTKNESCL | |
| | NSRETSFITNGSCLASRKTSFMMALCLSSIYEDLKMYQVEF | |
| | KTMNAKLLMDPKRQIFLDQNMLAVIDELMQALNFNSETVPQ | |
| | KSSLEEPDFYKTKIKLCILLHAFRIRAVTIDRVMSYLNAS | |
| P2A | ATNFSLLKQAGDVEENPGP | 73 |
| E2A | QCTNYALLKLAGDVESNPGP | 74 |
| F2A | VKQTLNFDLLKLAGDVESNPGP | 75 |
| GSDME | MFAKATRNFLKEVDAGGDLISVSHLNDSDKLQLLSLVTKKK | 76 |
| (mouse) | RYWCWQRPKYQILSATLEDVLTEGHCLSPVVVESDFVKYE | |
| E9Q5V3 | SKCENHKSGAIGTVVGKVKLNVGGKGVVESHSSFGTLRKQ | |
| | EVDVQQLIQDAVKRTVNMDNLVLQQVLESRNEVLCVLTQKI | |
| | MTTQKCVISEHVQSEETCGGMVGIQTKTIQVSATEDGTVTT | |
| | DTNVVLEIPAATTIAYGIMELFVKQDGQFEFCLLQGKHGGF | |
| | EHERKLDSVYLDPLAYREFAFLDMLDGGQGISSQDGPLRV | |
| | VKQATLHLERSFHPFAVLPAQQQRALFCVLQKILFDEELLR | |
| | ALEQVCDDVAGGLWSSQAVLAMEELTDSQQQDLTAFLQL | |
| | VGYRIQGEHPGPQDEVSNQKLFATAYFLVSALAEMPDNAT | |
| | VFLGTCCKLHVISSLCCLGG | |
| GSDMD | MPSAFEKVVKNVIKEVSGSRGDLIPVDSLRNSTSFRPYCLL | 77 |
| (mouse) | NRKFSSSRFWKPRYSCVNLSIKDILEPSAPEPEPECFGSFK | |
| | VSDVVDGNIQGRVMLSGMGEGKISGGAAVSDSSSASMNV | |
| | CILRVTQKTWETMQHERHLQQPENKILQQLRSRGDDLFVV | |
| | TEVLQTKEEVQITEVHSQEGSGQFTLPGALCLKGEGKGHQ | |
| | SRKKMVTIPAGSILAFRVAQLLIGSKWDILLVSDEKQRTFEP | |
| | SSGDRKAVGQRHHGLNVLAALCSIGKQLSLLSDGIDEEELI | |
| | EAADFQGLYAEVKACSSELESLEMELRQQILVNIGKILQDQ | |
| | PSMEALEASLGQGLCSGGQVEPLDGPAGCILECLVLDSGE | |
| | LVPELAAPIFYLLGALAVLSETQQQLLAKALETTVLSKQLEL | |
| | VKHVLEQSTPWQEQSSVSLPTVLLGDCWDEKNPTWVLLE | |
| | ECGLRLQVESPQVHWEPTSLIPTSALYASLFLLSSLGQKPC | |
| GSDMD-DEVD | MPSAFEKVVKNVIKEVSGSRGDLIPVDSLRNSTSFRPYCLL | 78 |
| (mouse) | NRKFSSSRFWKPRYSCVNLSIKDILEPSAPEPEPECFGSFK | |
| | VSDVVDGNIQGRVMLSGMGEGKISGGAAVSDSSSASMNV | |
| | CILRVTQKTWETMQHERHLQQPENKILQQLRSRGDDLFVV | |
| | TEVLQTKEEVQITEVHSQEGSGQFTLPGALCLKGEGKGHQ | |
| | SRKKMVTIPAGSILAFRVAQLLIGSKWDILLVSDEKQRTFEP | |
| | SSGDRKAVGQRHHGLNVLAALCSIGKQLSDEVDGIDEEELI | |
| | EAADFQGLYAEVKACSSELESLEMELRQQILVNIGKILQDQ | |
| | PSMEALEASLGQGLCSGGQVEPLDGPAGCILECLVLDSGE | |
| | LVPELAAPIFYLLGALAVLSETQQQLLAKALETTVLSKQLEL | |
| | VKHVLEQSTPWQEQSSVSLPTVLLGDCWDEKNPTWVLLE | |
| | ECGLRLQVESPQVHWEPTSLIPTSALYASLFLLSSLGQKPC | |
| Immunoglobulin H | MGWSCIILFLVATATGVHS | 81 |
| chain V-region signal | | |
| sequence | | |
| GSDME-IL12 | MFAKATRNFLKEVDAGGDLISVSHLNDSDKLQLLSLVTKKK | 84 |
| (mouse) | RYWCWQRPKYQILSATLEDVLTEGHCLSPVVVESDFVKYE | |
| | SKCENHKSGAIGTVVGKVKLNVGGKGVVESHSSFGTLRKQ | |
| | EVDVQQLIQDAVKRTVNMDNLVLQQVLESRNEVLCVLTQKI | |
| | MTTQKCVISEHVQSEETCGGMVGIQTKTIQVSATEDGTVTT | |
| | DTNVVLEIPAATTIAYGIMELFVKQDGQFEFCLLQGKHGGF | |
| | EHERKLDSVYLDPLAYREFAFLDMLDGGQGISSQDGPLRV | |
| | VKQATLHLERSFHPFAVLPAQQQRALFCVLQKILFDEELLR | |
| | ALEQVCDDVAGGLWSSQAVLAMEELTDSQQQDLTAFLQL | |
| | VGYRIQGEHPGPQDEVSNQKLFATAYFLVSALAEMPDNAT | |
| | VFLGTCCKLHVISSLCCLGGGSGEGRGSLLTCGDVEENPG | |
| | PMGWSCIILFLVATATGVHSMWELEKDVYVVEVDWTPDAP | |
| | GETVNLTCDTPEEDDITWTSDQRHGVIGSGKTLTITVKEFL | |
| | DAGQYTCHKGGETLSHSHLLLHKKENGIWSTEILKNFKNKT | |
| | FLKCEAPNYSGRFTCSWLVQRNMDLKFNIKSSSSSPDSRA | |
| | VTCGMASLSAEKVTLDQRDYEKYSVSCQEDVTCPTAEETL | |
| | PIELALEARQQNKYENYSTSFFIRDIIKPDPPKNLQMKPLKN | |
| | SQVEVSWEYPDSWSTPHSYFSLKFFVRIQRKKEKMKETEE | |
| | GCNQKGAFLVEKTSTEVQCKGGNVCVQAQDRYYNSSCSK | |
| | WACVPCRVRSGGGGSGGGGSGGGGSRVIPVSGPARCLS | |
| | QSRNLLKTTDDMVKTAREKLKHYSCTAEDIDHEDITRDQTS | |
| | TLKTCLPLELHKNESCLATRETSSTTRGSCLPPQKTSLMMT | |
| | LCLGSIYEDLKMYQTEFQAINAALQNHNHQQIILDKGMLVAI | |
| | DELMQSLNHNGETLRQKPPVGEADPYRVKMKLCILLHAFS | |
| | TRVVTINRVMGYLSSA | |
| hsGSDMD-DEVD- | MGSAFERVVRRVVQELDHGGEFIPVTSLQSSTGFQPYCLV | 86 |
| mmIL18DR | VRKPSSSWFWKPRYKCVNLSIKDILEPDAAEPDVQRGRSF | |
| | HFYDAMDGQIQGSVELAAPGQAKIAGGAAVSDSSSTSMNV | |
| | YSLSVDPNTWQTLLHERHLRQPEHKVLQQLRSRGDNVYV | |
| | VTEVLQTQKEVEVTRTHKREGSGRFSLPGATCLQGEGQG | |
| | HLSQKKTVTIPSGSTLAFRVAQLVIDSDLDVLLFPDKKQRTF | |
| | QPPATGHKRSTSEGAWPQLPSGLSMMRCLHNDEVDGVP | |
| | AEGAFTEDFQGLRAEVETISKELELLDRELCQLLLEGLEGVL | |
| | RDQLALRALEEALEQGQSLGPVEPLDGPAGAVLECLVLSS | |
| | GMLVPELAIPVVYLLGALTMLSETQHKLLAEALESQTLLGPL | |
| | ELVGSLLEQSAPWQERSTMSLPPGLLGNSWGEGAPAWVL | |
| | LDECGLELGEDTPHVCWEPQAQGRMCALYASLALLSGLS | |
| | QEPHGSGEGRGSLLTCGDVEENPGPHFGRLHCTTAVIRNI | |
| | NDQVLFVDKRQPVFEDMTDIDQSASEPQTRLIIYAYGDSRA | |
| | RGKAVTLSVKDSKMSTLSCKNKIISFEEMDPPENIDDIQSDL | |
| | IFFQKRVPGHNKMEFESSLYEGHFLACQKEDDAFKLILKKK | |
| | DENGDKSVMFTLTNLHQS | |
| hsGSDMD-DEVD- | MGSAFERVVRRVVQELDHGGEFIPVTSLQSSTGFQPYCLV | 87 |
| mmIL18DR-mmIL12 | VRKPSSSWFWKPRYKCVNLSIKDILEPDAAEPDVQRGRSF | |
| | HFYDAMDGQIQGSVELAAPGQAKIAGGAAVSDSSSTSMNV | |
| | YSLSVDPNTWQTLLHERHLRQPEHKVLQQLRSRGDNVYV | |
| | VTEVLQTQKEVEVTRTHKREGSGRFSLPGATCLQGEGQG | |
| | HLSQKKTVTIPSGSTLAFRVAQLVIDSDLDVLLFPDKKQRTF | |
| | QPPATGHKRSTSEGAWPQLPSGLSMMRCLHNDEVDGVP | |
| | AEGAFTEDFQGLRAEVETISKELELLDRELCQLLLEGLEGVL | |
| | RDQLALRALEEALEQGQSLGPVEPLDGPAGAVLECLVLSS | |
| | GMLVPELAIPVVYLLGALTMLSETQHKLLAEALESQTLLGPL | |
| | ELVGSLLEQSAPWQERSTMSLPPGLLGNSWGEGAPAWVL | |
| | LDECGLELGEDTPHVCWEPQAQGRMCALYASLALLSGLS | |
| | QEPHGSGEGRGSLLTCGDVEENPGPHFGRLHCTTAVIRNI | |
| | NDQVLFVDKRQPVFEDMTDIDQSASEPQTRLIIYAYGDSRA | |
| | RGKAVTLSVKDSKMSTLSCKNKIISFEEMDPPENIDDIQSDL | |
| | IFFQKRVPGHNKMEFESSLYEGHFLACQKEDDAFKLILKKK | |
| | DENGDKSVMFTLTNLHQSGSGATNFSLLKQAGDVEENPG | |
| | PMGWSCIILFLVATATGVHSMWELEKDVYVVEVDWTPDAP | |
| | GETVNLTCDTPEEDDITWTSDQRHGVIGSGKTLTITVKEFL | |
| | DAGQYTCHKGGETLSHSHLLLHKKENGIWSTEILKNFKNKT | |
| | FLKCEAPNYSGRFTCSWLVQRNMDLKFNIKSSSSSPDSRA | |
| | VTCGMASLSAEKVTLDQRDYEKYSVSCQEDVTCPTAEETL | |
| | PIELALEARQQNKYENYSTSFFIRDIIKPDPPKNLQMKPLKN | |
| | SQVEVSWEYPDSWSTPHSYFSLKFFVRIQRKKEKMKETEE | |
| | GCNQKGAFLVEKTSTEVQCKGGNVCVQAQDRYYNSSCSK | |
| | WACVPCRVRSGGGGSGGGGSGGGGSRVIPVSGPARCLS | |
| | QSRNLLKTTDDMVKTAREKLKHYSCTAEDIDHEDITRDQTS | |
| | TLKTCLPLELHKNESCLATRETSSTTRGSCLPPQKTSLMMT | |
| | LCLGSIYEDLKMYQTEFQAINAALQNHNHQQIILDKGMLVAI | |
| | DELMQSLNHNGETLRQKPPVGEADPYRVKMKLCILLHAFS | |
| | TRVVTINRVMGYLSSA | |
| hsGSDMD-DEVD- | MGSAFERVVRRVVQELDHGGEFIPVTSLQSSTGFQPYCLV | 88 |
| mmIL18DR-mmIL1 | VRKPSSSWFWKPRYKCVNLSIKDILEPDAAEPDVQRGRSF | |
| | HFYDAMDGQIQGSVELAAPGQAKIAGGAAVSDSSSTSMNV | |
| | YSLSVDPNTWQTLLHERHLRQPEHKVLQQLRSRGDNVYV | |
| | VTEVLQTQKEVEVTRTHKREGSGRFSLPGATCLQGEGQG | |
| | HLSQKKTVTIPSGSTLAFRVAQLVIDSDLDVLLFPDKKQRTF | |
| | QPPATGHKRSTSEGAWPQLPSGLSMMRCLHNDEVDGVP | |
| | AEGAFTEDFQGLRAEVETISKELELLDRELCQLLLEGLEGVL | |
| | RDQLALRALEEALEQGQSLGPVEPLDGPAGAVLECLVLSS | |
| | GMLVPELAIPVVYLLGALTMLSETQHKLLAEALESQTLLGPL | |
| | ELVGSLLEQSAPWQERSTMSLPPGLLGNSWGEGAPAWVL | |
| | LDECGLELGEDTPHVCWEPQAQGRMCALYASLALLSGLS | |
| | QEPHGSGEGRGSLLTCGDVEENPGPHFGRLHCTTAVIRNI | |
| | NDQVLFVDKRQPVFEDMTDIDQSASEPQTRLIIYAYGDSRA | |
| | RGKAVTLSVKDSKMSTLSCKNKIISFEEMDPPENIDDIQSDL | |
| | IFFQKRVPGHNKMEFESSLYEGHFLACQKEDDAFKLILKKK | |
| | DENGDKSVMFTLTNLHQSGSGATNFSLLKQAGDVEENPG | |
| | PVPIRQLHYRLRDEQQKSLVLSDPYELKALHLNGQNINQQV | |
| | IFSMSFVQGEPSNDKIPVALGLKGKNLYLSCVMKDGTPTLQ | |
| | LESVDPKQYPKKKMEKRFVFNKIEVKSKVEFESAEFPNWYI | |
| | STSQAEHKPVFLGNNSGQDIIDFTMESVSS | |
| hsGSDMD-DEVD- | MGSAFERVVRRVVQELDHGGEFIPVTSLQSSTGFQPYCLV | 89 |
| mmIL1-mmIL18- | VRKPSSSWFWKPRYKCVNLSIKDILEPDAAEPDVQRGRSF | |
| mmIFN-alpha-2 | HFYDAMDGQIQGSVELAAPGQAKIAGGAAVSDSSSTSMNV | |
| | YSLSVDPNTWQTLLHERHLRQPEHKVLQQLRSRGDNVYV | |
| | VTEVLQTQKEVEVTRTHKREGSGRFSLPGATCLQGEGQG | |
| | HLSQKKTVTIPSGSTLAFRVAQLVIDSDLDVLLFPDKKQRTF | |
| | QPPATGHKRSTSEGAWPQLPSGLSMMRCLHNDEVDGVP | |
| | AEGAFTEDFQGLRAEVETISKELELLDRELCQLLLEGLEGVL | |
| | RDQLALRALEEALEQGQSLGPVEPLDGPAGAVLECLVLSS | |
| | GMLVPELAIPVVYLLGALTMLSETQHKLLAEALESQTLLGPL | |
| | ELVGSLLEQSAPWQERSTMSLPPGLLGNSWGEGAPAWVL | |
| | LDECGLELGEDTPHVCWEPQAQGRMCALYASLALLSGLS | |
| | QEPHGSGATNFSLLKQAGDVEENPGPVPIRQLHYRLRDEQ | |
| | QKSLVLSDPYELKALHLNGQNINQQVIFSMSFVQGEPSNDK | |
| | IPVALGLKGKNLYLSCVMKDGTPTLQLESVDPKQYPKKKM | |
| | EKRFVFNKIEVKSKVEFESAEFPNWYISTSQAEHKPVFLGN | |
| | NSGQDIIDFTMESVSSGSGEGRGSLLTCGDVEENPGPNFG | |
| | RLHCTTAVIRNINDQVLFVDKRQPVFEDMTDIDQSASEPQT | |
| | RLIIYMYKDSEVRGLAVTLSVKDSKMSTLSCKNKIISFEEMD | |
| | PPENIDDIQSDLIFFQKRVPGHNKMEFESSLYEGHFLACQK | |
| | EDDAFKLILKKKDENGDKSVMFTLTNLHQSGSGQCTNYALL | |
| | KLAGDVESNPGPARLCAFLVMLIVMSYWSICSLGCDLPHTY | |
| | NLRNKRALKVLAQMRRLPFLSCLKDRQDFGFPLEKVDNQQ | |
| | IQKAQAIPVLRDLTQQTLNLFTSKASSAAWNATLLDSFQND | |
| | LHQQLNDLQTCLMQQVGVQEPPLTQEDALLAVRKYFHRIT | |
| | VYLREKKHSPCAWEVVRAEVWRALSSSVNLLPRLSEEKE | |
| hsGSDMD-DEVD- | MGSAFERVVRRVVQELDHGGEFIPVTSLQSSTGFQPYCLV | 90 |
| mmIL18-mmIL1- | VRKPSSSWFWKPRYKCVNLSIKDILEPDAAEPDVQRGRSF | |
| mmIFN-alpha-2 | HFYDAMDGQIQGSVELAAPGQAKIAGGAAVSDSSSTSMNV | |
| | YSLSVDPNTWQTLLHERHLRQPEHKVLQQLRSRGDNVYV | |
| | VTEVLQTQKEVEVTRTHKREGSGRFSLPGATCLQGEGQG | |
| | HLSQKKTVTIPSGSTLAFRVAQLVIDSDLDVLLFPDKKQRTF | |
| | QPPATGHKRSTSEGAWPQLPSGLSMMRCLHNDEVDGVP | |
| | AEGAFTEDFQGLRAEVETISKELELLDRELCQLLLEGLEGVL | |
| | RDQLALRALEEALEQGQSLGPVEPLDGPAGAVLECLVLSS | |
| | GMLVPELAIPVVYLLGALTMLSETQHKLLAEALESQTLLGPL | |
| | ELVGSLLEQSAPWQERSTMSLPPGLLGNSWGEGAPAWVL | |
| | LDECGLELGEDTPHVCWEPQAQGRMCALYASLALLSGLS | |
| | QEPHGSGEGRGSLLTCGDVEENPGPNFGRLHCTTAVIRNI | |
| | NDQVLFVDKRQPVFEDMTDIDQSASEPQTRLIIYMYKDSEV | |
| | RGLAVTLSVKDSKMSTLSCKNKIISFEEMDPPENIDDIQSDLI | |
| | FFQKRVPGHNKMEFESSLYEGHFLACQKEDDAFKLILKKK | |
| | DENGDKSVMFTLTNLHQSGSGATNFSLLKQAGDVEENPG | |
| | PVPIRQLHYRLRDEQQKSLVLSDPYELKALHLNGQNINQQV | |
| | IFSMSFVQGEPSNDKIPVALGLKGKNLYLSCVMKDGTPTLQ | |
| | LESVDPKQYPKKKMEKRFVFNKIEVKSKVEFESAEFPNWYI | |
| | STSQAEHKPVFLGNNSGQDIIDFTMESVSSGSGQCTNYAL | |
| | LKLAGDVESNPGPARLCAFLVMLIVMSYWSICSLGCDLPHT | |
| | YNLRNKRALKVLAQMRRLPFLSCLKDRQDFGFPLEKVDNQ | |
| | QIQKAQAIPVLRDLTQQTLNLFTSKASSAAWNATLLDSFCN | |
| | DLHQQLNDLQTCLMQQVGVQEPPLTQEDALLAVRKYFHRI | |
| | TVYLREKKHSPCAWEVVRAEVWRALSSSVNLLPRLSEEKE | |
| GSDMD-DEVD | MGSAFERVVRRVVQELDHGGEFIPVTSLQSSTGFQPYCLV | 107 |
| (human) | VRKPSSSWFWKPRYKCVNLSIKDILEPDAAEPDVQRGRSF | |
| | HFYDAMDGQIQGSVELAAPGQAKIAGGAAVSDSSSTSMNV | |
| | YSLSVDPNTWQTLLHERHLRQPEHKVLQQLRSRGDNVYV | |
| | VTEVLQTQKEVEVTRTHKREGSGRFSLPGATCLQGEGQG | |
| | HLSQKKTVTIPSGSTLAFRVAQLVIDSDLDVLLFPDKKQRTF | |
| | QPPATGHKRSTSEGAWPQLPSGLSMMRCLHNDEVDGVP | |
| | AEGAFTEDFQGLRAEVETISKELELLDRELCQLLLEGLEGVL | |
| | RDQLALRALEEALEQGQSLGPVEPLDGPAGAVLECLVLSS | |
| | GMLVPELAIPVVYLLGALTMLSETQHKLLAEALESQTLLGPL | |
| | ELVGSLLEQSAPWQERSTMSLPPGLLGNSWGEGAPAWVL | |
| | LDECGLELGEDTPHVCWEPQAQGRMCALYASLALLSGLS | |
| | QEPH | |
| GSDME | MFAKATRNFLKEVDAGGDLISVSHLNDSDKLQLLSLVTKKK | 109 |
| (mouse) | RYWCWQRPKYQILSATLEDVLTEGHCLSPVVVESDFVKYE | |
| Q9Z2D3 | SKCENHKSGAIGTVVGKVKLNVGGKGVVESHSSFGTLRKQ | |
| | EVDVQQLIQDAVKRTVNMDNLVLQQVLESRNEVLCVLTQKI | |
| | MTTQKCVISEHVQSEETCGGMVGIQTKTIQVSATEDGTVTT | |
| | DTNVVLEIPAATTIAYGIMELFVKQDGQFEFCLLQGKHGGF | |
| | EHERKLDSVYLDPLAYREFAFLDMLDGGQGISSQDGPLRV | |
| | VKQATLHLERSFHPFAVLPAQQQRALFCVLQKILFDEELLR | |
| | ALEQVCDDVAGGLWSSQAVLAMEELTDSQQQDLTAFLQL | |
| | VGYRIQGEHPGPQDEVSNQKLFATAYFLVSALAEMPDNAT | |
| | VFLGTCCKLHVISSLCCLLHALSDDSVCDFHNPTLAPLRDT | |
| | ERFGIVQRLFASADIALERMQFSAKATILKDSCIFPLILHITLS | |
| | GLSTLSKEHEEELCQSGHATGQD | |
| GSDME(Q9Z2D3)-IL12 | MFAKATRNFLKEVDAGGDLISVSHLNDSDKLQLLSLVTKKK | 110 |
| (mouse) | RYWCWQRPKYQILSATLEDVLTEGHCLSPVVVESDFVKYE | |
| | SKCENHKSGAIGTVVGKVKLNVGGKGVVESHSSFGTLRKQ | |
| | EVDVQQLIQDAVKRTVNMDNLVLQQVLESRNEVLCVLTQKI | |
| | MTTQKCVISEHVQSEETCGGMVGIQTKTIQVSATEDGTVTT | |
| | DTNVVLEIPAATTIAYGIMELFVKQDGQFEFCLLQGKHGGF | |
| | EHERKLDSVYLDPLAYREFAFLDMLDGGQGISSQDGPLRV | |
| | VKQATLHLERSFHPFAVLPAQQQRALFCVLQKILFDEELLR | |
| | ALEQVCDDVAGGLWSSQAVLAMEELTDSQQQDLTAFLQL | |
| | VGYRIQGEHPGPQDEVSNQKLFATAYFLVSALAEMPDNAT | |
| | VFLGTCCKLHVISSLCCLLHALSDDSVCDFHNPTLAPLRDT | |
| | ERFGIVQRLFASADIALERMQFSAKATILKDSCIFPLILHITLS | |
| | GLSTLSKEHEEELCQSGHATGQDGSGEGRGSLLTCGDVE | |
| | ENPGPMGWSCIILFLVATATGVHSMWELEKDVYVVEVDWT | |
| | PDAPGETVNLTCDTPEEDDITWTSDQRHGVIGSGKTLTITV | |
| | KEFLDAGQYTCHKGGETLSHSHLLLHKKENGIWSTEILKNF | |
| | KNKTFLKCEAPNYSGRFTCSWLVQRNMDLKFNIKSSSSSP | |
| | DSRAVTCGMASLSAEKVTLDQRDYEKYSVSCQEDVTCPTA | |
| | EETLPIELALEARQQNKYENYSTSFFIRDIIKPDPPKNLQMK | |
| | PLKNSQVEVSWEYPDSWSTPHSYFSLKFFVRIQRKKEKMK | |
| | ETEEGCNQKGAFLVEKTSTEVQCKGGNVCVQAQDRYYNS | |
| | SCSKWACVPCRVRSGGGGSGGGGSGGGGSRVIPVSGPA | |
| | RCLSQSRNLLKTTDDMVKTAREKLKHYSCTAEDIDHEDITR | |
| | DQTSTLKTCLPLELHKNESCLATRETSSTTRGSCLPPQKTS | |
| | LMMTLCLGSIYEDLKMYQTEFQAINAALQNHNHQQIILDKG | |
| | MLVAIDELMQSLNHNGETLRQKPPVGEADPYRVKMKLCILL | |
| | HAFSTRVVTINRVMGYLSSA | |

### Gasdermin and Interleukin12 (IL12) Constructs

In a further embodiment, the recombinant rhabdovirus further encodes for an IL12p35 and an IL12p40 subunit of IL12. Preferably, the IL12p35 subunit and the IL12p40 subunit are human.

Interleukin12 (IL12) is a heterodimeric molecule composed of an alpha chain (the IL12p35 subunit) and a beta chain (the IL12p40 subunit) covalently linked by a disulfide bridge to form the biologically active 70 kDa dimer. It is produced by antigen-presenting cells, such as dendritic cells and macrophages, and is crucial for the recruitment and effector functions of CD8+ T and NK cells. Therefore, IL12 is a major contributor to effective anti-tumor immune responses. IL12 signals through IL12Rβ1 and IL12Rβ2 receptors expressed on target cells, which allow downstream Jak2 and Tyk2 to promote the phosphorylation of and homo-dimerization of STAT4. Further studies demonstrated that IL12 is not only required for the activation of effector anti-tumor immune responses but can also directly inhibit immune suppression. Thus, the use of IL12 as a cancer immunotherapy could be beneficial in controlling tumor growth by activating anti-tumor cytotoxic immune responses. Overall, IL12 targets and modulates T cells, NK cells and antigen-presenting cells (APCs) that regulate the fate of the anti-tumor immune response against the cancer cells. However, systemic use of IL12 is severely limited by its toxicity.

In certain embodiments, the IL12 cytokine comprises an IL12p35 amino acid sequence as set forth in SEQ ID NO:1. In certain embodiments, the IL12 cytokine comprises an IL12p40 amino acid sequence as set forth in SEQ ID NO:2. In certain embodiments, the IL12 cytokine comprises an IL12p35 amino acid sequence as set forth in SEQ ID NO:1 and comprises an IL12p40 amino acid sequence as set forth in SEQ ID NO:2.

In another embodiment, as opposed to keeping IL12 as a native heterodimer, the IL12 cytokine is composed of a single-chain IL12 having the configuration (written from N-terminus to C-terminus) IL12p40-IL12p35 or IL12p35-IL12p40. In certain embodiments, the IL12p40-IL12p35 comprises an amino acid sequence as set forth in SEQ ID NO:3. In certain embodiments, the IL12p35-IL12p40 comprises an amino acid sequence as set forth in SEQ ID NO:4.

In a related embodiment, the subunits within the single-chain IL12 cytokine may be linked to each other via a linker, e.g. IL12p40 (linker) IL12p35 or IL12p35 (linker) IL12p40. The linker may be a peptide linker and especially any peptide linker as disclosed herein and preferably a GS linker. Hence, in a related embodiment the subunits in the single-chain IL12 cytokine comprising the amino acid sequence as set forth in any one of SEQ ID NOs: 3 or 4 are linked to each other via a linker as disclosed herein and preferably a GS linker. In a related embodiment, the GS linker has the following amino acid sequence GGGGSGGGGSGGGGS (SEQ ID NO:22). In a preferred embodiment, the single-chain IL12 cytokine is provided in the configuration IL12p40-15GS-IL12p35 (SEQ ID NO:5). In another embodiment, the single-chain IL12 cytokine is provided in the configuration IL12p35-15GS-IL12p40 (SEQ ID NO:6).

In another embodiment, the IL12p35 subunit of the IL12 comprises a polypeptide having at least 90%, 91%, 92%, 93%, 94%, 95%, 96%, 97%, 98%, or 99% identity to SEQ ID NO:1 and the IL12p40 subunit of the IL12 comprises a polypeptide having at least 90%, 91%, 92%, 93%, 94%, 95%, 96%, 97%, 98%, or 99% identity to SEQ ID NO:2, preferably the IL12p35 subunit comprises or consists of the polypeptide of SEQ ID NO:1 and the IL12p40 subunit comprises or consists of the polypeptide of SEQ ID NO:2.

In another embodiment, the IL12p40 subunit and the IL12p35 subunit are linked to each other via a linker that is rich in amino acid residues glycine and serine. In a related embodiment, the linker has a length of 5 to 20 amino acids and only includes the amino acids glycine and serine. In a preferred embodiment, the linker has the amino acid sequence of SEQ ID NO:22.

In a preferred embodiment, the IL12 comprises a single-chain IL12p40-IL12p35 polypeptide having at least 90%, 91%, 92%, 93%, 94%, 95%, 96%, 97%, 98%, or 99% identity to SEQ ID NO:5. In another embodiment, the IL12 comprises a single-chain IL12p35-IL12p40 polypeptide having at least 90%, 91%, 92%, 93%, 94%, 95%, 96%, 97%, 98%, or 99% identity to SEQ ID NO:6.

The IL12 cytokine may comprise a variant of the IL12p35 and/or IL12p40 sequence. The variant encodes for a protein that retains IL12 functional activity as compared to the wild type IL12. The variant may encode for an IL12 subunit or any single chain IL12 as disclosed herein. In one embodiment, the variant encodes for an IL12 subunit or any single-chain IL12 as show in any of SEQ ID NOs: 1-6, additionally having at least 1, 2, 3, 4, 5, 6, 7, 8, 9, 10, 11, 12, 13, 14, 15, 16, 17, 18, 19 or 20 amino acid mutation, deletion, substitution and/or addition compared to the amino acid sequence shown in any of SEQ ID NOs: 1-6.

Functional activity of IL12 can be measured on immune cells (human T-cells, NK-cells or splenocytes (hamster)) looking for either proliferation or IFN gamma secretion. IFN gamma secretion was tested e.g. in example 19 using an flow cytometry-based bead Assay.

In one embodiment, the IL12 further comprises a signal peptide sequence. In another embodiment, the IL12 does not comprise a signal peptide sequence.

The term “signal peptide” or “signal peptide sequence” describes a peptide sequence usually 10 to 30 amino acids in length and present at the N-terminal end of newly synthesized secretory or membrane polypeptides which directs the polypeptide across or into a cell membrane of the cell (the plasma membrane in prokaryotes and the endoplasmic reticulum membrane in eukaryotes). It is usually subsequently removed. In particular, the signal peptide may be capable of directing the polypeptide into a cell's secretory pathway.

It is to be understood, that for the present invention other (i.e., other than the wild-type) signal peptide sequences may be used together with IL12. Such other signal peptide sequences may replace the original wild-type signal peptide sequence. A signal peptide includes peptides that direct newly synthesized protein in the ribosome to the ER and further to the Golgi complex for transport to the plasma membrane or out of the cell. They generally include a string of hydrophobic amino acids and include immunoglobulin leader sequences as well as others known to those skilled in the art. Signal peptides include in particular peptides capable of being acted upon by signal peptidase, a specific protease located on the cisternal face of the endoplasmic reticulum. Signal peptides are well understood by those of skill in the art and may include any known signal peptide. The signal peptide is incorporated at the N-terminus of the protein and processing of the IL12 by signal peptidase produces the active biological form.

In one embodiment, the IL12 comprises a signal peptide sequence having the following sequence:

(SEQ ID NO: 68)
MCHQQLVISWFSLVFLASPLVA

or a signal peptide sequence having at least 70%, 72%, 74%, 76%, 78%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO:68.

An IL12 may also include a protein with a truncated signal peptide sequence. In this context truncated refers to a signal peptide sequence that is shorter than the original signal peptide sequence but still retains at least a portion of its functionality to act as a signal peptide. For example, the IL12 signal peptide sequence comprises or consists of amino acids 1-22 SEQ ID NO:68. An IL12 protein with a truncated signal peptide sequence could have 21, 20, 19, 18, 17, 16, 15, 14, 13, 12, 11, 10, 9, 8, 7, 6, 5, 4, 3, 2 or 1 of the amino acids 1-22 of SEQ ID NO:68.

An IL12 with a truncated signal peptide sequence could also be a protein comprising SEQ ID No: 3 to 6 or a sequence having at least 70%, 72%, 74%, 76%, 78%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 3 to 6 and in addition a signal peptide sequence that is shorter than the original signal peptide sequence. Again, by way of example signal peptide sequence could have 21, 20, 19, 18, 17, 16, 15, 14, 13, 12, 11, 10, 9, 8, 7, 6, 5, 4, 3, 2 or 1 of the amino acids 1-22 of SEQ ID NO: 68 or in a further example, the signal peptide could comprise or consist of the sequence as shown in SEQ ID NO:68.

In another embodiment, a signal peptide sequence is linked to the single-chain IL12p40-IL12p35 or IL12p35-IL12p40.

In a preferred embodiment, the signal peptide sequence comprises an amino acid sequence having at least 90% identity to SEQ ID NO:68, preferably being identical to SEQ ID NO:68.

In a further preferred embodiment, the single-chain IL12p40-IL12p35 comprises a polypeptide having at least 95% identity to SEQ ID NO:66, preferably being identical to SEQ ID NO:66.

In a further preferred embodiment, the single-chain IL12p35-IL12p40 comprises a polypeptide having at least 95% identity to SEQ ID NO:67, preferably being identical to SEQ ID NO:67.

TABLE 2

| Identifier | Sequence | SEQ ID NO: |
|---|---|---|
| IL12p35 (human) | RNLPVATPDPGMFPCLHHSQNLLRAVSNMLQKARQTLEFY | 1 |
| | PCTSEEIDHEDITKDKTSTVEACLPLELTKNESCLNSRETSFI | |
| | TNGSCLASRKTSFMMALCLSSIYEDLKMYQVEFKTMNAKLL | |
| | MDPKRQIFLDQNMLAVIDELMQALNFNSETVPQKSSLEEPD | |
| | FYKTKIKLCILLHAFRIRAVTIDRVMSYLNAS | |
| IL12p40 (human) | IWELKKDVYVVELDWYPDAPGEMVVLTCDTPEEDGITWTL | 2 |
| | DQSSEVLGSGKTLTIQVKEFGDAGQYTCHKGGEVLSHSLL | |
| | LLHKKEDGIWSTDILKDQKEPKNKTFLRCEAKNYSGRFTC | |
| | WWLTTISTDLTFSVKSSRGSSDPQGVTCGAATLSAERVRG | |
| | DNKEYEYSVECQEDSACPAAEESLPIEVMVDAVHKLKYEN | |
| | YTSSFFIRDIIKPDPPKNLQLKPLKNSRQVEVSWEYPDTWS | |
| | TPHSYFSLTFCVQVQGKSKREKKDRVFTDKTSATVICRKNA | |
| | SISVRAQDRYYSSSWSEWASVPCS | |
| IL12p40-IL12p35 | IWELKKDVYVVELDWYPDAPGEMVVLTCDTPEEDGITWTL | 3 |
| (human) | DQSSEVLGSGKTLTIQVKEFGDAGQYTCHKGGEVLSHSLL | |
| | LLHKKEDGIWSTDILKDQKEPKNKTFLRCEAKNYSGRFTC | |
| | WWLTTISTDLTFSVKSSRGSSDPQGVTCGAATLSAERVRG | |
| | DNKEYEYSVECQEDSACPAAEESLPIEVMVDAVHKLKYEN | |
| | YTSSFFIRDIIKPDPPKNLQLKPLKNSRQVEVSWEYPDTWS | |
| | TPHSYFSLTFCVQVQGKSKREKKDRVFTDKTSATVICRKNA | |
| | SISVRAQDRYYSSSWSEWASVPCSRNLPVATPDPGMFPC | |
| | LHHSQNLLRAVSNMLQKARQTLEFYPCTSEEIDHEDITKDK | |
| | TSTVEACLPLELTKNESCLNSRETSFITNGSCLASRKTSFM | |
| | MALCLSSIYEDLKMYQVEFKTMNAKLLMDPKRQIFLDQNML | |
| | AVIDELMQALNFNSETVPQKSSLEEPDFYKTKIKLCILLHAF | |
| | RIRAVTIDRVMSYLNAS | |
| IL12p35-IL12p40 | RNLPVATPDPGMFPCLHHSQNLLRAVSNMLQKARQTLEFY | 4 |
| (human) | PCTSEEIDHEDITKDKTSTVEACLPLELTKNESCLNSRETSFI | |
| | TNGSCLASRKTSFMMALCLSSIYEDLKMYQVEFKTMNAKLL | |
| | MDPKRQIFLDQNMLAVIDELMQALNFNSETVPQKSSLEEPD | |
| | FYKTKIKLCILLHAFRIRAVTIDRVMSYLNASIWELKKDVYVV | |
| | ELDWYPDAPGEMVVLTCDTPEEDGITWTLDQSSEVLGSGK | |
| | TLTIQVKEFGDAGQYTCHKGGEVLSHSLLLLHKKEDGIWST | |
| | DILKDQKEPKNKTFLRCEAKNYSGRFTCWWLTTISTDLTFS | |
| | VKSSRGSSDPQGVTCGAATLSAERVRGDNKEYEYSVECQ | |
| | EDSACPAAEESLPIEVMVDAVHKLKYENYTSSFFIRDIIKPD | |
| | PPKNLQLKPLKNSRQVEVSWEYPDTWSTPHSYFSLTFCVQ | |
| | VQGKSKREKKDRVFTDKTSATVICRKNASISVRAQDRYYSS | |
| | SWSEWASVPCS | |
| IL12p40- 15GS- | IWELKKDVYVVELDWYPDAPGEMVVLTCDTPEEDGITWTL | 5 |
| IL12p35 | DQSSEVLGSGKTLTIQVKEFGDAGQYTCHKGGEVLSHSLL | |
| (human) | LLHKKEDGIWSTDILKDQKEPKNKTFLRCEAKNYSGRFTC | |
| | WWLTTISTDLTFSVKSSRGSSDPQGVTCGAATLSAERVRG | |
| | DNKEYEYSVECQEDSACPAAEESLPIEVMVDAVHKLKYEN | |
| | YTSSFFIRDIIKPDPPKNLQLKPLKNSRQVEVSWEYPDTWS | |
| | TPHSYFSLTFCVQVQGKSKREKKDRVFTDKTSATVICRKNA | |
| | SISVRAQDRYYSSSWSEWASVPCSGGGGSGGGGSGGGG | |
| | SRNLPVATPDPGMFPCLHHSQNLLRAVSNMLQKARQTLEF | |
| | YPCTSEEIDHEDITKDKTSTVEACLPLELTKNESCLNSRETS | |
| | FITNGSCLASRKTSFMMALCLSSIYEDLKMYQVEFKTMNAK | |
| | LLMDPKRQIFLDQNMLAVIDELMQALNFNSETVPQKSSLEE | |
| | PDFYKTKIKLCILLHAFRIRAVTIDRVMSYLNAS | |
| IL12p35-15GS- | RNLPVATPDPGMFPCLHHSQNLLRAVSNMLQKARQTLEFY | 6 |
| IL12p40 | PCTSEEIDHEDITKDKTSTVEACLPLELTKNESCLNSRETSFI | |
| (human) | TNGSCLASRKTSFMMALCLSSIYEDLKMYQVEFKTMNAKLL | |
| | MDPKRQIFLDQNMLAVIDELMQALNFNSETVPQKSSLEEPD | |
| | FYKTKIKLCILLHAFRIRAVTIDRVMSYLNASGGGGGGGGS | |
| | GGGGSIWELKKDVYVVELDWYPDAPGEMVVLTCDTPEED | |
| | GITWTLDQSSEVLGSGKTLTIQVKEFGDAGQYTCHKGGEV | |
| | LSHSLLLLHKKEDGIWSTDILKDQKEPKNKTFLRCEAKNYS | |
| | GRFTCWWLTTISTDLTFSVKSSRGSSDPQGVTCGAATLSA | |
| | ERVRGDNKEYEYSVECQEDSACPAAEESLPIEVMVDAVHK | |
| | LKYENYTSSFFIRDIIKPDPPKNLQLKPLKNSRQVEVSWEYP | |
| | DTWSTPHSYFSLTFCVQVQGKSKREKKDRVFTDKTSATVI | |
| | CRKNASISVRAQDRYYSSSWSEWASVPCS | |
| IL12Rβ1 (human) | MEPLVTWVVPLLFLFLLSRQGAACRTSECCFQDPPYPDAD | 7 |
| P42701 | SGSASGPRDLRCYRISSDRYECSWQYEGPTAGVSHFLRC | |
| | CLSSGRCCYFAAGSATRLQFSDQAGVSVLYTVTLWVESW | |
| | ARNQTEKSPEVTLQLYNSVKYEPPLGDIKVSKLAGQLRME | |
| | WETPDNQVGAEVQFRHRTPSSPWKLGDCGPQDDDTESC | |
| | LCPLEMNVAQEFQLRRRQLGSQGSSWSKWSSPVCVPPE | |
| | NPPQPQVRFSVEQLGQDGRRRLTLKEQPTQLELPEGCQG | |
| | LAPGTEVTYRLQLHMLSCPCKAKATRTLHLGKMPYLSGAA | |
| | YNVAVISSNQFGPGLNQTWHIPADTHTEPVALNISVGTNGT | |
| | TMYWPARAQSMTYCIEWQPVGQDGGLATCSLTAPQDPDP | |
| | AGMATYSWSRESGAMGQEKCYYITIFASAHPEKLTLWSTV | |
| | LSTYHFGGNASAAGTPHHVSVKNHSLDSVSVDWAPSLLST | |
| | CPGVLKEYVVRCRDEDSKQVSEHPVQPTETQVTLSGLRA | |
| | GVAYTVQVRADTAWLRGVWSQPQRFSIEVQVSDWLIFFAS | |
| | LGSFLSILLVGVLGYLGLNRAARHLCPPLPTPCASSAIEFPG | |
| | GKETWQWINPVDFQEEASLQEALVVEMSWDKGERTEPLE | |
| | KTELPEGAPELALDTELSLEDGDRCKAKM | |
| IL12Rβ2 (human) | MAHTFRGCSLAFMFIITWLLIKAKIDACKRGDVTVKPSHVILL | 8 |
| Q99665 | GSTVNITCSLKPRQGCFHYSRRNKLILYKFDRRINFHHGHS | |
| | LNSQVTGLPLGTTLFVCKLACINSDEIQICGAEIFVGVAPEQ | |
| | PQNLSCIQKGEQGTVACTWERGRDTHLYTEYTLQLSGPKN | |
| | LTWQKQCKDIYCDYLDFGINLTPESPESNFTAKVTAVNSLG | |
| | SSSSLPSTFTFLDIVRPLPPWDIRIKFQKASVSRCTLYWRDE | |
| | GLVLLNRLRYRPSNSRLWNMVNVTKAKGRHDLLDLKPFTE | |
| | YEFQISSKLHLYKGSWSDWSESLRAQTPEEEPTGMLDVW | |
| | YMKRHIDYSRQQISLFWKNLSVSEARGKILHYQVTLQELTG | |
| | GKAMTQNITGHTSWTTVIPRTGNWAVAVSAANSKGSSLPT | |
| | RINIMNLCEAGLLAPRQVSANSEGMDNILVTWQPPRKDPS | |
| | AVQEYVVEWRELHPGGDTQVPLNWLRSRPYNVSALISENI | |
| | KSYICYEIRVYALSGDQGGCSSILGNSKHKAPLSGPHINAIT | |
| | EEKGSILISWNSIPVQEQMGCLLHYRIYWKERDSNSQPQLC | |
| | EIPYRVSQNSHPINSLQPRVTYVLWMTALTAAGESSHGNE | |
| | REFCLQGKANWMAFVAPSICIAIIMVGIFSTHYFQQKVFVLL | |
| | AALRPQWCSREIPDPANSTCAKKYPIAEEKTQLPLDRLLID | |
| | WPTPEDPEPLVISEVLHQVTPVFRHPPCSNWPQREKGIQG | |
| | HQASEKDMMHSASSPPPPRALQAESRQLVDLYKVLESRG | |
| | SDPKPENPACPWTVLPAGDLPTHDGYLPSNIDDLPSHEAP | |
| | LADSLEELEPQHISLSVFPSSSLHPLTFSCGDKLTLDQLKM | |
| | RCDSLML | |
| IL12p35 | RVIPVSGPARCLSQSRNLLKTTDDMVKTAREKLKHYSCTAE | 79 |
| (mouse) | DIDHEDITRDQTSTLKTCLPLELHKNESCLATRETSSTTRGS | |
| | CLPPQKTSLMMTLCLGSIYEDLKMYQTEFQAINAALQNHNH | |
| | QQIILDKGMLVAIDELMQSLNHNGETLRQKPPVGEADPYRV | |
| | KMKLCILLHAFSTRVVTINRVMGYLSSA | |
| IL12p40 | MWELEKDVYVVEVDWTPDAPGETVNLTCDTPEEDDITWT | 80 |
| (mouse) | SDQRHGVIGSGKTLTITVKEFLDAGQYTCHKGGETLSHSHL | |
| | LLHKKENGIWSTEILKNFKNKTFLKCEAPNYSGRFTCSWLV | |
| | QRNMDLKFNIKSSSSSPDSRAVTCGMASLSAEKVTLDQRD | |
| | YEKYSVSCQEDVTCPTAEETLPIELALEARQQNKYENYSTS | |
| | FFIRDIIKPDPPKNLQMKPLKNSQVEVSWEYPDSWSTPHSY | |
| | FSLKFFVRIQRKKEKMKETEEGCNQKGAFLVEKTSTEVQC | |
| | KGGNVCVQAQDRYYNSSCSKWACVPCRVRS | |
| IL12p40- | MWELEKDVYVVEVDWTPDAPGETVNLTCDTPEEDDITWT | 82 |
| GGGGSGGGGSGGGGS- | SDQRHGVIGSGKTLTITVKEFLDAGQYTCHKGGETLSHSHL | |
| IL12p35 | LLHKKENGIWSTEILKNFKNKTFLKCEAPNYSGRFTCSWLV | |
| (mouse) | QRNMDLKFNIKSSSSSPDSRAVTCGMASLSAEKVTLDQRD | |
| | YEKYSVSCQEDVTCPTAEETLPIELALEARQQNKYENYSTS | |
| | FFIRDIIKPDPPKNLQMKPLKNSQVEVSWEYPDSWSTPHSY | |
| | FSLKFFVRIQRKKEKMKETEEGCNQKGAFLVEKTSTEVQC | |
| | KGGNVCVQAQDRYYNSSCSKWACVPCRVRSGGGGSGG | |
| | GGSGGGGSRVIPVSGPARCLSQSRNLLKTTDDMVKTARE | |
| | KLKHYSCTAEDIDHEDITRDQTSTLKTCLPLELHKNESCLAT | |
| | RETSSTTRGSCLPPQKTSLMMTLCLGSIYEDLKMYQTEFQ | |
| | AINAALQNHNHQQIILDKGMLVAIDELMQSLNHNGETLRQK | |
| | PPVGEADPYRVKMKLCILLHAFSTRVVTINRVMGYLSSA | |
| SS-IL12p40- | MGWSCIILFLVATATGVHSMWELEKDVYVVEVDWTPDAPG | 83 |
| GGGGSGGGGSGGGGS- | ETVNLTCDTPEEDDITWTSDQRHGVIGSGKTLTITVKEFLDA | |
| IL12p35 | GQYTCHKGGETLSHSHLLLHKKENGIWSTEILKNFKNKTFL | |
| (mouse) | KCEAPNYSGRFTCSWLVQRNMDLKFNIKSSSSSPDSRAVT | |
| | CGMASLSAEKVTLDQRDYEKYSVSCQEDVTCPTAEETLPI | |
| | ELALEARQQNKYENYSTSFFIRDIIKPDPPKNLQMKPLKNS | |
| | QVEVSWEYPDSWSTPHSYFSLKFFVRIQRKKEKMKETEEG | |
| | CNQKGAFLVEKTSTEVQCKGGNVCVQAQDRYYNSSCSK | |
| | WACVPCRVRSGGGGSGGGGSGGGGSRVIPVSGPARCLS | |
| | QSRNLLKTTDDMVKTAREKLKHYSCTAEDIDHEDITRDQTS | |
| | TLKTCLPLELHKNESCLATRETSSTTRGSCLPPQKTSLMMT | |
| | LCLGSIYEDLKMYQTEFQAINAALQNHNHQQIILDKGMLVAI | |
| | DELMQSLNHNGETLRQKPPVGEADPYRVKMKLCILLHAFS | |
| | TRVVTINRVMGYLSSA | |

In a preferred embodiment, the recombinant rhabdovirus encodes in its genome at least one GSDM and further an IL12. Any of the aforementioned GSDM proteins and any of the aforementioned IL12 proteins may be encoded into the virus genome. The GSDM and the IL12 may be encoded at the same location in the virus genome or at different locations in the virus genome. The GSDM and the IL12 may be encoded as a single construct, i.e., the GSDM sequence is followed directly by the IL12 sequence or vice versa. The resulting single construct may then be transcribed as a single chain from the virus genome.

In a preferred embodiment, the GSDM is a GSDME and the IL12 is encoded as a single-chain IL12p40-IL12p35. In a further preferred embodiment, the GSDM is a GSDME and the IL12 is encoded as a single chain in the configuration IL-12p40-GGGGSGGGGSGGGGS-IL-12p35 with a leading signal peptide sequence (SEQ ID NO:66).

In a preferred embodiment, the GSDM and the IL12 further comprise a 2A self-cleaving peptide. The 2A self-cleaving peptide is positioned between the GSDM and the IL12 sequence. 2A self-cleaving peptides, or 2A peptides, are a class of 18-22 aa-long peptides, which can induce ribosomal skipping during translation of a protein in a cell.

In one embodiment, the 2A peptide contains a consensus sequence comprising or consisting of DxExNPGP (SEQ ID NO:69), wherein x can be any amino acid. In another embodiment, the 2A peptide is selected from the group consisting of a T2A, P2A, E2A, or F2A peptide. In one embodiment, the 2A peptide comprises or consists of any one of SEQ ID Nos: 70-71 and 73-75. In any of those embodiments, the 2A peptide may further comprise a short amino acid sequence of GSG at the N-terminal end. In a preferred embodiment, the 2A peptide is a T2A peptide and more preferably a T2A peptide comprising or consisting of SEQ ID NOs: 70 or 71.

In a preferred embodiment the IL12 is linked to a GSDME via a T2A peptide in the configuration GSDME-T2A-IL12. In a related embodiment, the IL12 comprises or consists of any one of SEQ ID NOs: 5 or 66, the T2A peptide comprises or consists of any one of SEQ ID NOs: 70-71, and the GSDME comprises or consists of SEQ ID NO:49. In a most preferred embodiment, the GSDME-T2A-IL12 comprises or consists of SEQ ID NO:72.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and an IL12p40 subunit and an ILp35 subunit of IL12, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and an IL12p40 subunit and an ILp35 subunit of IL12, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, and wherein the at least one GSDM is selected from the group consisting of (i) GSDMA, GSDMB, GSDMC, GSDMD, and GSDME, preferably from the group consisting of any one of SEQ ID NOs: 45-49, AND/OR (ii) GSDMA, GSDMB, GSDMC, GSDMD, and GSDME further comprising a cleavable peptide sequence not naturally occurring. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In a preferred embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and an IL12p40 subunit and an ILp35 subunit of IL12, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, and wherein the IL12p35 and the IL12p40 subunit of IL12 are linked in a single-chain having the configuration IL12p40-IL12p35 or IL12p35-IL12p40, preferably further comprising a GS linker between the IL12p40-IL12p35 or IL12p35-IL12p40, more preferably comprising the amino acid sequence of SEQ ID NO:5 or SEQ ID NO:66. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and an IL12p40 subunit and an ILp35 subunit of IL12, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, and wherein the at least one GSDM is selected from the group consisting of (i) GSDMA, GSDMB, GSDMC, GSDMD, and GSDME, preferably from the group consisting of any one of SEQ ID NOs: 45-49, AND/OR (ii) GSDMA, GSDMB, GSDMC, GSDMD, and GSDME further comprising a cleavable peptide sequence not naturally occurring, and the IL12p35 and the IL12p40 subunit of IL12 are linked in a single-chain having the configuration IL12p40-IL12p35 or IL12p35-IL12p40, preferably further comprising a GS linker between the IL12p40-IL12p35 or IL12p35-IL12p40, more preferably comprising the amino acid sequence of SEQ ID NO:5 or SEQ ID NO:66. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM, wherein the at least one GSDM is selected from the group consisting of (i) GSDMA, GSDMB, GSDMC, GSDMD, and GSDME, preferably from the group consisting of any one of SEQ ID NOs: 45-49, AND/OR (ii) GSDMA, GSDMB, GSDMC, GSDMD, and GSDME further comprising a cleavable peptide sequence not naturally occurring, and an IL12p35 and an IL12p40 subunit of IL12 linked in a single-chain having the configuration IL12p40-IL12p35 and comprising the amino acid sequence of SEQ ID NO: 5 or SEQ ID NO:66, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM comprising the amino acid sequence of SEQ ID NO:49, and an IL12p35 and an IL12p40 subunit of IL12 linked in a single-chain having the configuration IL12p40-IL12p35 and comprising the amino acid sequence of SEQ ID NO:5 or SEQ ID NO:66, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM comprising the amino acid sequence of SEQ ID NO:49, an IL12p35 and an IL12p40 subunit of IL12 linked in a single-chain having the configuration IL12p40-IL12p35 and comprising the amino acid sequence of SEQ ID NO:5 or SEQ ID NO:66, and a 2A peptide, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM comprising the amino acid sequence of SEQ ID NO:49, an IL12p35 and an IL12p40 subunit of IL12 linked in a single-chain having the configuration IL12p40-IL12p35 and comprising the amino acid sequence of SEQ ID NO:5 or SEQ ID NO:66, and a 2A self-cleaving peptide, preferably the 2A self-cleaving peptide is positioned between the GSDM and the IL12 sequence, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM comprising the amino acid sequence of SEQ ID NO:49, an IL12p35 and an IL12p40 subunit of IL12 linked in a single-chain having the configuration IL12p40-IL12p35 and comprising the amino acid sequence of SEQ ID NO:5 or SEQ ID NO:66, and a 2A self-cleaving peptide, preferably the 2A self-cleaving peptide is positioned between the GSDM and the IL12 sequence, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, and wherein the 2A self-cleaving peptide contains a consensus sequence comprising or consisting of DxExNPGP (SEQ ID NO:69), wherein x can be any amino acid. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM comprising the amino acid sequence of SEQ ID NO:49, an IL12p35 and an IL12p40 subunit of IL12 linked in a single-chain having the configuration IL12p40-IL12p35 and comprising the amino acid sequence of SEQ ID NO:5 or SEQ ID NO:66, and a 2A self-cleaving peptide, preferably the 2A self-cleaving peptide is positioned between the GSDM and the IL12 sequence, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, and wherein the 2A self-cleaving peptide is selected from the group consisting of a T2A, P2A, E2A, or F2A peptide. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM comprising the amino acid sequence of SEQ ID NO:49, an IL12p35 and an IL12p40 subunit of IL12 linked in a single-chain having the configuration IL12p40-IL12p35 and comprising the amino acid sequence of SEQ ID NO:5 or SEQ ID NO:66, and a 2A self-cleaving peptide, preferably the 2A self-cleaving peptide is positioned between the GSDM and the IL12 sequence, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, and wherein the 2A self-cleaving peptide is selected from the group consisting of any one of SEQ ID Nos: 70-71 and 73-75. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In a most preferred embodiment, a recombinant vesicular stomatitis virus encodes in its genome a vesicular stomatitis virus nucleoprotein (N), large protein (L), phosphoprotein (P), matrix protein (M), glycoprotein (G) and an amino acid sequence with at least 90%, 91%, 92%, 93%, 94%, 95%, 96%, 97%, 98% or 99% identity to SEQ ID NO: 72, preferably an amino acid sequence identical to SEQ ID NO:72, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV.

In a related most preferred embodiment, a recombinant vesicular stomatitis virus encodes in its genome a vesicular stomatitis virus nucleoprotein (N), large protein (L), phosphoprotein (P), matrix protein (M), glycoprotein (G) and an amino acid sequence with at least 90%, 91%, 92%, 93%, 94%, 95%, 96%, 97%, 98% or 99% identity to SEQ ID NO:72, preferably an amino acid sequence identical to SEQ ID NO: 72, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, and wherein
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

### Gasdermin and (Other) Cytokine Constructs

In a further embodiment, the recombinant rhabdovirus encodes in its genome at least one GSDM and further for at least one cytokine. In a related embodiment, the recombinant rhabdovirus encodes in its genome for at least one GSDM and further for one cytokine, two cytokines or three cytokines. Any of the aforementioned GSDM proteins may be encoded into the virus genome. The at least one cytokine may be selected from interleukins or interferons.

In one embodiment, the interleukin is interleukin18 (IL18). In one embodiment, the interleukin is interleukin1 (IL1). In a related embodiment, the IL1 is either IL1-alpha or IL1-beta. In another embodiment, the interferon (IFN) is a type-I interferon (IFN-type-I). In a related embodiment, the type-I interferon is interferon-alpha. In a preferred embodiment, the interferon-alpha is interferon-alpha-2.

The GSDM and the cytokine may be encoded at the same location in the virus genome or at different locations in the virus genome. The GSDM and the cytokine may be encoded as a single construct, i.e., the GSDM sequence is followed directly by the cytokine sequence or vice versa. The resulting single construct may then be transcribed as a single chain from the virus genome.

In a related embodiment the cytokine further comprises a leading signal peptide sequence.

The GSDM and the cytokine may further comprise a 2A self-cleaving peptide. The 2A self-cleaving peptide is positioned between the GSDM and the cytokine sequence. 2A self-cleaving peptides, or 2A peptides, are a class of 18-22 aa-long peptides, which can induce ribosomal skipping during translation of a protein in a cell.

In one embodiment, the 2A peptide contains a consensus sequence comprising or consisting of DxExNPGP (SEQ ID NO:69), wherein x can be any amino acid. In another embodiment, the 2A peptide is selected from the group consisting of a T2A, P2A, E2A, or F2A peptide. In one embodiment, the 2A peptide comprises or consists of any one of SEQ ID Nos: 70-71 and 73-75. In any of those embodiments, the 2A peptide may further comprise a short amino acid sequence of GSG at the N-terminal end. In a preferred embodiment, the 2A peptide is a T2A peptide and more preferably a T2A peptide comprising or consisting of SEQ ID NOs: 70 or 71.

In a preferred embodiment the cytokine is linked to a GSDM via a T2A peptide in the configuration GSDM-T2A-cytokine.

### GSDM and IL18

In one embodiment, the recombinant rhabdovirus encodes in its genome at least one GSDM and further an IL18. Any of the aforementioned GSDM proteins may be encoded into the virus genome. The GSDM and the IL18 may be encoded at the same location in the virus genome or at different locations in the virus genome. The GSDM and the IL18 may be encoded as a single construct, i.e., the GSDM sequence is followed directly by the IL18 sequence or vice versa. The resulting single construct may then be transcribed as a single chain from the virus genome. In a related embodiment, the IL18 comprises or consists of any one of SEQ ID NOs: 103-105. In a related embodiment the IL18 further comprises a leading signal peptide sequence.

In a preferred embodiment, the GSDM and the IL18 further comprise a 2A self-cleaving peptide. The 2A self-cleaving peptide is positioned between the GSDM and the IL18 sequence. 2A self-cleaving peptides, or 2A peptides, are a class of 18-22 aa-long peptides, which can induce ribosomal skipping during translation of a protein in a cell.

In one embodiment, the 2A peptide contains a consensus sequence comprising or consisting of DxExNPGP (SEQ ID NO:69), wherein x can be any amino acid. In another embodiment, the 2A peptide is selected from the group consisting of a T2A, P2A, E2A, or F2A peptide. In one embodiment, the 2A peptide comprises or consists of any one of SEQ ID Nos: 70-71 and 73-75. In any of those embodiments, the 2A peptide may further comprise a short amino acid sequence of GSG at the N-terminal end. In a preferred embodiment, the 2A peptide is a T2A peptide and more preferably a T2A peptide comprising or consisting of SEQ ID NOs: 70 or 71.

In a preferred embodiment the IL18 is linked to a GSDM via a T2A peptide in the configuration GSDM-T2A-IL18. In a related embodiment, the IL18 comprises or consists of any one of SEQ ID NOs: 103-105, the T2A peptide comprises or consists of any one of SEQ ID NOs: 70-71, and the GSDM comprises or consists of any one of SEQ ID NOs: 45-49 or 107.

In a preferred embodiment, the GSDM is a GSDME. In a preferred embodiment, the IL18 is linked to a GSDME via a T2A peptide in the configuration GSDME-T2A-IL18. In a related embodiment, the IL18 comprises or consists of any one of SEQ ID NOs: 103-105, the T2A peptide comprises or consists of any one of SEQ ID NOs: 70-71, and the GSDME comprises or consists of SEQ ID NO:49.

In another preferred embodiment, the GSDM is a GSDMD. In a related embodiment, the GSDMD comprises a cleavable peptide sequence not naturally occurring. In another embodiment, the GSDMD comprises a cleavable peptide sequence not naturally occurring, wherein the cleavable peptide sequence is specifically cleavable by caspase-3. In another embodiment, the GSDMD comprises a cleavable peptide sequence, wherein the cleavable peptide sequence comprises the consensus sequence DxxD (SEQ ID NO:62), wherein X can be any amino acid. In a related embodiment, the protease cleavable peptide sequence is specifically cleavable by caspase-3 and the cleavable peptide sequence comprises the consensus sequence DxxD (SEQ ID NO:62), wherein X can be any amino acid. In another embodiment, the GSDMD comprises a cleavable peptide sequence, wherein the cleavable peptide sequence is specifically cleavable by caspase-3, and wherein cleavage by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:54. In another embodiment, the GSDMD comprising a cleavable peptide sequence not naturally occurring, comprises an amino acid sequence as shown in SEQ ID NO:107.

In a preferred embodiment the IL18 is linked to a GSDMD via a T2A peptide in the configuration GSDMD-T2A-IL18. In a related embodiment, the IL18 comprises or consists of any one of SEQ ID NOs: 103-105, the T2A peptide comprises or consists of any one of SEQ ID NOs: 70-71, and the GSDMD comprises or consists of SEQ ID NO: 48 or 107.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and IL18, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and IL18, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, and wherein the at least one GSDM is selected from the group consisting of (i) GSDMA, GSDMB, GSDMC, GSDMD, and GSDME, preferably from the group consisting of any one of SEQ ID NOs: 45-49 or 107, AND/OR (ii) GSDMA, GSDMB, GSDMC, GSDMD, and GSDME further comprising a cleavable peptide sequence not naturally occurring. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and IL18, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, and wherein the IL18 is selected from any one of SEQ ID NOs: 103-105, and wherein the at least one GSDM is selected from the group consisting of (i) GSDMA, GSDMB, GSDMC, GSDMD, and GSDME, preferably from the group consisting of any one of SEQ ID NOs: 45-49 or 107, AND/OR (ii) GSDMA, GSDMB, GSDMC, GSDMD, and GSDME further comprising a cleavable peptide sequence not naturally occurring. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

### GSDM and IL18 and IL12

In another embodiment, the recombinant rhabdovirus encodes in its genome at least one GSDM and further an IL18 and an IL12. Any of the aforementioned GSDM proteins may be encoded into the virus genome. The GSDM, the IL18, and the IL12 may be encoded at the same location in the virus genome or at different locations in the virus genome. The GSDM, the IL18, and the IL12 may be encoded as a single construct, i.e., the GSDM sequence is followed directly by the IL18 sequence followed by the IL12 sequence or any alterations thereof, such as GSDM-IL18-IL12, GSDM-IL12-IL18, IL18-GSDM-IL12, IL18-IL12-GSDM, IL12-GSDM-IL18, IL12-IL18-GSDM. The resulting single construct may then be transcribed as a single chain from the virus genome. In a related embodiment, the IL18 comprises or consists of any one of SEQ ID NOs: 103-105. Any of the earlier described IL12 constructs may be used here as well. In a related embodiment, the IL12 comprises or consists of any one of SEQ ID NOs: 3-6. In a related embodiment the IL18 and/or the IL12 further comprises a leading signal peptide sequence.

In a preferred embodiment, the GSDM, the IL18 and the IL12 further comprise one or more 2A self-cleaving peptide(s). The 2A self-cleaving peptide is positioned between the GSDM and the IL18 sequence and/or between the IL18 sequence and the IL12 sequence. Preferably, a 2A self-cleaving peptide is positioned both between the GSDM and the IL18 sequence and the IL18 sequence and the IL12 sequence. 2A self-cleaving peptides, or 2A peptides, are a class of 18-22 aa-long peptides, which can induce ribosomal skipping during translation of a protein in a cell.

In one embodiment, the 2A peptide contains a consensus sequence comprising or consisting of DxExNPGP (SEQ ID NO:69), wherein x can be any amino acid. In another embodiment, the 2A peptide is selected from the group consisting of a T2A, P2A, E2A, or F2A peptide. In one embodiment, the 2A peptide comprises or consists of any one of SEQ ID Nos: 70-71 and 73-75. In any of those embodiments, the 2A peptide may further comprise a short amino acid sequence of GSG at the N-terminal end. In a preferred embodiment, the 2A peptide is a T2A peptide and more preferably a T2A peptide comprising or consisting of SEQ ID NOs: 70 or 71.

In a preferred embodiment the IL18 is linked to a GSDM via a T2A peptide and the IL18 is linked to the IL12 in the configuration GSDM-T2A-IL18-T2A-IL12. In a related embodiment, the IL12 comprises or consists of any one of SEQ ID NOs: 5 or 66, the IL18 comprises or consists of any one of SEQ ID NOs: 103-105, the T2A peptide comprises or consists of any one of SEQ ID NOs: 70-71, and the GSDM comprises or consists of any one of SEQ ID NOs: 45-49 or 107.

In a preferred embodiment, the GSDM is a GSDME. In a preferred embodiment the IL18 is linked to the GSDME via a T2A peptide and the IL18 is linked to the IL12 in the configuration GSDME-T2A-IL18-T2A-IL12. In a related embodiment, the IL12 comprises or consists of any one of SEQ ID NOs: 5 or 66, the IL18 comprises or consists of any one of SEQ ID NOs: 103-105, the T2A peptide comprises or consists of any one of SEQ ID NOs: 70-71, and the GSDME comprises or consists of SEQ ID NOs: 49.

In another preferred embodiment, the GSDM is a GSDMD. In a related embodiment, the GSDMD comprises a cleavable peptide sequence not naturally occurring. In another embodiment, the GSDMD comprises a cleavable peptide sequence not naturally occurring, wherein the cleavable peptide sequence is specifically cleavable by caspase-3. In another embodiment, the GSDMD comprises a cleavable peptide sequence, wherein the cleavable peptide sequence comprises the consensus sequence DxxD (SEQ ID NO:62), wherein X can be any amino acid. In a related embodiment, the protease cleavable peptide sequence is specifically cleavable by caspase-3 and the cleavable peptide sequence comprises the consensus sequence DxxD (SEQ ID NO:62), wherein X can be any amino acid. In another embodiment, the GSDMD comprises a cleavable peptide sequence, wherein the cleavable peptide sequence is specifically cleavable by caspase-3, and wherein cleavage by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:54. In another embodiment, the GSDMD comprising a cleavable peptide sequence not naturally occurring comprises an amino acid sequence as shown in SEQ ID NO:107.

In a preferred embodiment the IL18 is linked to the GSDMD via a T2A peptide and the IL18 is linked to the IL12 in the configuration GSDMD-T2A-IL18-T2A-IL12. In a related embodiment, the IL12 comprises or consists of any one of SEQ ID NOs: 5 or 66, the IL18 comprises or consists of any one of SEQ ID NOs: 103-105, the T2A peptide comprises or consists of any one of SEQ ID NOs: 70-71, and the GSDMD comprises or consists of SEQ ID NO: 48 or 107.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and IL18 and IL12, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and IL18 and IL12, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, and wherein the at least one GSDM is selected from the group consisting of (i) GSDMA, GSDMB, GSDMC, GSDMD, and GSDME, preferably from the group consisting of any one of SEQ ID NOs: 45-49 or 107, AND/OR (ii) GSDMA, GSDMB, GSDMC, GSDMD, and GSDME further comprising a cleavable peptide sequence not naturally occurring. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and IL18 and IL12, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, wherein the IL18 is selected from any one of SEQ ID NOs: 103-105, and wherein the at least one GSDM is selected from the group consisting of (i) GSDMA, GSDMB, GSDMC, GSDMD, and GSDME, preferably from the group consisting of any one of SEQ ID NOs: 45-49 or 107, AND/OR (ii) GSDMA, GSDMB, GSDMC, GSDMD, and GSDME further comprising a cleavable peptide sequence not naturally occurring. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and IL18 and IL12, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, wherein the IL18 is selected from any one of SEQ ID NOs: 103-105, wherein the IL12 is selected from any one of SEQ ID NOs: 5 or 66, and wherein the at least one GSDM is selected from the group consisting of (i) GSDMA, GSDMB, GSDMC, GSDMD, and GSDME, preferably from the group consisting of any one of SEQ ID NOs: 45-49 or 107, AND/OR (ii) GSDMA, GSDMB, GSDMC, GSDMD, and GSDME further comprising a cleavable peptide sequence not naturally occurring. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and IL18 and IL12, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, and wherein the IL12 is selected from any one of SEQ ID NOs: 5 or 66, and wherein the at least one GSDM is selected from the group consisting of (i) GSDMA, GSDMB, GSDMC, GSDMD, and GSDME, preferably from the group consisting of any one of SEQ ID NOs: 45-49 or 107, AND/OR (ii) GSDMA, GSDMB, GSDMC, GSDMD, and GSDME further comprising a cleavable peptide sequence not naturally occurring. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29

### GSDM and IL18 and IL1

In another embodiment, the recombinant rhabdovirus encodes in its genome at least one GSDM and further an IL18 and an IL1. Any of the aforementioned GSDM proteins may be encoded into the virus genome. The GSDM, the IL18, and the IL1 may be encoded at the same location in the virus genome or at different locations in the virus genome. The GSDM, the IL18, and the IL1 may be encoded as a single construct, i.e., the GSDM sequence is followed directly by the IL18 sequence followed by the IL1 sequence or any alterations thereof, such as GSDM-IL18-IL1, GSDM-IL1-IL18, IL18-GSDM-IL1, IL18-IL1-GSDM, IL1-GSDM-IL18, IL1-IL18-GSDM. The resulting single construct may then be transcribed as a single chain from the virus genome. In a related embodiment, the IL18 comprises or consists of any one of SEQ ID NOs: 103-105. In a related embodiment, the IL1 comprises or consists of any one of SEQ ID NOs: 99-102. In a related embodiment the IL18 and/or the IL1 further comprises a leading signal peptide sequence.

In a preferred embodiment, the GSDM, the IL18 and the IL1 further comprise one or more 2A self-cleaving peptide(s). The 2A self-cleaving peptide is positioned between the GSDM and the IL18 sequence and/or between the IL18 sequence and the IL1 sequence. Preferably, a 2A self-cleaving peptide is positioned both between the GSDM and the IL18 sequence and the IL18 sequence and the IL1 sequence. 2A self-cleaving peptides, or 2A peptides, are a class of 18-22 aa-long peptides, which can induce ribosomal skipping during translation of a protein in a cell.

In one embodiment, the 2A peptide contains a consensus sequence comprising or consisting of DxExNPGP (SEQ ID NO:69), wherein x can be any amino acid. In another embodiment, the 2A peptide is selected from the group consisting of a T2A, P2A, E2A, or F2A peptide. In one embodiment, the 2A peptide comprises or consists of any one of SEQ ID Nos: 70-71 and 73-75. In any of those embodiments, the 2A peptide may further comprise a short amino acid sequence of GSG at the N-terminal end. In a preferred embodiment, the 2A peptide is a T2A peptide and more preferably a T2A peptide comprising or consisting of SEQ ID NOs: 70 or 71.

In a preferred embodiment the IL18 is linked to a GSDM via a T2A peptide and the IL18 is linked to the IL1 in the configuration GSDM-T2A-IL18-T2A-IL1. In a related embodiment, the IL1 comprises or consists of any one of SEQ ID NOs: 99-102, the IL18 comprises or consists of any one of SEQ ID NOs: 103-105, the T2A peptide comprises or consists of any one of SEQ ID NOs: 70-71, and the GSDM comprises or consists of any one of SEQ ID NOs: 45-49 or 107.

In a preferred embodiment, the GSDM is a GSDME. In a preferred embodiment the IL18 is linked to the GSDME via a T2A peptide and the IL18 is linked to the IL1 in the configuration GSDME-T2A-IL18-T2A-IL1. In a related embodiment, the IL1 comprises or consists of any one of SEQ ID NOs: 99-102, the IL18 comprises or consists of any one of SEQ ID NOs: 103-105, the T2A peptide comprises or consists of any one of SEQ ID NOs: 70-71, and the GSDME comprises or consists of SEQ ID NOs: 49.

In another preferred embodiment, the GSDM is a GSDMD. In a related embodiment, the GSDMD comprises a cleavable peptide sequence not naturally occurring. In another embodiment, the GSDMD comprises a cleavable peptide sequence not naturally occurring, wherein the cleavable peptide sequence is specifically cleavable by caspase-3. In another embodiment, the GSDMD comprises a cleavable peptide sequence, wherein the cleavable peptide sequence comprises the consensus sequence DxxD (SEQ ID NO:62), wherein X can be any amino acid. In a related embodiment, the protease cleavable peptide sequence is specifically cleavable by caspase-3 and the cleavable peptide sequence comprises the consensus sequence DxxD (SEQ ID NO:62), wherein X can be any amino acid. In another embodiment, the GSDMD comprises a cleavable peptide sequence, wherein the cleavable peptide sequence is specifically cleavable by caspase-3, and wherein cleavage by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:54. In another embodiment, the GSDMD comprising a cleavable peptide sequence not naturally occurring comprises an amino acid sequence as shown in SEQ ID NO:107

In a preferred embodiment the IL18 is linked to the GSDMD via a T2A peptide and the IL18 is linked to the IL1 in the configuration GSDMD-T2A-IL18-T2A-IL1. In a related embodiment, the IL1 comprises or consists of any one of SEQ ID NOs: 99-102, the IL18 comprises or consists of any one of SEQ ID NOs: 103-105, the T2A peptide comprises or consists of any one of SEQ ID NOs: 70-71, and the GSDMD comprises or consists of SEQ ID NO: 48 or 107.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and IL18 and IL1, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and IL18 and IL1, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, and wherein the at least one GSDM is selected from the group consisting of (i) GSDMA, GSDMB, GSDMC, GSDMD, and GSDME, preferably from the group consisting of any one of SEQ ID NOs: 45-49 or 107, AND/OR (ii) GSDMA, GSDMB, GSDMC, GSDMD, and GSDME further comprising a cleavable peptide sequence not naturally occurring. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and IL18 and IL1, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, wherein the IL18 is selected from any one of SEQ ID NOs: 103-105, and wherein the at least one GSDM is selected from the group consisting of (i) GSDMA, GSDMB, GSDMC, GSDMD, and GSDME, preferably from the group consisting of any one of SEQ ID NOs: 45-49 or 107, AND/OR (ii) GSDMA, GSDMB, GSDMC, GSDMD, and GSDME further comprising a cleavable peptide sequence not naturally occurring. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and IL18 and IL1, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, wherein the IL18 is selected from any one of SEQ ID NOs: 103-105, wherein the IL1 is selected from any one of SEQ ID NOs: 99-102, and wherein the at least one GSDM is selected from the group consisting of (i) GSDMA, GSDMB, GSDMC, GSDMD, and GSDME, preferably from the group consisting of any one of SEQ ID NOs: 45-49 or 107, AND/OR (ii) GSDMA, GSDMB, GSDMC, GSDMD, and GSDME further comprising a cleavable peptide sequence not naturally occurring. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and IL18 and IL1, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, and wherein the IL1 is selected from any one of SEQ ID NOs: 99-102, and wherein the at least one GSDM is selected from the group consisting of (i) GSDMA, GSDMB, GSDMC, GSDMD, and GSDME, preferably from the group consisting of any one of SEQ ID NOs: 45-49 or 107, AND/OR (ii) GSDMA, GSDMB, GSDMC, GSDMD, and GSDME further comprising a cleavable peptide sequence not naturally occurring. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29

### GSDM, IL18, IL1 and IFN-alpha-2

In another embodiment, the recombinant rhabdovirus encodes in its genome at least one GSDM and further an IL18, an IL1, and an IFN-alpha-2. Any of the aforementioned GSDM proteins may be encoded into the virus genome. The GSDM, the IL18, the IL1, and the IFN-alpha-2 may be encoded at the same location in the virus genome or at different locations in the virus genome. The GSDM, the IL18, the IL1, and the IFN-alpha-2 may be encoded as a single construct, i.e., the GSDM sequence is followed directly by the IL18 sequence, followed by the IL1 sequence, followed by the IFN-alpha-2 sequence, or any alterations thereof, such as GSDM-IL18-IL1-IFN-alpha-2, GSDM-IL1-IL18-IFN-alpha-2, GSDM-IL18-IFN-alpha-2-IL1, GSDM-IFN-alpha-2-IL18-IL1, GSDM-IFN-alpha-2-IL1-IL18, IL18-GSDM-IL1-IFN-alpha-2, IL18-IL1-GSDM-IFN-alpha-2, IL18-GSDM-IFN-alpha-2-IL1, IL18-IFN-alpha-2-GSDM-IL1, IL18-IFN-alpha-2-IL1-GSDM, IL1-IL18-GSDM-IFN-alpha-2, IL1-GSDM-IL18-IFN-alpha-2, IL1-IL18-IFN-alpha-2-GSDM, IL1-IFN-alpha-2-IL18-GSDM, IL1-IFN-alpha-2-GSDM-IL18, IFN-alpha-2-IL18-IL1-GSDM, IFN-alpha-2-IL1-IL18-GSDM, IFN-alpha-2-IL18-GSDM-IL1, IFN-alpha-2-GSDM-IL18-IL1, IFN-alpha-2-GSDM-IL1-IL18.

The resulting single construct may then be transcribed as a single chain from the virus genome. In a related embodiment, the IL18 comprises or consists of any one of SEQ ID NOs: 103-105. In a related embodiment, the IL1 comprises or consists of any one of SEQ ID NOs: 99-102. In a related embodiment, the IFN-alpha-2 comprises or consists of SEQ ID NOs: 106. In a related embodiment the IL18 and/or the IL1 and/or the IFN-alpha-2 further comprises a leading signal peptide sequence.

In a preferred embodiment, the GSDM, the IL18, the IL1, and the IFN-alpha-2 further comprise one or more 2A self-cleaving peptide(s). 2A self-cleaving peptides, or 2A peptides, are a class of 18-22 aa-long peptides, which can induce ribosomal skipping during translation of a protein in a cell.

In one embodiment, the 2A peptide contains a consensus sequence comprising or consisting of DxExNPGP (SEQ ID NO:69), wherein x can be any amino acid. In another embodiment, the 2A peptide is selected from the group consisting of a T2A, P2A, E2A, or F2A peptide. In one embodiment, the 2A peptide comprises or consists of any one of SEQ ID Nos: 70-71 and 73-75. In any of those embodiments, the 2A peptide may further comprise a short amino acid sequence of GSG at the N-terminal end. In a preferred embodiment, the 2A peptide is a T2A peptide and more preferably a T2A peptide comprising or consisting of SEQ ID NOs: 70 or 71.

In a preferred embodiment the GSDM, the IL18, the IL1, and the IFN-alpha-2 are linked via a T2A peptide in the configuration GSDM-T2A-IL18-T2A-IL1-T2A-IFN-alpha-2 or GSDM-T2A-IL1-T2A-IL18-T2A-IFN-alpha-2. In a related embodiment, the IL1 comprises or consists of any one of SEQ ID NOs: 99-102, the IL18 comprises or consists of any one of SEQ ID NOs: 103-105, the IFN-alpha-2 comprises or consists of SEQ ID NOs: 106, the T2A peptide comprises or consists of any one of SEQ ID NOs: 70-71, and the GSDM comprises or consists of any one of SEQ ID NOs: 45-49 or 107.

In a preferred embodiment, the GSDM is a GSDME. In a preferred embodiment the GSDME, the IL18, the IL1, and the IFN-alpha-2 are linked via a T2A peptide in the configuration GSDME-T2A-IL18-T2A-IL1-T2A-IFN-alpha-2 or GSDME-T2A-IL1-T2A-IL18-T2A-IFN-alpha-2. In a related embodiment, the IL1 comprises or consists of any one of SEQ ID NOs: 99-102, the IL18 comprises or consists of any one of SEQ ID NOs: 103-105, the IFN-alpha-2 comprises or consists of SEQ ID NOs: 106, the T2A peptide comprises or consists of any one of SEQ ID NOs: 70-71, and the GSDME comprises or consists of SEQ ID NOs: 49.

In another preferred embodiment, the GSDM is a GSDMD. In a related embodiment, the GSDMD comprises a cleavable peptide sequence not naturally occurring. In another embodiment, the GSDMD comprises a cleavable peptide sequence not naturally occurring, wherein the cleavable peptide sequence is specifically cleavable by caspase-3. In another embodiment, the GSDMD comprises a cleavable peptide sequence, wherein the cleavable peptide sequence comprises the consensus sequence DxxD (SEQ ID NO:62), wherein X can be any amino acid. In a related embodiment, the protease cleavable peptide sequence is specifically cleavable by caspase-3 and the cleavable peptide sequence comprises the consensus sequence DxxD (SEQ ID NO:62), wherein X can be any amino acid. In another embodiment, the GSDMD comprises a cleavable peptide sequence, wherein the cleavable peptide sequence is specifically cleavable by caspase-3, and wherein cleavage by caspase-3 will result in a GSDM-NT domain having at least 95%, 90%, 85%, 80%, 75%, 70%, 65%, 60%, 55%, 50%, 45%, 40%, 35%, 30%, 25% or 20% activity of a GSDM-NT according to SEQ ID NO:54. In another embodiment, the GSDMD comprising a cleavable peptide sequence not naturally occurring comprises an amino acid sequence as shown in SEQ ID NO:107

In a preferred embodiment, the GSDM is a GSDMD. In a preferred embodiment the GSDMD, the IL18, the IL1, and the IFN-alpha-2 are linked via a T2A peptide in the configuration GSDMD-T2A-IL18-T2A-IL1-T2A-IFN-alpha-2 or GSDMD-T2A-IL1-T2A-IL18-T2A-IFN-alpha-2. In a related embodiment, the IL1 comprises or consists of any one of SEQ ID NOs: 99-102, the IL18 comprises or consists of any one of SEQ ID NOs: 103-105, the IFN-alpha-2 comprises or consists of SEQ ID NOs: 106, the T2A peptide comprises or consists of any one of SEQ ID NOs: 70-71, and the GSDMD comprises or consists of SEQ ID NOs: 48 or 107.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and IL18 and IL1 and IFN-alpha-2, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and IL18 and IL1 and IFN-alpha-2, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, and wherein the at least one GSDM is selected from the group consisting of (i) GSDMA, GSDMB, GSDMC, GSDMD, and GSDME, preferably from the group consisting of any one of SEQ ID NOs: 45-49 or 107, AND/OR (ii) GSDMA, GSDMB, GSDMC, GSDMD, and GSDME further comprising a cleavable peptide sequence not naturally occurring. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and IL18 and IL1 and IFN-alpha-2, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, wherein the IL18 is selected from any one of SEQ ID NOs: 103-105, and wherein the at least one GSDM is selected from the group consisting of (i) GSDMA, GSDMB, GSDMC, GSDMD, and GSDME, preferably from the group consisting of any one of SEQ ID NOs: 45-49 or 107, AND/OR (ii) GSDMA, GSDMB, GSDMC, GSDMD, and GSDME further comprising a cleavable peptide sequence not naturally occurring. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and IL18 and IL1 and IFN-alpha-2, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, wherein the IL18 is selected from any one of SEQ ID NOs: 103-105, wherein the IL1 is selected from any one of SEQ ID NOs: 99-102, and wherein the at least one GSDM is selected from the group consisting of (i) GSDMA, GSDMB, GSDMC, GSDMD, and GSDME, preferably from the group consisting of any one of SEQ ID NOs: 45-49 or 107, AND/OR (ii) GSDMA, GSDMB, GSDMC, GSDMD, and GSDME further comprising a cleavable peptide sequence not naturally occurring. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and IL18 and IL1 and IFN-alpha-2, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, wherein the IL1 is selected from any one of SEQ ID NOs: 99-102, and wherein the at least one GSDM is selected from the group consisting of (i) GSDMA, GSDMB, GSDMC, GSDMD, and GSDME, preferably from the group consisting of any one of SEQ ID NOs: 45-49 or 107, AND/OR (ii) GSDMA, GSDMB, GSDMC, GSDMD, and GSDME further comprising a cleavable peptide sequence not naturally occurring. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and IL18 and IL1 and IFN-alpha-2, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, wherein the IFN-alpha-2 comprises an amino acid sequence as shown in SEQ ID NO:106, and wherein the at least one GSDM is selected from the group consisting of (i) GSDMA, GSDMB, GSDMC, GSDMD, and GSDME, preferably from the group consisting of any one of SEQ ID NOs: 45-49 or 107, AND/OR (ii) GSDMA, GSDMB, GSDMC, GSDMD, and GSDME further comprising a cleavable peptide sequence not naturally occurring. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and IL18 and IL1 and IFN-alpha-2, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, wherein the IFN-alpha-2 comprises an amino acid sequence as shown in SEQ ID NO:106, wherein the IL18 is selected from any one of SEQ ID NOs: 103-105, and wherein the at least one GSDM is selected from the group consisting of (i) GSDMA, GSDMB, GSDMC, GSDMD, and GSDME, preferably from the group consisting of any one of SEQ ID NOs: 45-49 or 107, AND/OR (ii) GSDMA, GSDMB, GSDMC, GSDMD, and GSDME further comprising a cleavable peptide sequence not naturally occurring. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and IL18 and IL1 and IFN-alpha-2, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, wherein the IFN-alpha-2 comprises an amino acid sequence as shown in SEQ ID NO:106, wherein the IL1 is selected from any one of SEQ ID NOs: 99-102, and wherein the at least one GSDM is selected from the group consisting of (i) GSDMA, GSDMB, GSDMC, GSDMD, and GSDME, preferably from the group consisting of any one of SEQ ID NOs: 45-49 or 107, AND/OR (ii) GSDMA, GSDMB, GSDMC, GSDMD, and GSDME further comprising a cleavable peptide sequence not naturally occurring. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

In one embodiment, a recombinant vesicular stomatitis virus encodes in its genome a vesicular stomatitis virus nucleoprotein (N), large protein (L), phosphoprotein (P), matrix protein (M), glycoprotein (G) and at least one GSDM and IL18 and IL1 and IFN-alpha-2, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV, wherein the IFN-alpha-2 comprises an amino acid sequence as shown in SEQ ID NO:106, wherein the IL1 is selected from any one of SEQ ID NOs: 99-102, wherein the IL18 is selected from any one of SEQ ID NOs: 103-105, and wherein the at least one GSDM is selected from the group consisting of (i) GSDMA, GSDMB, GSDMC, GSDMD, and GSDME, preferably from the group consisting of any one of SEQ ID NOs: 45-49 or 107, AND/OR (ii) GSDMA, GSDMB, GSDMC, GSDMD, and GSDME further comprising a cleavable peptide sequence not naturally occurring. In a related embodiment,
the nucleoprotein (N) comprises an amino acid as set forth in SEQ ID NO: 28 or a functional variant at least 80%, 85%, 90%, 92%, 94%, 96%, 98% identical to SEQ ID NO:28
wherein the phosphoprotein (P) comprises an amino acid as set forth in SEQ ID NO:29 or a functional variant at least 80%, 85%, 90%, 92%, 94%, 96%, 98% identical to SEQ ID NO:29
wherein the large protein (L) comprises an amino acid as set forth in SEQ ID NO: 30 or a functional variant at least 80%, 85%, 90%, 92%, 94%, 96%, 98% identical to SEQ ID NO:30
the matrix protein (M) comprises an amino acid as set forth in SEQ ID NO: 31 or a functional variant at least 80%, 85%, 90%, 92%, 94%, 96%, 98% identical to SEQ ID NO:31.

TABLE 3

| Identifier | Sequence | SEQ ID NO: |
|---|---|---|
| IL1a | MAKVPDLFEDLKNCYSENEDYSSAIDHLSLNQKSFYDASY | 91 |
| (mouse) | GSLHETCTDQFVSLRTSETSKMSNFTFKESRVTVSATSSN | |
| | GKILKKRRLSFSETFTEDDLQSITHDLEETIQPRSAPYTYQS | |
| | DLRYKLMKLVRQKFVMNDSLNQTIYQDVDKHYLSTTWLND | |
| | LQQEVKFDMYAYSSGGDDSKYPVTLKISDSQLFVSAQGED | |
| | QPVLLKELPETPKLITGSETDLIFFWKSINSKNYFTSAAYPEL | |
| | FIATKEQSRVHLARGLPSMTDFQIS | |
| mature IL1a | SAPYTYQSDLRYKLMKLVRQKFVMNDSLNQTIYQDVDKHY | 92 |
| (mouse) | LSTTWLNDLQQEVKFDMYAYSSGGDDSKYPVTLKISDSQL | |
| | FVSAQGEDQPVLLKELPETPKLITGSETDLIFFWKSINSKNY | |
| | FTSAAYPELFIATKEQSRVHLARGLPSMTDFQIS | |
| IL1b | MATVPELNCEMPPFDSDENDLFFEVDGPQKMKGCFQTFD | 93 |
| (mouse) | LGCPDESIQLQISQQHINKSFRQAVSLIVAVEKLWQLPVSFP | |
| | WTFQDEDMSTFFSFIFEEEPILCDSWDDDDNLLVCDVPIRQ | |
| | LHYRLRDEQQKSLVLSDPYELKALHLNGQNINQQVIFSMSF | |
| | VQGEPSNDKIPVALGLKGKNLYLSCVMKDGTPTLQLESVD | |
| | PKQYPKKKMEKRFVFNKIEVKSKVEFESAEFPNWYISTSQA | |
| | EHKPVFLGNNSGQDIIDFTMESVSS | |
| mature IL1b | VPIRQLHYRLRDEQQKSLVLSDPYELKALHLNGQNINQQVI | 94 |
| (mouse) | FSMSFVQGEPSNDKIPVALGLKGKNLYLSCVMKDGTPTLQ | |
| | LESVDPKQYPKKKMEKRFVFNKIEVKSKVEFESAEFPNWYI | |
| | STSQAEHKPVFLGNNSGQDIIDFTMESVS | |
| IL18 | MAAMSEDSCVNFKEMMFIDNTLYFIPEENGDLESDNFGRL | 95 |
| (mouse) | HCTTAVIRNINDQVLFVDKRQPVFEDMTDIDQSASEPQTRLI | |
| | IYMYKDSEVRGLAVTLSVKDSKMSTLSCKNKIISFEEMDPP | |
| | ENIDDIQSDLIFFQKRVPGHNKMEFESSLYEGHFLACQKED | |
| | DAFKLILKKKDENGDKSVMFTLTNLHQS | |
| mature IL18 | NFGRLHCTTAVIRNINDQVLFVDKRQPVFEDMTDIDQSASE | 96 |
| (mouse) | PQTRLIIYMYKDSEVRGLAVTLSVKDSKMSTLSCKNKIISFE | |
| | EMDPPENIDDIQSDLIFFQKRVPGHNKMEFESSLYEGHFLA | |
| | CQKEDDAFKLILKKKDENGDKSVMFTLTNLHQS | |
| mature IL18DR | HFGRLHCTTAVIRNINDQVLFVDKRQPVFEDMTDIDQSASE | 97 |
| (mouse) | PQTRLIIYAYGDSRARGKAVTLSVKDSKMSTLSCKNKIISFE | |
| | EMDPPENIDDIQSDLIFFQKRVPGHNKMEFESSLYEGHFLA | |
| | CQKEDDAFKLILKKKDENGDKSVMFTLTNLHQS | |
| IFN-alpha-2 | MARLCAFLVMLIVMSYWSICSLGCDLPHTYNLRNKRALKVL | 98 |
| (mouse) | AQMRRLPFLSCLKDRQDFGFPLEKVDNQQIQKAQAIPVLR | |
| | DLTQQTLNLFTSKASSAAWNATLLDSFCNDLHQQLNDLQT | |
| | CLMQQVGVQEPPLTQEDALLAVRKYFHRITVYLREKKHSP | |
| | CAWEVVRAEVWRALSSSVNLLPRLSEEKE | |
| IL1a | MAKVPDMFEDLKNCYSENEEDSSSIDHLSLNQKSFYHVSY | 99 |
| (human) | GPLHEGCMDQSVSLSISETSKTSKLTFKESMVVVATNGKVL | |
| | KKRRLSLSQSITDDDLEAIANDSEEEIIKPRSAPFSFLSNVKY | |
| | NFMRIIKYEFILNDALNQSIIRANDQYLTAAALHNLDEAVKFD | |
| | MGAYKSSKDDAKITVILRISKTQLYVTAQDEDQPVLLKEMP | |
| | EIPKTITGSETNLLFFWETHGTKNYFTSVAHPNLFIATKQDY | |
| | WVCLAGGPPSITDFQILENQA | |
| mature IL1a | SAPFSFLSNVKYNFMRIIKYEFILNDALNQSIIRANDQYLTAA | 100 |
| (human) | ALHNLDEAVKFDMGAYKSSKDDAKITVILRISKTQLYVTAQD | |
| | EDQPVLLKEMPEIPKTITGSETNLLFFWETHGTKNYFTSVA | |
| | HPNLFIATKQDYWVCLAGGPPSITDFQILENQA | |
| IL1b | MAEVPELASEMMAYYSGNEDDLFFEADGPKQMKCSFQDL | 101 |
| (human) | DLCPLDGGIQLRISDHHYSKGFRQAASVVVAMDKLRKMLV | |
| | PCPQTFQENDLSTFFPFIFEEEPIFFDTWDNEAYVHDAPVR | |
| | SLNCTLRDSQQKSLVMSGPYELKALHLQGQDMEQQVVFS | |
| | MSFVQGEESNDKIPVALGLKEKNLYLSCVLKDDKPTLQLES | |
| | VDPKNYPKKKMEKRFVFNKIEINNKLEFESAQFPNWYISTS | |
| | QAENMPVFLGGTKGGQDITDFTMQFVSS | |
| mature IL1b | APVRSLNCTLRDSQQKSLVMSGPYELKALHLQGQDMEQQ | 102 |
| (human) | VVFSMSFVQGEESNDKIPVALGLKEKNLYLSCVLKDDKPTL | |
| | QLESVDPKNYPKKKMEKRFVFNKIEINNKLEFESAQFPNWY | |
| | ISTSQAENMPVFLGGTKGGQDITDFTMQFVSS | |
| IL18 | MAAEPVEDNCINFVAMKFIDNTLYFIAEDDENLESDYFGKLE | 103 |
| (human) | SKLSVIRNLNDQVLFIDQGNRPLFEDMTDSDCRDNAPRTIFI | |
| | ISMYKDSQPRGMAVTISVKCEKISTLSCENKIISFKEMNPPD | |
| | NIKDTKSDIIFFQRSVPGHDNKMQFESSSYEGYFLACEKER | |
| | DLFKLILKKEDELGDRSIMFTVQNED | |
| mature IL18 | YFGKLESKLSVIRNLNDQVLFIDQGNRPLFEDMTDSDCRDN | 104 |
| (human) | APRTIFIISMYKDSQPRGMAVTISVKCEKISTLSCENKIISFKE | |
| | MNPPDNIKDTKSDIIFFQRSVPGHDNKMQFESSSYEGYFLA | |
| | CEKERDLFKLILKKEDELGDRSIMFTVQNED | |
| mature IL18DR | YFGKLESKLSVIRNLNDQVLFIDQGNRPLFEDMTDSDCRDN | 105 |
| (human) | APRTIFIISKYSDSLARGLAVTISVKCEKISTLSCENKIISFKE | |
| | MNPPDNIKDTKSDIIFFQRDVPGHSRKMQFESSSYEGYFLA | |
| | CEKERDLFKLILKKEDELGDRSIMFTVQNED | |
| IFN-alpha-2 | MALTFALLVALLVLSCKSSCSVGCDLPQTHSLGSRRTLMLL | 106 |
| (human) | AQMRRISLFSCLKDRHDFGFPQEEFGNQFQKAETIPVLHE | |
| | MIQQIFNLFSTKDSSAAWDETLLDKFYTELYQQLNDLEACVI | |
| | QGVGVTETPLMKEDSILAVRKYFQRITLYLKEKKYSPCAWE | |
| | VVRAEIMRSFSLSTNLQESLRSKE | |

### Linkers

Methods of linking molecules are well known in the art. The linker may be a peptide linker or a non-peptide linker. If the linker is a peptide linker, it may be composed of one or more amino acids. For peptide linkers, typically a small linker sequence of glycine and serine (termed a GS mini-linker) amino acids are used. The number of amino acids in the linker can vary, from 4 (GGGS) (SEQ ID NO:9), 6 (GGSGGS) (SEQ ID NO:10), 10 (GGGGSGGGGS) (SEQ ID NO:11), 15 (GGGGSGGGGSGGGGS) (SEQ ID NO:12), 20 (GGGGSGGGGSGGGGSGGGGS) (SEQ ID NO:13) or more.

In some embodiments, the linker is between 5 and 20 amino acids in length. In other embodiments, the linker is rich in amino acid residues G and S. In another embodiment, the linker is between 5 and 20 amino acids in length and is rich in amino acid residues G and S. In another embodiment, the linker only includes the amino acid residues G and S. In another embodiment, the linker is between 2 and 20 amino acids in length and only includes the the amino acid residues G and S.

Peptide linkers, as envisaged herein, are (poly)peptide linkers of at least 1 amino acid in length. Preferably, the linkers are 1 to 100 amino acids in length. More preferably, the linkers are 5 to 50 amino acids in length, more preferably 10 to 40 amino acids in length, and even more preferably, the linkers are 15 to 30 amino acids in length. Non-limiting examples of often used small linkers include sequences of glycine and serine amino acids, termed GS mini-linker. Preferred examples of linker sequences are Gly/Ser linkers of different length such as (glyₓser_{y})_{z} linkers, including (gly₄ser)₃, (gly₄ser)₄, (gly₄ser), (gly₃ser), gly₃, and (gly₃ser₂)₃. The number of amino acids in these linkers can vary, for example, they can be 4 (e.g., GGGS) (SEQ ID NO: 9), 6 (e.g., GGSGGS) (SEQ ID NO:10), 7 (e.g., GGGSGGS), or multiples thereof, such as e.g. two or three or more repeats of these four/six amino acids. Most preferably, such GS mini-linkers have 20 amino acids and the sequence GGGGSGGGGSGGGGSGGGGS (SEQ ID NO:13). Further examples of such linkers include GGGGSGGGG (SEQ ID NO:14), GSGG (SEQ ID NO:15), or GGGGSGGGGSGGGGSGGGGSGGGGS (SEQ ID NO:16).

Further examples of linkers include the following:

5GS linker:
(SEQ ID NO: 17)
GGGGS
7GS linker:
(SEQ ID NO: 18)
SGGSGGS
8GS linker:
(SEQ ID NO: 19)
GGGSGGGS
9GS linker:
(SEQ ID NO: 20)
GGGGSGGGS
10GS linker:
(SEQ ID NO: 21)
GGGGSGGGGS
15GS linker:
(SEQ ID NO: 22)
GGGGSGGGGSGGGGS
18GS linker:
(SEQ ID NO: 23)
GGGGSGGGGSGGGGGGGS
20GS linker:
(SEQ ID NO: 24)
GGGGSGGGGSGGGGSGGGGS
25GS linker:
(SEQ ID NO: 25)
GGGGSGGGGSGGGGSGGGGSGGGGS
30GS linker:
(SEQ ID NO: 26)
GGGGSGGGGSGGGGGGGGSGGGGSGGGGS
35GS linker:
(SEQ ID NO: 27)
GGGGSGGGGSGGGGSGGGGSGGGGSGGGGSGGGGS

Said linker can be also a variant as described in Holliger et al. (1993), Proc. Natl. Acad. Sci. USA 90:6444-6448. Other linkers that can be used for the present invention are described by Alfthan et al. (1995), Protein Eng. 8:725-731, Choi et al. (2001), Eur. J. Immunol. 31:94-106, Hu et al. (1996), Cancer Res. 56:3055-3061, Kipriyanov et al. (1999), J. Mol. Biol. 293:41-56 and Roovers et al. (2001), Cancer Immunol. Immunother. 50:51-59.

In a preferred embodiment, the linker is selected from any of the aforementioned GS linkers. In a further preferred embodiment, the linker is between 2 and 20 amino acids in length and only includes the amino acid residues G and S.

### Rhabdoviruses

The family of rhabdoviruses includes 18 genera and 134 species with negative-sense, single-stranded RNA genomes of approximately 10-16 kb (Walke et al., ICTV Virus Taxonomy Profile: Rhabdoviridae, Journal of General Virology, 99:447-448 (2018)).

Characterizing features of members of the family of rhabdoviruses include one or more of the following: A bullet-shaped or bacilliform particle 100-430 nm in length and 45-100 nm in diameter comprised of a helical nucleocapsid surrounded by a matrix layer and a lipid envelope, wherein some rhabdoviruses have non-enveloped filamentous viruses. A negative-sense, single-stranded RNA of 10.8-16.1 kb, which are mostly unsegmented. A genome encoding for at least 5 genes encoding the structural proteins nucleoprotein (N), large protein (L), phosphoprotein (P), matrix protein (M), and glycoprotein (G).

As used herein a rhabdovirus can belong to the genus of: almendravirus, curiovirus, cytorhabdovirus, dichorhavirus, ephemerovirus, Hapavirus, ledantevirus, lyssavirus, novirhabdovirus, nucleorhabdovirus, perhabdovirus, sigmavirus, sprivivirus, sripuvirus, tibrovirus, tupavirus, varicosavirus or vesiculovirus.

Within the genus mentioned herein the rhabdovirus can belong to any of the listed species. The genus of almendravirus includes: arboretum almendravirus, balsa almendravirus, Coot Bay almendravirus, Puerto Almendras almendravirus, Rio Chico almendravirus; the genus of curiovirus includes: curionopolis curiovirus, Iriri curiovirus, Itacaiunas curiovirus, Rochambeau curiovirus; the genus of cythorhabdovirus includes: Alfalfa dwarf cytorhabdovirus, Barley yellow striate mosaic cytorhabdovirus, Broccoli necrotic yellows cytorhabdovirus, Colocasia bobone disease-associated cytorhabdovirus, Festuca leaf streak cytorhabdovirus, Lettuce necrotic yellows cytorhabdovirus, Lettuce yellow mottle cytorhabdovirus, Northern cereal mosaic cytorhabdovirus, Sonchus cytorhabdovirus 1, Strawberry crinkle cytorhabdovirus, Wheat American striate mosaic cytorhabdovirus; the genus of dichorhavirus includes: Coffee ringspot dichorhavirus, Orchid fleck dichorhavirus; the genus of ephemerovirus includes: Adelaide River ephemerovirus, Berrimah ephemerovirus, Bovine fever ephemerovirus, Kimberley ephemerovirus, Koolpinyah ephemerovirus, Kotonkan ephemerovirus, Obodhiang ephemerovirus, Yata ephemerovirus; the genus of hapavirus includes: Flanders hapavirus, Gray Lodge hapavirus, Hart Park hapavirus, Joinjakaka hapavirus, Kamese hapavirus, La Joya hapavirus, Landjia hapavirus, Manitoba hapavirus, Marco hapavirus, Mosqueiro hapavirus, Mossuril hapavirus, Ngaingan hapavirus, Ord River hapavirus, Parry Creek hapavirus, Wongabel hapavirus; the genus of ledantevirus includes: Barur ledantevirus, Fikirini ledantevirus, Fukuoka ledantevirus, Kanyawara ledantevirus, Kern Canyon ledantevirus, Keuraliba ledantevirus, Kolente ledantevirus, Kumasi ledantevirus, Le Dantec ledantevirus, Mount Elgon bat ledantevirus, Nishimuro ledantevirus, Nkolbisson ledantevirus, Oita ledantevirus, Wuhan ledantevirus, Yongjia ledantevirus; the genus of lyssavirus includes: Aravan lyssavirus, Australian bat lyssavirus, Bokeloh bat lyssavirus, Duvenhage lyssavirus, European bat 1 lyssavirus, European bat 2 lyssavirus, Gannoruwa bat lyssavirus, Ikoma lyssavirus, Irkut lyssavirus, Khujand lyssavirus, Lagos bat lyssavirus, Lleida bat lyssavirus, Mokola lyssavirus, Rabies lyssavirus, Shimoni bat lyssavirus, West Caucasian bat lyssavirus; the genus of novirhabdovirus includes: Hirame novirhabdovirus, Piscine novirhabdovirus, Salmonid novirhabdovirus, Snakehead novirhabdovirus; the genus of nucleorhabdovirus includes: Datura yellow vein nucleorhabdovirus, Eggplant mottled dwarf nucleorhabdovirus, Maize fine streak nucleorhabdovirus, Maize Iranian mosaic nucleorhabdovirus, Maize mosaic nucleorhabdovirus, Potato yellow dwarf nucleorhabdovirus, Rice yellow stunt nucleorhabdovirus, Sonchus yellow net nucleorhabdovirus, Sowthistle yellow vein nucleorhabdovirus, Taro vein chlorosis nucleorhabdovirus; the genus of perhabdovirus includes: Anguillid perhabdovirus, Perch perhabdovirus, Sea trout perhabdovirus; the genus of sigmavirus includes: Drosophila affinis sigmavirus, Drosophila ananassae sigmavirus, Drosophila immigrans sigmavirus, Drosophila melanogaster sigmavirus, Drosophila obscura sigmavirus, Drosophila tristis sigmavirus, Muscina stabulans sigmavirus; the genus of sprivivirus includes: Carp sprivivirus, Pike fry sprivivirus; the genus of Sripuvirus includes: Almpiwar sripuvirus, Chaco sripuvirus, Niakha sripuvirus, Sena Madureira sripuvirus, Sripur sripuvirus; the genus of tibrovirus includes: Bas-Congo tibrovirus, Beatrice Hill tibrovirus, Coastal Plains tibrovirus, Ekpoma 1 tibrovirus, Ekpoma 2 tibrovirus, Sweetwater Branch tibrovirus, tibrogargan tibrovirus; the genus of tupavirus includes: Durham tupavirus, Klamath tupavirus, Tupaia tupavirus; the genus of varicosavirus includes: Lettuce big-vein associated varicosavirus; the genus of vesiculovirus includes: Alagoas vesiculovirus, American bat vesiculovirus, Carajas vesiculovirus, Chandipura vesiculovirus, Cocal vesiculovirus, Indiana vesiculovirus, Isfahan vesiculovirus, Jurona vesiculovirus, Malpais Spring vesiculovirus, Maraba vesiculovirus, Morreton vesiculovirus, New Jersey vesiculovirus, Perinet vesiculovirus, Piry vesiculovirus, Radi vesiculovirus, Yug Bogdanovac vesiculovirus, or Moussa virus.

Preferably, the recombinant rhabdovirus of the invention is an oncolytic rhabdovirus. In this respect, oncolytic has its regular meaning known in the art and refers to the ability of a rhabdovirus to infect and lyse (break down) cancer cells but not normal cells (to any significant extend). Preferably, the oncolytic rhabdovirus is capable of replication within cancer cells. Oncolytic activity may be tested in different assay systems known to the skilled artisan (an exemplary in vitro assay is described by Muik et al., Cancer Res., 74(13), 3567-78, 2014). It is to be understood that an oncolytic rhabdovirus may infect and lyse only specific types of cancer cells. Also, the oncolytic effect may vary depending on the type of cancer cells.

In a preferred embodiment, the rhabdovirus belongs to the genus of vesiculovirus. Vesiculovirus species have been defined primarily by serological means coupled with phylogenetic analysis of the genomes. Biological characteristics such as host range and mechanisms of transmission are also used to distinguish viral species within the genus. As such, the genus of vesiculovirus form a distinct monophyletic group well-supported by Maximum Likelihood trees inferred from complete L sequences.

The term “recombinant” refers to a virus, more particularly a rhabdovirus, comprising an exogenous nucleic acid sequence inserted in its genome, which is not naturally present in the parent virus. A recombinant virus thus refers to a nucleic acid or virus made by an artificial combination of two or more segments of nucleic acid sequence of synthetic or semisynthetic origin which does not occur in nature or is linked to another nucleic acid in an arrangement not found in nature. The artificial combination is most commonly accomplished by artificial manipulation of isolated segments of nucleic acids, using well-established genetic engineering techniques. Generally, a “recombinant” rhabdovirus virus as described herein refers to rhabdoviruses that are produced by standard genetic engineering methods, e.g., rhabdoviruses of the present invention are thus genetically engineered or genetically modified viruses. The term “recombinant rhabdovirus” thus includes viruses, which have stably integrated recombinant nucleic acid in their genome.

Viruses assigned to different species within the genus vesiculovirus may have one or more of the following characteristics: A) a minimum amino acid sequence divergence of 20% in L; B) a minimum amino acid sequence divergence of 10% in N; C) a minimum amino acid sequence divergence of 15% in G; D) can be distinguished in serological tests; and E) occupy different ecological niches as evidenced by differences in hosts and or arthropod vectors.

Preferred is the vesicular stomatitis virus (VSV) and in particular the VSV-GP (recombinant with GP of LCMV). Advantageous properties of the VSV-GP include one or more of the following: very potent and fast killer (<8h); oncolytic virus; systemic application possible; reduced neurotropism/neurotoxicity; it reproduces lytically and induces immunogenic cell death; does not replicate in healthy human cells, due to interferon (IFN) response; strong activation of innate immunity; about 3 kb space for immunomodulatory cargos and antigens; recombinant with an arenavirus glycoprotein from the Lympho-Chorio-Meningitis-Virus (LCMV); favorable safety features in terms of reduced neurotoxicity and less sensitive to neutralizing antibody responses and complement destruction as compared to the wild type VSV (VSV-G); specifically replicates in tumor cells, which have lost the ability to mount and respond to anti-viral innate immune responses (e.g. type-I IFN signaling); abortive replication in “healthy cells” so is rapidly excluded from normal tissues; viral replication in tumor cells leads to the induction of immunogenic cell death, release of tumor associated antigens, local inflammation and the induction of anti-tumor immunity.

In a preferred embodiment the recombinant vesicular stomatitis virus encodes in its genome at least for a vesicular stomatitis virus nucleoprotein (N) comprising an amino acid sequence as set forth in SEQ ID NO:28 or a functional variant at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:28, a phosphoprotein (P) comprising an amino acid sequence as set forth in SEQ ID NO:29 or a functional variant at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:29, a large protein (L) comprising an amino acid sequence as set forth in SEQ ID NO:30 or a functional variant at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:30, and a matrix protein (M) comprising an amino acid sequence as set forth in SEQ ID NO: 31 or a functional variant at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 31.

It is understood by the skilled artisan that modifications to the vesicular stomatitis virus nucleoprotein (N), large protein (L), phosphoprotein (P), matrix protein (M), or glycoprotein (G) sequence can be made without losing the basic functions of those proteins. Such functional variants as used herein retain all or part of their basic function or activity. The protein L for example is the polymerase and has an essential function during transcription and replication of the virus. A functional variant thereof must retain at least part of this ability. A good indication for retention of basic functionality or activity is the successful production of viruses, including these functional variants, that are still capable to replicate and infect tumor cells. Production of viruses and testing for infection and replication in tumor cells may be tested in different assay systems known to the skilled artisan (an exemplary in vitro assay is described by Muik et al., Cancer Res., 74(13), 3567-78, 2014).

In a preferred embodiment the recombinant vesicular stomatitis virus encodes in its genome at least for a vesicular stomatitis virus nucleoprotein (N), large protein (L), phosphoprotein (P), matrix protein (M), glycoprotein (G) and at least one GSDM or a functional variant thereof, wherein the large protein (L) comprises an amino acid sequence having a sequence identity≥80% of SEQ ID NO:30.

In a preferred embodiment the recombinant vesicular stomatitis virus encodes in its genome at least for a vesicular stomatitis virus nucleoprotein (N), large protein (L), phosphoprotein (P), matrix protein (M), glycoprotein (G) and at least one GSDM or a functional variant thereof, wherein the nucleoprotein (N) comprises an amino acid sequence having a sequence identity≥90% of SEQ ID NO:28.

In a further preferred embodiment the recombinant vesicular stomatitis virus encodes in its genome at least for a vesicular stomatitis virus nucleoprotein (N), large protein (L), phosphoprotein (P), matrix protein (M), glycoprotein (G) and at least one GSDM or a functional variant thereof, wherein the large protein (L) comprises an amino acid sequence having a sequence identity equal or greater 80% of SEQ ID NO: 30 and the nucleoprotein (N) comprises an amino acid sequence having a sequence identity≥90% of SEQ ID NO:28.

The invention is further embodied by a recombinant vesicular stomatitis virus, encoding in its genome at least for a vesicular stomatitis virus nucleoprotein (N), large protein (L), phosphoprotein (P), matrix protein (M), glycoprotein (G) and at least one GSDM or a functional variant thereof, preferably comprising human GSDM. In a preferred embodiment, the GSDM is GSDME, preferably human GSDME. In a further preferred embodiment, the genome encodes for GSDME and further for IL12.

In one embodiment, the recombinant vesicular stomatitis virus does not code for a mutated matrix protein (M) and in particular does not code for VSVA51M which has a codon deletion in the gene coding for the M protein, at amino acid position 51.

It is to be understood that a recombinant rhabdovirus of the invention may encode in its genome further cargos, such as tumor antigens, further chemokines, or other immunomodulatory elements.

In a further embodiment the recombinant rhabdovirus of the invention additionally encodes in its genome a sodium iodide symporter protein (NIS). Expression of NIS and co-incubation with e.g. ¹²⁵I allows the use of NIS as imaging reporter (Carlson et al., Current Gene Therapy, 12, 33-47, 2012).

TABLE 4

| Identifier | Sequence | SEQ ID NO: |
|---|---|---|
| VSV N | MSVTVKRIIDNTVVVPKLPANEDPVEYPADYFRKSKEIPLYI | 28 |
| | NTTKSLSDLRGYVYQGLKSGNVSIIHVNSYLYGALKDIRGKL | |
| | DKDWSSFGINIGKAGDTIGIFDLVSLKALDGVLPDGVSDAS | |
| | RTSADDKWLPLYLLGLYRVGRTQMPEYRKKLMDGLTNQC | |
| | KMINEQFEPLVPEGRDIFDVWGNDSNYTKIVAAVDMFFHM | |
| | FKKHECASFRYGTIVSRFKDCAALATFGHLCKITGMSTEDV | |
| | TTWILNREVADEMVQMMLPGQEIDKADSYMPYLIDFGLSS | |
| | KSPYSSVKNPAFHFWGQLTALLLRSTRARNARQPDDIEYT | |
| | SLTTAGLLYAYAVGSSADLAQQFCVGDNKYTPDDSTGGLT | |
| | TNAPPQGRDVVEWLGWFEDQNRKPTPDMMQYAKRAVMS | |
| | LQGLREKTIGKYAKSEFDK | |
| VSV P | MDNLTKVREYLKSYSRLDQAVGEIDEIEAQRAEKSNYELFQ | 29 |
| | EDGVEEHTKPSYFQAADDSDTESEPEIEDNQGLYAPDPEA | |
| | EQVEGFIQGPLDDYADEEVDVVFTSDWKQPELESDEHGKT | |
| | LRLTSPEGLSGEQKSQWLSTIKAVVQSAKYWNLAECTFEA | |
| | SGEGVIMKERQITPDVYKVTPVMNTHPSQSEAVSDVWSLS | |
| | KTSMTFQPKKASLQPLTISLDELFSSRGEFISVGGDGRMSH | |
| | KEAILLGLRYKKLYNQARVKYSL | |
| VSV L | MEVHDFETDEFNDFNEDDYATREFLNPDERMTYLNHADY | 30 |
| | NLNSPLISDDIDNLIRKFNSLPIPSMWDSKNWDGVLEMLTS | |
| | CQANPIPTSQMHKWMGSWLMSDNHDASQGYSFLHEVDK | |
| | EAEITFDVVETFIRGWGNKPIEYIKKERWTDSFKILAYLCQK | |
| | FLDLHKLTLILNAVSEVELLNLARTFKGKVRRSSHGTNICRIR | |
| | VPSLGPTFISEGWAYFKKLDILMDRNFLLMVKDVIIGRMQTV | |
| | LSMVCRIDNLFSEQDIFSLLNIYRIGDKIVERQGNFSYDLIKM | |
| | VEPICNLKLMKLARESRPLVPQFPHFENHIKTSVDEGAKIDR | |
| | GIRFLHDQIMSVKTVDLTLVIYGSFRHWGHPFIDYYTGLEKL | |
| | HSQVTMKKDIDVSYAKALASDLARIVLFQQFNDHKKWFVN | |
| | GDLLPHDHPFKSHVKENTWPTAAQVQDFGDKWHELPLIKC | |
| | FEIPDLLDPSIIYSDKSHSMNRSEVLKHVRMNPNTPIPSKKV | |
| | LQTMLDTKATNWKEFLKEIDEKGLDDDDLIIGLKGKERELKL | |
| | AGRFFSLMSWKLREYFVITEYLIKTHFVPMFKGLTMADDLT | |
| | AVIKKMLDSSSGQGLKSYEAICIANHIDYEKWNNHQRKLSN | |
| | GPVFRVMGQFLGYPSLIERTHEFFEKSLIYYNGRPDLMRVH | |
| | NNTLINSTSQRVCWQGQEGGLEGLRQKGWSILNLLVIQRE | |
| | AKIRNTAVKVLAQGDNQVICTQYKTKKSRNVVELQGALNQ | |
| | MVSNNEKIMTAIKIGTGKLGLLINDDETMQSADYLNYGKIPIF | |
| | RGVIRGLETKRWSRVTCVTNDQIPTCANIMSSVSTNALTVA | |
| | HFAENPINAMIQYNYFGTFARLLLMMHDPALRQSLYEVQDK | |
| | IPGLHSSTFKYAMLYLDPSIGGVSGMSLSRFLIRAFPDPVTE | |
| | SLSFWRFIHVHARSEHLKEMSAVFGNPEIAKFRITHIDKLVE | |
| | DPTSLNIAMGMSPANLLKTEVKKCLIESRQTIRNQVIKDATIY | |
| | LYHEEDRLRSFLWSINPLFPRFLSEFKSGTFLGVADGLISLF | |
| | QNSRTIRNSFKKKYHRELDDLIVRSEVSSLTHLGKLHLRRG | |
| | SCKMWTCSATHADTLRYKSWGRTVIGTTVPHPLEMLGPQ | |
| | HRKETPCAPCNTSGFNYVSVHCPDGIHDVFSSRGPLPAYL | |
| | GSKTSESTSILQPWERESKVPLIKRATRLRDAISWFVEPDS | |
| | KLAMTILSNIHSLTGEEWTKRQHGFKRTGSALHRFSTSRMS | |
| | HGGFASQSTAALTRLMATTDTMRDLGDQNFDFLFQATLLY | |
| | AQITTTVARDGWITSCTDHYHIACKSCLRPIEEITLDSSMDY | |
| | TPPDVSHVLKTWRNGEGSWGQEIKQIYPLEGNWKNLAPA | |
| | EQSYQVGRCIGFLYGDLAYRKSTHAEDSSLFPLSIQGRIRG | |
| | RGFLKGLLDGLMRASCCQVIHRRSLAHLKRPANAVYGGLIY | |
| | LIDKLSVSPPFLSLTRSGPIRDELETIPHKIPTSYPTSNRDMG | |
| | VIVRNYFKYQCRLIEKGKYRSHYSQLWLFSDVLSIDFIGPFSI | |
| | STTLLQILYKPFLSGKDKNELRELANLSSLLRSGEGWEDIHV | |
| | KFFTKDILLCPEEIRHACKFGIAKDNNKDMSYPPWGRESRG | |
| | TITTIPVYYTTTPYPKMLEMPPRIQNPLLSGIRLGQLPTGAH | |
| | YKIRSILHGMGIHYRDFLSCGDGSGGMTAALLRENVHSRGI | |
| | FNSLLELSGSVMRGASPEPPSALETLGGDKSRCVNGETC | |
| | WEYPSDLCDPRTWDYFLRLKAGLGLQIDLIVMDMEVRDSS | |
| | TSLKIETNVRNYVHRILDEQGVLIYKTYGTYICESEKNAVTIL | |
| | GPMFKTVDLVQTEFSSSQTSEVYMVCKGLKKLIDEPNPDW | |
| | SSINESWKNLYAFQSSEQEFARAKKVSTYFTLTGIPSQFIPD | |
| | PFVNIETMLQIFGVPTGVSHAAALKSSDRPADLLTISLFYMAI | |
| | ISYYNINHIRVGPIPPNPPSDGIAQNVGIAITGISFWLSLMEK | |
| | DIPLYQQCLAVIQQSFPIRWEAVSVKGGYKQKWSTRGDGL | |
| | PKDTRISDSLAPIGNWIRSLELVRNQVRLNPFNEILFNQLCR | |
| | TVDNHLKWSNLRRNTGMIEWINRRISKEDRSILMLKSDLHE | |
| | ENSWRD | |
| VSV M | MSSLKKILGLKGKGKKSKKLGIAPPPYEEDTSMEYAPSAPID | 31 |
| | KSYFGVDEMDTYDPNQLRYEKFFFTVKMTVRSNRPFRTYS | |
| | DVAAAVSHWDHMYIGMAGKRPFYKILAFLGSSNLKATPAVL | |
| | ADQGQPEYHAHCEGRAYLPHRMGKTPPMLNVPEHFRRPF | |
| | NIGLYKGTIELTMTIYDDESLEAAPMIWDHFNSSKFSDFREK | |
| | ALMFGLIVEKKASGAWVLDSIGHFK | |
| LCMV GP | MGQIVTMFEALPHIIDEVINIVIIVLIIITSIKAVYNFATCGILALV | 32 |
| | SFLFLAGRSCGMYGLNGPDIYKGVYQFKSVEFDMSHLNLT | |
| | MPNACSANNSHHYISMGSSGLELTFTNDSILNHNFCNLTSA | |
| | FNKKTFDHTLMSIVSSLHLSIRGNSNHKAVSCDFNNGITIQY | |
| | NLSFSDPQSAISQCRTFRGRVLDMFRTAFGGKYMRSGWG | |
| | WAGSDGKTTWCSQTSYQYLIIQNRTWENHCRYAGPFGMS | |
| | RILFAQEKTKFLTRRLAGTFTWTLSDSSGVENPGGYCLTK | |
| | WMILAAELKCFGNTAVAKCNVNHDEEFCDMLRLIDYNKAAL | |
| | SKFKQDVESALHVFKTTVNSLISDQLLMRNHLRDLMGVPY | |
| | CNYSKFWYLEHAKTGETSVPKCWLVTNGSYLNETHFSDQI | |
| | EQEADNMITEMLRKDYIKRQGSTPLALMDLLMFSTSAYLISI | |
| | FLHLVKIPTHRHIKGGSCPKPHRLTNKGICSCGAFKVPGVK | |
| | TIWKRR | |
| Dandenong GP | MGQLITMFEALPHIIDEVINIVIIVLVIITSIKAVYNFATCGIIALIS | 33 |
| | FCLLAGRSCGLYGVTGPDIYKGLYQFKSVEFNMSQLNLTM | |
| | PNACSANNSHHYISMGKSGLELTFTNDSIISHNFCNLTDGF | |
| | KKKTFDHTLMSIVASLHLSIRGNTNYKAVSCDFNNGITIQYN | |
| | LSFSDAQSAINQCRTFRGRVLDMFRTAFGGKYMRSGYGW | |
| | KGSDGKTTWCSQTSYQYLIIQNRTWENHCEYAGPFGLSRV | |
| | LFAQEKTKFLTRRLAGTFTWTLSDSSGTENPGGYCLTKWM | |
| | LIAAELKCFGNTAVAKCNINHDEEFCDMLRLIDYNKAALKKF | |
| | KEDVESALHLFKTTVNSLISDQLLMRNHLRDLMGVPYCNYS | |
| | KFWYLEHVKTGDTSVPKCWLVSNGSYLNETHFSDQIEQEA | |
| | DNMITEMLRKDYIKRQGSTPLALMDLLMFSTSAYLISVFLHL | |
| | MKIPTHRHIKGGTCPKPHRLTSKGICSCGAFKVPGVKTVWK | |
| | RR | |
| Mopeia GP | MGQIVTFFQEVPHILEEVMNIVLMTLSILAILKGIYNVMTCGII | 34 |
| | GLITFLFLCGRSCSSIYKDNYEFFSLDLDMSSLNATMPLSCS | |
| | KNNSHHYIQVGNETGLELTLTNTSIIDHKFCNLSDAHRRNLY | |
| | DKALMSILTTFHLSIPDFNQYEAMSCDFNGGKISIQYNLSHS | |
| | NYVDAGNHCGTIANGIMDVFRRMYWSTSLSVASDISGTQCI | |
| | QTDYKYLIIQNTSWEDHCMFSRPSPMGFLSLLSQRTRNFYI | |
| | SRRLLGLFTWTLSDSEGNDMPGGYCLTRSMLIGLDLKCFG | |
| | NTAIAKCNQAHDEEFCDMLRLFDFNKQAISKLRSEVQQSIN | |
| | LINKAVNALINDQLVMRNHLRDLMGIPYCNYSKFWYLNDTR | |
| | TGRTSLPKCWLVTNGSYLNETQFSTEIEQEANNMFTDMLR | |
| | KEYEKRQSTTPLGLVDLFVFSTSFYLISVFLHLIKIPTHRHIK | |
| | GKPCPKPHRLNHMAICSCGFYKQPGLPTQWKR | |
| deltaM51 | MSSLKKILGLKGKGKKSKKLGIAPPPYEEDTSMEYAPSAPID | 85 |
| | KSYFGVDEDTYDPNQLRYEKFFFTVKMTVRSNRPFRTYSD | |
| | VAAAVSHWDHMYIGMAGKRPFYKILAFLGSSNLKATPAVLA | |
| | DQGQPEYHAHCEGRAYLPHRMGKTPPMLNVPEHFRRPFN | |
| | IGLYKGTIELTMTIYDDESLEAAPMIWDHFNSSKFSDFREKA | |
| | LMFGLIVEKKASGAWVLDSIGHFK | |

In a preferred embodiment of the invention the RNA genome of the recombinant rhabdovirus of the invention comprises or consists of a sequence as shown in SEQ ID NO:111. Furthermore, the RNA genome of the recombinant rhabdovirus of the invention may also consist of or comprise those sequences, wherein nucleic acids of the RNA genome are exchanged according to the degeneration of the genetic code, without leading to an alteration of the respective amino acid sequence. In a further preferred embodiment, the RNA genome of the recombinant rhabdovirus of the invention comprises or consists of a coding sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:111.

### Pseudotyped Rhabdovirus

It is known that certain wildtype rhabdovirus strains such as wildtype VSV strains are considered to be neurotoxic. It is also reported that infected individuals are able to rapidly mount a strong humoral response with high antibody titers directed mainly against the glycoprotein. Neutralizing antibodies targeting the glycoprotein G of rhabdoviruses in general and VSV specifically are able to limit virus spread and thereby mediate protection of individuals from virus re-infection. Virus neutralization, however, limits repeated application of the rhabdovirus to the cancer patient.

To eliminate these drawbacks the rhabdovirus wildtype glycoprotein G may be replaced with the glycoprotein from another virus. In this respect replacing the glycoprotein refers to (i) replacement of the gene coding for the wild type glycoprotein G with the gene coding for the glycoprotein GP of another virus, and/or (ii) replacement of the wild type glycoprotein G with the glycoprotein GP of another virus.

In a preferred embodiment the rhabdovirus glycoprotein G is replaced with the glycoprotein GP of the lymphocytic choriomeningitis virus (LCMV), preferably with the strain WE-HPI. In an even more preferred embodiment, the rhabdovirus is a vesicular stomatitis virus with the glycoprotein GP of the lymphocytic choriomeningitis virus (LCMV), preferably with the strain WE-HPI. Such VSV is for example described in WO2010/040526 and named VSV-GP. Advantages offered are (i) the loss of VSV-G mediated neurotoxicity and (ii) a lack of vector neutralization by antibodies (as shown in mice).

The glycoprotein GP of the lymphocytic choriomeningitis virus (LCMV) may be GP1 or GP2. The invention includes glycoproteins from different LCMV strains. In particular, LCMV-GP can be derived from LCMV wild-type or LCMV strains LCMV-WE, LCMV-WE-HPI, LCMV-WE-HPI opt. In a preferred embodiment, the gene coding for the glycoprotein GP of the LCMV encodes for a protein with an amino acid sequence as shown in SEQ ID NO:32 or an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 32 while the functional properties of the recombinant rhabdovirus comprising a glycoprotein GP encoding an amino acid sequence as shown in SEQ ID NO:32 are maintained.

In another embodiment the recombinant rhabdovirus glycoprotein G is replaced with the glycoprotein GP of the Dandenong virus (DANDV) or Mopeia (MOPV) virus. In a more preferred embodiment, the recombinant rhabdovirus is a vesicular stomatitis virus wherein the glycoprotein G is replaced with the glycoprotein GP of the Dandenong virus (DANDV) or Mopeia (MOPV) virus. Advantages offered are (i) the loss of VSV-G mediated neurotoxicity and (ii) a lack of vector neutralization by antibodies (as shown in mice).

The Dandenong virus (DANDV) is an old world arenavirus. To date, there is only a single strain known to the person skilled in the art, which comprise a glycoprotein GP and which may be employed within the present invention as donor of the glycoprotein GP comprised in the recombinant rhabdovirus of the invention. The DANDV glycoprotein GP comprised in the recombinant rhabdovirus of the invention has more than 6 glycosylation sites, in particular 7 glycosylation sites. An exemplary preferred glycoprotein GP is that as comprised in DANDV as accessible under Genbank number EU136038. In one embodiment, the gene coding for the glycoprotein GP of the DNADV encodes for an amino acid sequence as shown in SEQ ID NO:33 or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO:33 while the functional properties of the recombinant rhabdovirus comprising a glycoprotein GP encoding an amino acid sequence as shown in SEQ ID NO:33 are maintained.

The Mopeia virus (MOPV) is an old world arenavirus. There are several strains known to the person skilled in the art, which comprise a glycoprotein GP and which may be employed within the present invention as donor of the glycoprotein GP comprised in the recombinant rhabdovirus of the invention. The MOPV glycoprotein GP comprised in the recombinant rhabdovirus of the invention has more than 6 glycosylation sites, in particular 7 glycosylation sites. An exemplary preferred glycoprotein GP is that as comprised in Mopeia virus as accessible under Genbank number AY772170. In one embodiment, the gene coding for glycoprotein GP of the MOPV encodes for an amino acid sequence as shown in SEQ ID NO:34 or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO:34 while the functional properties of the recombinant rhabdovirus comprising a glycoprotein GP encoding an amino acid sequence as shown in SEQ ID NO:34 are maintained.

As used herein, the terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence. To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In some embodiments, the two sequences that are compared are the same length after gaps are introduced within the sequences, as appropriate (e.g., excluding additional sequence extending beyond the sequences being compared).

The determination of percent identity or percent similarity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. BLAST nucleotide searches can be performed with the NBLAST program, score=100, wordlength=12, to obtain nucleotide sequences homologous to a nucleic acid encoding a protein of interest. BLAST protein searches can be performed with the XBLAST program, score=50, wordlength=3, to obtain amino acid sequences homologous to protein of interest. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. USA 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. Alternatively, protein sequence alignment may be carried out using the CLUSTAL W algorithm, as described by Higgins et al., 1996, Methods Enzymol. 266:383-402.

### Pharmaceutical Compositions

The actual pharmaceutically effective amount or therapeutic dosage will of course depend on factors known by those skilled in the art such as age and weight of the patient, route of administration and severity of disease. In any case the recombinant rhabdovirus will be administered at dosages and in a manner which allows a pharmaceutically effective amount to be delivered based upon patient's unique condition.

Generally, for the treatment and/or alleviation of the diseases, disorders and conditions mentioned herein and depending on the specific disease, disorder or condition to be treated, the potency of the specific recombinant rhabdovirus of the invention to be used, the specific route of administration and the specific pharmaceutical formulation or composition used, the recombinant rhabdovirus of the invention will generally be administered for example, twice a week, weekly, or in monthly doses, but can significantly vary, especially, depending on the before-mentioned parameters. Thus, in some cases it may be sufficient to use less than the minimum dose given above, whereas in other cases the upper limit may have to be exceeded. When administering large amounts it may be advisable to divide them up into a number of smaller doses spread over the day.

To be used in therapy, the recombinant rhabdovirus of the invention is formulated into pharmaceutical compositions appropriate to facilitate administration to animals or humans. Typical formulations can be prepared by mixing the recombinant virus with physiologically acceptable carriers, excipients or stabilizers, in the form of aqueous solutions or aqueous or non-aqueous suspensions. Carriers, excipients, modifiers or stabilizers are nontoxic at the dosages and concentrations employed. They include buffer systems such as phosphate, citrate, acetate and other inorganic or organic acids and their salts; antioxidants including ascorbic acid and methionine; preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; proteins, such as serum albumin, gelatin, or immunoglobulins; hydrophilic polymers such as polyvinylpyrrolidone or polyethylene glycol (PEG); amino acids such as glycine, glutamine, asparagine, histidine, arginine, or lysine; monosaccharides, disaccharides, oligosaccharides or polysaccharides and other carbohydrates including glucose, mannose, sucrose, trehalose, dextrins or dextrans; chelating agents such as EDTA; sugar alcohols such as, mannitol or sorbitol; salt-forming counter-ions such as sodium; metal complexes (e.g., Zn-protein complexes); and/or ionic or non-ionic surfactants such as TWEEN™ (polysorbates), PLURONICS™ or fatty acid esters, fatty acid ethers or sugar esters. The excipients may also have a release-modifying or absorption-modifying function.

The pharmaceutical composition may be provided as a liquid, a frozen liquid or in a lyophilized form. The frozen liquid may be stored at temperatures between about 0° C. and about −85° C. including temperatures between −70° C. and −85° C. and of about −15° C., −16° C., −17° C., −18° C., −19° C., −20° C., −21° C., −22° C., −23° C., −24° C. or about −25° C.

The recombinant rhabdovirus or pharmaceutical composition of the invention need not be, but is optionally, formulated with one or more agents currently used to prevent or treat the disorder in question. The effective amount of such other agents depends on the amount of recombinant antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of the recombinant rhabdovirus or pharmaceutical composition of the invention (when used alone or in combination with one or more other additional therapeutic agents) will depend on the type of disease to be treated, the type of recombinant rhabdovirus, the severity and course of the disease, whether the recombinant rhabdovirus is administered for preventive or therapeutic purposes, previous therapy, the patient's clinical history and response to the recombinant rhabdovirus, and the discretion of the attending physician. The recombinant rhabdovirus or pharmaceutical composition of the invention suitably administered to the patient at one time or over a series of treatments.

Depending on the type and severity of the disease, about 106 to 1013 infectious particles measured by TCID50 of the recombinant rhabdovirus can be an initial candidate dosage for administration to the patient, whether, for example, by one or more separate administrations, or by continuous infusion. For repeated administrations over several days or longer, depending on the condition, the treatment would generally be sustained until a desired suppression of disease symptoms occurs. One exemplary dosage of the recombinant rhabdovirus would be in the range from about 10⁶ to 10¹³ infectious particles measured by TCID50. Thus, one or more doses of about 10⁶, 10⁷, 10⁸, 10⁹, 10¹⁰, 10¹¹, 10¹², or 10¹³ infectious particles measured by TCID50 (or any combination thereof) may be administered to the patient. Such doses may be administered intermittently, e.g. every week or every three weeks (e.g. such that the patient receives from about two to about twenty, or e.g. about six doses of the recombinant rhabdovirus). An initial higher loading dose, followed by one or more lower doses or vice versa may be administered. However, other dosage regimens may be useful. The progress of this therapy is easily monitored by conventional techniques and assays.

The efficacy of the recombinant rhabdovirus of the invention, and of compositions comprising the same, can be tested using any suitable in vitro assay, cell-based assay, in vivo assay and/or animal model known per se, or any combination thereof, depending on the specific disease involved. Suitable assays and animal models will be clear to the skilled person, and for example include the assays and animal models used in the Examples below.

The actual pharmaceutically effective amount or therapeutic dosage will of course depend on factors known by those skilled in the art such as age and weight of the patient, route of administration and severity of disease. In any case the recombinant rhabdovirus of the invention will be administered at dosages and in a manner which allows a pharmaceutically effective amount to be delivered based upon patient's unique condition.

Alternatively, the recombinant rhabdovirus or pharmaceutical composition of the invention may be delivered in a volume of from about 50 μl to about 100 ml including all numbers within the range, depending on the size of the area to be treated, the viral titer used, the route of administration, and the desired effect of the method.

For intratumoral administration the volume is preferably between about 50 μl to about 5 ml including volumes of about 100 μl, 200 μl, 300 μl, 400 μl, 500 μl, 600 μl, 700 μl, 800 μl, 900 μl, 1000 μl, 1100 μl, 1200 μl, 1300 μl, 1400 μl, 1500 μl, 1600 μl, 1700 μl, 1800 μl, 1900 μl, 2000 μl, 2500 μl, 3000 μl, 3500 μl, 4000 μl, or about 4500 μl. In a preferred embodiment the volume is about 1000 μl.

For systemic administration, e.g. by infusion of the recombinant rhabdovirus the volumes may be naturally higher. Alternatively, a concentrated solution of the recombinant rhabdovirus could be diluted in a larger volume of infusion solution directly before infusion.

In particular for intravenous administration the volume is preferably between 1 ml and 100 ml including volumes of about 2 ml, 3 ml, 4 ml, 5 ml, 6 ml, 7 ml, 8 ml, 9 ml, 10 ml, 11 ml, 12 ml, 13 ml, 14 ml, 15 ml, 16 ml, 17 ml, 18 ml, 19 ml, 20 ml, 25 ml, 30 ml, 35 ml, 40 ml, 45 ml, 50 ml, 55 ml, 60 ml, 70 ml, 75 ml, 80 ml, 85 ml, 90 ml, 95 ml, or about 100 ml. In a preferred embodiment the volume is between about 5 ml and 15 ml, more preferably the volume is about 6 ml, 7 ml, 8 ml, 9 ml, 10 ml, 11 ml, 12 ml, 13 ml, or about 14 ml.

Preferably the same formulation is used for intratumoral administration and intravenous administration. The doses and/or volume ratio between intratumoral and intravenous administration may be about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19 or about 1:20. For example, a doses and/or volume ratio of 1:1 means that the same doses and/or volume is administered intratumorally as well as intravenously, whereas e.g. a doses and/or volume ratio of about 1:20 means an intravenous administration dose and/or volume that is twenty times higher than the intratumoral administration dose and/or volume. Preferably, the doses and/or volume ratio between intratumoral and intravenous administration is about 1:9.

An effective concentration of a recombinant rhabdovirus desirably ranges between about 10⁸ and 10¹⁴ vector genomes per milliliter (vg/mL). The infectious units may be measured as described in Mclaughlin et al., J Virol.; 62(6):1963-73 (1988). Preferably, the concentration is from about 1.5×10⁹ to about 1.5×10¹³, and more preferably from about 1.5×10⁹ to about 1.5×10¹¹. In one embodiment, the effective concentration is about 1.5×10⁹. In another embodiment, the effective concentration is about 1.5×10¹⁰. In another embodiment, the effective concentration is about 1.5×10¹¹. In yet another embodiment, the effective concentration is about 1.5×10¹². In another embodiment, the effective concentration is about 1.5×10¹³. In another embodiment, the effective concentration is about 1.5×10¹⁴. It may be desirable to use the lowest effective concentration in order to reduce the risk of undesirable effects. Still other dosages in these ranges may be selected by the attending physician, taking into account the physical state of the subject, preferably human, being treated, the age of the subject, the particular type of cancer and the degree to which the cancer, if progressive, has developed.

An effective target concentration of a recombinant rhabdovirus may be expressed with the TCID50. The TCID50 can be calculated for example by using the method of Spearman-Karber. Desirably ranges include an effective target concentration between 1×10⁶/ml and 1×10¹⁴/ml TCID50. Preferably, the effective target concentration is from about 1×10⁶ to about 1×10¹²/ml, and more preferably from about 1×10⁶ to about 1×10¹¹/ml. In one embodiment, the effective target concentration is about 1×10¹⁰/ml. In a preferred embodiment the target concentration is 5×10¹⁰/ml. In another embodiment, the effective target concentration is about 1.5×10¹¹/ml. In one embodiment, the effective target concentration is about 1×10¹²/ml. In another embodiment, the effective target concentration is about 1.5×10¹³/ml.

An effective target dose of a recombinant rhabdovirus may also be expressed with the TCID₅₀. Desirably ranges include a target dose between 1×10⁶ and 1×10¹⁴ TCID₅₀. Preferably, the target dose is from about 1×10⁶ to about 1×10¹³, and more preferably from about 1×10⁶ to about 1×10¹². In one embodiment, the effective concentration is about 1×10¹⁰. In a preferred embodiment, the effective concentration is about 1×10¹¹. In one embodiment, the effective concentration is about 1×10¹². In another embodiment, the effective concentration is about 1×10¹³.

In another aspect, a kit or kit-of-parts containing materials useful for the treatment, prevention and/or diagnosis of the disorders described herein is provided. The kit or kit-of-parts comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the disorder and may have a sterile access port (for example the container may be an intravenous solution bag or a vial having a stopper pierceable by a hypodermic injection needle). At least one active agent in the composition is the recombinant rhabdovirus or pharmaceutical composition of the invention. The label or package insert indicates that the composition is used for treating the condition of choice.

Moreover, the kit or kit-of-parts may comprise (a) a first container with a composition contained therein, wherein the composition comprises the recombinant rhabdovirus or pharmaceutical composition of the invention; and (b) a second container with a composition contained therein, wherein the composition comprises a further cytotoxic or otherwise therapeutic agent, such as a PD-1 pathway inhibitor or SMAC mimetic. The kit or kit-of-parts in this embodiment of the invention may further comprise a package insert indicating that the compositions can be used to treat a particular condition, in particular cancer. Alternatively, or additionally, the kit or kit-of-parts may further comprise a second (or third) container comprising a pharmaceutically-acceptable buffer, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

In a further aspect, a recombinant rhabdovirus of the invention is used in combination with a device useful for the administration of the recombinant rhabdovirus, such as a syringe, injector pen, micropump, or other device. Preferably, a recombinant rhabdovirus of the invention is comprised in a kit of parts, for example also including a package insert with instructions for the use of the recombinant rhabdovirus.

### Medical Uses

A further aspect of the invention provides a recombinant rhabdovirus of the invention for use in medicine.

The recombinant rhabdovirus of the invention efficiently induces tumor cell lysis combined with immunogenic cell death and stimulation of innate immune cells in the tumor microenvironment. Accordingly, the recombinant rhabdovirus of the invention are useful for the treatment and/or prevention of cancer.

In a further aspect, the recombinant rhabdovirus of the invention can be used in a method for treating and/or preventing cancer, comprising administering a therapeutically effective amount of a recombinant rhabdovirus to an individual suffering from cancer, thereby ameliorating one or more symptoms of cancer.

In yet a further aspect the invention further provides for the use of a recombinant rhabdovirus according to the invention for the manufacture of a medicament for treatment and/or prevention of cancer.

In yet a further aspect, the recombinant rhabdovirus of the invention can be used in a method for treating and/or preventing breast cancer, triple negative breast cancer, colorectal cancer, gastric cancer, gastrointestinal cancer, lung cancer or head & neck cancer, comprising administering a therapeutically effective amount of a recombinant rhabdovirus to an individual suffering from breast cancer, triple negative breast cancer, colorectal cancer, gastric cancer, gastrointestinal cancer, lung cancer or head & neck cancer, thereby ameliorating one or more symptoms of gastrointestinal cancer, lung cancer or head & neck cancer.

For the prevention or treatment of a disease, the appropriate dosage of recombinant rhabdovirus will depend on a variety of factors such as the type of disease to be treated, as defined above, the severity and course of the disease, whether the recombinant rhabdovirus is administered for preventive or therapeutic purposes, previous therapy, the patient's clinical history and response to the recombinant rhabdovirus, and the discretion of the attending physician. The recombinant rhabdovirus is suitably administered to the patient at one time or over a series of treatments.

In one aspect, the cancer is a solid cancer. The solid cancer may be reproductive cancer, ovarian cancer, testicular cancer, endocrine cancer, gastrointestinal cancer, pancreatic cancer, pancreatic adenocarcinoma, liver cancer, kidney cancer, colon cancer, colorectal cancer, bladder cancer, bladder urothelial carcinoma, muscle invasive bladder cancer (MIBC), non-muscle invasive bladder cancer (NMIBC), prostate cancer or carcinoma, skin cancer, (metastatic) melanoma, respiratory cancer, lung cancer, non-small cell lung cancer, small cell lung cancer, (metastatic) breast cancer or carcinoma, (metastatic) triple negative breast cancer (TNBC), head & neck cancer, head and neck squamous-cell carcinoma (HNSCC), bone cancer, gastric cancer, brain cancer, endometrial cancer, vaginal cancer, anal cancer, oropharyngeal squamous cell carcinoma, gastroesophageal junction adenocarcinoma, esophageal carcinoma, gastro esophageal junction (GEJ) cancer, oesophageal and gastroesophageal junction cancer, adenocarcinoma of the GEJ, hepatocellular carcinoma, cholangiocarcinoma, squamous cell carcinoma, and glioblastoma. Preferred is the treatment of breast cancer, triple negative breast cancer, gastric cancer or colorectal cancer.

In yet a further aspect the invention further provides for the use of a recombinant rhabdovirus according to the invention for use in combination and/or as an add-on (simultaneously, concurrently or sequentially) for the neoadjuvant treatment of cancers.

In yet a further aspect the invention further provides for the use of a recombinant rhabdovirus according to the invention for use in combination (simultaneously, concurrently or sequentially) with standard of care (e.g. such as Pembrolizumab+chemotherapy).

In yet a further aspect the invention further provides for the use of a recombinant rhabdovirus according to the invention for use in the neoadjuvant treatment of triple negative Breast Cancer (TNBC), preferably independent of PD-L1 status.

In yet a further aspect the invention further provides for the use of a recombinant rhabdovirus according to the invention for use in patients with TNBC independent of PD-L1 status.

In yet a further aspect the invention further provides for the use of a recombinant rhabdovirus according to the invention for use in combination with radiotherapy. In a related aspect, the recombinant rhabdovirus according to the invention is used in combination with radiotherapy (simultaneously, concurrently or sequentially) for treatment of TNBC.

The recombinant rhabdovirus is administered by any suitable means, including oral, parenteral, subcutaneous, intratumoral, intravenous, intradermal, intraperitoneal, intrapulmonary, intracranial and intranasal. Parenteral infusions include intramuscular, intravenous, intraarterial, intraperitoneal, or subcutaneous administration. In addition, the recombinant rhabdovirus is suitably administered by pulse infusion. In one aspect, the dosing is given by injections, most preferably intravenous or subcutaneous injections, depending in part on whether the administration is brief or chronic.

Depending on the specific recombinant rhabdovirus of the invention and its specific pharmacokinetic and other properties, it may be administered daily, every second, third, fourth, fifth or sixth day, weekly, monthly, and the like. An administration regimen could include long-term, weekly treatment. By “long-term” is meant at least two weeks and preferably months, or years of duration.

The treatment schedule may include various regimens and in typical will require multiple doses administered to the patient over a period of one, two, three or four weeks optionally followed by one or more further rounds of treatment. In one aspect, the recombinant rhabdovirus of the invention is administered to the patient in up to 1, 2, 3, 4, 5, or 6 doses within a given period of time.

The term “suppression” is used herein in the same context as “amelioration” and “alleviation” to mean a lessening or diminishing of one or more characteristics of the disease. The recombinant rhabdovirus or pharmaceutical composition of the invention will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the recombinant rhabdovirus to be administered will be governed by such considerations, and is the minimum amount necessary to prevent, ameliorate, or treat clinical symptoms of cancer, in particular the minimum amount which is effective to these disorders.

In another aspect the recombinant rhabdovirus of the invention can be administered multiple times and in several doses. In one aspect, the first dose of the recombinant rhabdovirus is administered intratumorally and subsequent doses of the recombinant rhabdovirus are administered intravenously. In a further aspect, the first dose and at least one or more following doses of the recombinant rhabdovirus is/are administered intratumorally and subsequent doses of the recombinant rhabdovirus are administered intravenously.

In another aspect, the first dose of the recombinant rhabdovirus is administered intravenously and subsequent doses of the recombinant rhabdovirus are administered intratumorally.

In another aspect, the recombinant rhabdovirus is administered intravenously and subsequent doses of the recombinant rhabdovirus are administered intratumorally.

In another aspect, the recombinant rhabdovirus is administered at each time point intravenously and intratumorally.

In another aspect, the recombinant rhabdovirus is administered intratumorally. In another aspect, the recombinant rhabdovirus is administered intravenously.

As stated above, the recombinant rhabdovirus of the invention have much utility for stimulating an immune response against cancer cells. The strong immune activating potential was observed to be restricted to the tumor microenvironment. Thus, in a preferred aspect, the recombinant rhabdovirus of the invention may be administered systemically to a patient. Systemic applicability is a crucial attribute, as many cancers are highly metastasized, and it will permit the treatment of difficult to access as well as non-accesible tumor leasions. Due to this unique immune stimulating properties the recombinant rhabdovirus according to the invention are especially useful for treatment of metastasizing tumors.

Some patients develop resistance to checkpoint inhibitor therapy, and it was observed that such patients seem to accumulate mutations in the IFN pathway. Therefore, in one aspect, the recombinant rhabdovirus of the invention and in particular the recombinant vesicular stomatitis virus of the invention is useful for the treatment of patients who developed a resistance to checkpoint inhibitor therapy. Due to the unique immune promoting properties of the recombinant rhabdovirus and in particular the recombinant vesicular stomatitis virus of the invention such treated patients may become eligible for continuation of checkpoint inhibitor therapy.

In a preferred embodiment, the recombinant rhabdovirus of the invention and in particular the recombinant vesicular stomatitis virus of the invention is useful for the treatment of patients with non-small cell lung cancer which have completed checkpoint inhibitor therapy with either a PD-1 or PD-L1 inhibitor, e.g. antagonistic antibodies to PD-1 or PD-L1.

It is understood that any of the above pharmaceutical formulations or therapeutic methods may be carried out using any one of the inventive recombinant rhabdovirus or pharmaceutical compositions.

### Combinations

The present invention also provides combination treatments/methods providing certain advantages compared to treatments/methods currently used and/or known in the prior art. These advantages may include in vivo efficacy (e.g. improved clinical response, extend of the response, increase of the rate of response, duration of response, disease stabilization rate, duration of stabilization, time to disease progression, progression free survival (PFS) and/or overall survival (OS), later occurrence of resistance and the like), safe and well tolerated administration and reduced frequency and severity of adverse events.

The recombinant rhabdovirus of the invention may be used in combination with other pharmacologically active ingredients, such as state-of-the-art or standard-of-care compounds, such as e.g. cytostatic or cytotoxic substances, cell proliferation inhibitors, anti-angiogenic substances, steroids, immune modulators/checkpoint inhibitors, and the like. The recombinant rhabdovirus of the invention may also be used in combination with radiotherapy.

Cytostatic and/or cytotoxic active substances which may be administered in combination with recombinant rhabdovirus of the invention include, without being restricted thereto, hormones, hormone analogues and antihormones, aromatase inhibitors, LHRH agonists and antagonists, inhibitors of growth factors (growth factors such as for example platelet derived growth factor (PDGF), fibroblast growth factor (FGF), vascular endothelial growth factor (VEGF), epidermal growth factor (EGF), insuline-like growth factors (IGF), human epidermal growth factor (HER, e.g. HER2, HER3, HER4) and hepatocyte growth factor (HGF)), inhibitors are for example (anti-)growth factor antibodies, (anti-)growth factor receptor antibodies and tyrosine kinase inhibitors, such as for example cetuximab, gefitinib, afatinib, nintedanib, imatinib, lapatinib, bosutinib and trastuzumab; antimetabolites (e.g. antifolates such as methotrexate, raltitrexed, pyrimidine analogues such as 5-fluorouracil (5-FU), gemcitabine, irinotecan, doxorubicin, TAS-102, capecitabine and gemcitabine, purine and adenosine analogues such as mercaptopurine, thioguanine, cladribine and pentostatin, cytarabine (ara C), fludarabine); antitumor antibiotics (e.g. anthracyclins); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); angiogenesis inhibitors, including bevacizumab, ramucirumab and aflibercept, tubuline inhibitors; DNA synthesis inhibitors, PARP inhibitors, topoisomerase inhibitors (e.g. epipodophyllotoxins such as for example etoposide and etopophos, teniposide, amsacrin, topotecan, irinotecan, mitoxantrone), serine/threonine kinase inhibitors (e.g. PDK1 inhibitors, Raf inhibitors, A-Raf inhibitors, B-Raf inhibitors, C-Raf inhibitors, mTOR inhibitors, mTORC1/2 inhibitors, PI3K inhibitors, PI3Kα inhibitors, dual mTOR/PI3K inhibitors, STK33 inhibitors, AKT inhibitors, PLK1 inhibitors (such as volasertib), inhibitors of CDKs, including CDK9 inhibitors, Aurora kinase inhibitors), tyrosine kinase inhibitors (e.g. PTK2/FAK inhibitors), protein protein interaction inhibitors, MEK inhibitors, ERK inhibitors, FLT3 inhibitors, BRD4 inhibitors, IGF-1R inhibitors, Bcl-xL inhibitors, Bcl-2 inhibitors, Bcl-2/Bcl-xL inhibitors, ErbB receptor inhibitors, BCR-ABL inhibitors, ABL inhibitors, Src inhibitors, rapamycin analogs (e.g. everolimus, temsirolimus, ridaforolimus, sirolimus), androgen synthesis inhibitors, androgen receptor inhibitors, DNMT inhibitors, HDAC inhibitors, ANG1/2 inhibitors, CYP17 inhibitors, radiopharmaceuticals, immunotherapeutic agents such as immune checkpoint inhibitors (e.g. CTLA4, PD1, PD-L1, LAG3, and TIM3 binding molecules/immunoglobulins, such as ipilimumab, nivolumab, pembrolizumab) and various chemotherapeutic agents such as amifostin, anagrelid, clodronat, filgrastin, interferon, interferon alpha, leucovorin, rituximab, procarbazine, levamisole, mesna, mitotane, pamidronate and porfimer; proteasome inhibitors (such as Bortezomib); Smac and BH3 mimetics; agents restoring p53 functionality including mdm2-p53 antagonist; inhibitors of the Wnt/beta-catenin signaling pathway; FIt3L as well as Flt3-stimulating antibodies or ligand mimetics; SIRPalpha & CD47 blocking therapeutics; and/or cyclin-dependent kinase 9 inhibitors.

The recombinant rhabdovirus of the invention can be used in combination treatment with a PD-1 pathway inhibitor. Such a combined treatment may be given as a non-fixed (e.g. free) combination of the substances or in the form of a fixed combination, including kit-of-parts.

In this context, “combination” or “combined” within the meaning of this invention includes, without being limited, a product that results from the mixing or combining of more than one active agent and includes both fixed and non-fixed (e.g. free) combinations (including kits) and uses, such as e.g. the simultaneous, concurrent, sequential, successive, alternate or separate use of the components or agents. The term “fixed combination” means that the active agents are both administered to a patient simultaneously in the form of a single entity or dosage. The term “non-fixed combination” means that the active agents are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active agents.

The invention provides for a recombinant rhabdovirus in combination with a PD-1 pathway inhibitor for use in the treatment of cancers as described herein, preferably for the treatment of solid cancers.

The invention also provides for the use of a recombinant rhabdovirus in combination with a PD-1 pathway inhibitor for the manufacture of a medicament for treatment and/or prevention of cancers as described herein, preferably for the treatment of solid cancers.

The invention further provides for a method for treating and/or preventing cancer, comprising administering a therapeutically effective amount of a recombinant rhabdovirus of the invention, and a PD-1 pathway inhibitor to an individual suffering from cancer, thereby ameliorating one or more symptoms of cancer. The recombinant rhabdovirus of the invention and the PD-1 pathway inhibitor may be administered concomitantly, sequentially or alternately.

The recombinant rhabdovirus of the invention and the PD-1 pathway inhibitor may be administered by the same administration routes or via different administration routes. Preferably, the PD-1 pathway inhibitor is administered intravenously and the recombinant rhabdovirus of the invention is administered intratumorally. In another embodiment, the PD-1 pathway inhibitor is administered intravenously and the recombinant rhabdovirus of the invention is administered at least once intratumorally and subsequent doses of the recombinant rhabdovirus are administered intravenously. The subsequent doses may be administered 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or 31 days after the initial intratumoral administration. In a preferred embodiment the PD-1 pathway inhibitor is administered 21 days after the initial intratumoral administration.

Particularly preferred are treatments with the recombinant rhabdovirus of the invention in combination with immunotherapeutic agents, including anti-PD-1, anti-PD-L1 agents and/or anti LAG3 agents, such as pembrolizumab and nivolumab and antibodies as disclosed in WO2017/198741.

A combination as herein provided comprises (i) a recombinant rhabdovirus of the invention and (ii) a PD-1 pathway inhibitor, preferably an antagonistic antibody which is directed against PD-1 or PD-L1. Further provided is the use of such a combination for the treatment of cancers as described herein.

In another aspect a combination treatment is provided comprising the use of (i) a recombinant rhabdovirus of the invention and (ii) a PD-1 pathway inhibitor. In such combination treatment the recombinant rhabdovirus of the invention may be administered concomitantly, sequentially or alternately with the PD-1 pathway inhibitor.

For example, “concomitant” administration includes administering the active agents within the same general time period, for example on the same day(s) but not necessarily at the same time. Alternate administration includes administration of one agent during a time period, for example over the course of a few days or a week, followed by administration of the other agent during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent during a second time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g. according to the agents used and the condition of the subject.

Sequential treatment schedules include administration of the recombinant rhabdovirus of the invention followed by administration of the PD-1 pathway inhibitor. Sequential treatment schedules also include administration of the PD-1 pathway inhibitor followed by administration of the recombinant rhabdovirus of the invention. Sequential treatment schedules may include administrations 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or 31 days after each other.

A PD-1 pathway inhibitor within the meaning of this invention and all of its embodiments is a compound that inhibits the interaction of PD-1 with its receptor(s). A PD-1 pathway inhibitor is capable to impair the PD-1 pathway signaling, preferably mediated by the PD-1 receptor. The PD-1 inhibitor may be any inhibitor directed against any member of the PD-1 pathway capable of antagonizing PD-1 pathway signaling. The inhibitor may be an antagonistic antibody targeting any member of the PD-1 pathway, preferably directed against PD-1 receptor, PD-L1 or PD-L2. Also, the PD-1 pathway inhibitor may be a fragment of the PD-1 receptor or the PD-1 receptor blocking the activity of PD1 ligands.

PD-1 antagonists are well-known in the art, e.g. reviewed by Li et al., Int. J. Mol. Sci. 2016, 17, 1151 (incorporated herein by reference). Any PD-1 antagonist, especially antibodies, such as those disclosed by Li et al. as well as the further antibodies disclosed herein below, can be used according to the invention. Preferably, the PD-1 antagonist of this invention and all its embodiments is selected from the group consisting of the following antibodies:
ezabenlimab (BI754091);
pembrolizumab (anti-PD-1 antibody);
nivolumab (anti-PD-1 antibody);
pidilizumab (anti-PD-1 antibody);
PDR-001 (anti-PD-1 antibody);
PD1-1, PD1-2, PD1-3, PD1-4, and PD1-5 as disclosed herein below (anti-PD-1 antibodies)
atezolizumab (anti-PD-L1 antibody);
avelumab (anti-PD-L1 antibody);
durvalumab (anti-PD-L1 antibody).

Pembrolizumab (formerly also known as lambrolizumab; trade name Keytruda; also known as MK-3475) disclosed e.g. in Hamid, O. et al. (2013) New England Journal of Medicine 369 (2): 134-44, is a humanized IgG4 monoclonal antibody that binds to PD-1; it contains a mutation at C228P designed to prevent Fc-mediated cytotoxicity. Pembrolizumab is e.g. disclosed in U.S. Pat. No. 8,354,509 and WO2009/114335. It is approved by the FDA for the treatment of patients suffering from unresectable or metastatic melanoma and patients with metastatic NSCLC.

Nivolumab (CAS Registry Number: 946414-94-4; BMS-936558 or MDX1106b) is a fully human IgG4 monoclonal antibody which specifically blocks PD-1, lacking detectable antibody-dependent cellular toxicity (ADCC). Nivolumab is e.g. disclosed in U.S. Pat. No. 8,008,449 and WO2006/121168. It has been approved by the FDA for the treatment of patients suffering from unresectable or metastatic melanoma, metastatic NSCLC and advanced renal cell carcinoma.

Pidilizumab (CT-011; Cure Tech) is a humanized IgG1k monoclonal antibody that binds to PD-1. Pidilizumab is e.g. disclosed in WO2009/101611.

PDR-001 or PDR001 is a high-affinity, ligand-blocking, humanized anti-PD-1 IgG4 antibody that blocks the binding of PD-L1 and PD-L2 to PD-1. PDR-001 is disclosed in WO2015/112900 and WO2017/019896.

Antibodies PD1-1 to PD1-5 are antibody molecules defined by the sequences as shown in Table 1, wherein HC denotes the (full length) heavy chain and LC denotes the (full length) light chain:

TABLE 5

| SEQ ID | Sequence | |
|---|---|---|
| NO: | name | Amino acid sequence |
| 35 | HC of | EVMLVESGGGLVQPGGSLRLSCTASGFTFSASAMSWVRQAPGKGLEWVAYI |
| | PD1-1 | SGGGGDTYYSSSVKGRFTISRDNAKNSLYLQMNSLRAEDTAVYYCARHSNV |
| | | NYYAMDYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCNVDH |
| | | KPSNTKVDKRVESKYGPPCPPCPAPEFLGGPSVFLFPPKPKDTLMISRTPEVTC |
| | | VVVDVSQEDPEVQFNWYVDGVEVHNAKTKPREEQFNSTYRVVSVLTVLHQ |
| | | DWLNGKEYKCKVSNKGLPSSIEKTISKAKGQPREPQVYTLPPSQEEMTKNQV |
| | | SLTCLVKGFYPSDIAVEWESNGQPENNYKTTPPVLDSDGSFFLYSRLTVDKSR |
| | | WQEGNVFSCSVMHEALHNHYTQKSLSLSLG |
| 36 | LC of | EIVLTQSPATLSLSPGERATMSCRASENIDTSGISFMNWYQQKPGQAPKLLIYV |
| | PD1-1 | ASNQGSGIPARFSGSGSGTDFTLTISRLEPEDFAVYYCQQSKEVPWTFGQGTK |
| | | LEIKRTVAAPSVFIFPPSDEQLKSGTASVVCLLNNFYPREAKVQWKVDNALQS |
| | | GNSQESVTEQDSKDSTYSLSSTLTLSKADYEKHKVYACEVTHQGLSSPVTKSF |
| | | NRGEC |
| 37 | HC of | EVMLVESGGGLVQPGGSLRLSCTASGFTFSASAMSWVRQAPGKGLEWVAYI |
| | PD1-2 | SGGGGDTYYSSSVKGRFTISRDNAKNSLYLQMNSLRAEDTAVYYCARHSNP |
| | | NYYAMDYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCNVDH |
| | | KPSNTKVDKRVESKYGPPCPPCPAPEFLGGPSVFLFPPKPKDTLMISRTPEVTC |
| | | VVVDVSQEDPEVQFNWYVDGVEVHNAKTKPREEQFNSTYRVVSVLTVLHQ |
| | | DWLNGKEYKCKVSNKGLPSSIEKTISKAKGQPREPQVYTLPPSQEEMTKNQV |
| | | SLTCLVKGFYPSDIAVEWESNGQPENNYKTTPPVLDSDGSFFLYSRLTVDKSR |
| | | WQEGNVFSCSVMHEALHNHYTQKSLSLSLG |
| 38 | LC of | EIVLTQSPATLSLSPGERATMSCRASENIDTSGISFMNWYQQKPGQAPKLLIYV |
| | PD1-2 | ASNQGSGIPARFSGSGSGTDFTLTISRLEPEDFAVYYCQQSKEVPWTFGQGTK |
| | | LEIKRTVAAPSVFIFPPSDEQLKSGTASVVCLLNNFYPREAKVQWKVDNALQS |
| | | GNSQESVTEQDSKDSTYSLSSTLTLSKADYEKHKVYACEVTHQGLSSPVTKSF |
| | | NRGEC |
| 39 | HC of | EVMLVESGGGLVQPGGSLRLSCTASGFTFSKSAMSWVRQAPGKGLEWVAYI |
| | PD1-3 | SGGGGDTYYSSSVKGRFTISRDNAKNSLYLQMNSLRAEDTAVYYCARHSNV |
| | | NYYAMDYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCNVDH |
| | | KPSNTKVDKRVESKYGPPCPPCPAPEFLGGPSVFLFPPKPKDTLMISRTPEVTC |
| | | VVVDVSQEDPEVQFNWYVDGVEVHNAKTKPREEQFNSTYRVVSVLTVLHQ |
| | | DWLNGKEYKCKVSNKGLPSSIEKTISKAKGQPREPQVYTLPPSQEEMTKNQV |
| | | SLTCLVKGFYPSDIAVEWESNGQPENNYKTTPPVLDSDGSFFLYSRLTVDKSR |
| | | WQEGNVFSCSVMHEALHNHYTQKSLSLSLG |
| 40 | LC of | EIVLTQSPATLSLSPGERATMSCRASENIDVSGISFMNWYQQKPGQAPKLLIY |
| | PD1-3 | VASNQGSGIPARFSGSGSGTDFTLTISRLEPEDFAVYYCQQSKEVPWTFGQGT |
| | | KLEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPVT |
| | | KSFNRGEC |
| 41 | HC of | EVMLVESGGGLVQPGGSLRLSCTASGFTFSKSAMSWVRQAPGKGLEWVAYI |
| | PD1-4 | SGGGGDTYYSSSVKGRFTISRDNAKNSLYLQMNSLRAEDTAVYYCARHSNV |
| | | NYYAMDYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCNVDH |
| | | KPSNTKVDKRVESKYGPPCPPCPAPEFLGGPSVFLFPPKPKDTLMISRTPEVTC |
| | | VVVDVSQEDPEVQFNWYVDGVEVHNAKTKPREEQFNSTYRVVSVLTVLHQ |
| | | DWLNGKEYKCKVSNKGLPSSIEKTISKAKGQPREPQVYTLPPSQEEMTKNQV |
| | | SLTCLVKGFYPSDIAVEWESNGQPENNYKTTPPVLDSDGSFFLYSRLTVDKSR |
| | | WQEGNVFSCSVMHEALHNHYTQKSLSLSLG |
| 42 | LC of | EIVLTQSPATLSLSPGERATMSCRASENIDVSGISFMNWYQQKPGQAPKLLIY |
| | PD1-4 | VASNQGSGIPARFSGSGSGTDFTLTISRLEPEDFAVYYCQQSKEVPWTFGQGT |
| | | KLEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPVT |
| | | KSFNRGEC |
| 43 | HC of | EVMLVESGGGLVQPGGSLRLSCTASGFTFSKSAMSWVRQAPGKGLEWVAYI |
| | PD1-5 | SGGGGDTYYSSSVKGRFTISRDNAKNSLYLQMNSLRAEDTAVYYCARHSNV |
| | | NYYAMDYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCNVDH |
| | | KPSNTKVDKRVESKYGPPCPPCPAPEFLGGPSVFLFPPKPKDTLMISRTPEVTC |
| | | VVVDVSQEDPEVQFNWYVDGVEVHNAKTKPREEQFNSTYRVVSVLTVLHQ |
| | | DWLNGKEYKCKVSNKGLPSSIEKTISKAKGQPREPQVYTLPPSQEEMTKNQV |
| | | SLTCLVKGFYPSDIAVEWESNGQPENNYKTTPPVLDSDGSFFLYSRLTVDKSR |
| | | WQEGNVFSCSVMHEALHNHYTQKSLSLSLG |
| 44 | LC of | EIVLTQSPATLSLSPGERATMSCRASENIDVSGISFMNWYQQKPGQAPKLLIY |
| | PD1-5 | VASNQGSGIPARFSGSGSGTDFTLTISRLEPEDFAVYYCQQSKEVPWTFGQGT |
| | | KLEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPVT |
| | | KSFNRGEC |

Specifically, the anti-PD-1 antibody molecule described herein above has:
(PD1-1:) a heavy chain comprising the amino acid sequence of SEQ ID NO:35 and a light chain comprising the amino acid sequence of SEQ ID NO:36; or
(PD1-2:) a heavy chain comprising the amino acid sequence of SEQ ID NO:37 and a light chain comprising the amino acid sequence of SEQ ID NO:38; or
(PD1-3:) a heavy chain comprising the amino acid sequence of SEQ ID NO:39 and a light chain comprising the amino acid sequence of SEQ ID NO:40; or
(PD1-4:) a heavy chain comprising the amino acid sequence of SEQ ID NO:41 and a light chain comprising the amino acid sequence of SEQ ID NO:42; or
(PD1-5:) a heavy chain comprising the amino acid sequence of SEQ ID NO:43 and a light chain comprising the amino acid sequence of SEQ ID NO:44.

Atezolizumab (Tecentriq, also known as MPDL3280A) is a phage-derived human IgG1k monoclonal antibody targeting PD-L1 and is described e.g. in Deng et al. mAbs 2016; 8:593-603. It has been approved by the FDA for the treatment of patients suffering from urothelial carcinoma.

Avelumab is a fully human anti-PD-L1 IgG1 monoclonal antibody and described in e.g. Boyerinas et al. Cancer Immunol. Res. 2015; 3:1148-1157.

Durvalumab (MEDI4736) is a human IgG1k monoclonal antibody with high specificity to PD-L1 and described in e.g. Stewart et al. Cancer Immunol. Res. 2015; 3:1052-1062 or in Ibrahim et al. Semin. Oncol. 2015; 42:474-483.

Further PD-1 antagonists disclosed by Li et al. (supra), or known to be in clinical trials, such as AMP-224, MEDI0680 (AMP-514), REGN2810, BMS-936559, JS001-PD-1, SHR-1210, BMS-936559, TSR-042, JNJ-63723283, MEDI4736, MPDL3280A, and MSB0010718C, may be used as alternative or in addition to the above mentioned antagonists.

The INNs as used herein are meant to also encompass all biosimilar antibodies having the same, or substantially the same, amino acid sequences as the originator antibody, including but not limited to those biosimilar antibodies authorized under 42 USC § 262 subsection (k) in the US and equivalent regulations in other jurisdictions.

PD-1 antagonists listed above are known in the art with their respective manufacture, therapeutic use and properties.

In one embodiment the PD-1 antagonist is ezabenlimab.

In one embodiment the PD-1 antagonist is pembrolizumab.

In another embodiment the PD-1 antagonist is nivolumab.

In another embodiment the PD-1 antagonist is pidilizumab.

In another embodiment the PD-1 antagonist is atezolizumab.

In another embodiment the PD-1 antagonist is avelumab.

In another embodiment the PD-1 antagonist is durvalumab.

In another embodiment the PD-1 antagonist is PDR-001.

In another embodiment the PD-1 antagonist is PD1-1.

In another embodiment the PD-1 antagonist is PD1-2.

In another embodiment the PD-1 antagonist is PD1-3.

In another embodiment the PD-1 antagonist is PD1-4.

In another embodiment the PD-1 antagonist is PD1-5.

In a preferred embodiment relating to the combination treatments the recombinant rhabdovirus is a recombinant vesicular stomatitis virus encoding in its genome a vesicular stomatitis virus nucleoprotein (N), large protein (L), phosphoprotein (P), matrix protein (M), glycoprotein (G) and at least one GSDM comprising the amino acid of sequence of SEQ ID NO:49, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV.

In a preferred embodiment relating to the combination treatments the recombinant rhabdovirus is a recombinant vesicular stomatitis virus encoding in its genome a vesicular stomatitis virus nucleoprotein (N), large protein (L), phosphoprotein (P), matrix protein (M), glycoprotein (G) and at least one GSDM comprising the amino acid of sequence of SEQ ID NO:49, and an IL12p35 and an IL12p40 subunit of IL12 linked in a single-chain having the configuration IL12p40-IL12p35 and comprising the amino acid sequence of SEQ ID NO:5 or SEQ ID NO:66, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV.

In a preferred embodiment relating to the combination treatments the recombinant rhabdovirus is a recombinant vesicular stomatitis virus encoding in its genome a vesicular stomatitis virus nucleoprotein (N), large protein (L), phosphoprotein (P), matrix protein (M), glycoprotein (G) and an amino acid sequence with at least 90% identity to SEQ ID NO:72, preferably an amino acid sequence identical to SEQ ID NO:72, wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV.

In an embodiment relating to the aforementioned preferred embodiments, the nucleoprotein (N) comprises an amino acid as set forth in SEQ ID NO: 28 or a functional variant at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 28, the phosphoprotein (P) comprises an amino acid as set forth in SEQ ID NO: 29 or a functional variant at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 29, the large protein (L) comprises an amino acid as set forth in SEQ ID NO:30 or a functional variant at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:30, and the matrix protein (M) comprises an amino acid as set forth in SEQ ID NO:31 or a functional variant at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:31.

### Virus Generation, Production and Virus Producing Cell

The invention also provides a virus producing cell, characterized in that the cell produces a recombinant rhabdovirus or recombinant vesicular stomatitis virus according to the invention.

The cell may be of any origin and may be present as isolated cell or as a cell comprised in a cell population. It is preferred that the cell producing a recombinant rhabdovirus or recombinant vesicular stomatitis virus is a mammalian cell. In a more preferred embodiment, the virus producing cell of the invention is characterized in that the mammalian cell is a multipotent adult progenitor cell (MAPC), a neural stem cell (NSC), a mesenchymal stem cell (MSC), a HeLa cell, a HEK cell, any HEK293 cell (e.g. HEK293F or HEK293T), a Chinese hamster ovary cell (CHO), a baby hamster kidney (BHK) cell or a Vero cell or a bone marrow derived tumor infiltrating cell (BM-TIC).

Alternatively, the virus producing cell may be a human cell, monkey cell, mouse cell or hamster cell. The skilled person is aware of methods suitable for use in testing whether a given cell produces a virus and, thus, whether a particular cell falls within the scope of this invention. In this respect, the amount of virus produced by the cell of the invention is not particularly limited. Preferred viral titers are ≥1×10⁷ TCID50/ml or ≥1×10⁸ genome copies/ml in the crude supernatants of the given cell culture after infection without further downstream processing.

In a particular embodiment, the virus producing cell of the invention is characterized in that the cell comprises one or more expression cassettes for the expression of at least one of the genes selected from the group consisting of genes n, l, p and m coding for proteins N, L, P and M of the VSV and a gene gp coding for LCMV-GP, Dandenong-GP or Mopeia-GP glycoprotein.

Virus producing cells in the meaning of the invention include classical packaging cells for the production of recombinant rhabdovirus from non-replicable vectors as well as producer cells for the production of recombinant rhabdovirus from vectors capable of reproduction. Packaging cells usually comprise one or more plasmids for the expression of essential genes which lack in the respective vector to be packaged and/or are necessary for the production of virus. Such cells are known to the skilled person who can select appropriate cell lines suitable for the desired purpose.

Recombinant rhabdovirus of the invention can be produced according to methods known to the skilled artisan and include without limitation (1) using cDNAs transfected into a cell or (2) a combination of cDNAs transfected into a helper cell, or (3) cDNAs transfected into a cell, which is further infected with a helper/minivirus providing in trans the remaining components or activities needed to produce either an infectious or non-infectious recombinant rhabdovirus. Using any of these methods (e.g., helper/minivirus, helper cell line, or cDNA transfection only), the minimum components required are a DNA molecule containing the cis-acting signals for (1) encapsidation of the genomic (or antigenomic) RNA by the Rhabdovirus N protein, P protein and L protein and (2) replication of a genomic or antigenomic (replicative intermediate) RNA equivalent.

A replicating element or replicon is a strand of RNA minimally containing at the 5′ and 3′ ends the leader sequence and the trailer sequence of a rhabdovirus. In the genomic sense, the leader is at the 3′ end and the trailer is at the 5′ end. Any RNA-placed between these two replication signals will in turn be replicated. The leader and trailer regions further must contain the minimal cis-acting elements for purposes of encapsidation by the N protein and for polymerase binding which are necessary to initiate transcription and replication. For preparing recombinant rhabdovirus a minivirus containing the G gene would also contain a leader region, a trailer region and a G gene with the appropriate initiation and termination signals for producing a G protein mRNA. If the minivirus further comprises an M gene, the appropriate initiation and termination signals for producing the M protein mRNA must also present.

For any gene contained within the recombinant rhabdovirus genome, the gene would be flanked by the appropriate transcription initiation and termination signals which will allow expression of those genes and production of the protein products (Schnell et al., Journal of Virology, p. 2318-2323, 1996). To produce “non-infectious” recombinant rhabdovirus, the recombinant rhabdovirus must have the minimal replicon elements and the N, P, and L proteins and it must contain the M gene. This produces virus particles that are budded from the cell but are non-infectious particles. To produce “infectious” particles, the virus particles must additionally comprise proteins that can mediate virus particle binding and fusion, such as through the use of an attachment protein or receptor ligand. The native receptor ligand of rhabdoviruses is the G protein.

Any cell that would permit assembly of the recombinant rhabdovirus can be used. One method to prepare infectious virus particles comprises an appropriate cell line infected with a plasmid encoding for a T7 RNA polymerase or other suitable bacteriophage polymerase such as the T3 or SP6 polymerases. The cells may then be transfected with individual cDNA containing the genes encoding the G, N, P, L and M rhabdovirus proteins. These cDNAs will provide the proteins for building a recombinant rhabdovirus particle. Cells can be transfected by any method known in the art.

Also transfected into the cell line is a “polycistronic cDNA” containing the rhabdovirus genomic RNA equivalent. If the infectious, recombinant rhabdovirus particle is intended to be lytic in an infected cell, then the genes encoding for the N, P, M and L proteins must be present as well as any heterologous nucleic acid segment. If the infectious, recombinant rhabdovirus particle is not intended to be lytic, then the gene encoding the M protein is not included in the polycistronic DNA. By “polycistronic cDNA” it is meant a cDNA comprising at least transcription units containing the genes which encode the N, P and L proteins. The recombinant rhabdovirus polycistronic DNA may also contain a gene encoding a protein variant or polypeptide fragment thereof, or a therapeutic nucleic acid or protein. Alternatively, any protein to be initially associated with the viral particle first produced or fragment thereof may be supplied in trans.

Also contemplated is a polycistronic cDNA comprising a gene encoding for a GSDM. The polycistronic cDNA contemplated may contain a gene encoding a protein variant, a gene encoding a reporter, a therapeutic nucleic acid, and/or either the N-P-L genes or the N-P-L-M genes. The first step in generating a recombinant rhabdovirus is expression of an RNA that is a genomic or antigenomic equivalent from a cDNA. Then that RNA is packaged by the N protein and then replicated by the P/L proteins. The recombinant virus thus produced can be recovered. If the G protein is absent from the recombinant RNA genome, then it is typically supplied in trans. If both the G and the M proteins are absent, then both are supplied in trans. For preparing “non-infectious rhabdovirus” particles, the procedure may be the same as above, except that the polycistronic cDNA transfected into the cells would contain the N, P and L genes of the rhabdovirus only. The polycistronic cDNA of non-infectious rhabdovirus particles may additionally contain a gene encoding a protein.

Transfected cells are usually incubated for at least 24 h at the desired temperature, usually about 37° C. For non-infectious virus particles, the supernatant is collected and the virus particles isolated. For infectious virus particles, the supernatant containing virus is harvested and transferred to fresh cells. The fresh cells are incubated for approximately 48 h, and the supernatant is collected.

A typical rhabdovirus genome encodes for at least five structural proteins in the order of 3′-N-P-M-G-L-5′. The genome might contain further short intergenic regions or additional genes between the structural proteins and therefore might vary in length and organization.

According to the invention the GSDM gene and the cytokine gene can be introduced into any location of the rhabdovirus genome. The following example may apply mutatis mutandis also for the other cytokines as disclosed herein.

For example, the GSDM gene and the IL12 gene can be introduced into any location of the rhabdovirus genome. Depending on the insertion site the transcription efficiency of the GSDM gene and/or the IL12 gene can be influenced. In general, transcription efficiency of the GSDM and/or IL12 gene decreases from 3′ insertion to 5′ prime insertion.

As explained before, the GSDM and the IL12 may be encoded as a single construct, i.e., the GSDM and the IL12 sequence (optionally comprising a 2A peptide) will be transcribed as a single chain from the virus genome.

Such a single construct may be inserted into the following genome locations: 3′-GSDM-IL12-N-P-M-G-L-5′, 3′-N-GSDM-IL12-P-M-G-L-5′, 3′-N-P-GSDM-IL12-M-G-L-5′, 3′-N-P-M-GSDM-IL12-G-L-5′, 3′-N-P-M-G-GSDM-IL12-L-5′ or 3′-N-P-M-G-L-GSDM-IL12-5′. In a preferred embodiment the GSDM-IL12 gene is inserted between the G protein and the L protein.

After infection of tumor cells, the GSDM and/or IL12 gene(s) encoded in the genome of the recombinant rhabdovirus is transcribed into positive sense RNA and then translated into the respective protein by the tumor cell. The term “encoding” or “coding” refers to the inherent property of specific sequences of nucleotides in a nucleic acid to serve as templates for synthesis of other polymers and macromolecules in biological processes having a defined sequence of nucleotides (e.g. RNA molecules) or amino acids and the biological properties resulting therefrom. Accordingly, a gene codes for a protein if the desired protein is produced in a cell or another biological system by transcription and subsequent translation of the mRNA. Both the coding strand, the nucleotide sequence of which is identical to the mRNA sequence and the non-coding strand may serve as the template for the transcription of a gene and can be referred to as encoding the protein or other product of that gene. Nucleic acids and nucleotide sequences that encode proteins may include introns.

The transcription of the GSDM and/or IL12 gene(s) is preferably not under the control of its own promoter and only strictly linked to viral replication ensuring thereby targeted expression of the gene(s) to the location of viral replication and spread (tumor). Thus, the transcription of the GSDM and/or IL12 gene(s) is not controlled by additional elements such as promoters or inducible gene expression elements.

It will be appreciated that a nucleic acid sequence may be varied with or without changing the primary sequence of the encoded polypeptide. A nucleic acid that encodes a protein includes any nucleic acids that have different nucleotide sequences but encode the same amino acid sequence of the protein due to the degeneracy of the genetic code. It is within the knowledge of the skilled artisan to choose a nucleic acid sequence that will result in the expression of a GSDM and/or IL12 and in particular to any specific GSDM and/or IL12 as disclosed herein. Nucleic acid molecules encoding the amino acid sequences are prepared by a variety of methods known in the art. These methods include, but are not limited to, isolation from a natural source or preparation by oligonucleotide-mediated (or site-directed) mutagenesis, PCR mutagenesis, and cassette mutagenesis of an earlier prepared protein.

Other features and advantages of the present invention will become apparent from the following more detailed Examples which illustrate, by way of example, the principles of the invention.

### EXAMPLES

### Methods

### Viral Rescue—Generation of VSV-GP(-Cargo)

Replication competent VSV-GP(-cargo) virus variants are generated by means of reverse genetics (cloning the genes of interest (GOI), virus rescue and repeated plaque purification) from bacterial plasmids that contain the cDNA for the complete viral genome of VSV-GP(-cargo). pVSV-GP(-cargo) plasmids are based on the plasmid pVSV-XN1 (Schnell et al.), which contains the complete cDNA genome of VSV Indiana serotype under the control of the T7 promoter. To generate pVSV-GP(-cargo) variants, the whole sequence for the VSV G envelope protein is substituted by the codon optimized sequence of GP envelope protein from Lymphocytic choriomeningitis virus (LCMV, WE-HPI strain). Optionally, a synthetic nucleic acid coding for cargo gene(s) is inserted between the glycoprotein GP and the viral polymerase L by Gibson assembly. The cargo(s) may be encoded as a single chain with or without a leading signal peptide sequence. Transcription of the cargo construct(s) in the context of viral infection is ensured by an extra VSV start signal sequence at the 3′ end and of an additional stop signal sequence at the 5′ end of the cargo open reading frame.

Infectious viruses are recovered (or rescued) from the plasmid cDNAs by transfection of HEK293T or any other VSV permissive cell line by standard transfection methods (e.g. CaPO₄ precipitation, liposomal DNA delivery). Briefly, HEK293T cells are transfected with pSF-CAG-amp-based expression plasmids encoding the VSV proteins N, P, and L as well as a codon-optimized T7-polymerase. Additionally, the plasmid coding the viral genomic cDNA of VSV-GP(-cargo) is co-transfected. In a first step of the rescue process, the T7 polymerase transcribes the virus RNA genome from the plasmid coded virus cDNA. In a second step, VSV-L and -P proteins, which are exogenously expressed from the co-transfected plasmids, further amplify the viral RNA genomes. The viral RNA genomes are co-transcriptionally encapsulated by the VSV-N protein. Additionally, the P/L polymerase complex allows transcription of the full set of viral gene products N, P, M, GP and L as well as the optionally inserted cargo. The viral RNA genomes are subsequently packaged into infectious VSV particles containing the ribonucleoprotein, the matrix protein and the viral envelope GP. Virus particles are released from the cells by budding.

Rescued viruses are initially passaged on permissive cell lines such as e.g. HEK293T. Several rounds of plaque purification are performed before generation of a virus seed stock by standard methods. Briefly, HEK293T cells are infected with serial ten-fold dilutions of the rescued pre-seeds. After approximately two hours, cell monolayers are washed twice and overlaid with media containing 0.8% of low melt agarose. 24 h to 48 h post infection, plaques are picked, and virus is used for an additional round of plaque-purification or virus seed stocks are generated.

VSV-GP without additional cargo(s), VSG-GP with other cargos or VSV-GP-ΔM51 variants are generated mutatis mutandis according to this protocol. For example, the genome of the oncolytic virus VSV-GP is engineered to encode for gasdermin E (GSDME) and IL12 genes to locally express both proteins at the tumor site during viral replication. Replication competent VSV-GP-GSDME-IL12 virus variants are generated by means of reverse genetics (cloning the genes of interest (GOI), virus rescue and repeated plaque purification) from bacterial plasmids that contain the cDNA for the complete viral genome of VSV-GP and human GSDME and IL12. pVSV-GP-GSDME-IL12 plasmids are based on the plasmid pVSV-XN1 (Schnell et al.), which contains the complete cDNA genome of VSV Indiana serotype under the control of the T7 promoter. To generate pVSV-GP-GSDME-IL12 variants, the whole sequence for the VSV G envelope protein is substituted by the codon optimized sequence of GP envelope protein from Lymphocytic choriomeningitis virus (LCMV, WE-HPI strain). Additionally, a synthetic nucleic acid coding for GSDME and IL12 genes, both genes separated by a T2A sequence, is inserted between the glycoprotein GP and the viral polymerase L by Gibson assembly. The IL12 is encoded as a single chain in the configuration IL12p40-GGGGSGGGGSGGGGS-IL12p35 with a leading signal peptide sequence. Transcription of the GSDME-IL12 construct in the context of viral infection is ensured by an extra VSV start signal sequence at the 3′ end and of an additional stop signal sequence at the 5′ end of the GSDME-IL12 open reading frame. GSDME and IL12 protein are cleaved at the T2A sequence after expression and translation.

Infectious viruses are recovered (or rescued) from the plasmid cDNAs by transfection of HEK293T or any other VSV permissive cell line by standard transfection methods (e.g. CaPO₄ precipitation, liposomal DNA delivery). Briefly, HEK293T cells are transfected with pSF-CAG-amp-based expression plasmids encoding the VSV proteins N, P, and L as well as a codon-optimized T7-polymerase. Additionally, the plasmid coding the viral genomic cDNA of VSV-GP-GSDME-IL12 is co-transfected. In a first step of the rescue process, the T7 polymerase transcribes the virus RNA genome from the plasmid coded virus cDNA. In a second step, VSV-L and -P proteins, which are exogenously expressed from the co-transfected plasmids, further amplify the viral RNA genomes. The viral RNA genomes are co-transcriptionally encapsulated by the VSV-N protein. Additionally, the P/L polymerase complex allows transcription of the full set of viral gene products N, P, M, GP and L as well as the inserted GSDME-IL12. The viral RNA genomes are subsequently packaged into infectious VSV particles containing the ribonucleoprotein, the matrix protein and the viral envelope GP. Virus particles are released from the cells by budding.

TABLE 6

| | | |
|---|---|---|
| GSDME-IL12 | MFAKATRNFLREVDADGDLIAVSNLNDSDKLQLLSLVTKKK | 72 |
| (human) | RFWCWQRPKYQFLSLTLGDVLIEDQFPSPVVVESDFVKYE | |
| | GKFANHVSGTLETALGKVKLNLGGSSRVESQSSFGTLRKQ | |
| | EVDLQQLIRDSAERTINLRNPVLQQVLEGRNEVLCVLTQKIT | |
| | TMQKCVISEHMQVEEKCGGIVGIQTKTVQVSATEDGNVTK | |
| | DSNVVLEIPAATTIAYGVIELYVKLDGQFEFCLLRGKQGGFE | |
| | NKKRIDSVYLDPLVFREFAFIDMPDAAHGISSQDGPLSVLK | |
| | QATLLLERNFHPFAELPEPQQTALSDIFQAVLFDDELLMVLE | |
| | PVCDDLVSGLSPTVAVLGELKPRQQQDLVAFLQLVGCSLQ | |
| | GGCPGPEDAGSKQLFMTAYFLVSALAEMPDSAAALLGTCC | |
| | KLQIIPTLCHLLRALSDDGVSDLEDPTLTPLKDTERFGIVQRL | |
| | FASADISLERLKSSVKAVILKDSKVFPLLLCITLNGLCALGRE | |
| | HSGSGEGRGSLLTCGDVEENPGPMCHQQLVISWFSLVFL | |
| | ASPLVAIWELKKDVYVVELDWYPDAPGEMVVLTCDTPEED | |
| | GITWTLDQSSEVLGSGKTLTIQVKEFGDAGQYTCHKGGEV | |
| | LSHSLLLLHKKEDGIWSTDILKDQKEPKNKTFLRCEAKNYS | |
| | GRFTCWWLTTISTDLTFSVKSSRGSSDPQGVTCGAATLSA | |
| | ERVRGDNKEYEYSVECQEDSACPAAEESLPIEVMVDAVHK | |
| | LKYENYTSSFFIRDIIKPDPPKNLQLKPLKNSRQVEVSWEYP | |
| | DTWSTPHSYFSLTFCVQVQGKSKREKKDRVFTDKTSATVI | |
| | CRKNASISVRAQDRYYSSSWSEWASVPCSGGGGSGGGG | |
| | SGGGGSRNLPVATPDPGMFPCLHHSQNLLRAVSNMLQKA | |
| | RQTLEFYPCTSEEIDHEDITKDKTSTVEACLPLELTKNESCL | |
| | NSRETSFITNGSCLASRKTSFMMALCLSSIYEDLKMYQVEF | |
| | KTMNAKLLMDPKRQIFLDQNMLAVIDELMQALNFNSETVPQ | |
| | KSSLEEPDFYKTKIKLCILLHAFRIRAVTIDRVMSYLNAS | |
| GSDMD-DEVD | MPSAFEKVVKNVIKEVSGSRGDLIPVDSLRNSTSFRPYCLL | 78 |
| (mouse) | NRKFSSSRFWKPRYSCVNLSIKDILEPSAPEPEPECFGSFK | |
| | VSDVVDGNIQGRVMLSGMGEGKISGGAAVSDSSSASMNV | |
| | CILRVTQKTWETMQHERHLQQPENKILQQLRSRGDDLFVV | |
| | TEVLQTKEEVQITEVHSQEGSGQFTLPGALCLKGEGKGHQ | |
| | SRKKMVTIPAGSILAFRVAQLLIGSKWDILLVSDEKQRTFEP | |
| | SSGDRKAVGQRHHGLNVLAALCSIGKQLSDEVDGIDEEELI | |
| | EAADFQGLYAEVKACSSELESLEMELRQQILVNIGKILQDQ | |
| | PSMEALEASLGQGLCSGGQVEPLDGPAGCILECLVLDSGE | |
| | LVPELAAPIFYLLGALAVLSETQQQLLAKALETTVLSKQLEL | |
| | VKHVLEQSTPWQEQSSVSLPTVLLGDCWDEKNPTWVLLE | |
| | ECGLRLQVESPQVHWEPTSLIPTSALYASLFLLSSLGQKPC | |
| GSDME-IL12 | MFAKATRNFLKEVDAGGDLISVSHLNDSDKLQLLSLVTKKK | 84 |
| (mouse) | RYWCWQRPKYQILSATLEDVLTEGHCLSPVVVESDFVKYE | |
| | SKCENHKSGAIGTVVGKVKLNVGGKGVVESHSSFGTLRKQ | |
| | EVDVQQLIQDAVKRTVNMDNLVLQQVLESRNEVLCVLTQKI | |
| | MTTQKCVISEHVQSEETCGGMVGIQTKTIQVSATEDGTVTT | |
| | DTNVVLEIPAATTIAYGIMELFVKQDGQFEFCLLQGKHGGF | |
| | EHERKLDSVYLDPLAYREFAFLDMLDGGQGISSQDGPLRV | |
| | VKQATLHLERSFHPFAVLPAQQQRALFCVLQKILFDEELLR | |
| | ALEQVCDDVAGGLWSSQAVLAMEELTDSQQQDLTAFLQL | |
| | VGYRIQGEHPGPQDEVSNQKLFATAYFLVSALAEMPDNAT | |
| | VFLGTCCKLHVISSLCCLGGGSGEGRGSLLTCGDVEENPG | |
| | PMGWSCIILFLVATATGVHSMWELEKDVYVVEVDWTPDAP | |
| | GETVNLTCDTPEEDDITWTSDQRHGVIGSGKTLTITVKEFL | |
| | DAGQYTCHKGGETLSHSHLLLHKKENGIWSTEILKNFKNKT | |
| | FLKCEAPNYSGRFTCSWLVQRNMDLKFNIKSSSSSPDSRA | |
| | VTCGMASLSAEKVTLDQRDYEKYSVSCQEDVTCPTAEETL | |
| | PIELALEARQQNKYENYSTSFFIRDIIKPDPPKNLQMKPLKN | |
| | SQVEVSWEYPDSWSTPHSYFSLKFFVRIQRKKEKMKETEE | |
| | GCNQKGAFLVEKTSTEVQCKGGNVCVQAQDRYYNSSCSK | |
| | WACVPCRVRSGGGGSGGGGSGGGGSRVIPVSGPARCLS | |
| | QSRNLLKTTDDMVKTAREKLKHYSCTAEDIDHEDITRDQTS | |
| | TLKTCLPLELHKNESCLATRETSSTTRGSCLPPQKTSLMMT | |
| | LCLGSIYEDLKMYQTEFQAINAALQNHNHQQIILDKGMLVAI | |
| | DELMQSLNHNGETLRQKPPVGEADPYRVKMKLCILLHAFS | |
| | TRVVTINRVMGYLSSA | |
| hsGSDMD-DEVD- | MGSAFERVVRRVVQELDHGGEFIPVTSLQSSTGFQPYCLV | 86 |
| mmIL18DR | VRKPSSSWFWKPRYKCVNLSIKDILEPDAAEPDVQRGRSF | |
| | HFYDAMDGQIQGSVELAAPGQAKIAGGAAVSDSSSTSMNV | |
| | YSLSVDPNTWQTLLHERHLRQPEHKVLQQLRSRGDNVYV | |
| | VTEVLQTQKEVEVTRTHKREGSGRFSLPGATCLQGEGQG | |
| | HLSQKKTVTIPSGSTLAFRVAQLVIDSDLDVLLFPDKKQRTF | |
| | QPPATGHKRSTSEGAWPQLPSGLSMMRCLHNDEVDGVP | |
| | AEGAFTEDFQGLRAEVETISKELELLDRELCQLLLEGLEGVL | |
| | RDQLALRALEEALEQGQSLGPVEPLDGPAGAVLECLVLSS | |
| | GMLVPELAIPVVYLLGALTMLSETQHKLLAEALESQTLLGPL | |
| | ELVGSLLEQSAPWQERSTMSLPPGLLGNSWGEGAPAWVL | |
| | LDECGLELGEDTPHVCWEPQAQGRMCALYASLALLSGLS | |
| | QEPHGSGEGRGSLLTCGDVEENPGPHFGRLHCTTAVIRNI | |
| | NDQVLFVDKRQPVFEDMTDIDQSASEPQTRLIIYAYGDSRA | |
| | RGKAVTLSVKDSKMSTLSCKNKIISFEEMDPPENIDDIQSDL | |
| | IFFQKRVPGHNKMEFESSLYEGHFLACQKEDDAFKLILKKK | |
| | DENGDKSVMFTLTNLHQS | |
| hsGSDMD-DEVD- | MGSAFERVVRRVVQELDHGGEFIPVTSLQSSTGFQPYCLV | 87 |
| mmIL18DR-mmIL12 | VRKPSSSWFWKPRYKCVNLSIKDILEPDAAEPDVQRGRSF | |
| | HFYDAMDGQIQGSVELAAPGQAKIAGGAAVSDSSSTSMNV | |
| | YSLSVDPNTWQTLLHERHLRQPEHKVLQQLRSRGDNVYV | |
| | VTEVLQTQKEVEVTRTHKREGSGRFSLPGATCLQGEGQG | |
| | HLSQKKTVTIPSGSTLAFRVAQLVIDSDLDVLLFPDKKQRTF | |
| | QPPATGHKRSTSEGAWPQLPSGLSMMRCLHNDEVDGVP | |
| | AEGAFTEDFQGLRAEVETISKELELLDRELCQLLLEGLEGVL | |
| | RDQLALRALEEALEQGQSLGPVEPLDGPAGAVLECLVLSS | |
| | GMLVPELAIPVVYLLGALTMLSETQHKLLAEALESQTLLGPL | |
| | ELVGSLLEQSAPWQERSTMSLPPGLLGNSWGEGAPAWVL | |
| | LDECGLELGEDTPHVCWEPQAQGRMCALYASLALLSGLS | |
| | QEPHGSGEGRGSLLTCGDVEENPGPHFGRLHCTTAVIRNI | |
| | NDQVLFVDKRQPVFEDMTDIDQSASEPQTRLIIYAYGDSRA | |
| | RGKAVTLSVKDSKMSTLSCKNKIISFEEMDPPENIDDIQSDL | |
| | IFFQKRVPGHNKMEFESSLYEGHFLACQKEDDAFKLILKKK | |
| | DENGDKSVMFTLTNLHQSGSGATNFSLLKQAGDVEENPG | |
| | PMGWSCIILFLVATATGVHSMWELEKDVYVVEVDWTPDAP | |
| | GETVNLTCDTPEEDDITWTSDQRHGVIGSGKTLTITVKEFL | |
| | DAGQYTCHKGGETLSHSHLLLHKKENGIWSTEILKNFKNKT | |
| | FLKCEAPNYSGRFTCSWLVQRNMDLKFNIKSSSSSPDSRA | |
| | VTCGMASLSAEKVTLDQRDYEKYSVSCQEDVTCPTAEETL | |
| | PIELALEARQQNKYENYSTSFFIRDIIKPDPPKNLQMKPLKN | |
| | SQVEVSWEYPDSWSTPHSYFSLKFFVRIQRKKEKMKETEE | |
| | GCNQKGAFLVEKTSTEVQCKGGNVCVQAQDRYYNSSCSK | |
| | WACVPCRVRSGGGGSGGGGSGGGGSRVIPVSGPARCLS | |
| | QSRNLLKTTDDMVKTAREKLKHYSCTAEDIDHEDITRDQTS | |
| | TLKTCLPLELHKNESCLATRETSSTTRGSCLPPQKTSLMMT | |
| | LCLGSIYEDLKMYQTEFQAINAALQNHNHQQIILDKGMLVAI | |
| | DELMQSLNHNGETLRQKPPVGEADPYRVKMKLCILLHAFS | |
| | TRVVTINRVMGYLSSA | |
| hsGSDMD-DEVD- | MGSAFERVVRRVVQELDHGGEFIPVTSLQSSTGFQPYCLV | 88 |
| mmIL18DR-mmIL1 | VRKPSSSWFWKPRYKCVNLSIKDILEPDAAEPDVQRGRSF | |
| | HFYDAMDGQIQGSVELAAPGQAKIAGGAAVSDSSSTSMNV | |
| | YSLSVDPNTWQTLLHERHLRQPEHKVLQQLRSRGDNVYV | |
| | VTEVLQTQKEVEVTRTHKREGSGRFSLPGATCLQGEGQG | |
| | HLSQKKTVTIPSGSTLAFRVAQLVIDSDLDVLLFPDKKQRTF | |
| | QPPATGHKRSTSEGAWPQLPSGLSMMRCLHNDEVDGVP | |
| | AEGAFTEDFQGLRAEVETISKELELLDRELCQLLLEGLEGVL | |
| | RDQLALRALEEALEQGQSLGPVEPLDGPAGAVLECLVLSS | |
| | GMLVPELAIPVVYLLGALTMLSETQHKLLAEALESQTLLGPL | |
| | ELVGSLLEQSAPWQERSTMSLPPGLLGNSWGEGAPAWVL | |
| | LDECGLELGEDTPHVCWEPQAQGRMCALYASLALLSGLS | |
| | QEPHGSGEGRGSLLTCGDVEENPGPHFGRLHCTTAVIRNI | |
| | NDQVLFVDKRQPVFEDMTDIDQSASEPQTRLIIYAYGDSRA | |
| | RGKAVTLSVKDSKMSTLSCKNKIISFEEMDPPENIDDIQSDL | |
| | IFFQKRVPGHNKMEFESSLYEGHFLACQKEDDAFKLILKKK | |
| | DENGDKSVMFTLTNLHQSGSGATNFSLLKQAGDVEENPG | |
| | PVPIRQLHYRLRDEQQKSLVLSDPYELKALHLNGQNINQQV | |
| | IFSMSFVQGEPSNDKIPVALGLKGKNLYLSCVMKDGTPTLQ | |
| | LESVDPKQYPKKKMEKRFVFNKIEVKSKVEFESAEFPNWYI | |
| | STSQAEHKPVFLGNNSGQDIIDFTMESVSS | |
| hsGSDMD-DEVD- | MGSAFERVVRRVVQELDHGGEFIPVTSLQSSTGFQPYCLV | 89 |
| mmIL1-mmIL18- | VRKPSSSWFWKPRYKCVNLSIKDILEPDAAEPDVQRGRSF | |
| mmIFN-alpha-2 | HFYDAMDGQIQGSVELAAPGQAKIAGGAAVSDSSSTSMNV | |
| | YSLSVDPNTWQTLLHERHLRQPEHKVLQQLRSRGDNVYV | |
| | VTEVLQTQKEVEVTRTHKREGSGRFSLPGATCLQGEGQG | |
| | HLSQKKTVTIPSGSTLAFRVAQLVIDSDLDVLLFPDKKQRTF | |
| | QPPATGHKRSTSEGAWPQLPSGLSMMRCLHNDEVDGVP | |
| | AEGAFTEDFQGLRAEVETISKELELLDRELCQLLLEGLEGVL | |
| | RDQLALRALEEALEQGQSLGPVEPLDGPAGAVLECLVLSS | |
| | GMLVPELAIPVVYLLGALTMLSETQHKLLAEALESQTLLGPL | |
| | ELVGSLLEQSAPWQERSTMSLPPGLLGNSWGEGAPAWVL | |
| | LDECGLELGEDTPHVCWEPQAQGRMCALYASLALLSGLS | |
| | QEPHGSGATNFSLLKQAGDVEENPGPVPIRQLHYRLRDEQ | |
| | QKSLVLSDPYELKALHLNGQNINQQVIFSMSFVQGEPSNDK | |
| | IPVALGLKGKNLYLSCVMKDGTPTLQLESVDPKQYPKKKM | |
| | EKRFVFNKIEVKSKVEFESAEFPNWYISTSQAEHKPVFLGN | |
| | NSGQDIIDFTMESVSSGSGEGRGSLLTCGDVEENPGPNFG | |
| | RLHCTTAVIRNINDQVLFVDKRQPVFEDMTDIDQSASEPQT | |
| | RLIIYMYKDSEVRGLAVTLSVKDSKMSTLSCKNKIISFEEMD | |
| | PPENIDDIQSDLIFFQKRVPGHNKMEFESSLYEGHFLACQK | |
| | EDDAFKLILKKKDENGDKSVMFTLTNLHQSGSGQCTNYALL | |
| | KLAGDVESNPGPARLCAFLVMLIVMSYWSICSLGCDLPHTY | |
| | NLRNKRALKVLAQMRRLPFLSCLKDRQDFGFPLEKVDNQQ | |
| | IQKAQAIPVLRDLTQQTLNLFTSKASSAAWNATLLDSFQND | |
| | LHQQLNDLQTCLMQQVGVQEPPLTQEDALLAVRKYFHRIT | |
| | VYLREKKHSPCAWEVVRAEVWRALSSSVNLLPRLSEEKE | |
| hsGSDMD-DEVD- | MGSAFERVVRRVVQELDHGGEFIPVTSLQSSTGFQPYCLV | 90 |
| mmIL18-mmIL1- | VRKPSSSWFWKPRYKCVNLSIKDILEPDAAEPDVQRGRSF | |
| mmIFN-alpha-2 | HFYDAMDGQIQGSVELAAPGQAKIAGGAAVSDSSSTSMNV | |
| | YSLSVDPNTWQTLLHERHLRQPEHKVLQQLRSRGDNVYV | |
| | VTEVLQTQKEVEVTRTHKREGSGRFSLPGATCLQGEGQG | |
| | HLSQKKTVTIPSGSTLAFRVAQLVIDSDLDVLLFPDKKQRTF | |
| | QPPATGHKRSTSEGAWPQLPSGLSMMRCLHNDEVDGVP | |
| | AEGAFTEDFQGLRAEVETISKELELLDRELCQLLLEGLEGVL | |
| | RDQLALRALEEALEQGQSLGPVEPLDGPAGAVLECLVLSS | |
| | GMLVPELAIPVVYLLGALTMLSETQHKLLAEALESQTLLGPL | |
| | ELVGSLLEQSAPWQERSTMSLPPGLLGNSWGEGAPAWVL | |
| | LDECGLELGEDTPHVCWEPQAQGRMCALYASLALLSGLS | |
| | QEPHGSGEGRGSLLTCGDVEENPGPNFGRLHCTTAVIRNI | |
| | NDQVLFVDKRQPVFEDMTDIDQSASEPQTRLIIYMYKDSEV | |
| | RGLAVTLSVKDSKMSTLSCKNKIISFEEMDPPENIDDIQSDLI | |
| | FFQKRVPGHNKMEFESSLYEGHFLACQKEDDAFKLILKKK | |
| | DENGDKSVMFTLTNLHQSGSGATNFSLLKQAGDVEENPG | |
| | PVPIRQLHYRLRDEQQKSLVLSDPYELKALHLNGQNINQQV | |
| | IFSMSFVQGEPSNDKIPVALGLKGKNLYLSCVMKDGTPTLQ | |
| | LESVDPKQYPKKKMEKRFVFNKIEVKSKVEFESAEFPNWYI | |
| | STSQAEHKPVFLGNNSGQDIIDFTMESVSSGSGQCTNYAL | |
| | LKLAGDVESNPGPARLCAFLVMLIVMSYWSICSLGCDLPHT | |
| | YNLRNKRALKVLAQMRRLPFLSCLKDRQDFGFPLEKVDNQ | |
| | QIQKAQAIPVLRDLTQQTLNLFTSKASSAAWNATLLDSFCN | |
| | DLHQQLNDLQTCLMQQVGVQEPPLTQEDALLAVRKYFHRI | |
| | TVYLREKKHSPCAWEVVRAEVWRALSSSVNLLPRLSEEKE | |
| GSDMD-DEVD | MGSAFERVVRRVVQELDHGGEFIPVTSLQSSTGFQPYCLV | 107 |
| (human) | VRKPSSSWFWKPRYKCVNLSIKDILEPDAAEPDVQRGRSF | |
| | HFYDAMDGQIQGSVELAAPGQAKIAGGAAVSDSSSTSMNV | |
| | YSLSVDPNTWQTLLHERHLRQPEHKVLQQLRSRGDNVYV | |
| | VTEVLQTQKEVEVTRTHKREGSGRFSLPGATCLQGEGQG | |
| | HLSQKKTVTIPSGSTLAFRVAQLVIDSDLDVLLFPDKKQRTF | |
| | QPPATGHKRSTSEGAWPQLPSGLSMMRCLHNDEVDGVP | |
| | AEGAFTEDFQGLRAEVETISKELELLDRELCQLLLEGLEGVL | |
| | RDQLALRALEEALEQGQSLGPVEPLDGPAGAVLECLVLSS | |
| | GMLVPELAIPVVYLLGALTMLSETQHKLLAEALESQTLLGPL | |
| | ELVGSLLEQSAPWQERSTMSLPPGLLGNSWGEGAPAWVL | |
| | LDECGLELGEDTPHVCWEPQAQGRMCALYASLALLSGLS | |
| | QEPH | |
| Gasdermin D | MGSAFERVVRRVVQELDHGGEFIPVTSLQSSTGFQPYCLV | 48 |
| P57764 | VRKPSSSWFWKPRYKCVNLSIKDILEPDAAEPDVQRGRSF | |
| (human) | HFYDAMDGQIQGSVELAAPGQAKIAGGAAVSDSSSTSMNV | |
| | YSLSVDPNTWQTLLHERHLRQPEHKVLQQLRSRGDNVYV | |
| | VTEVLQTQKEVEVTRTHKREGSGRFSLPGATCLQGEGQG | |
| | HLSQKKTVTIPSGSTLAFRVAQLVIDSDLDVLLFPDKKQRTF | |
| | QPPATGHKRSTSEGAWPQLPSGLSMMRCLHNFLTDGVPA | |
| | EGAFTEDFQGLRAEVETISKELELLDRELCQLLLEGLEGVL | |
| | RDQLALRALEEALEQGQSLGPVEPLDGPAGAVLECLVLSS | |
| | GMLVPELAIPVVYLLGALTMLSETQHKLLAEALESQTLLGPL | |
| | ELVGSLLEQSAPWQERSTMSLPPGLLGNSWGEGAPAWVL | |
| | LDECGLELGEDTPHVCWEPQAQGRMCALYASLALLSGLS | |
| | QEPH | |
| Gasdermin E | MFAKATRNFLREVDADGDLIAVSNLNDSDKLQLLSLVTKKK | 49 |
| O60443 | RFWCWQRPKYQFLSLTLGDVLIEDQFPSPVVVESDFVKYE | |
| (human) | GKFANHVSGTLETALGKVKLNLGGSSRVESQSSFGTLRKQ | |
| | EVDLQQLIRDSAERTINLRNPVLQQVLEGRNEVLCVLTQKIT | |
| | TMQKCVISEHMQVEEKCGGIVGIQTKTVQVSATEDGNVTK | |
| | DSNVVLEIPAATTIAYGVIELYVKLDGQFEFCLLRGKQGGFE | |
| | NKKRIDSVYLDPLVFREFAFIDMPDAAHGISSQDGPLSVLK | |
| | QATLLLERNFHPFAELPEPQQTALSDIFQAVLFDDELLMVLE | |
| | PVCDDLVSGLSPTVAVLGELKPRQQQDLVAFLQLVGCSLQ | |
| | GGCPGPEDAGSKQLFMTAYFLVSALAEMPDSAAALLGTCC | |
| | KLQIIPTLCHLLRALSDDGVSDLEDPTLTPLKDTERFGIVQRL | |
| | FASADISLERLKSSVKAVILKDSKVFPLLLCITLNGLCALGRE | |
| | HS | |
| VSV N | MSVTVKRIIDNTVVVPKLPANEDPVEYPADYFRKSKEIPLYI | 28 |
| | NTTKSLSDLRGYVYQGLKSGNVSIIHVNSYLYGALKDIRGKL | |
| | DKDWSSFGINIGKAGDTIGIFDLVSLKALDGVLPDGVSDAS | |
| | RTSADDKWLPLYLLGLYRVGRTQMPEYRKKLMDGLTNQC | |
| | KMINEQFEPLVPEGRDIFDVWGNDSNYTKIVAAVDMFFHM | |
| | FKKHECASFRYGTIVSRFKDCAALATFGHLCKITGMSTEDV | |
| | TTWILNREVADEMVQMMLPGQEIDKADSYMPYLIDFGLSS | |
| | KSPYSSVKNPAFHFWGQLTALLLRSTRARNARQPDDIEYT | |
| | SLTTAGLLYAYAVGSSADLAQQFCVGDNKYTPDDSTGGLT | |
| | TNAPPQGRDVVEWLGWFEDQNRKPTPDMMQYAKRAVMS | |
| | LQGLREKTIGKYAKSEFDK | |
| VSV P | MDNLTKVREYLKSYSRLDQAVGEIDEIEAQRAEKSNYELFQ | 29 |
| | EDGVEEHTKPSYFQAADDSDTESEPEIEDNQGLYAPDPEA | |
| | EQVEGFIQGPLDDYADEEVDVVFTSDWKQPELESDEHGKT | |
| | LRLTSPEGLSGEQKSQWLSTIKAVVQSAKYWNLAECTFEA | |
| | SGEGVIMKERQITPDVYKVTPVMNTHPSQSEAVSDVWSLS | |
| | KTSMTFQPKKASLQPLTISLDELFSSRGEFISVGGDGRMSH | |
| | KEAILLGLRYKKLYNQARVKYSL | |
| VSV L | MEVHDFETDEFNDFNEDDYATREFLNPDERMTYLNHADY | 30 |
| | NLNSPLISDDIDNLIRKENSLPIPSMWDSKNWDGVLEMLTS | |
| | CQANPIPTSQMHKWMGSWLMSDNHDASQGYSFLHEVDK | |
| | EAEITFDVVETFIRGWGNKPIEYIKKERWTDSFKILAYLCQK | |
| | FLDLHKLTLILNAVSEVELLNLARTFKGKVRRSSHGTNICRIR | |
| | VPSLGPTFISEGWAYFKKLDILMDRNFLLMVKDVIIGRMQTV | |
| | LSMVCRIDNLFSEQDIFSLLNIYRIGDKIVERQGNFSYDLIKM | |
| | VEPICNLKLMKLARESRPLVPQFPHFENHIKTSVDEGAKIDR | |
| | GIRFLHDQIMSVKTVDLTLVIYGSFRHWGHPFIDYYTGLEKL | |
| | HSQVTMKKDIDVSYAKALASDLARIVLFQQFNDHKKWFVN | |
| | GDLLPHDHPFKSHVKENTWPTAAQVQDFGDKWHELPLIKC | |
| | FEIPDLLDPSIIYSDKSHSMNRSEVLKHVRMNPNTPIPSKKV | |
| | LQTMLDTKATNWKEFLKEIDEKGLDDDDLIIGLKGKERELKL | |
| | AGRFFSLMSWKLREYFVITEYLIKTHFVPMFKGLTMADDLT | |
| | AVIKKMLDSSSGQGLKSYEAICIANHIDYEKWNNHQRKLSN | |
| | GPVFRVMGQFLGYPSLIERTHEFFEKSLIYYNGRPDLMRVH | |
| | NNTLINSTSQRVCWQGQEGGLEGLRQKGWSILNLLVIQRE | |
| | AKIRNTAVKVLAQGDNQVICTQYKTKKSRNVVELQGALNQ | |
| | MVSNNEKIMTAIKIGTGKLGLLINDDETMQSADYLNYGKIPIF | |
| | RGVIRGLETKRWSRVTCVTNDQIPTCANIMSSVSTNALTVA | |
| | HFAENPINAMIQYNYFGTFARLLLMMHDPALRQSLYEVQDK | |
| | IPGLHSSTFKYAMLYLDPSIGGVSGMSLSRFLIRAFPDPVTE | |
| | SLSFWRFIHVHARSEHLKEMSAVFGNPEIAKFRITHIDKLVE | |
| | DPTSLNIAMGMSPANLLKTEVKKCLIESRQTIRNQVIKDATIY | |
| | LYHEEDRLRSFLWSINPLFPRFLSEFKSGTFLGVADGLISLF | |
| | QNSRTIRNSFKKKYHRELDDLIVRSEVSSLTHLGKLHLRRG | |
| | SCKMWTCSATHADTLRYKSWGRTVIGTTVPHPLEMLGPQ | |
| | HRKETPCAPCNTSGFNYVSVHCPDGIHDVFSSRGPLPAYL | |
| | GSKTSESTSILQPWERESKVPLIKRATRLRDAISWFVEPDS | |
| | KLAMTILSNIHSLTGEEWTKRQHGFKRTGSALHRFSTSRMS | |
| | HGGFASQSTAALTRLMATTDTMRDLGDQNFDFLFQATLLY | |
| | AQITTTVARDGWITSCTDHYHIACKSCLRPIEEITLDSSMDY | |
| | TPPDVSHVLKTWRNGEGSWGQEIKQIYPLEGNWKNLAPA | |
| | EQSYQVGRCIGFLYGDLAYRKSTHAEDSSLFPLSIQGRIRG | |
| | RGFLKGLLDGLMRASCCQVIHRRSLAHLKRPANAVYGGLIY | |
| | LIDKLSVSPPFLSLTRSGPIRDELETIPHKIPTSYPTSNRDMG | |
| | VIVRNYFKYQCRLIEKGKYRSHYSQLWLFSDVLSIDFIGPFSI | |
| | STTLLQILYKPFLSGKDKNELRELANLSSLLRSGEGWEDIHV | |
| | KFFTKDILLCPEEIRHACKFGIAKDNNKDMSYPPWGRESRG | |
| | TITTIPVYYTTTPYPKMLEMPPRIQNPLLSGIRLGQLPTGAH | |
| | YKIRSILHGMGIHYRDFLSCGDGSGGMTAALLRENVHSRGI | |
| | FNSLLELSGSVMRGASPEPPSALETLGGDKSRCVNGETC | |
| | WEYPSDLCDPRTWDYFLRLKAGLGLQIDLIVMDMEVRDSS | |
| | TSLKIETNVRNYVHRILDEQGVLIYKTYGTYICESEKNAVTIL | |
| | GPMFKTVDLVQTEFSSSQTSEVYMVCKGLKKLIDEPNPDW | |
| | SSINESWKNLYAFQSSEQEFARAKKVSTYFTLTGIPSQFIPD | |
| | PFVNIETMLQIFGVPTGVSHAAALKSSDRPADLLTISLFYMAI | |
| | ISYYNINHIRVGPIPPNPPSDGIAQNVGIAITGISFWLSLMEK | |
| | DIPLYQQCLAVIQQSFPIRWEAVSVKGGYKQKWSTRGDGL | |
| | PKDTRISDSLAPIGNWIRSLELVRNQVRLNPFNEILFNQLCR | |
| | TVDNHLKWSNLRRNTGMIEWINRRISKEDRSILMLKSDLHE | |
| | ENSWRD | |
| VSV M | MSSLKKILGLKGKGKKSKKLGIAPPPYEEDTSMEYAPSAPID | 31 |
| | KSYFGVDEMDTYDPNQLRYEKFFFTVKMTVRSNRPFRTYS | |
| | DVAAAVSHWDHMYIGMAGKRPFYKILAFLGSSNLKATPAVL | |
| | ADQGQPEYHAHCEGRAYLPHRMGKTPPMLNVPEHFRRPF | |
| | NIGLYKGTIELTMTIYDDESLEAAPMIWDHFNSSKFSDFREK | |
| | ALMFGLIVEKKASGAWVLDSIGHFK | |
| LCMV GP | MGQIVTMFEALPHIIDEVINIVIIVLIIITSIKAVYNFATCGILALV | 32 |
| | SFLFLAGRSCGMYGLNGPDIYKGVYQFKSVEFDMSHLNLT | |
| | MPNACSANNSHHYISMGSSGLELTFTNDSILNHNFCNLTSA | |
| | FNKKTFDHTLMSIVSSLHLSIRGNSNHKAVSCDFNNGITIQY | |
| | NLSFSDPQSAISQCRTFRGRVLDMFRTAFGGKYMRSGWG | |
| | WAGSDGKTTWCSQTSYQYLIIQNRTWENHCRYAGPFGMS | |
| | RILFAQEKTKFLTRRLAGTFTWTLSDSSGVENPGGYCLTK | |
| | WMILAAELKCFGNTAVAKCNVNHDEEFCDMLRLIDYNKAAL | |
| | SKFKQDVESALHVFKTTVNSLISDQLLMRNHLRDLMGVPY | |
| | CNYSKFWYLEHAKTGETSVPKCWLVTNGSYLNETHFSDQI | |
| | EQEADNMITEMLRKDYIKRQGSTPLALMDLLMFSTSAYLISI | |
| | FLHLVKIPTHRHIKGGSCPKPHRLTNKGICSCGAFKVPGVK | |
| | TIWKRR | |
| Gasdermin D | MGSAFERVVRRVVQELDHGGEFIPVTSLQSSTGFQPYCLV | 54 |
| GSDM-NT | VRKPSSSWFWKPRYKCVNLSIKDILEPDAAEPDVQRGRSF | |
| P57764 | HFYDAMDGQIQGSVELAAPGQAKIAGGAAVSDSSSTSMNV | |
| (human) | YSLSVDPNTWQTLLHERHLRQPEHKVLQQLRSRGDNVYV | |
| | VTEVLQTQKEVEVTRTHKREGSGRFSLPGATCLQGEGQG | |
| | HLSQKKTVTIPSGSTLAFRVAQLVIDSDLDVLLFPDKKQRTF | |
| | QPPATGHKRSTSEGAWPQLPSGLSMMRCLHNFLTD | |
| Gasdermin E | MFAKATRNFLREVDADGDLIAVSNLNDSDKLQLLSLVTKKK | 55 |
| GSDM-NT | RFWCWQRPKYQFLSLTLGDVLIEDQFPSPVVVESDFVKYE | |
| O60443 | GKFANHVSGTLETALGKVKLNLGGSSRVESQSSFGTLRKQ | |
| (human) | EVDLQQLIRDSAERTINLRNPVLQQVLEGRNEVLCVLTQKIT | |
| | TMQKCVISEHMQVEEKCGGIVGIQTKTVQVSATEDGNVTK | |
| | DSNVVLEIPAATTIAYGVIELYVKLDGQFEFCLLRGKQGGFE | |
| | NKKRIDSVYLDPLVFREFAFIDMPD | |
| GSDME | MFAKATRNFLKEVDAGGDLISVSHLNDSDKLQLLSLVTKKK | 76 |
| (mouse) | RYWCWQRPKYQILSATLEDVLTEGHCLSPVVVESDFVKYE | |
| | SKCENHKSGAIGTVVGKVKLNVGGKGVVESHSSFGTLRKQ | |
| | EVDVQQLIQDAVKRTVNMDNLVLQQVLESRNEVLCVLTQKI | |
| | MTTQKCVISEHVQSEETCGGMVGIQTKTIQVSATEDGTVTT | |
| | DTNVVLEIPAATTIAYGIMELFVKQDGQFEFCLLQGKHGGF | |
| | EHERKLDSVYLDPLAYREFAFLDMLDGGQGISSQDGPLRV | |
| | VKQATLHLERSFHPFAVLPAQQQRALFCVLQKILFDEELLR | |
| | ALEQVCDDVAGGLWSSQAVLAMEELTDSQQQDLTAFLQL | |
| | VGYRIQGEHPGPQDEVSNQKLFATAYFLVSALAEMPDNAT | |
| | VFLGTCCKLHVISSLCCLGG | |
| GSDMD | MPSAFEKVVKNVIKEVSGSRGDLIPVDSLRNSTSFRPYCLL | 77 |
| (mouse) | NRKFSSSRFWKPRYSCVNLSIKDILEPSAPEPEPECFGSFK | |
| | VSDVVDGNIQGRVMLSGMGEGKISGGAAVSDSSSASMNV | |
| | CILRVTQKTWETMQHERHLQQPENKILQQLRSRGDDLFVV | |
| | TEVLQTKEEVQITEVHSQEGSGQFTLPGALCLKGEGKGHQ | |
| | SRKKMVTIPAGSILAFRVAQLLIGSKWDILLVSDEKQRTFEP | |
| | SSGDRKAVGQRHHGLNVLAALCSIGKQLSLLSDGIDEEELI | |
| | EAADFQGLYAEVKACSSELESLEMELRQQILVNIGKILQDQ | |
| | PSMEALEASLGQGLCSGGQVEPLDGPAGCILECLVLDSGE | |
| | LVPELAAPIFYLLGALAVLSETQQQLLAKALETTVLSKQLEL | |
| | VKHVLEQSTPWQEQSSVSLPTVLLGDCWDEKNPTWVLLE | |
| | ECGLRLQVESPQVHWEPTSLIPTSALYASLFLLSSLGQKPC | |
| deltaM51 | MSSLKKILGLKGKGKKSKKLGIAPPPYEEDTSMEYAPSAPID | 85 |
| | KSYFGVDEDTYDPNQLRYEKFFFTVKMTVRSNRPFRTYSD | |
| | VAAAVSHWDHMYIGMAGKRPFYKILAFLGSSNLKATPAVLA | |
| | DQGQPEYHAHCEGRAYLPHRMGKTPPMLNVPEHFRRPFN | |
| | IGLYKGTIELTMTIYDDESLEAAPMIWDHFNSSKFSDFREKA | |
| | LMFGLIVEKKASGAWVLDSIGHFK | |
| E7/E6/E2 | MHGDTPTLHEYMLDLQPETTDLYGYGQLNDSSEEEDEIDG | 108 |
| | PAGQAEPDRAHYNIVTFCCKCDSTLRLCVQSTHVDIRTLED | |
| | LLMGTLGIVCPICSQKPGSGATNFSLLKQAGDVEENPGPM | |
| | HQKRTAMFQDPQERPRKLPQLCTELQTTIHDIILECVYCKQ | |
| | QLLRREVYDFAFRDLCIVYRGGNPYAVCDKCLKFYSKISEY | |
| | RHYCYSLYGTTLEQQYNKPLCDGLIRCINCQKPLCPEEKQR | |
| | HLDKKQRFHNIRGRWTGRCMSCCRSSRTRRETQLGSGEG | |
| | RGSLLTCGDVEENPGPMETLCQRLNVCQDKILTHYENDST | |
| | DLRDHIDYWKHMRLECAIYYKAREMGFKHINHQVVPTLAVS | |
| | KNKALQAIELQLTLETIYNSQYSNEKWTLQDVSLEVYLTAPT | |
| | GCIKKHGYTVEVQFDGDICNTMHYTNWTHIYICEEASVTVV | |
| | EGQVDYYGLYYVHEGIRTYFVQFKDDAEKYSKNKVWEVHA | |
| | GGQVILCPTSVFSSNEVSSPEIIRQHLANHPAATHTKAVALG | |
| | TEETQTTIQRPRSEPDTGNPCHTTKLLHRDSVDSAPILTAF | |
| | NSSHKGRINCNSNTTPIVHLKGDANTLKCLRYRFKKHCTLY | |
| | TAVSSTWHWTGHNVKHKSAIVTLTYDSEWQRDQFLSQVKI | |
| | PKTITVSTGFMSI | |
| GSDME | MFAKATRNFLKEVDAGGDLISVSHLNDSDKLQLLSLVTKKK | 109 |
| (mouse) | RYWCWQRPKYQILSATLEDVLTEGHCLSPVVVESDFVKYE | |
| Q9Z2D3 | SKCENHKSGAIGTVVGKVKLNVGGKGVVESHSSFGTLRKQ | |
| | EVDVQQLIQDAVKRTVNMDNLVLQQVLESRNEVLCVLTQKI | |
| | MTTQKCVISEHVQSEETCGGMVGIQTKTIQVSATEDGTVTT | |
| | DTNVVLEIPAATTIAYGIMELFVKQDGQFEFCLLQGKHGGF | |
| | EHERKLDSVYLDPLAYREFAFLDMLDGGQGISSQDGPLRV | |
| | VKQATLHLERSFHPFAVLPAQQQRALFCVLQKILFDEELLR | |
| | ALEQVCDDVAGGLWSSQAVLAMEELTDSQQQDLTAFLQL | |
| | VGYRIQGEHPGPQDEVSNQKLFATAYFLVSALAEMPDNAT | |
| | VFLGTCCKLHVISSLCCLLHALSDDSVCDFHNPTLAPLRDT | |
| | ERFGIVQRLFASADIALERMQFSAKATILKDSCIFPLILHITLS | |
| | GLSTLSKEHEEELCQSGHATGQD | |
| GSDME(Q9Z2D3)- | MFAKATRNFLKEVDAGGDLISVSHLNDSDKLQLLSLVTKKK | 110 |
| IL12 (mouse) | RYWCWQRPKYQILSATLEDVLTEGHCLSPVVVESDFVKYE | |
| | SKCENHKSGAIGTVVGKVKLNVGGKGVVESHSSFGTLRKQ | |
| | EVDVQQLIQDAVKRTVNMDNLVLQQVLESRNEVLCVLTQKI | |
| | MTTQKCVISEHVQSEETCGGMVGIQTKTIQVSATEDGTVTT | |
| | DTNVVLEIPAATTIAYGIMELFVKQDGQFEFCLLQGKHGGF | |
| | EHERKLDSVYLDPLAYREFAFLDMLDGGQGISSQDGPLRV | |
| | VKQATLHLERSFHPFAVLPAQQQRALFCVLQKILFDEELLR | |
| | ALEQVCDDVAGGLWSSQAVLAMEELTDSQQQDLTAFLQL | |
| | VGYRIQGEHPGPQDEVSNQKLFATAYFLVSALAEMPDNAT | |
| | VFLGTCCKLHVISSLCCLLHALSDDSVCDFHNPTLAPLRDT | |
| | ERFGIVQRLFASADIALERMQFSAKATILKDSCIFPLILHITLS | |
| | GLSTLSKEHEEELCQSGHATGQDGSGEGRGSLLTCGDVE | |
| | ENPGPMGWSCIILFLVATATGVHSMWELEKDVYVVEVDWT | |
| | PDAPGETVNLTCDTPEEDDITWTSDQRHGVIGSGKTLTITV | |
| | KEFLDAGQYTCHKGGETLSHSHLLLHKKENGIWSTEILKNF | |
| | KNKTFLKCEAPNYSGRFTCSWLVQRNMDLKFNIKSSSSSP | |
| | DSRAVTCGMASLSAEKVTLDQRDYEKYSVSCQEDVTCPTA | |
| | EETLPIELALEARQQNKYENYSTSFFIRDIIKPDPPKNLQMK | |
| | PLKNSQVEVSWEYPDSWSTPHSYFSLKFFVRIQRKKEKMK | |
| | ETEEGCNQKGAFLVEKTSTEVQCKGGNVCVQAQDRYYNS | |
| | SCSKWACVPCRVRSGGGGSGGGGSGGGGSRVIPVSGPA | |
| | RCLSQSRNLLKTTDDMVKTAREKLKHYSCTAEDIDHEDITR | |
| | DQTSTLKTCLPLELHKNESCLATRETSSTTRGSCLPPQKTS | |
| | LMMTLCLGSIYEDLKMYQTEFQAINAALQNHNHQQIILDKG | |
| | MLVAIDELMQSLNHNGETLRQKPPVGEADPYRVKMKLCILL | |
| | HAFSTRVVTINRVMGYLSSA | |

### Validation of Viral Replication (Fitness)—TCID₅₀/Cell Killing

HEK293F cells grown in suspension culture in Freestyle™ 293 Expression Medium (ThermoFisher Scientific) are infected with a low MOI (0.0005) of either VSV-GP or VSV-GP-GSDME-IL12. On the day of infection, the cells have a confluence of 60-70%. One well is counted (Countess™ cell counter, Invitrogen) before infecting the other wells with 0.005 MOI of one of the virus constructs. Culture supernatants (3 mL total volume) are harvested, and samples are analyzed 8 h, 16 h, 24 h, 32 h, 40 h and 48 h post infection for viral replication and cell killing. Viral replication is assessed using detection of viral genomes by qPCR in the supernatant of the cultures at the indicated timepoints. Virus induced cell killing is assessed by counting the viable cells in culture samples at the indicated time points.

### TCID₅₀ Assay

In 96-well plates 1×10⁴ BHK-21 cells in 100 μL supplemented GMEM (Gibco) are seeded per well. 24 h later, the adherent cells are infected with eleven 0.5×log 10 serial dilutions of the virus or the diluent alone (negative control) before incubation for three days at 37° C., 5% CO₂. Brightfield images of the cell culture wells are taken with the Tecan Spark Reader (Tecan) using a 4× objective. Whether the imaged wells are CPE positive or negative is assessed either by eye (i.e. visually) or automatically (i.e. via automated image analysis). The final TCID50/mL is calculated by the formula of Spearman-Kärber (1,2). Six serial dilution replicates are assessed for each virus sample with each serial dilution on a separate plate for six plates total. Based on those six replicates the TCID50/mL is calculated as described above.

Determination VSV-GP Genomic Copies by qPCR

RNA from cell culture supernatants is extracted using the MagMax-96 Viral RNA Isolation Kit (ThermoFisher, #AM1836) according to manufacturer's instructions. Genomic VSV-N copies are measured using iTaq Universal Probes One-Step Kit (BioRad, #1725141) with VSV-N primers (forward: 5′-AGT-ACC-GGA-GGA-TTG-ACG-ACT-AAT-3′, reverse: 5′-TCA-AAC-CAT-CCG-AGC-CAT-TC-3′) and probe (5′-ACC-GCC-ACA-AGG-CAG-AGA-TGT-GGT-3′). Amplification protocol: 50° C. for 10 min, 95° C. for 2 min and 40 cycles of 95° C. 15 s and 60° C. 30 s. A standard curve is set up by using VSV RNA in a concentration from 107 to 101 copies/ml. All qPCR samples are measured in technical triplicates.

### Western Blot Analysis

Tumor cells are infected at an MOI of 3. At indicated timepoints, cells are harvested and pelleted by centrifugation at 5.000 rpm, in a benchtop centrifuge. Cells are then lysed in RIPA buffer (Thermo Fisher, #89901) containing a protease inhibitor cocktail (Sigma Aldrich, #11836170001) and incubated in a ThemoMixer (Eppendorf) at 4° C. for 30 min shaking at 800 rpm. Cell debris are removed by centrifugation at 12.000 rpm for 20 min at 4° C. before protein extracts are analyzed on a Jess Simple Western System (BioTechne) using the following antibodies and reagents: primary antibodies, anti-DFNA5/GSDME (Abcam, ab215191); GSDMD (Cell Signaling Technology, #E9S1X, #E504N); ACTB (Cell Signaling Technology, #9662); VSV polyclonal rabbit serum (gift from S. Finke, Friedrich-Loeffler-Institute, Isle of Riems, Germany); for antibody detection the anti-Rabbit Detection or anti-Mouse detection modules (Biotechne, #DM-001, #DM-002) are used.

### In Vivo Experiments

Six to eight-week-old female mice are obtained from Charles River Laboratories (Wilmington, MA, Unites States). Tumors are implanted by subcutaneous injection in the right flank. Tumor size is measured with a caliper and volume is calculated using the formula: tumor volume [mm³]=(length [mm])×(width [mm]) 2×0.5. Treatment commences when the mean tumor volume reaches a size of 80-150 mm³. Virus solutions are used for intravenous (100 μl) or intratumoral (20 μl) injection. Mice are sacrificed when their tumor volume reach 1,500 mm³ or tumors show signs of ulcerations. Animals are euthanized by overdose on gas anesthesia (isoflurane) followed by cervical dislocation or exsanguination.

### Flow Cytometric Analysis

Tumor-draining lymph nodes (tdLN), spleens and tumors are harvested three and seven days after virus treatment. A single cell suspension is prepared from tdLNs and spleens by passing them through a 70 μm cell sieve using the rubber stamp of a 3 ml plastic syringe. For dissociation of tumors, a combination of enzymatic digestion with murine tumor dissociation kit (Miltenyi Biotec) and mechanical dissociation on a OctoDissociator (program 37_mTDK_1) according to the manufacturer's instruction is used. Cells are then washed with PBS, cell pellet is suspended in a defined volume of PBS and stained with a live/dead discrimination dye. After blocking of Fc-receptors with a Fc-receptor blocking reagent (Biolegend), cell suspension from tdLN is stained with antibodies characterizing dendritic cell differentiation whereas spleen and tumors are stained with antibodies characterizing activation and killing capacity of (antigen-specific) T cells.

The staining panels are as follows: A) dendritic cells in tdLNs: PDCA-1 Brilliant Violet 421 (clone 921, Biolegend), Ly-6C Starbright Violet 515 (clone ER-MP20, Bio-Rad), CD103 Brilliant Violet 605 (clone 2E7, Biolegend), F4/80 Starbright Violet 670 (clone CI: A3-1, Bio-Rad), CD80 and CD86 Brilliant Violet 786 (clones GL1 and 16-D10A1, both BD Biosciences), MHCII FITC (clone M5/114.15.2, Biolegend), CD25 Starbright Blue 675 (clone PC61.5.3, Bio-Rad), CCR7 PE (clone REA685, Miltenyi Biotec), Ly-6G PE-Cy5 (clone 1A8, Biolegend), CD11c PE-Cy7 (clone REA754, Miltenyi Biotec), XCR1 APC (clone REA707, Miltenyi Biotec), CD8α Alexa 700 (clone 53-6.7, Biolegend), CD3 APC-Cy7 (clone 17A2, Biolegend), CD19 APC-Cy7 (clone 6D5, Biolegend), CD225 APC-Cy7 (clone REA815, Miltenyi Biotec), CD45 APC/Fire810 (clone 30-F11, Biolegend); B) T cells in spleens and tumors: CD107a Brilliant Violet 421 (clone 1D4B, Biolegend), CD8α Starbright Violet 515 (clone KT15, Bio-Rad), CD103 Brilliant Violet 605 (clone 2E7, Biolegend), CD3 Starbright Violet 670 (clone KT3, Bio-Rad), CD4 Starbright Violet 710 (clone RM4-5, Bio-Rad), CD49b Brilliant Violet 786 (clone HMa2, BD Biosciences), Dextramer H-2Kb VSV NP-RGYVYQGL-FITC (Immudex), CD25 Starbright Blue 675 (clone PC61.5.3, Bio-Rad), Dextramer H-2 Db HPV 16 E7-RAHYNIVTF-PE (Immudex), Perforin PE/Dazzle 594 (clone S16009A, Biolegend), Granzyme B PE-Cy5.5 (clone NGZB, eBiosciences), IFN-γ PE-Cy7 (clone XMG1.2, Biolegend), TNF-α APC (clone MP6-XT22, Biolegend), CD39 Alexa 700 (clone Y23-1185, BD Biosciences), CD69 APC-Cy7 (clone H1.2F3, Biolegend), CD19 APC-Cy7 (clone 6D5, Biolegend), CD225 APC-Cy7 (clone REA815, Miltenyi Biotec), CD45 APC/Fire810 (clone 30-F11, Biolegend). Counting beads (Invitrogen) are added to the final suspension and cells are analyzed on a ZE5 4-laser flow cytometer (Bio-Rad). Data are analyzed using FlowJo software and visualized using GraphPad Prism.

### Cytokine ELISAs

Cytokine levels in virus-infected cell culture supernatants are determined by commercially available enzyme-linked immunosorbent assays (ELISAs) according to the manufacturer's protocol. Human IL12 is measured with the IL12p70 Human ELISA kit (Invitrogen, #BMS238) or the Human IL12p70 DuoSet ELISA kit (R&D Systems, #DY1270). Mouse IL12 is measured with the IL12 mouse ELISA kit (Invitrogen, BMS616) or the mouse IL12p70 DuoSet ELISA kit (R&D Systems, #DY419). For measurement of mouse IL1a, IL18 and IFNalpha ELISA kits are used as follows: mouse IL1 alpha ELISA Kit (ThermoFisher, #BMS611), mouse IL18 DuoSet ELISA (R&D Systems, #DY7625-05), and mouse IFNalpha ELISA Kit (R&D systems, #42120-1).

### Particle Separation by HPLC-SEC and Characterization by Multi-Angle Light Scattering (MALS).

### Sample Preparation

Samples are measured undiluted unless stated otherwise; if dilutions are required, a Tris-buffered, NaCl and L-Arg containing solution is used. Samples are transferred to Quan Recovery polypropylene vials with a high-performance surface (Waters, USA) and kept in the HPLC sample manager at 8° C. until injection. Total Recovery glass vials (Waters, USA) are also tested for comparison.

### HPLC-SEC Separation

Material: Acquity Arc Bio HPLC system equipped with a 2998 photodiode array (PDA) detector (Waters, Milford, MA, USA) and the software Empower 3 FR 5 (Waters, USA) for data acquisition and integration. The path length of the PDA flow cell is 10 mm. Multi-angle light scattering (MALS) Dawn detector controlled by Astra 8.1 (Wyatt Technology, Santa Barbara, CA, USA) integrated in the HPLC detector flow path. Analytical SEC columns with mean pore sizes of 50 nm (Tosoh TSKgel G4000PW in the standard stainless steel column housing and custom manufactured in a BioAssist version in polyetheretherketone (PEEK) housing) and over 100 nm (Tosoh TSKgel BioAssist G6PW) (Tosoh Bioscience, Griesheim, Germany).

Column housings made of stainless steel and a PEEK (BioAssist version) are tested during the method development. A temperature-controlled column oven keeps the columns at 25° C. The use of Waters Fraction Manager-Analytical (WFM-A) enables the optional collection of elution fractions. The mobile phase used after screenings consists of 50 mM Tris-HCl, 200 mM NaCl, 150 mM L-Arg, and 0.1 wt-% Dimethyl sulfoxide (DMSO), pH 8.0, prepared with MilliQ purified water and 0.22 μm filtered (Corning, Glendale, AZ, USA). A constant flow rate of 0.5 mL/min is applied, and the column is equilibrated for at least five column volumes before sample injections (sample volume of 10 μL).

Particle characterization. Due to its size, intact virus elutes in the exclusion peak which is characterized by online UV and MALS measurements as well as orthogonal offline analytical methods.

### Live-Cell Imaging of the Cell Death Phenotype

Live-cell imaging is performed with an automated Incucyte S3 system (EssenBiosciences/Sartorius). 1×10³-1×10⁴ 4T1 mouse breast or CT26CL.25 IFNAR−/− colorectal cancer cells are seeded 24 hours prior to image acquisition in a 96-well plate format. Tumor cells are stained according to the manufacturer's recommendations using the following reagents: IncuCyte® Caspase-3/7 Green Apoptosis Assay Reagent (Sartorius, #4440), IncuCyte® AnnexinV Red Reagent for apoptosis (Sartorius, #4641), and IncuCyte® Cytotox Red or Green Reagent for counting dead cells (Sartorius, #4632 or #4633). Tumor cells are infected with virus at MOIs ranging from 0.01 to 10 and placed in the Incucyte system at 37° C., 5% CO₂ in a humidified atmosphere shortly after. Images at 10× magnification are recorded every 10 to 120 minutes for 72 hours, depending on the assay. Image analysis includes fluorescent detection of dye uptake and phenotypic changes after applying TopHat filtering and confluency masking. Results are presented as the number of stained cells normalized to confluency, unless stated otherwise. All measurements are performed in triplicate and represented as means±SEM.

Measurement of Extracellular ATP (eATP)

ATP release into the cell culture supernatant is quantified using a bioluminescence-based RealTime-Glo™ Extracellular ATP Assay (Promega, #GA5010) according to the supplier's recommendations. In brief, 2000-5000 cells are seeded per 96-well cell culture plate (Corning, #3610) in a CO₂-independent growth media (Gibco, #18045088). After 24 h, cells are infected at an MOI of 10 and the 4×-reconstituted RealTime-Glo™ Extracellular ATP Assay reagent is added to the virus infected cells. For control, cells are left untreated. Luminescence is recorded every 10-15 minutes for 24 h in a multimode plate reader (Tecan Spark 3M) at 37° C. Luminescence (RLU) values are analyzed and plotted as n=3 replicates±SEM using GraphPad Prism software (Vers.9.5.0).

### NanoString Analysis

Tumors are homogenized with the SpeedMill PLUS (Analytik Jena, Jena, Germany) and RNA is extracted using Phenol: Chloroform: Isoamyl Alcohol (25:24:1) (Sigma-Aldrich, USA) and MagMAX-96 Total RNA Isolation Kit (Thermo Fisher) following manufacturer's instructions. Extracted RNA is analyzed for differential expression by means of the nCounter PanCancer Immune Profiling Panel and the nCounter FLEX Analysis System (NanoString Technologies, Seattle, WA, USA). Profiled data are pre-processed following the manufacturer's recommendations (3,4).

### Manufacturing VSV-GP-(Cargo) Drug Substance

HEK293F cells adapted to suspension culture are grown in BalanCD media (Irvine Scientific) supplemented with GlutaMax (ThermoFisher). Cells are infected with virus 48 hours post-seeding using an MOI of 0.0005. Harvest occurs ˜34 hours post-infection followed by clarification. Nuclease is added to the clarified harvest material to digest host cell DNA before viral capture by cation exchange chromatography. Bound viral particles are eluted in a salt step gradient and stored overnight in the refrigerator. VSV-GP-(cargo) preparations are further purified by multi-modal chromatography. Peak fractions are pooled and buffer exchanged into final formulation buffer followed by sterile filtration. The resulting drug substance is aliquoted and stored at −80° C. before use. All other viral preparations if not stated otherwise, are manufactured mutatis mutandis according to this protocol.

### Patient Derived Human Slice Culture Assay and Downstream Analysis

Sample collection. Following sample acquisition and immediate transport to the laboratory, CRC biospecimens are stored over night at 4° C. in MACS® tissue storage solution, 1% penicillin-streptomycin and protease inhibitor cocktail (1:100) before sectioned using a vibratome. Tissue slices are preserved just after sectioning (baseline sample) and submitted for pathological evaluation. Only samples with histologic features suggestive of tumor lesions and low numbers of dead cells are evaluated as adequate for subsequent analysis, retrospectively. Vibratome slices from different levels of the biospecimen are directly cultured for up to 72 h in a free-floating (FF) environment. Within the free-floating culture, the specimen is fully surrounded by nutrients and medium, cytokines growth factors produced by the tissue are more homogeneously distributed and therefore is well suited to study immunomodulatory actions after virus infection. During the culturing period, metabolic activity is assessed by microscopy and T-cell function is tested. In addition, cultured samples are submitted for pathological evaluation by means of H&E and immunofluorescence studies are performed.

Histopathological assessment. Tumor slices are fixed in formalin, washed with phosphate-buffered saline (PBS) and embedded in Histogel (ThermoFisher Scientific #HG-400-012). Samples are then processed for paraffin embedding. Thin sections (4 μm) are cut with a rotating microtome (Thermo Scientific Microm HM 355S) with ˜10 sections per sample. Slides are stained with hematoxylin/eosin (HE) and assessed by a trained pathologist. Tumor cell numbers, cell numbers of the tumor microenvironment including immune cells, fibroblasts, endothelial cells and dead cells, which are still identifiable as cells, are quantified using digital pathology. Clinical biopsies of CRC may reveal a wide spectrum of pathohistological findings, only specimens with histologically confirmed tumor content and low numbers of dead cells are included in the analysis. Because the true nature of the specimen is unknown at the time the biopsy is taken, all samples not complying with the above criteria are retrospectively discarded.

Oncolytic virus treatment and permissivity testing. To receive a representative picture of the whole human biospecimens, slices from different layers are allocated to different treatment groups and the respective baseline samples are preserved. All treatment arms comprise replicates from different layers of the biospecimen. Subsequently, patient-derived CRC slices are treated with 1×10⁷ TCID50 VSV-GP or variants thereof. Mock treated slices serve as negative control. Samples are cultured at 37° C., 5% CO₂ in a humidified incubator. After an infection period of 24 to 72 h, tissue integrity and presence of the Katushka signal within each single slice of the respective group is analyzed using a fluorescence microscope (Cell Observer, Zeiss). A five-tiered system is utilized to score for permissivity: 1-single Katushka-positive cells or one small positive patch (<5 cells), 2-positive patch (around 20 cells) and few interspersed positive cells, 3-huge positive cluster (>50 cells), 4-multiple positive clusters/big patch and a lot of Katushka-positive cells, 5-entire slice encompasses Katushka-positive signal. Only cases displaying an intact morphology in the mock treated controls are included. A case is called permissive (=production of infectious progeny=Katushka-positive) at 72 h post culturing, if more than half of the replicates score higher than 2. At 72 h, samples are fixed and further processed for paraffin embedding.

Immune stimulation and measurement of IL12 and IFNγ secretion. To determine IFN secretion during T-cell stimulation or virus treatment, patient-derived CRC slices are cultured under free floating conditions in a 48-well plate in 400 μl media. Four to six slices from different layers of the biospecimen are allocated to different groups before treatment. One group is incubated with human a-CD3 (1 μg/ml, clone [OKT3], BioLegend) and human a-CD28 (0.5 μg/ml, clone [CD28.2], BioLegend) antibodies, IgG (α-mouse IgG2a (clone [MOPC-173] BioLegend) and α-mouse IgG1 (clone [MOPC-21] BioLegend)) treated in replicates serve as negative control. Supernatant of individual slice cultures are analyzed for IL12 and INFγ secretion 72 h after treatment. The secreted amount of IL12 and IFNγ is measured using an flow cytometry-based bead Assay (Legendplex, #740390) according to the manufacturer's instructions.

Microscopy and image processing. H&E stained slides are scanned using the Pannoramic Scan II (3D Histech) and pictures are exported using the Panoramic Viewer Software. Immunofluorescence is captured using an inverted fluorescence microscope (Cell Observer, Zeiss).

### Example 1

### VSV-GP Infection Leads to Caspase-3/7 Activation in Murine Colorectal Tumor Cells

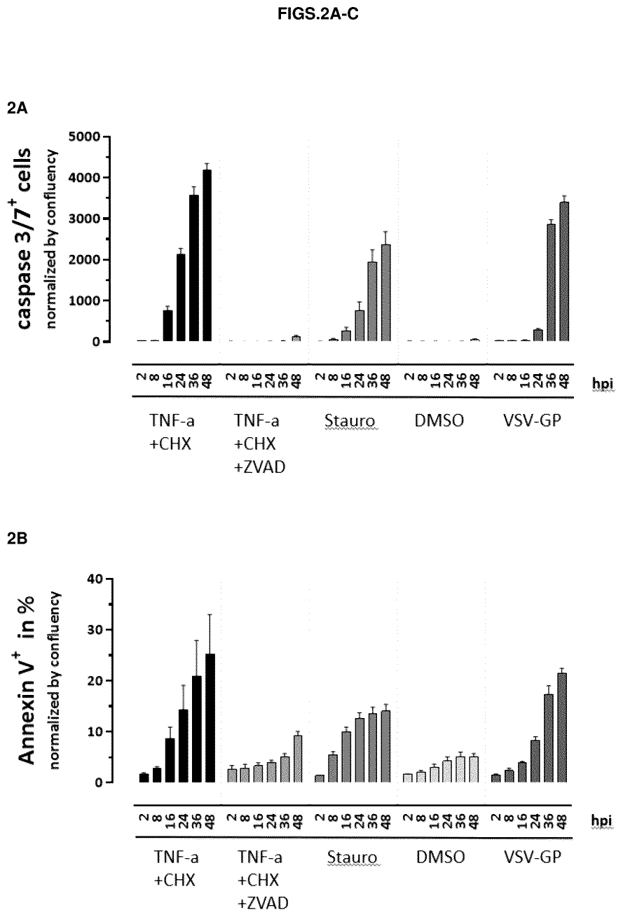

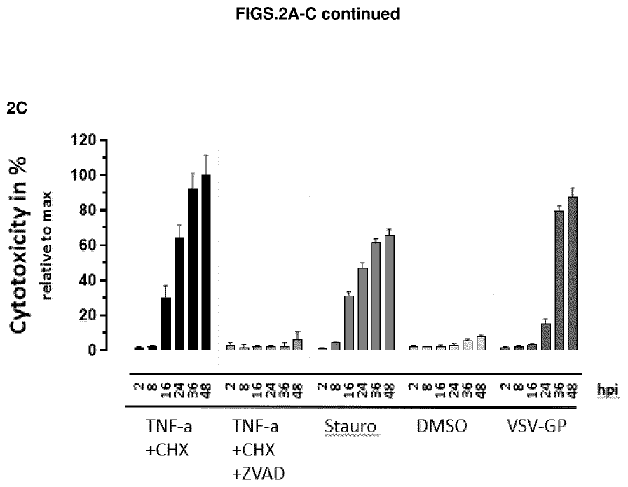

### FIGS. 2A-C

To examine whether the oncolytic VSV-GP can activate caspase-3 (Cas3) in tumor cells, CT26CI.25 mouse tumor cells were seeded into a 96-well cell-culture plate in 80 μl of complete DMEM media supplemented with 10% FBS per well. The next day, 20 μl of Incucyte® Caspase-3/7 Green Dye (Satorius), Incucyte® Annexin V Red Dye (1:200), or Incucyte® Cytotox Green Dye were added to a final concentration of 5 μM (Cas3/7 Green) or 250 nM (Cytotox Green), respectively, shortly before infection with VSV-GP at an MOI between 0.01 and 10. As a positive control, apoptotic cell death was induced by treating cells with 1 μM of staurosporine or 10 ng/ml TNFα and 10 μg/ml cycloheximide. As a negative control, cells were treated with the DMSO vehicle or caspases were blocked by the pan-caspase inhibitor 100 μM Z-VAD-fmk. Cas3/7 activation, AnnexinV, and cytotoxicity are monitored using an Incucyte S3 live cell imaging system (Satorius) for 72 h.

In CT26.CI25 cells infected at a multiplicity of infection (MOI) of 0.001, Cas3/7 were detected starting at 16 hpi. VSV-GP activated Cas3/7 as efficiently as 1 UM Staurosporine (FIG. 2A). Annexin V staining was detected as early as 8 hpi and increased over time (FIG. 2B), indicating phosphatidylserine exposure at the outer leaflet of the cellular membrane. At 16 hpi, the first signs of cytotoxicity caused by either Staurosporin or TNFα/CHX were detected. Cell killing by VSV-GP started around 24 hpi (FIG. 2C).

In conclusion, these findings demonstrate that VSV-GP can initiate the intrinsic apoptosis pathway, leading to the activation of Cas3 and Cas7. The increase in Annexin V staining indicates phosphatidylserine exposure on the outer membrane leaflet. The induction of apoptosis also resulted in an increased uptake of the CytoToxGreen dye, indicating the cytopathic effect was caused by the vector.

### Example 2

VSV-GP Infection Leads to Processing of GSDME into its Active Form in Cell Lines that Overexpresses GSDME (GSDMEoe)

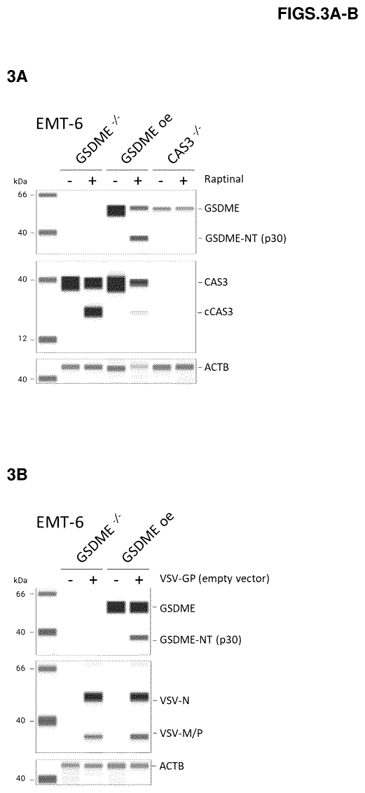

### FIGS. 3A-B

EMT6 GSDME knock-out (GSDME^{-/-}) or GSDME overexpressing (GSDME oe) mouse breast cancer cells were treated with 10 μM Raptinal for 2 h (FIG. 3A), or were infected with VSV-GP at an MOI of 3 for 24 h. For control, cells were left untreated. Additionally, we used a Cas3 knock-out EMT-6 cell line (Cas3^{-/-}) to show Cas3-dependency of GSDME cleavage (FIG. 3A).

Western blot analysis revealed that GSDMEoe cells treated with 10 μM Raptinal or infected with VSV-GP displayed a 30 kDa (p30) signal after treatment, corresponding to the active, pore-forming N-terminal domain of GSDME (FIG. 3A for Raptinal and FIG. 3B for VSV-GP, upper panel). In contrast, untreated or mock-infected control cells did not show any cleaved GSDME when compared to untreated or VSV-GP infected cells. The absence of GSDME p30 in Cas3^{-/-} EMT-6 cells, which endogenously express GSDME, indicated that GSDME cleavage was dependent on caspases, presumably Cas3. VSV-N, VSV-P/M and ACTB detection (FIGS. 3A and 3B, lower panels) were used as controls to confirm comparable levels of VSV-GP infection and protein loading, respectively.

In conclusion, VSV-GP is capable of inducing GSDME cleavage in cell lines that either endogenously or recombinantly expressed GSDME.

### Example 3

### Low Expression Levels of GSDME in Mouse Cancer Cell Lines Reflect the State in Human Cancers

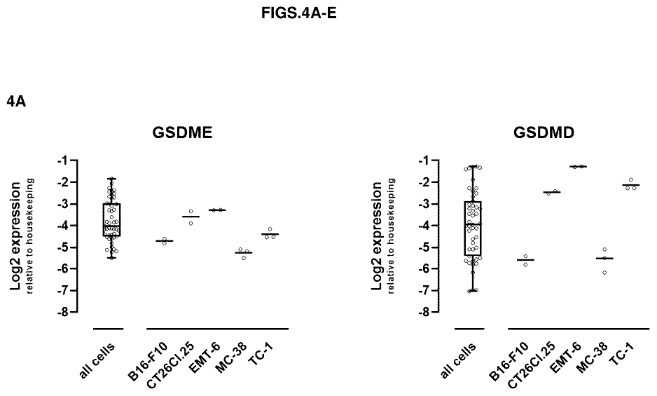

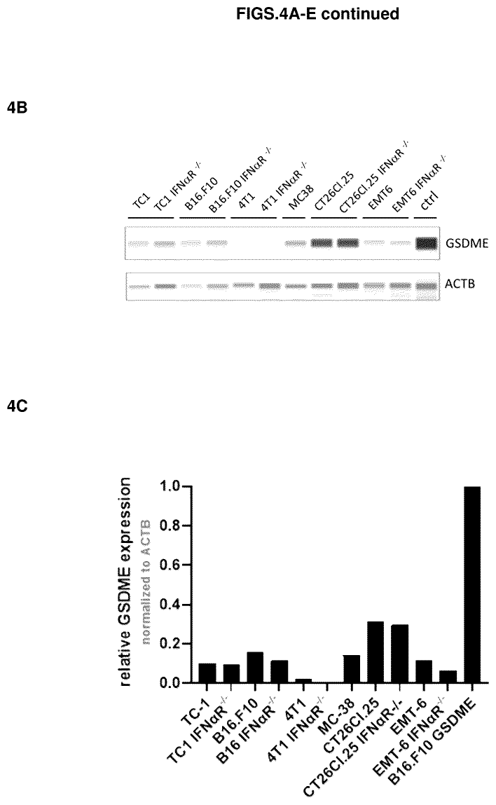

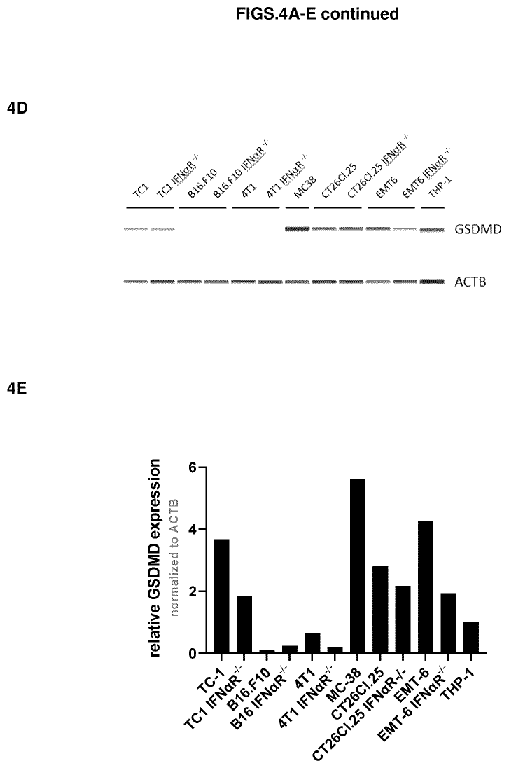

### FIGS. 4A-E

Both epigenetic silencing and loss-of-function mutations in GSDME could play crucial roles in the development and progression of various cancers.

To investigate this, several syngeneic mouse cancer cell lines were analyzed using next-generation sequencing (FIG. 4A) and western blotting (FIGS. 4B-E) to determine whether downregulation of GSDME or GSDMD expression is also commonly observed in mouse cancer cell lines.

Compared to CT26CI.25 cells, most mouse cancer cell lines exhibited low levels of GSDME expression, with 4T1 showing no detectable protein expression in the western blot (FIGS. 4B and C). In contrast to GSDME, except for B16.F10 or 4T1 expressing no or only low levels of GSDME, the majority of tumor cell lines displayed comparable or higher levels of GSDMD expression when compared to endogenous GSDMD expression in THP-1 cells. It is important to note that, although a species cross-reactive GSDMD mAB was utilized, GSDMD levels in human THP-1 cells used for control relative quantification may not be absolutely accurate.

In summary, it was found that GSDME gene expression was low in mouse tumor cells, which may reflect a mechanism by which tumors evade immunogenic cell death.

### Example 4

Active, Pore-Forming N-Terminal Domains of GSDMs Cannot be Expressed from Oncolytic Viruses-Full Length GSDME Did not have any Negative Impact on Virus Replication

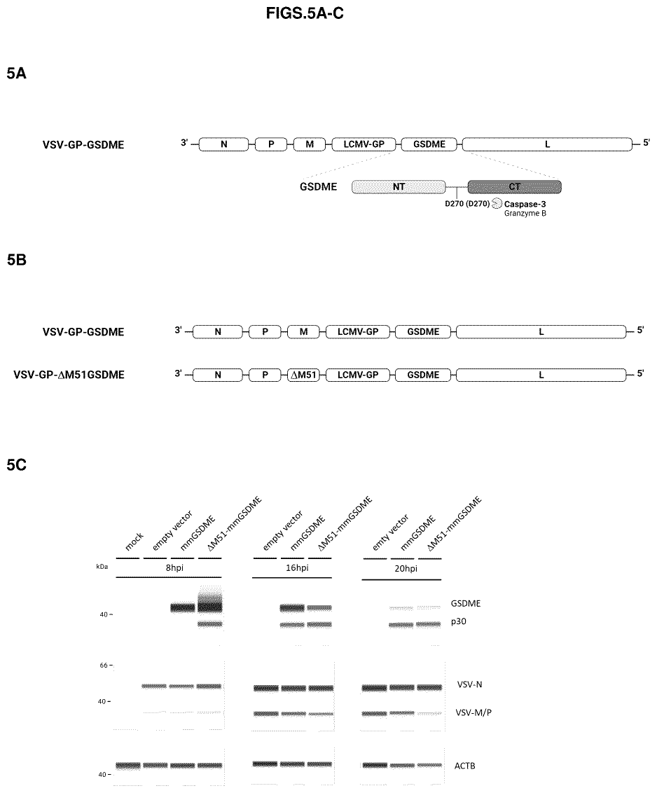

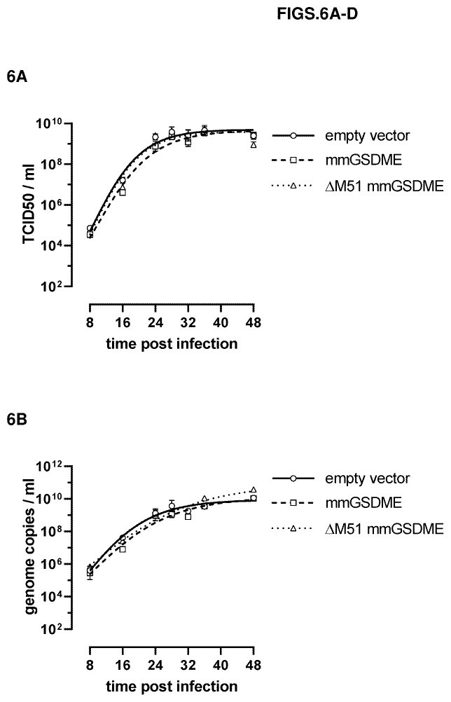

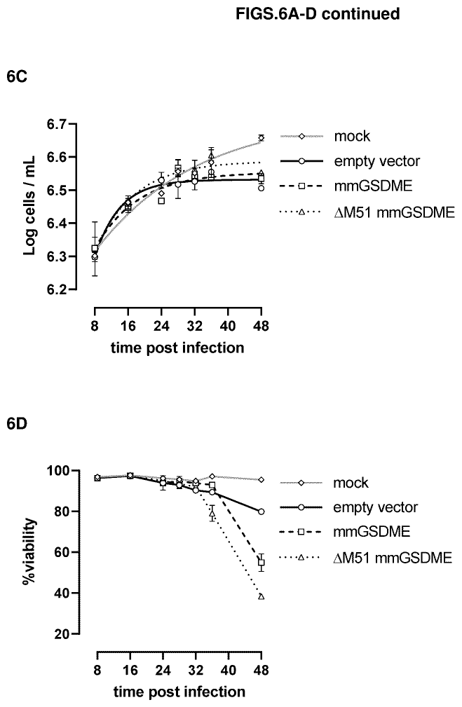

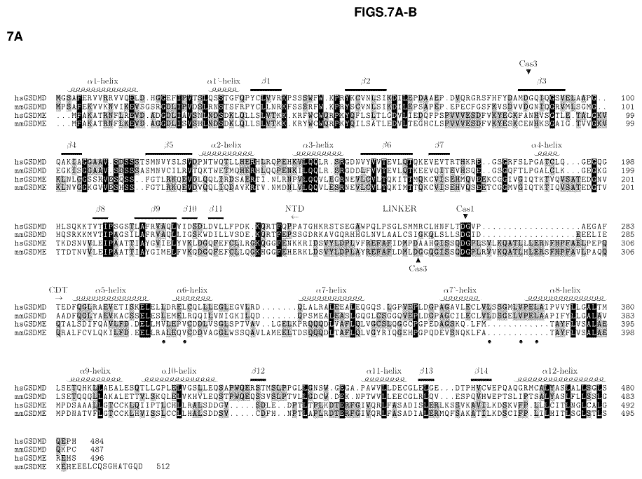

### FIGS. 5A-C and FIGS. 6A-D

Exogenous expression of the active N-terminal domain of any gasdermin family member—excluding Pelviakin (PJVK, DFNB59, which lacks pore-forming activity since a pore-forming function for the PJVKNT domain has not yet been demonstrated)—inevitably leads to cell death due to pore-forming activity. We attempted to produce VSV-GP expressing the active N-terminal domain of GSDMD (GSDMD-NT). However, the rapid onset of cell death induced by GSDMD-NT prevented recovery of the virus after co-transfection of the full-length VSV-GP-GSDMD-NT genome plasmid with VSV-N, -P, and -L helper plasmids into HEK293T cells (results not shown). Consequently, all attempts to produce a virus coding for an active GSDMD-NT domain have failed, and we assume this will be true for all active pore-forming domains of other gasdermin proteins.

As a next step, we explored the possibility of expressing full-length gasdermin in VSV-GP. We chose human (hs) (SEQ ID NO:49) and mouse (mm) (SEQ ID NO: 76) GSDME to be expressed from either VSV-GP or VSV-GP AM51 backbone, as both virus backbones can activate the intrinsic or extrinsic apoptotic pathway, leading to activation of caspase-3 and subsequent cleavage of GSDME, as shown in Examples 1 and 2. Surprisingly, we could recover both VSV-GP-GSDME and VSV-GP-ΔM51-GSDME viruses from recombinant DNA plasmids coding for the VSV-GP genome and the human or mouse GSDME (FIGS. 5A and 5B).

Western blot analysis of VSV-GP-mmGSDME or VSV-GP-ΔM51-mmGSDME infected GSDME low expressing 4T1-IFNAR1−/− mouse breast cancer cells (see Example 3, FIGS. 4B and 4C) showed that mmGSDME was indeed expressed and cleaved from both virus backbones, VSV-GP and VSV-GP-ΔM51. At 8, 16, and 20 hours post-infection (hpi), induction of mmGSDME cleavage was faster in VSV-GPΔM51-mmGSDME than in VSV-GP-mmGSDME infected cells.

However, due to the role of GSDMs in innate immunity and as a host defense mechanism against viruses, bacteria, and protozoan parasites, there were concerns that the virally overexpressed and activated GSDME might affect replication kinetics and infectious virus titers.

Surprisingly, when HEK293F suspension cells were infected with VSV-GP-mmGSDME or VSV-GP-ΔM51-mmGSDME at an MOI of 0.0005 TCID₅₀, no effect on replication kinetics and virus titers were observed at indicated timepoints (FIGS. 6A-D). Infectious TCID50 titers (FIG. 6A) and genomic copies (FIG. 6B) in the cell culture supernatant were comparable to parental VSV-GP (empty vector). Although total cell numbers were comparable in all infected samples (FIG. 6C), cell viability in cell cultures infected with viruses that exogenously expressed mmGSDME, started to drop significantly faster between 36 and 48 hpi when compared to the empty vector control (FIG. 6D). The decrease in viability can be explained by the facilitated uptake of the cell death stain across the membrane due to GSDME-NT pore formation and was slightly more prominent in VSV-GP-ΔM51-mmGSDME infected cultures.

In summary, full-length GSDME can be expressed by VSV-GP without impacting viral fitness. VSV-GP induced GSDME cleavage and activation by triggering the intrinsic or extrinsic apoptotic pathway, leading to a faster onset of cell death when compared to VSV-GP infected cells.

### Example 5

Human GSDMD can be Engineered with a Caspase-3 Cleavage Site And is Cleaved after VSV-GP Induced Apoptosis in Mouse and Human Cancer Cell Lines.

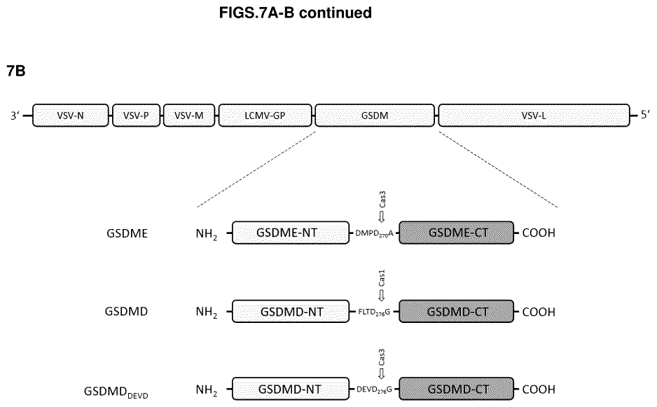

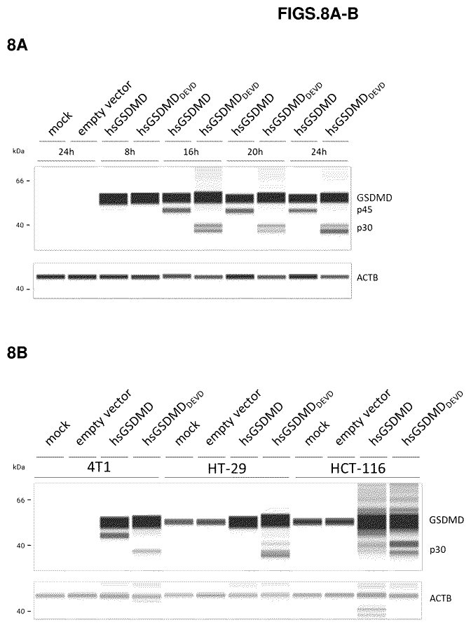

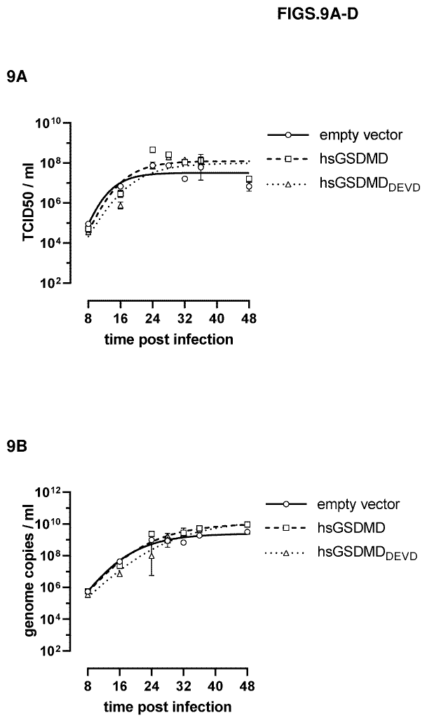

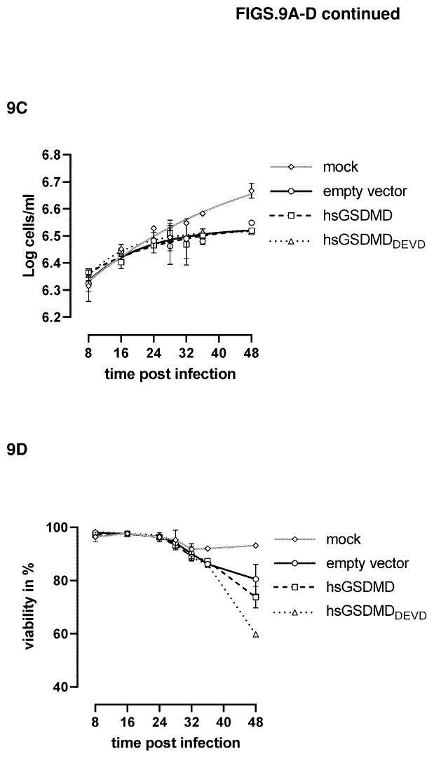

### FIGS. 7A-B, FIGS. 8A-D and FIGS. 9A-B

Since the gasdermin family shares sequence and structural similarities among its members, rational engineering of the proteolytic cleavage sites might be applied to other members of the gasdermin family.

FIG. 7A displays a structure-based sequence alignment between human (hs) and mouse (mm) gasdermin E and D. The N-terminal, pore-forming domain is not only highly conserved between mouse and human GSDMD or GSDME but also among the gasdermin family members. The highest variability is observed in the linker region separating the pore-forming N-terminal domain from the autoinhibiting C-terminal domain. The flexible linker domain contains the Cas3 cleavage site in GSDME and a Cas1 cleavage site in GSDMD, respectively.

GSDMD was engineered by replacing the minimally conserved Cas1 cleavage site FLTD with the canonical Cas3 cleavable tetrapeptide DEVD. Wildtype hsGSDMD (SEQ ID NO:48) and engineered hsGSDMD_{DEVD} (SEQ ID NO:107) were inserted into VSV-GP between the LCMV Glycoprotein and VSV polymerase L as independent transcriptional units (FIG. 7B).

4T1 mouse breast cancer cells confirmed to be negative for GSDME or low for endogenous GSDMD by western blotting (see Example 3, FIGS. 4B and 4C) were infected with either VSV-GP-hsGSDMD or -hsGSDMD_{DEVD} at a MOI of 3. Cell extracts were prepared at 8, 16, 20, and 24 hours post-infection. Virus-expressed GSDMD_{DEVD} can be cleaved and activated in VSV-GP-hsGSDMD_{DEVD} infected 4T1 cells after 16 hpi (FIG. 8A), whereas wildtype GSDMD containing the Cas1 cleavable tetrapeptide FLTD cannot be cleaved, indicating that virus-triggered apoptosis and Cas3 activation are responsible for GSDMD_{DEVD} activation. The additional peptide of approximately 45 kDa (p45) in samples infected with VSV-GP-hsGSDMD at 16 to 24 h is assumed to be an inactivated form of GSDMD cleaved by Cas3 at a secondary cleavage site at amino acid position 87 within the N-terminal domain. In VSV-GP-GSDMD_{DEVD} infected cells, p45 disappears. We cannot exclude that the additional Cas3 cleavage signal renders the GSDMD-NT inactive; however, 4T1 cells infected with GSDMD_{DEVD} virus show two signals at approximately 30 kDa, assuming that both the active GSDMD-NT (p30) and inactivated GSDMD-NT can be detected. A mutant GSDMD_{DEVD} D87A version, which cannot be cleaved by CAS3, confirmed this hypothesis (data not shown).

To exclude that GSDMD or GSDMD_{DEVD} expression impairs viral fitness, we performed multi-step replication kinetics of VSV-GP-hsGSDMD and VSV-GP-hsGSDMD_{DEVD}. HEK293F cells were infected at an MOI of 0.0005 TCID₅₀, and infectious TCID₅₀ (FIG. 9A), genomic titers (FIG. 9B), total cell counts (FIG. 9C), and viability (FIG. 9D) were monitored over time. We could not detect a significant difference compared to parental VSV-GP. Additionally, cell viability of VSV-GP-GSDMD, comparable to parental VSV-GP at 48 hpi, indicates that wildtype GSDMD, which is not cleaved by Cas3, is not activated during the course of infection. Only HEK293F cells infected with VSV-GP-hsGSDMD_{DEVD} showed a drop in cell viability at 48 hpi (FIG. 9D), as seen for VSV-GP-GSDME (FIG. 6D). However, cell viability by GSDMD_{DEVD} is less strongly impaired when compared to VSV-GP-GSDME, indicating that Cas3 cleavage might be less efficient in the engineered GSDMD_{DEVD}, or the additional Cas3 cleavage site at aa position 87 within the GSDMD-NT domain reduces the ability to kill infected cells efficiently by reducing active GSDMD-NT levels.

In summary, human GSDMD can be engineered to become a Cas3 substrate. Using the Cas3-cleavable version GSDMD_{DEVD}, activation of GSDMD can be triggered by VSV-GP, as shown for GSDME in Example 4. VSV-GP replication and viral infectious titers are not impaired by viral GSDMD_{DEVD} expression; however, the efficiency of GSDMD_{DEVD} activation and pore formation at cellular membranes might be less efficient since the secondary Cas3 cleavage site at aa position 87 within the GSDMD N-terminal domain might reduce GSDMD-NT protein levels and serve as a negative feedback loop for GSDMD activation.

### Example 6

Both, Virus Derived GSDME and GSDMD, are not Incorporated into Virus Particles and do not Interfere with Virus Particle Stability

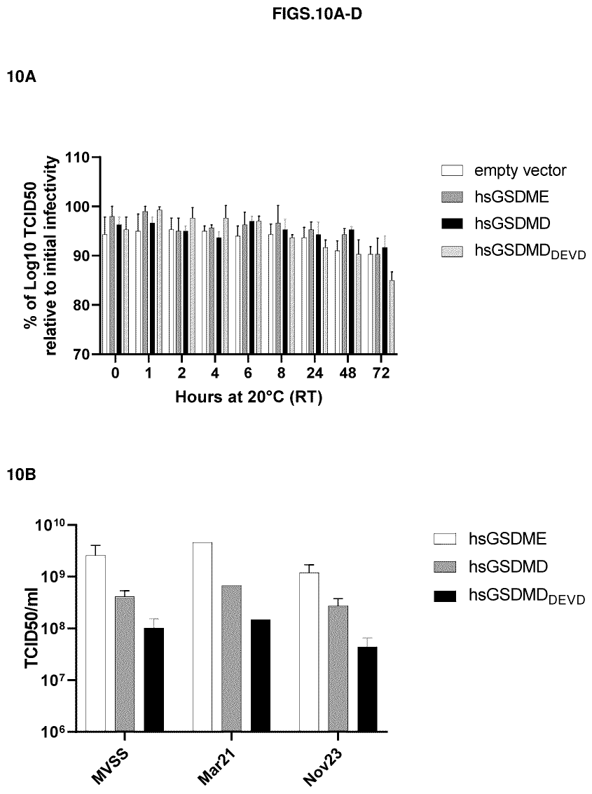

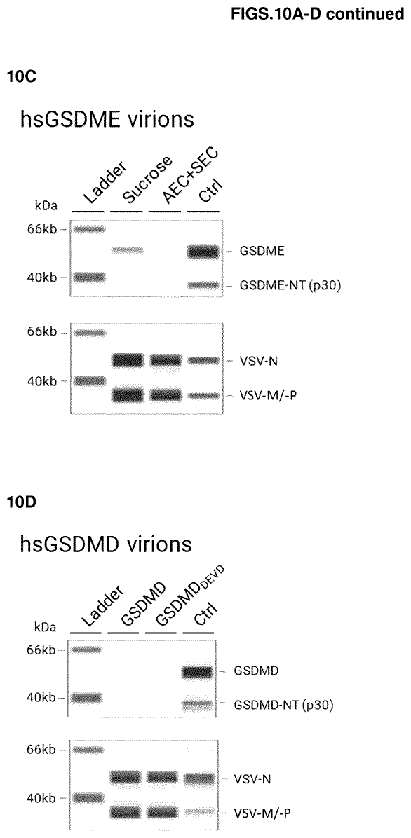

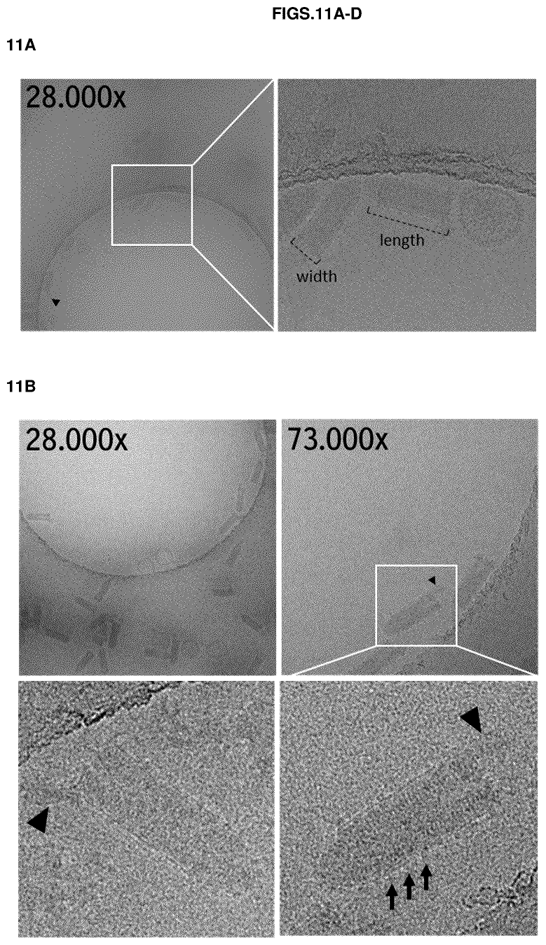

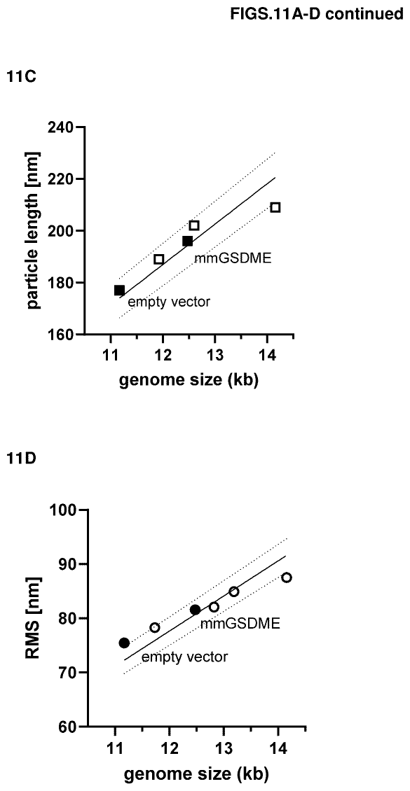

### FIGS. 10A-D and FIGS. 11A-D

GSDME or GSDMD pore formation at the plasma membrane may interfere with virus egress from the cell surface membrane, or particle budding might lead to the incorporation of activated gasdermin into the virus particle, thereby impacting infectivity or virion stability.

However, we did not observe any impact of GSDME or GSDMD expression on virus replication and infectious titers (Example 4 and 5, FIGS. 6A-D and FIGS. 8.A-D), nor on particle stability (FIG. 10A-B). TCID50 titers remained stable for at least 24 h at room temperature (RT) before they started to slightly decrease between 48 and 72 h at RT. Expression of GSDME or GSDMD in VSV-GP did not impact viral titers when compared to the empty vector (FIG. 10A). Only for GSDMD DEVD particles, we observed a loss of infectious titer of approximately 10% after 48 h at RT. Additionally, we did not observe any loss of infectious titer in a long-term stability experiment when virus stocks were stored at −80° C. for up to four years (FIG. 10B).

Analysis of purified virions suggests that virus budding at the plasma membrane might not take place at the same location, as we could not detect any GSDME or GSDMD by western blotting (FIG. 10C-E). In addition, cryo-EM analysis of virus particles did not confirm the existence of gasdermin pores in the viral envelope (FIG. 11A-B). An enlarged particle length (FIG. 11C) and larger gyration radius using Multi-Angle Light Scattering (MALS) (FIG. 11D) were observed, due to the longer RNA genome containing the genetic information of the recombinant gasdermin.

In summary, gasdermin protein could be expressed from a VSV-GP virus. The expression of gasdermin did not negatively impact virus replication, infectious titer, or virus particle stability. Viral envelopes were free of gasdermin pores, as evidenced by western blotting and cryo-EM.

### Example 7

### VSV-GP-GSDME Infected Tumor Cells Displayed a Pyroptotic Phenotype

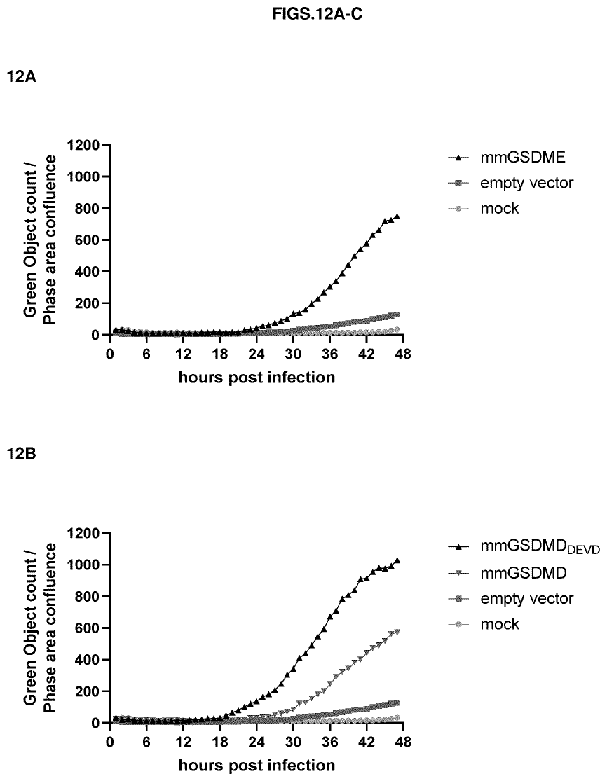

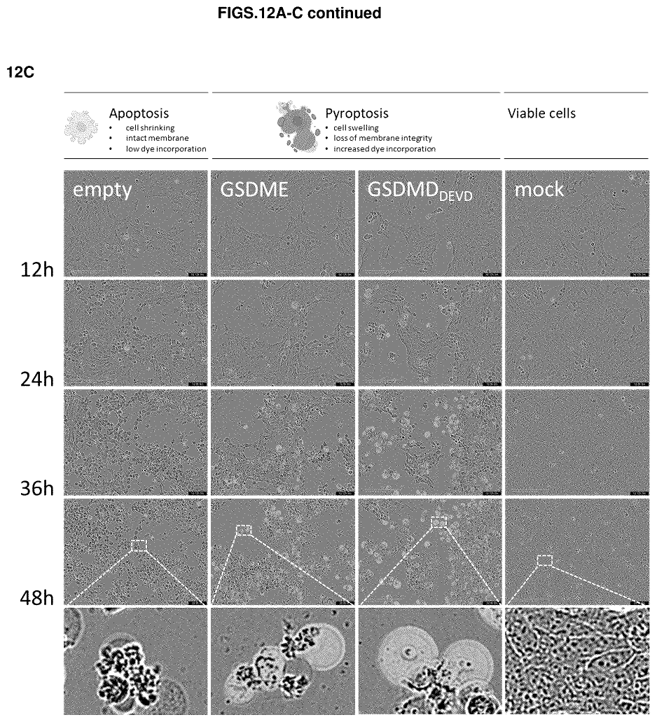

### FIGS. 12A-C

The cellular phenotype of pyroptotic cell death is distinct from that of apoptosis. Both types of regulated cell death are distinguished by specific phenotypic changes in the dying cells, particularly those induced by GSDMs. Apoptosis is characterized by cell shrinkage, chromatin condensation, nuclear fragmentation, membrane blebbing, and the formation of apoptotic bodies. In contrast, pyroptosis exhibits a different set of phenotypic changes, such as loss of membrane integrity, cell swelling, and nuclear fragmentation.

Live cell imaging was employed using the Incucyte S3 system (Sartorius) to further investigate the differences between VSV-GP and VSV-GP-GSDME. A cell death stain that needs to cross the membrane barrier before it enters and intercalates into nuclear DNA was used to measure membrane integrity. This approach allows for the observation and comparison of phenotypic changes in real-time, specifically looking for cell swelling (indicative of pyroptosis) or membrane blebbing (indicative of apoptosis), as well as the loss of membrane integrity and the onset of cell death.

Briefly, 4T1 mouse breast cancer cells were infected at an MOI of 3 with either VSV-GP-GSDME or VSV-GP. Untreated cells (mock) serve as a control. The experiment was conducted in a 96-well plate with 1×10⁴ cells per well. Cyto ToxGreen (Sartorius) cell death stain at 1 mM is added shortly before infection. Cell death was observed at 10× magnification every 10 minutes. Cytotox uptake and phenotypic changes was analyzed after applying TopHat filtering.

Compared to mock and VSV-GP, VSV-GP-mmGSDME (GSDME=SEQ ID NO: 76) or mmGSDMD_{DEVD} (SEQ ID NO:78) infected cells showed cell swelling, while VSV-GP exhibited apoptotic blebbing (FIG. 12C). Pyroptotic cells infected with either mmGSDME (FIG. 12A) or mmGSDMD_{DEVD} (FIG. 12B) did not show a faster onset of cell death, but increased uptake of cell death stain indicated a more prominent loss of membrane integrity compared to apoptotic dying cells in the control.

In summary, VSV-GP-GSDME displayed a pyroptotic phenotype, while VSV-GP showed an apoptotic phenotype. Virus-expressed GSDME is not only activated by caspase-3 (see example 5) but also forms a functional pore that leads to cell swelling and loss of membrane integrity. Similar results were observed with the colorectal cancer cell line CT26.CI25 containing an IFNAR1 knock-out (not shown).

### Example 8

Release of Danger Associated Molecular Patterns (DAMPs) Characterizes Immunogenic Cell Death after Pyroptotic Stimuli from VSV-GP-GSDME and VSV-GP-GSDMD_{DEVD}: Impact of OV Infection on Phosphatidylserine Exposure.

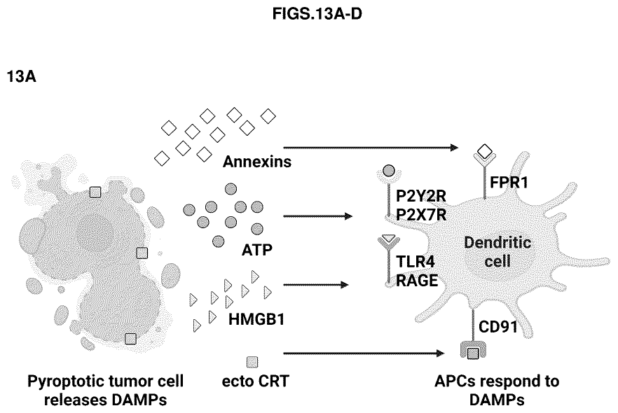

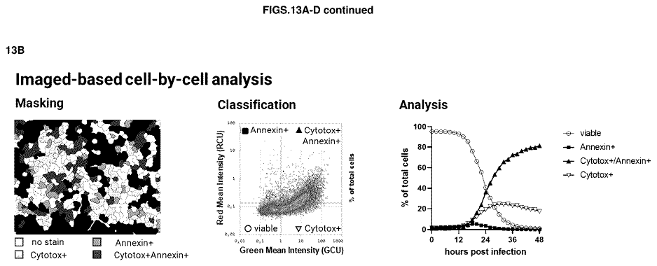

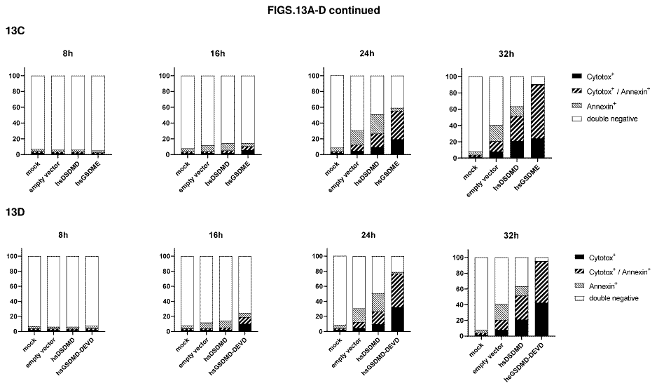

### FIGS. 13A-D

A key characteristic of Immunogenic Cell Death (ICD) is the sequential release of Danger-Associated Molecular Patterns (DAMPs), such as ATP, HMGB1, HSP70, along with the exposure of calreticulin (CRT) and phosphatidylserine (PtS) on the cell membrane. DAMPs function as effective chemo-attractants for immune cells, serving as “find-me” signals and as triggers for phagocytosis or “eat-me” signals. Moreover, DAMPs have co-stimulatory properties and act as secondary signals that can boost the migratory and co-stimulatory capacity of tumor-resident dendritic cells. DAMPs can be efficiently released through pores formed by gasdermin or after membrane rupture, which depends on pore formation and Ninjurin1 (Ninj1) activation at the plasma membrane (FIG. 13A).

The majority of phosphatidylserine can be found in the inner leaflet of the cellular membrane. After cell death stimuli such as apoptosis, phosphatidylserine is exposed on the outer surface of the membrane and serves as an “eat-me” signal for antigen-presenting cells such as dendritic cells or macrophages. It can be detected by the binding of Annexins to the outer membrane. We investigated whether infection of tumor cells with VSV-GP-GSDME or VSV-GP-GSDMD_{DEVD} affects the kinetics of phosphatidylserine membrane exposure and the level of AnnexinV staining. We utilized an automated live-cell imaging system (Incucyte) for image-based cell-by-cell analysis. To detect phosphatidylserine on infected cells, 4T1 breast cancer cells were stained with AnnexinV-Red. Membrane leakage by gasdermin pores and secondary necrosis were monitored additionally with the nuclear CytotoxGreen dye. Cells were incubated at 37° C., 5% CO₂, in a humidified atmosphere. Image acquisition with the automated microscope system was done every 10 minutes for 48 h. The images were analyzed using cell-by-cell and fluorescent masking before classification and time-resolved analysis of AnnexinV-, Cytotox-, and double-positive cell numbers (FIG. 13B).

In 4T1 cells infected with VSV-GP (empty vector) or VSV-GP-hsGSDMD (GSDMD=SEQ ID NO:48), which expresses the GSDMD that cannot be activated by Cas3, a clear AnnexinV single-positive population of 20% was observed, indicating early apoptosis starting between 16 and 24 hpi. However, in 4T1 cells infected with VSV-GP-hsGSDME (GSDME-SEQ ID NO:49), the AnnexinV-positive population was low at 24 hours or mostly absent after 32 hpi. There was a loss of membrane integrity by GSDME and a more rapid onset of cell death, as indicated by higher numbers of CytotoxGreen single- and CytotoxGreen/AnnexinV-Red double-positive cells at 24 and 32 hpi (FIG. 13C). Similar results were obtained when comparing VSV-GP-hsGSDMD and VSV-GP-hsGSDMD_{DEVD} (GSDMD_{DEVD}=SEQ ID NO:107). The genetically modified, Cas3 cleavable hsGSDMD_{DEVD} did not show any signs of early apoptosis indicated by AnnexinV single-positive cells but showed a higher percentage of CytotoxGreen positivity at 16-32 hpi (FIG. 13D).

Although we cannot exclude the possibility that the 36 kDa AnnexinV-Red dye crosses the cell membrane via the gasdermin pore after infection and binds to phosphatidylserine at the inner membrane leaflet, thus PtS is not exposed as an “eat-me” signal, this may not be possible due to the highly negatively charged nature of the N-terminal AnnexinV domain, which might hamper crossing the predominantly negatively charged GSDMD pore conduit by electrostatic repulsion.

In conclusion, the expression of GSDME or GSDMD_{DEVD} by VSV-GP resulted in a reduction of early apoptotic cells, indicating a change in the phenotype of cell death caused by the viral expression of the gasdermins. The high degree of AnnexinV staining observed within the population of pyroptotic dying cells (as depicted in FIG. 12C) suggests a potentially stronger DAMP signal for phagocytosis by tumor-resident dendritic cells from the dying tumor cells.

### Example 9

Evaluating the Release of DAMPs Through eATP Measurement Following VSV-GP-GSDME and -GSDMD_{DEVD} Infection to Demonstrate Immunogenic Cell Death Traits.

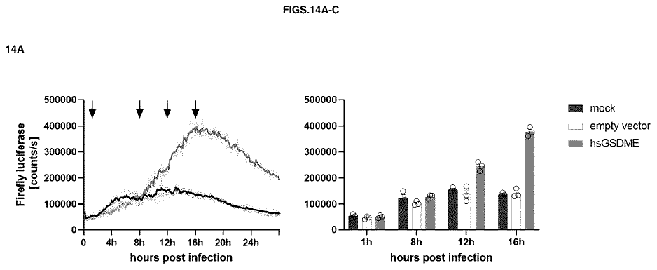

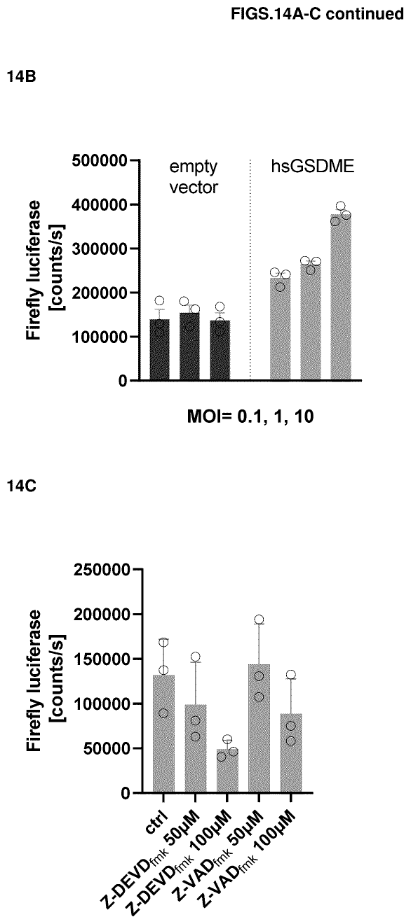

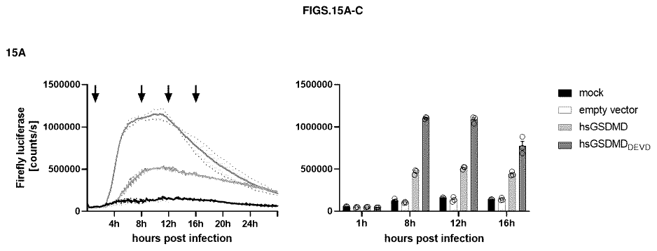

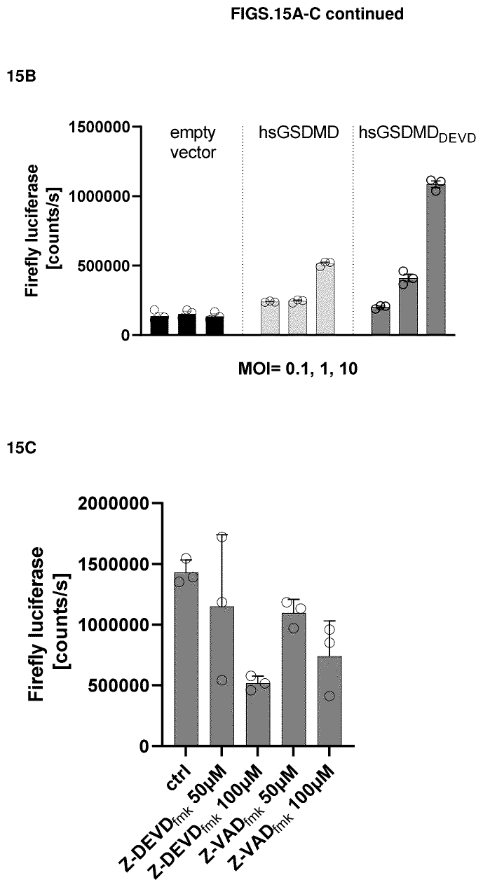

### FIGS. 14A-C & FIGS. 15A-C

ATP release was examined in the context of virus infection. 4T1 mouse breast cancer cells were infected at indicated MOIs with VSV-GP expressing either hsGSDME (SEQ ID NO:49), hsGSDMD (SEQ ID NO:48), or the Cas3 cleavable hsGSDMD_{DEVD} (SEQ ID NO:107) (FIG. 14 and FIG. 15). ATP release was measured using a Luciferase-based reporter assay that detects extracellular ATP (eATP) in the cell culture supernatant in real-time (RealTime-Glo™ Extracellular ATP Assay, Promega), following the manufacturer's recommendations.

The eATP release of VSV-GP-hsGSDME infected cells peaked between 16-18 hours post-infection. 4T1 cells infected with VSV-GP (empty vector) or left untreated (mock) did not exhibit an increased eATP leakage from the cultured cells (FIG. 14A). A comparison of the Cas3 cleavable hsGSDMD_{DEVD} virus to the wildtype parental virus, used as a control, revealed that the hsGSDMD_{DEVD} infected cells showed an increased release of eATP compared to the Cas1 cleavable wildtype GSDMD, the empty vector, or mock, peaking between 10-12 hpi (FIG. 15A). The release of eATP by either hsGSDME or hsGSDMD_{DEVD} at the time of maximum release (16 h for GSDME and 12 h for GSDMD, respectively) depends on the multiplicity of infection and increases from MOI=0.01 to 1.0 (FIGS. 14B and 15B). To confirm that the release of eATP depends on the activation of the gasdermin NT domain and subsequent pore formation by caspases, caspase activity was blocked using 50 PM and 100 UM of the caspase inhibitors Z-DEVDfmk and Z-VADfmk. Both inhibitors were able to reduce the eATP signal at indicated time points in a dose-dependent manner (FIGS. 14C and 15C), suggesting that the formation of an active GSDM pore is a prerequisite for the release of ATP.

In conclusion, both virus-expressed gasdermins, GSDME and GSDMD_{DEVD}, which are activated by Cas3, can form pores at the cellular membrane leading to an increased release of extracellular ATP. The released ATP can subsequently act as a DAMP contributing to the overall immunogenic response (see Examples 10 and 11).

### Example 10

Improved Tumor Control by Low Dose Treatment with VSV-GP-hsGSDME

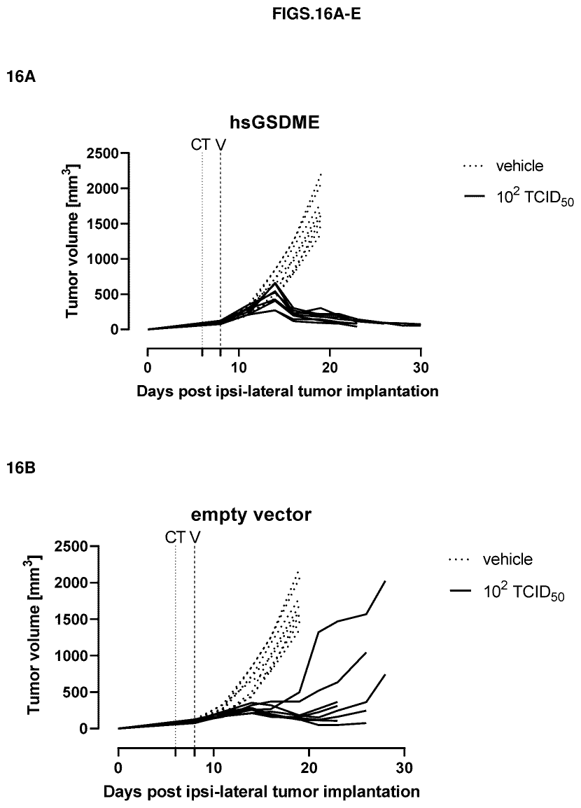

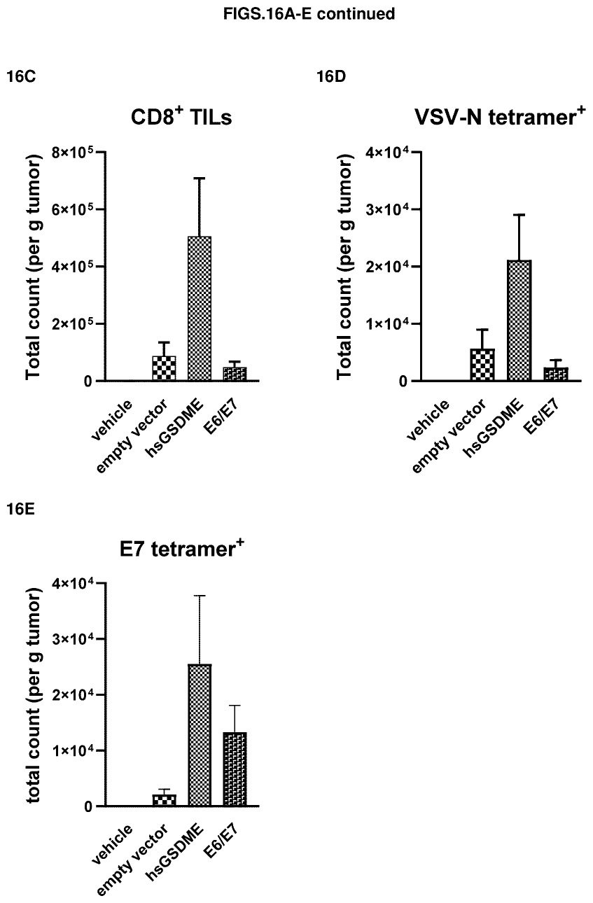

### FIGS. 16A-E

In this experiment, the therapeutic potency of VSV-GP-hsGSDME (GSDME=SEQ ID NO:49) was tested, and possible mechanisms of action were investigated.

For this purpose, tumors were implanted in C57BL/6J mice by subcutaneously injecting TC-1 tumor cells (1×10⁵ cells), deficient for the interferon alpha 1 receptor (TC-1-IFNAR1−/−), into the right flank. Following the same procedure, mice were injected subcutaneously in the left flank with 1×10⁵ TC-1 cells on Day 6 (CT=contralateral tumor). A virus treatment (10² TCID50) was given intratumorally, and animals were evaluated for tumor growth over time.

Only the VSV-GP-hsGSDME treatment could control the tumor growth of the treated ipsilateral tumors, while the growth of the contralateral non-treated tumors was not impacted by the treatments (FIGS. 16A and B).

Following the same procedure but without the implantation of a contralateral tumor, on day 7 post-treatment, tumors were collected and a single-cell suspension was prepared for flow cytometric analysis. Total counts of tumor-infiltrating CD8 T cells were strongly increased with hsGSDME expressing but not with the empty VSV-GP virus (FIG. 16C). In addition, hsGSDME expression led to increased counts of virus-specific (anti-VSV-N) and tumor-specific (anti-E7) CD8 T-cells. In particular, tumor-specific T-cell counts were even higher after hsGSDME treatment compared to a VSV-GP carrying the tumor-antigen E7/E6/E2 (SEQ ID NO:108) (FIGS. 16D-E).

In summary, the addition of hsGSDME to VSV-GP as a therapeutic cargo has the potential to enhance tumor control in permissive preclinical settings when administered intratumorally at a low dose, as it is associated with increased levels of tumor-infiltrating immune cells and improved anti-viral and anti-tumoral immunity.

### Example 11

### Benefit of Anti-PD-1 Co-Treatment in VSV-GP-hsGSDME Treated B16-F10 Melanoma

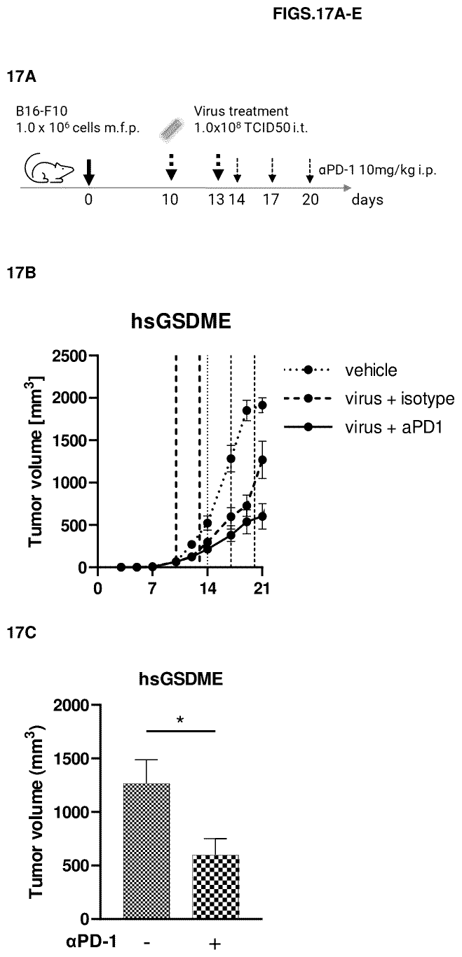

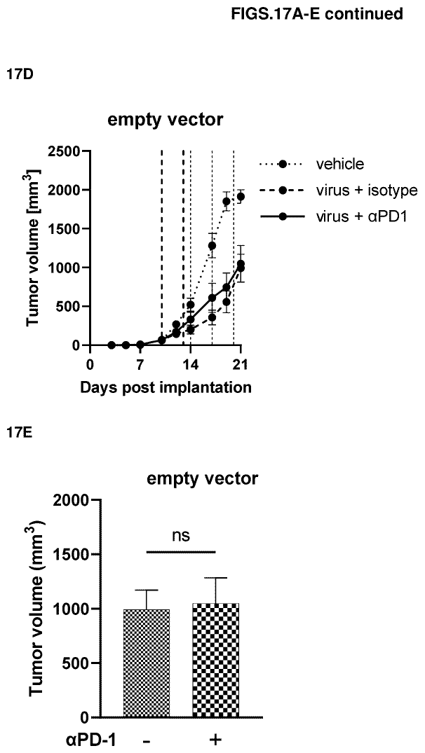

### FIGS. 17A-E & 18A-B

A study was conducted employing VSV-GP-hsGSDME (GSDME=SEQ ID NO: 49) to test whether cancer cells may exhibit increased responsiveness to anti-PD-1 therapy due to pyroptosis-induced inflammation.

In the study, B16F10 tumor cells (1×10⁶ cells) were implanted into the mammary fat pad tissue (2nd position) and treated intratumorally with the virus (108 TCID50) twice, three days apart. Subsequent intraperitoneal treatments (10 mg/kg anti-PD1) were administered three days apart (FIG. 17A). Mean tumor volume graphs (mean±SEM) indicated increased sensitivity to co-treatment with anti-PD1 for VSV-GP-GSDME treated animals (FIGS. 17B and C). Co-treatment with anti-PD1 led to a significant reduction in tumor volume on day 21 (FIG. 17C). A one-way ANOVA was performed (*p<0.05). In summary, the present experiment revealed that VSV-GP-hsGSDME effectively sensitizes a previously resistant preclinical tumor model to anti-PD1 treatment.

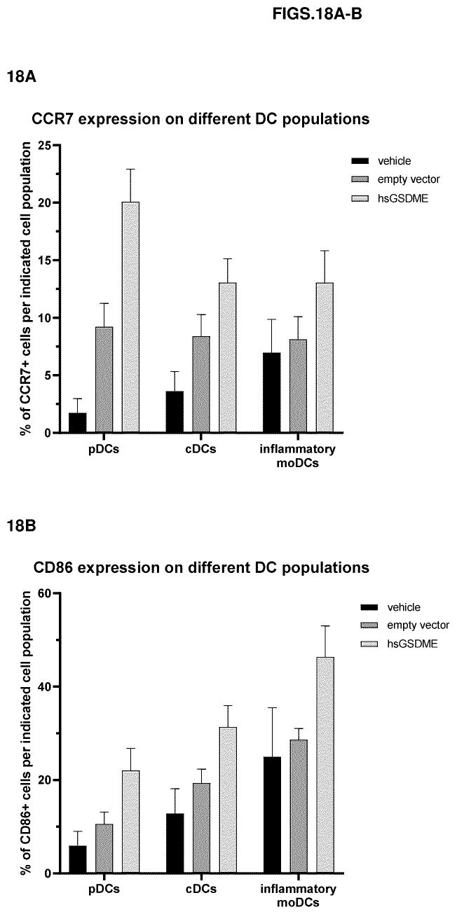

Additionally, the migratory and costimulatory capacity of different dendritic cell populations were analyzed. Cells were harvested on day seven post-viral treatment (but without PD-1 treatment) and analyzed by flow cytometry (FIGS. 18A-B). The frequency of A) migratory (CCR7-positive) and B) costimulatory (CD86-positive) cells among pDCs (plasmacytoid dendritic cells), cDCs (conventional dendritic cells), and inflammatory moDCs (monocyte-derived dendritic cells) were markedly increased in VSV-GP-GSDME treated mice compared to VSV-GP treatment.

### Example 12

In Vivo Data (EMT6): Mouse Treated with VSV-GP, VSV-GP-hsGSDME Or VSV-GP (deltaM51)-hsGSDME

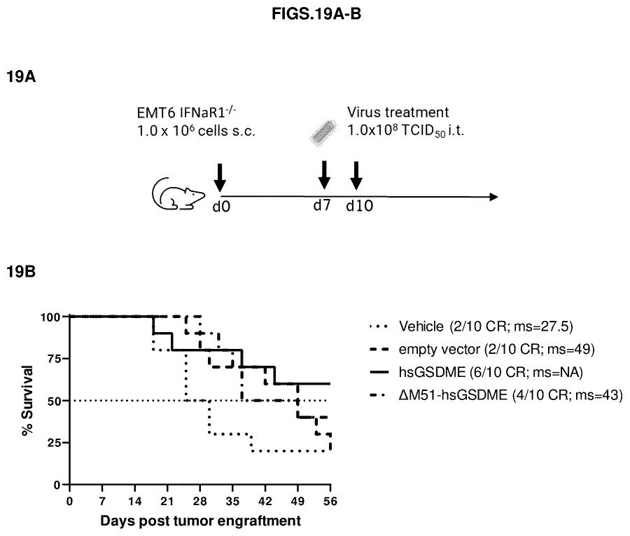

### FIGS. 19A-B

This study aimed to evaluate the potential therapeutic benefits of oncolytic virus (OV)-mediated GSDME expression in tumors with endogenous expression of GSDME. The following results provide insights into the survival benefits and tumor growth reduction associated with VSV-GP-GSDME treatment, highlighting its potential applicability across a range of indications with differing GSDME expression levels.

EMT-6 tumor cells, deficient for the interferon alpha 1 receptor (EMT-6-IFNAR1−/−), were implanted subcutaneously (1×10⁶ cells), treated twice with virus (10⁸ TCID50) three days apart (FIG. 19A). Kaplan-Meier survival curves showed a survival benefit for VSV-GP-hsGSDME (GSDME=SEQ ID NO:49) compared to the other treatment groups (FIG. 19B). Log-rank (Mantel-Cox) test was performed (p=ns).

Tumors exhibiting elevated levels of GSDME expression also responded to oncolytic virus (OV)-mediated GSDME expression. This finding broadens the potential therapeutic benefits to encompass not only indications with reduced expression of functional GSDME but also those with higher expression levels.

### Example 13

Combination of VSV-GP-GSDME with Various Cytokines, Including IL1, IL12, IL18, and IFNα

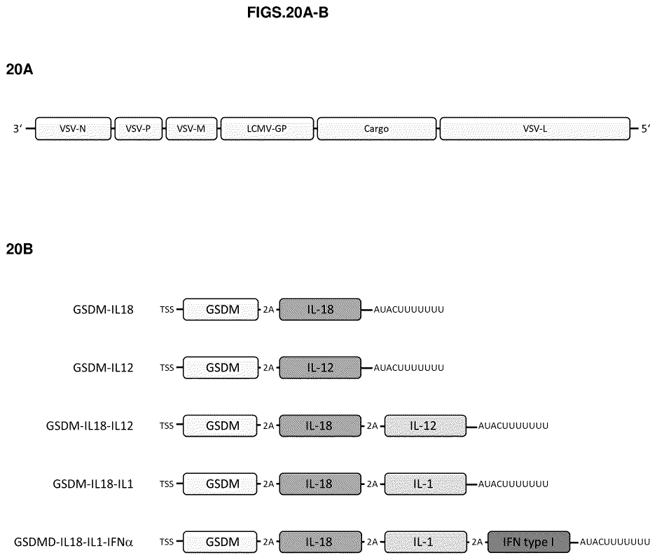

### FIGS. 20A-B and 21A-G

To further improve the immune response, Cas3 cleavable gasdermin-expressing VSV-GPs were combined with various cytokines, including IL1, IL12, IL18, and IFNα (FIGS. 20A and B). These cytokines were cloned alone or in combination into a VSV-GP that already expressed either GSDME or GSDMD_{DEVD} (GSDM) (FIG. 20A).

The rationale for combining the mature form of IL1 and IL18 with GSDMD_{DEVD} is to mimic not only pyroptotic cell death in VSV-GP infected cells but also express cytokines that are normally released by the GSDMD pore after activation of the canonical inflammasome pathway, which are missing since VSV-GP is not able to trigger Cas1 activation, a prerequisite for the processing of preIL-1α/β and IL-18.

Additionally, type I interferon (IFNα) was introduced because it is consensus that innate responses triggered by other vesiculovirus-based OVs such as VSV or Maraba virus strongly affect the outcome of an adaptive immune response but are absent in VSV-GP since it expresses the wildtype variant of the VSV matrix protein, which blocks cellular mRNA transport of cellular genes, including type I IFNs and IFN-stimulated antiviral genes.

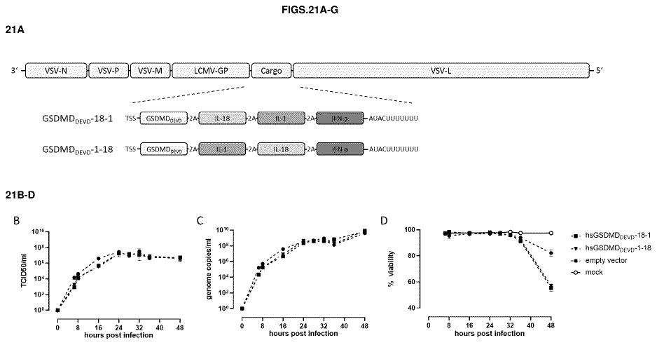

To allow expression of up to three additional genes by the VSV-GP-GSDMD_{DEVD} vector, we separated the peptide-coding sequences of mature IL-1α, mature-IL18, and IFNα2 with picornavirus-derived 2A sites from Thosea asigna virus (T2A), porcine Teschovirus (T2A), and encephalomyocarditis virus (E2A) from GSDMD_{DEVD}. To exclude positional effects on expression of the peptides, we generated shuffled versions of the viruses (FIG. 21).

Replication kinetics in HEK293F cells of both viruses, GSDMD_{DEVD}-18-1 (SEQ ID NO:90) and GSDMD_{DEVD}-1-18 (SEQ ID NO:89), did not show any impact on infectious or genomic titers when compared to the empty vector (FIGS. 21A and B). Additionally, the viability of cell cultures infected with viruses that contain the GSDMD_{DEVD} showed a clear drop caused by the early onset of cell death by the activated GSDMD (FIG. 21C). This observation is in line with previous experiments using a virus armed only with GSDMD_{DEVD} (FIG. 9D). To test whether position influences the release of the virally expressed cytokines, we performed ELISAs for IL18, IL1, and IFNα2 on cell culture supernatant at indicated time points (FIGS. 21E-F). From 24 h to 36 h post-infection, we observed a gradual increase in all the cytokines, with levels ranging from 10 to 100 ng/ml supernatant, and we could not detect a strong positional effect on IL1 and IL18 expression and release. However, we could not fully exclude that incomplete processing of the polypeptide by the 2A sites has an influence on the total amount of cytokines in the cell culture supernatants.

In summary, we can include up to three immunostimulatory cytokines in addition to a gasdermin with the potential to further improve OV therapy without hampering viral fitness. Additionally, positional effects of the placed cytokine cargoes are negligible, and all cytokines were expressed at comparable levels.

### Example 14

Combination of GSDME with IL12

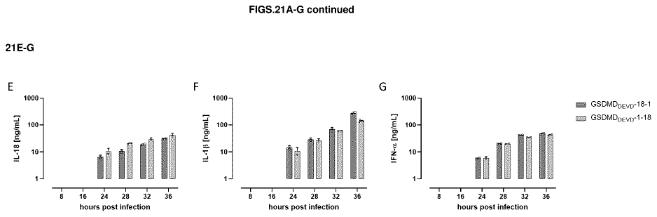

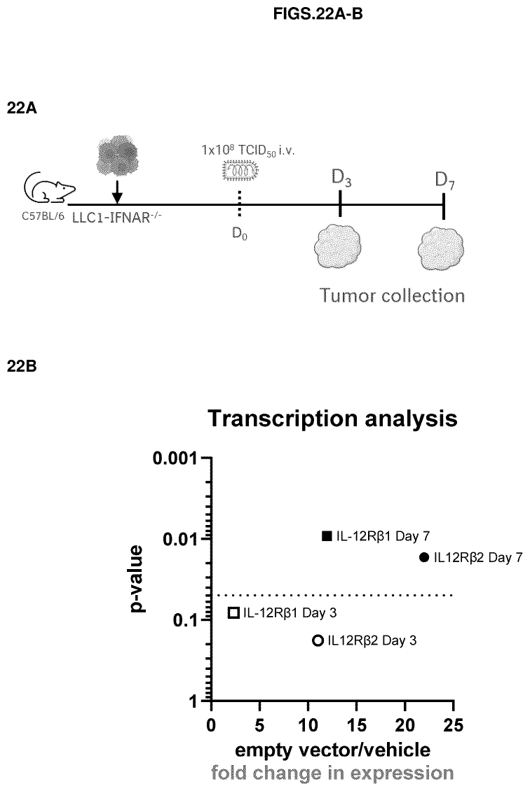

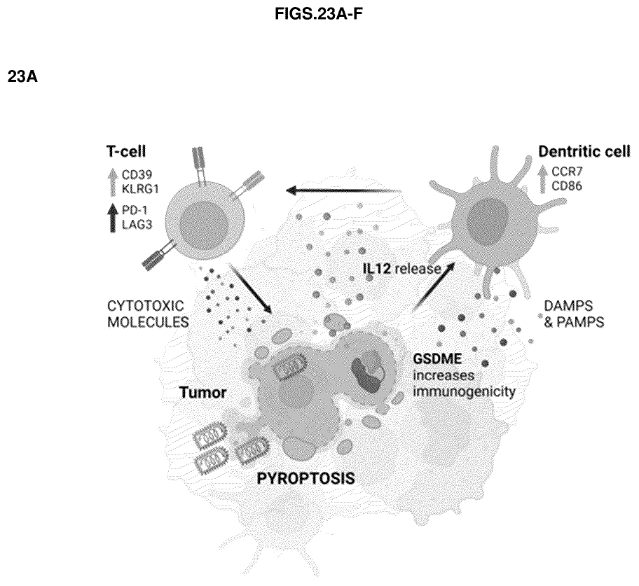

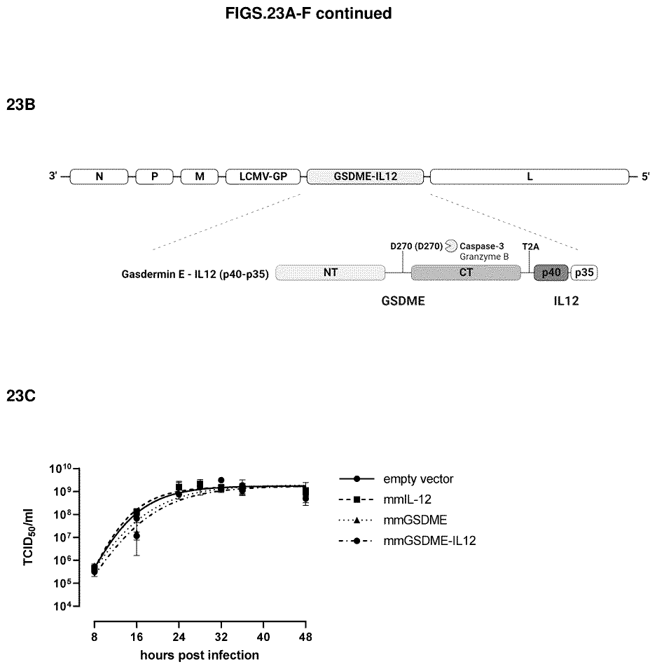

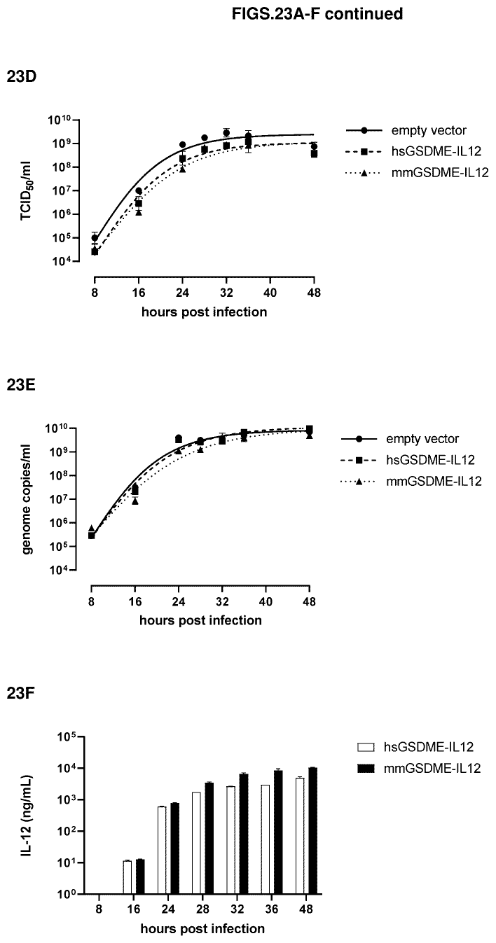

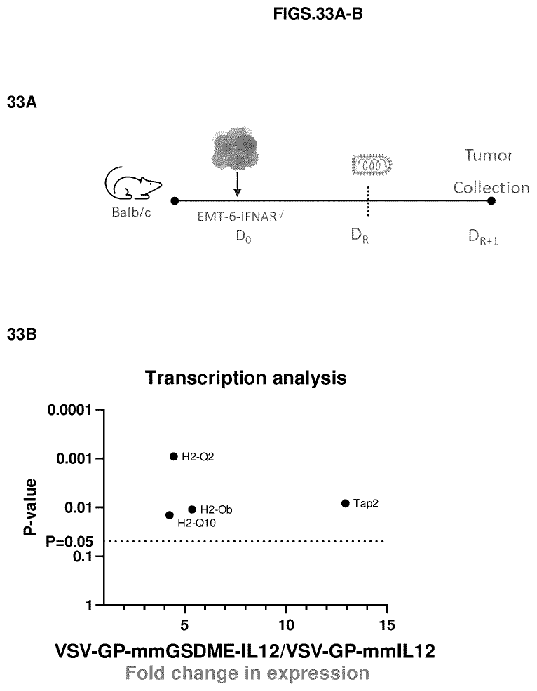

### FIGS. 22A-B and FIGS. 23A-F and FIGS. 33A-B

Localized inflammation and the ensuing recruitment of immune cells, resulting from viral replication of VSV-GP within the tumor microenvironment (TME), contribute to a marked upregulation of the interleukin12 (IL12) receptor complex that consists of IL12Rβ1 and IL12Rβ2 subunits (FIG. 22). Therefore, we hypothesized that adding IL12 to VSV-GP-GSDME will further improve the efficacy of the oncolytic virus.

The GSDME payload of VSV-GP increases immunogenicity by inducing pyroptotic cell death in infected tumor cells, leading to the release of PAMPs, DAMPs, and tumor antigens. The release of PAMPs and DAMPs increases CD86 and CCR7 expression on DC subsets, enhancing their ability to migrate from the tumor to the tumor-draining lymph node and to present tumor-derived antigens, priming a potent anti-tumor CD8+ T cell response. This was also supported by the gene ontology cluster linked to antigen processing and presentation being upregulated with the GSDME cargo addition to VSV-GP-IL12 treatment (FIGS. 33A-B). Interleukin12 (IL12) not only affects DCs but also enhances the T cell responses in the tumor, promoting CD8+ T cells with a Long-Lived Effector Cell (LLEC) phenotype, characterized by CD39 and KLRG1 expression. Additionally, LAG3 and PD-1 upregulation on T cells potentially sensitizes them for CPI treatment (FIG. 23A).

The open reading frame of the heterodimeric IL12 was linked to the GSDME coding sequence by a Thosea asigna virus-derived 2A peptide. The p40 and p35 subunits of IL12 are connected by a flexible (GGGS) 3 linker. GSDME-IL12 (SEQ ID NO: 72) is placed between the LCMV glycoprotein and the VSV polymerase L. Additional transcription start- and stop-signals up- and downstream of the GSDME-IL12 fusion protein were added to the intergenic regions to allow efficient transcription of the GSDME-IL12 coding RNA by the viral polymerase (FIG. 23B).

Replication kinetics of single cargo VSV-GP-IL12 (IL12=SEQ ID NO:66), -hsGSDME (GSDME=SEQ ID NO:49), and dual-cargo-hsGSDME-IL12 (SEQ ID NO: 72) viruses (FIG. 23C), as well as a comparison of human (hs) GSDME-IL12 (SEQ ID NO: 72) and mouse (mm) GSDME-IL12 (SEQ ID NO:84) viruses (FIGS. 23D, E): HEK293F cells were infected at an MOI=0.0005. Infectious titers were measured by TCID50 assay on BHK21 cells (FIGS. 23C, D), while genomic titers (FIG. 23E) were determined by VSV-N-specific qPCR from supernatants. For control, parental VSV-GP was used. Data points are displayed as the mean value of two biological replicates. TCID50 was performed with two technical, and VSV-N qPCR with three technical replicates. Expression of hs- and mmIL12 in supernatants of infected HEK293F cells at indicated time points was measured by ELISA in duplicates (FIG. 23F). We could detect increasing amounts of IL12 over time, with 8-10 ng at 48 hpi for VSV-GP-mmGSDME-IL12 and VSV-GP-mmGSDME.

In summary, the open reading frame of IL12 can be linked to the GSDME coding sequence by a Thosea asigna virus-derived 2A site, with additional transcription VSV start and stop signals added for efficient transcription. Replication kinetics of mouse and human GSDME-IL12 viruses were studied in HEK293F cells, and IL12 expression was measured over time, showing identical replication and comparable amounts of IL12 at 48 hours post-infection.

### Example 15

### Manufacturing Drug Substance of VSV-GP-GSDME-IL12

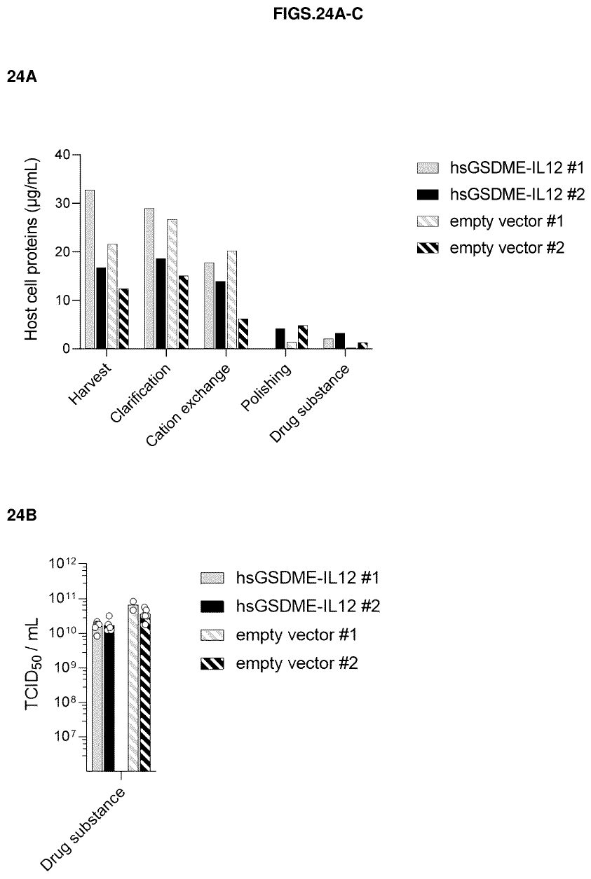

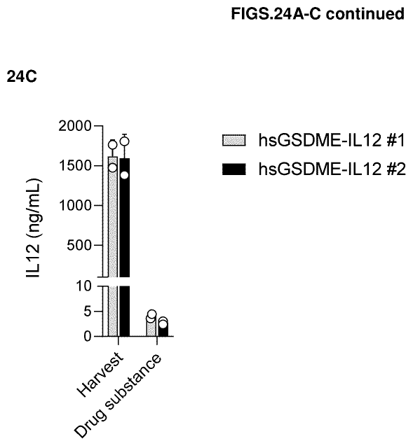

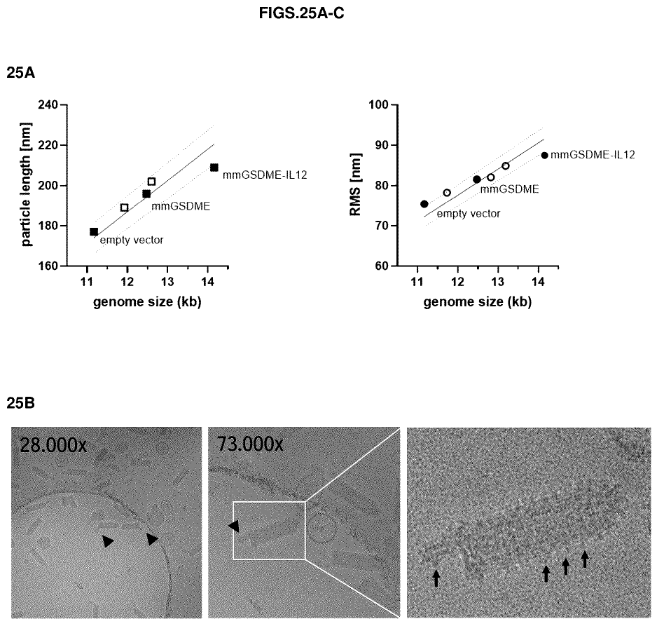

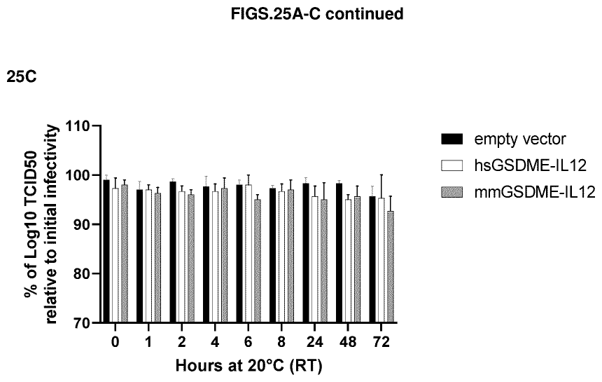

### FIGS. 24A-C and FIGS. 25A-C

A significant challenge in transitioning OV-based cancer immunotherapies from the laboratory to clinical application is manufacturability. Particularly, enveloped viruses are challenging to produce in sufficient quantities and high quality that meet the criteria for clinical use in humans. However, GSDME is not inert to HEK293F cells, and the harvest of crude supernatant containing the virus occurs when increased cell death is observed after VSV-GP-GSDME infection (see Example 4). GSDME affects HEK293F cells and increased levels of cell death are observed after VSV-GP-GSDME infection at the time of harvest (see Example 4). The early onset and the membrane ruptures associated with pyroptotic cell death led to higher levels of contaminants such as host cell proteins and DNA. In turn, these impurities might affect the downstream purification of the drug substance or the drug substance itself. Additionally, IL12 from the upstream process itself constitutes a contaminant and needs to be removed from the drug substance since IL12, when administered systemically via the intravenous route, can cause liver toxicity in patients.

Accordingly, we used the process established for VSV-GP to purify the human version of VSV-GP-hsGSDME-IL12 (GSDME-IL12=SEQ ID NO:72) and compared host cell contaminants and IL12 levels at different stages of the manufacturing process. We detected higher levels of host cell proteins at the early steps of the downstream process when comparing hsGSDME-IL12 to the parental VSV-GP (empty vector). However, after polishing and in the final drug substance, levels of contaminants were comparably low (FIG. 24A). Although initial host cell contaminants were higher for VSV-GP-hsGSDME-IL12, this did not significantly influence the absolute infectious TCID50 titer (FIG. 24B). The IL12 concentration in the final drug substance is 400-fold lower when compared to the initial cell culture harvest (FIG. 24C). Additionally, we compared the process performance of VSV-GP-hsGSDME-IL 12 to that for the mouse surrogate VSV-GP-mmGSDME-IL12. Looking at infectious TCID50 or genomic copies, we could not detect any significant differences in any of the process steps, thus ensuring full comparability of the mouse GSDME-IL12 used in preclinical research and toxicity studies (data not shown).

Finally, we examined virus particles of VSV-GP-GSDME-IL12 after HPLC-SEC separation by multi-angle light scattering (MALS) and cryoEM (FIG. 25). For virus particles with smaller cargoes up to 2 kb the data show good correlation between particle and genome sizes as measured by cryoEM (particle length) and MALS (RMS). However, for larger cargoes such as VSV-GP-mmGSDME-IL12, virus particles are slightly smaller than expected, assuming a linear relationship. For RMS measurements, this may be due to a lack of linearity of the method at higher genome sizes. Smaller physical particle length may also result from higher frequencies of incompletely packed virions. cryoEM images showed that the total numbers of “tailed” particles for VSV-GP-mmGSDME are increased relative to VSV-GP (FIGS. 11A and B). However, images clearly show that the tails consist of condensed ribonucleoprotein wrapped by the virion's membrane containing trimeric GP glycoprotein spikes.

In summary, VSV-GP-GSDME-IL12 can be manufactured using the process established for VSV-GP and yields comparable results. Despite higher initial levels of host cell contaminants for VSV-GP-hsGSDME-IL12, these could be largely removed, and the final drug substance had comparably low levels of contaminants. Also, the mouse surrogate VSV-GP-mmGSDME-IL12 process performance was fully comparable. Although we could detect higher numbers of tailed virions by cryoEM analysis, we could not detect any loss of infectivity.

### Example 16

### Evaluation of Efficacy and Induced Immune Responses by VSV-GP-GSDME-IL12 Treatment in a Mixed TC-1 Tumor Model

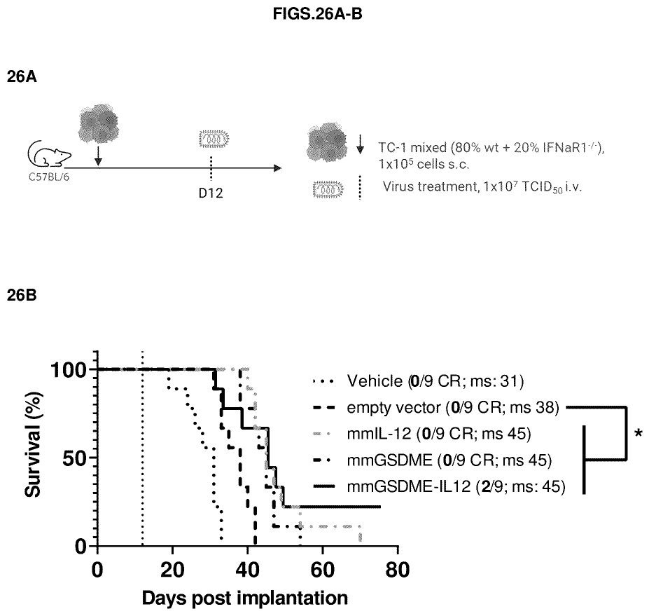

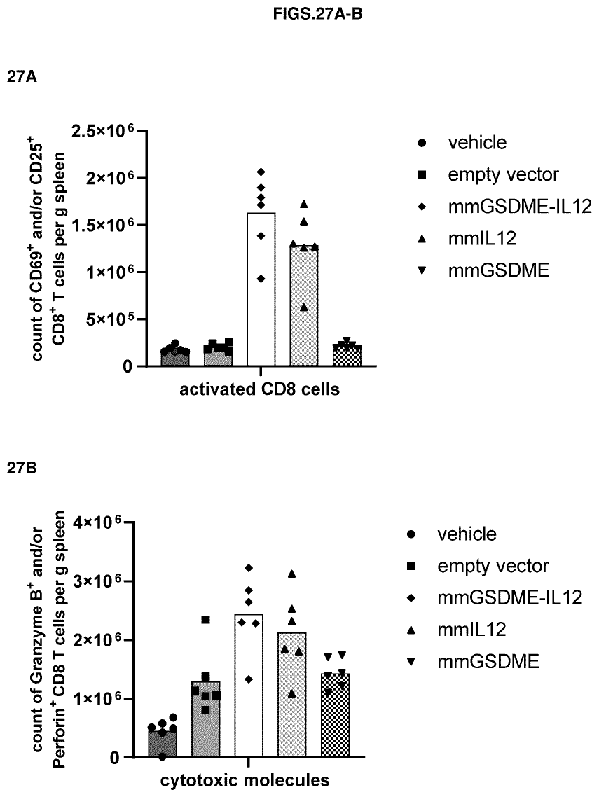

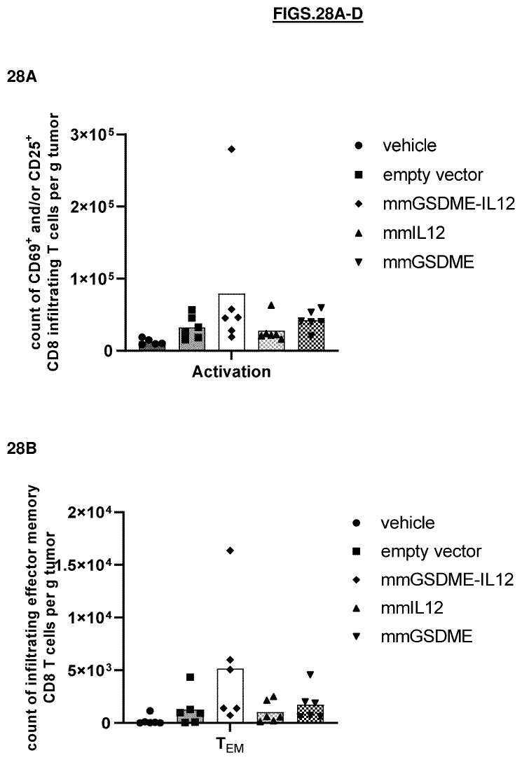

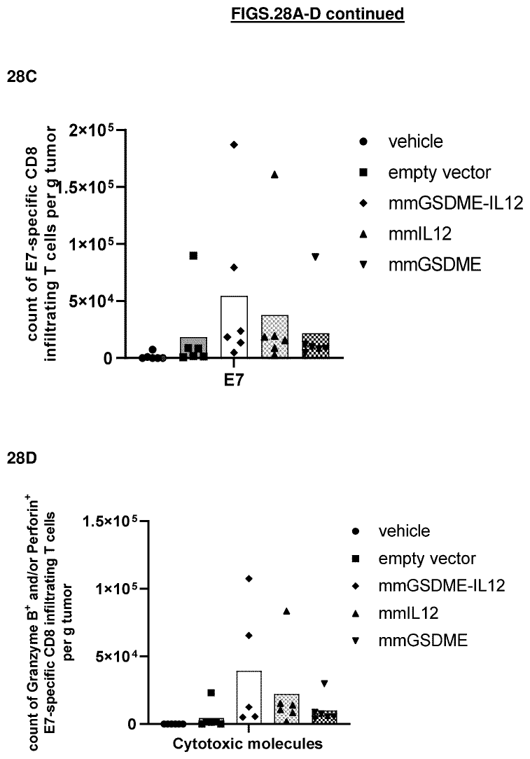

### FIGS. 26A-B, FIGS. 27A-B, and FIGS. 28A-D

We aimed to evaluate the possibility of improving the efficacy of GSDME by adding a second cargo, namely IL12. VSV-GP, VSV-GP-mmIL12 (IL12-SEQ ID NO: 83), VSV-GP-mmGSDME (GSDME=SEQ ID NO:76), and VSV-GP-mmGSDME-IL12 (GSDME-IL12=SEQ ID NO:84) constructs were generated and compared in a mice tumor model carrying mixed TC-1 tumors. The TC-1 tumor model exhibits resistance to checkpoint inhibitors (CPI) and possesses lower immunogenicity compared to other syngeneic tumor models, including MC-38, CT26, and EMT6.

Tumors were implanted in C57BL/6J mice by subcutaneously injecting a mixture of tumor cells (1×10⁵ cells) into the right flank (FIG. 26A). The mixture of tumor cells consisted of 80% wildtype TC-1 and 20% TC-1-IFNAR1−/− (deficient for interferon alpha 1 receptor expression). A virus treatment (1×10⁷ TCID50) was given (black dotted line) and animals were evaluated for survival over time. Kaplan-Meier survival curves showed a survival benefit for the different VSV-GP cargo-variants treatments compared to VSV-GP. Log-rank (Mantel-Cox) test was performed (*p<0.05).

Even in this hard-to-treat tumor model, the advantages of integrating both GSDME and IL12 became evident. VSV-GP-mmGSDME-IL12 treatment was the sole intervention that led to complete responses (FIG. 26B).

As immunogenic cell death improves the costimulatory capacity of dendritic cells, activation of T cells should be enhanced. To prove the latter, T cells in the spleen and the tumor were analyzed by flow cytometry to understand their contribution to enhanced survival after VSV-GP-GSDME-IL12 treatment.

Systemic effect of the cargo viruses on T cells was analyzed using single-cell suspensions from spleens harvested on day 3 after virus treatment. Activation of CD8 T cells in the spleen was strongly increased with VSV-GP-mmGSDME-IL12 and to a lesser extent with VSV-GP-mmIL12 (FIG. 27A). In addition, the number of splenic CD8 T cells expressing the cytotoxic molecules Granzyme B and Perforin, which are essential in cell-mediated killing, were increased with the single cargos but even more pronounced with the dual cargo virus (FIG. 27B).

Tumors harvested on day 7 after virus treatment were analyzed regarding the quality of the CD8 T cell response. Cell counts of activated CD8 T cells were not only increased in the tumor tissue with VSV-GP-mmGSDME-IL12 (FIG. 28A). The CD8 T cell population also showed an increase in effector memory T cell count (TEM), suggesting a long-lasting and recallable immune response (FIG. 28B). Among the CD8 T cells, both single and the dual cargo viruses led to increased counts of tumor-infiltrating E7-specific cells, with the dual cargo showing the highest counts (FIG. 28C). The functional characterization of these cells also revealed the strongest increase in E7-specific cells that express Granzyme B and Perforin, indicating the enhanced cytotoxic potential of these tumor-specific T cells (FIG. 28D).

Collectively, incorporating both GSDME and IL12 into VSV-GP culminates in a superior anti-tumor immune response and an improved therapeutic outcome.

### Example 17

### Intra-Tumoral and Intravenous Treatments at Different Doses in the CT26.CI25 Tumor Model

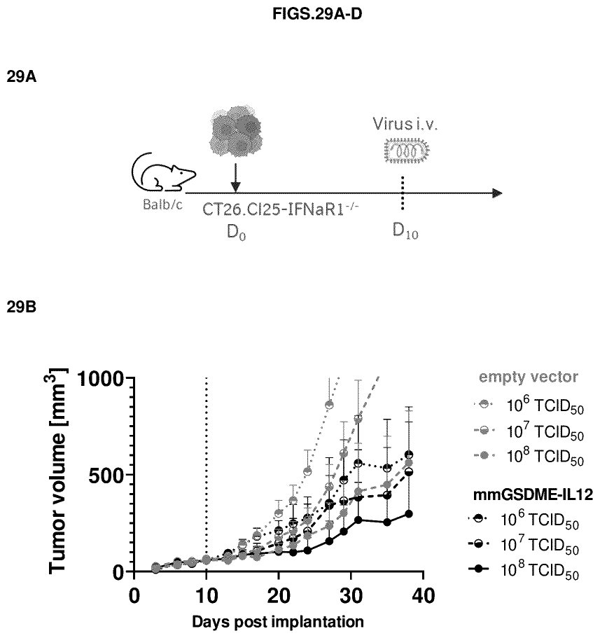

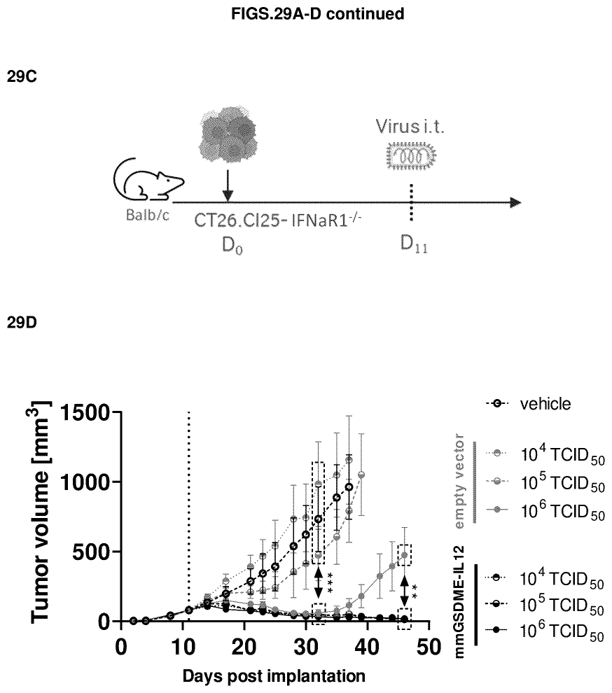

### FIGS. 29A-D

In order to evaluate the advantages of incorporating IL12 and GSDME as therapeutic cargos for intra-tumoral and intravenous administration, animals with CT26.CI26−IFNAR1−/− tumors were subjected to treatment using different dosages of VSV-GP-mmGSDME-IL12 (GSDME-IL12=SEQ ID NO:84) and VSV-GP.

Tumors were implanted in BALB/c mice by subcutaneously injecting the tumor cells (1×10⁶ CT26.CI25-IFNAR1−/− cells) into the right flank. A virus treatment was given intratumorally (FIG. 29C/D) or intravenously (FIG. 29A/B) (vertical black dotted line) and animals were evaluated for survival over time. Tumor volume over time (mean±SEM) showed a significant benefit in tumor growth inhibition for VSV-GP-mmGSDME-IL12 for intra-tumoral as well as intravenous treatments, with intra-tumoral treatment being especially efficacious at all doses compared to VSV-GP dosed at 105 and 10⁴ TCID50 (FIG. 29B). One-way ANOVA test was performed at day 32 (***p<0.001).

In summary, quite unexpectedly, the VSV-GP-mmGSDME-IL12 treatment demonstrated superior efficacy, and intra-tumoral even at a dosage 100 times lower than the VSV-GP treatment.

### Example 18

### Abscopal Therapeutic Effect in Bilateral Tumor Setting

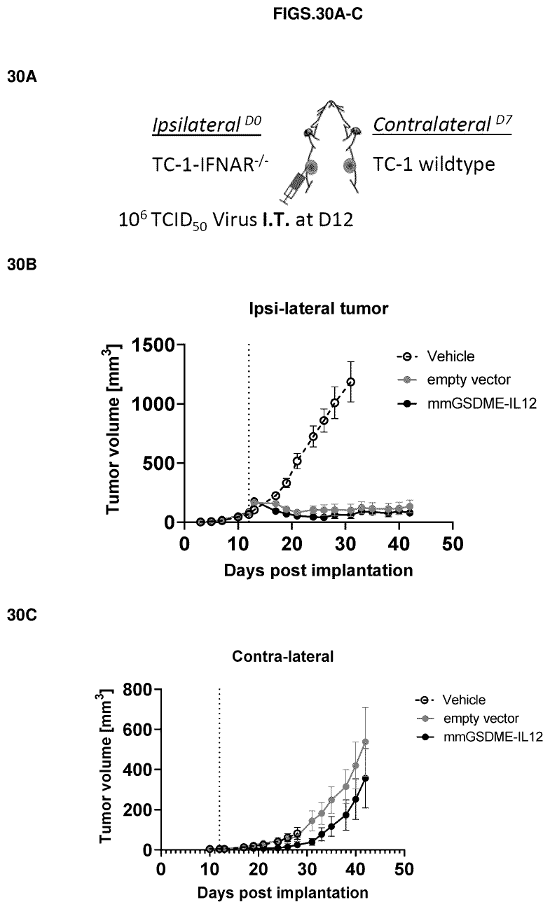

### FIGS. 30A-C

To assess the potential of VSV-GP-mmGSDME-IL12 (GSDME-IL12=SEQ ID NO:84) in eliciting abscopal effects upon localized administration, we engrafted oncolytic-sensitive (interferon-resistant) TC1-IFNAR1−/− cancer cells on the right flank, followed by the implantation of more resistant (interferon-sensitive) wild-type TC-1 cancer cells on the left flank after a 7-day interval. Subsequently, the TC1-IFNAR1−/− tumor was subjected to intra-tumoral treatment, and the subjects were monitored for tumor progression over time. Although the treated ipsilateral tumor exhibited equal levels of lytic control, a disparity in tumor growth regulation was observed in the contralateral tumor. In this context, VSV-GP-mmGSDME-IL12 treatment resulted in an enhanced control of tumor growth over time.

In conclusion, this investigation substantiates the capacity of VSV-GP-mmGSDME-IL12 therapy to provoke abscopal therapeutic efficacy through the activity of its therapeutic cargos.

### Example 19

### VSV-GP-GSDME-IL12 Infects Patient-Derived CRC Tissue, Leading to GSDME Activation and IL12 Receptor Engagement

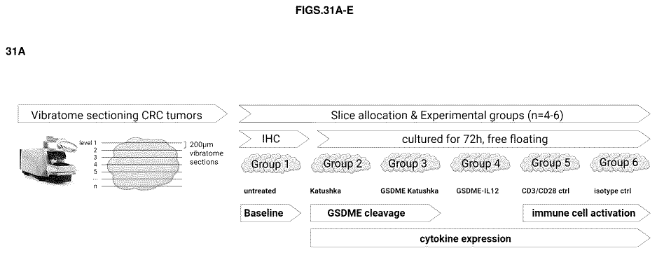

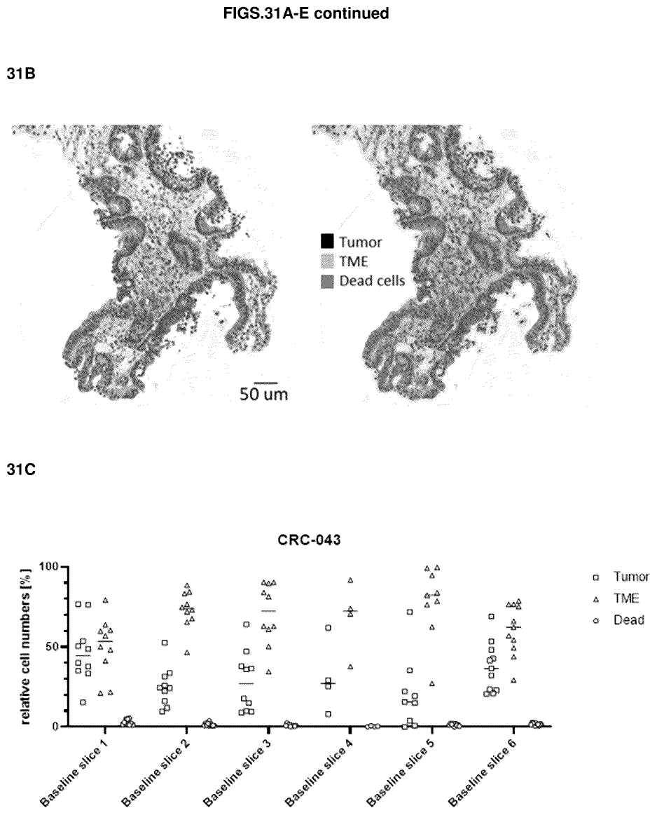

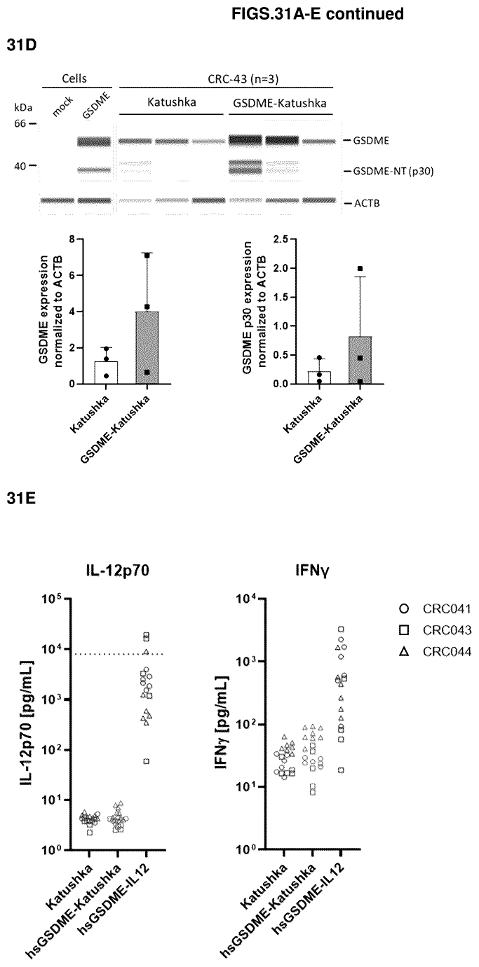

### FIGS. 31A-E

Cancer etiology and architecture are quite diverse and complex, hindering the prediction of whether a patient could respond to a particular cancer immunotherapy or combination treatment. A simultaneously arising caveat is the challenge in translating from pre-clinical, cell-based in vitro systems, as well as syngeneic murine tumor models, towards the heterogeneous architecture of the human tumor ecosystems. To bridge this gap, we employed a patient-derived tumor slice culturing system to assess virus permissivity, activation of exogenously expressed GSDME, and the immunomodulatory effects of IL12.

Patient-derived, freshly resected colorectal cancers were cut into 200 μm thick tissue slices and allocated sequentially to the different treatment groups depicted in FIG. 31A. For baseline characterization of tumor content, tumor microenvironment (TME) composition, and viability, every seventh tumor slice was fixed, paraffin-embedded, and stained by Hematoxylin/Eosin for histopathology assessment by a trained pathologist (FIGS. 31B/C). To confirm the presence and functionality of immune cell infiltrate, we stimulated T cell receptors by CD3/CD28 antibody crosslinking and downstream measurement of IFNγ by a flow cytometry-based Legendplex assay. Isotypes were used for control (not shown).

Remaining slices (n=4-6 slices/group) were infected with either VSV-GP-Katushka (Katushka), VSV-GP-hsGSDME-Katushka (GSDME-Katushka; GSDME=SEQ ID NO:49), or VSV-GP-hsGSDME-IL12 (GSDME-IL12=SEQ ID NO:72) at a dose of 1×10⁷ TCID50/slice or were left untreated (mock) for control. Virus permissivity was assessed during an in-life from 24 to 72 hours post-infection by fluorescence microscopy of the red fluorescent Katushka protein (not shown). Highly permissive tumor slices, characterized by several patches of Katushka-positive areas within the tumor slices, were harvested at 72 hpi, and proteins were extracted using a standard extraction protocol before western blot detection of full-length and cleaved GSDME. Expression was quantified relative to β-actin (see methods section). For technical control, we used protein extracts from mock or VSV-GP-hsGSDME infected cells.

Expression and cleavage of GSDME could be detected in VSV-GP-hsGSDME-Katushka infected patient-derived CRC slice cultures (exemplary shown for CRC-43 in FIG. 31D). Due to tumor heterogeneity, in one out of three slides, we could not detect GSDME expression and cleavage at levels higher than the endogenous GSDME levels. However, quantification of GSDME and cleaved GSDME-NT corresponds to the expression of the fluorescent Katushka reporter from the in-life phase.

IL12 and IFNγ Legendplex assay from collected cell culture supernatants at 72 hpi from three patient-derived CRCs in 4-6 replicates clearly show expression of IL12 by VSV-GP-hsGSDME-IL12 in most of the permissive samples. Additionally, virally expressed IL12 can trigger IL12 receptor signaling, resulting in elevated levels of IFNγ in the supernatant of VSV-GP-hsGSDME-IL12 but not VSV-GP-Katushka or VSV-GP-hsGSDME-Katushka infected CRC slices (FIG. 31E).

Taken together, VSV-GP-hsGSDME-IL12 can infect human CRC specimens. Infection leads to expression of GSDME and IL12. Virally expressed GSDME is cleaved after VSV-GP triggers the apoptotic pathways into its pore-forming active N-terminal domain. Additionally, expressed IL12 is secreted and can trigger the IL12 receptor pathway, with the result of IFNγ production by tumor-resident immune cells that express the IL12 receptor complex.

### Example 20

In Vivo Data (EMT6): Mouse Treated with VSV-GP, VSV-GP-mmGSDME-IL12

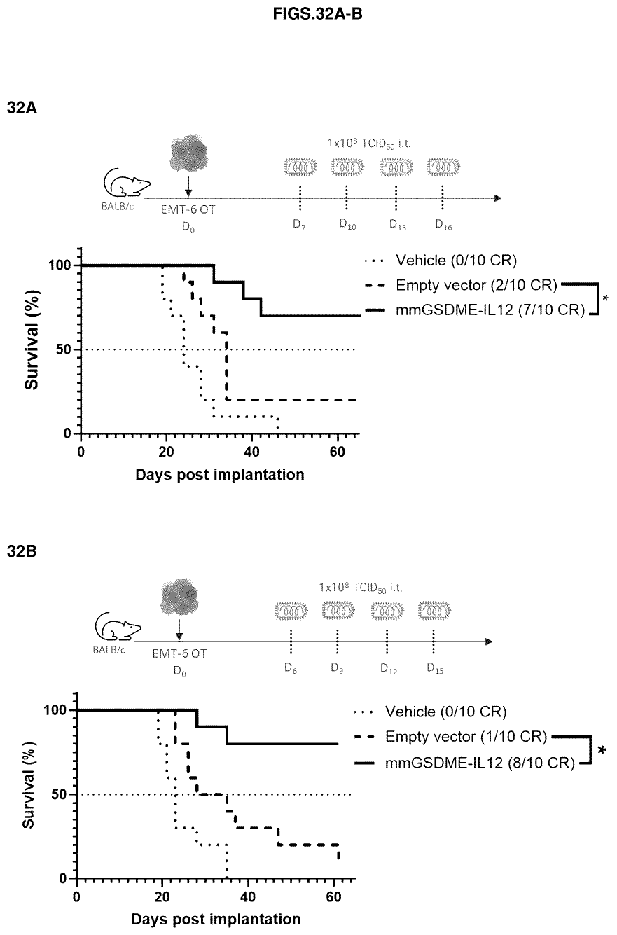

### FIGS. 32A-B

These studies aimed to evaluate the potential therapeutic benefits of oncolytic virus (OV)-mediated GSDME-IL12 expression (FIG. 32A-GSDME-IL12=SEQ ID NO: 84; FIG. 32B—GSDME(Q9Z2D3)-IL12=SEQ ID NO: 110) in the breast cancer tumor model EMT-6. The following results provide insights into the survival benefits associated with two splice variants of murine GSDME (SEQ ID NO: 76; SEQ ID NO: 109) within the context of VSV-GP-GSDME-IL12 treatment, highlighting the similarity in activity between both splice variants.

EMT-6 tumor cells were implanted subcutaneously (2.5×10⁵ cells), treated four times with virus (108 TCID50) three days apart. Kaplan-Meier survival curves showed a survival benefit for VSV-GP-mmGSDME-IL12 compared to the other treatment groups FIG. 32A-GSDME-IL12=SEQ ID NO: 84; FIG. 32B-GSDME(Q9Z2D3)-IL12=SEQ ID NO: 110). Log-rank (Mantel-Cox) test was performed (*p<0.05).

### Example 21

### The Role of CD8⁺ T-Cells in the Efficacy of VSV-GP-GSDME-IL12

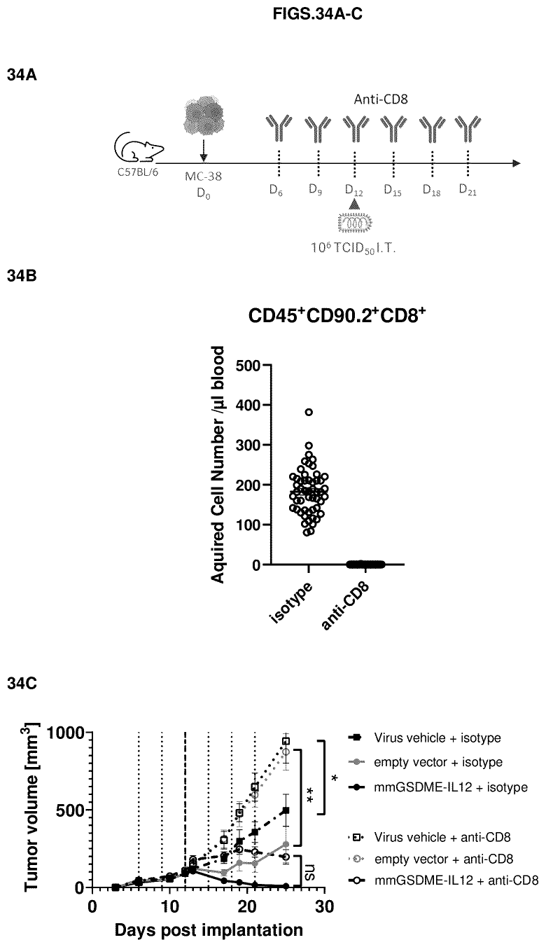

### FIGS. 34A-C

To confirm that tumor specific CD8⁺ T cells induced by the respective oncolytic virus treatment contributed to suppressing the growth of the treated tumors, CD8+ cells were depleted by anti-CD8 monoclonal antibody. Depletion of CD8⁺ cells completely reversed the therapeutic effects of the VSV-GP therapy. However, the VSV-GP-mmGSDME-IL12 (GSDME(Q9Z2D3)-IL12=SEQ ID NO: 110) treatment demonstrated a more potent suppression of tumor growth. Interestingly, the depletion of CD8⁺ cells only partially reversed the therapeutic activity of this treatment (as shown in FIG. 34). These findings underscore the significance of CD8⁺ T cells in inhibiting tumor growth. However, they are not the sole contributors to the effectiveness of VSV-GP-mmGSDME-IL12, indicating the presence of other contributing factors. This is in line with existing data previously reporting that IFN-γ and a cascade of other secondary and tertiary pro-inflammatory cytokines induced by IL-12 have a direct toxic effect on the tumor cells or may activate potent anti-angiogenic mechanisms.

### Example 22

### VSV-GP-GSDME Infected Tumor Cells Show Reduced Apoptotic Pathway Activation and Nuclear DNA Fragmentation

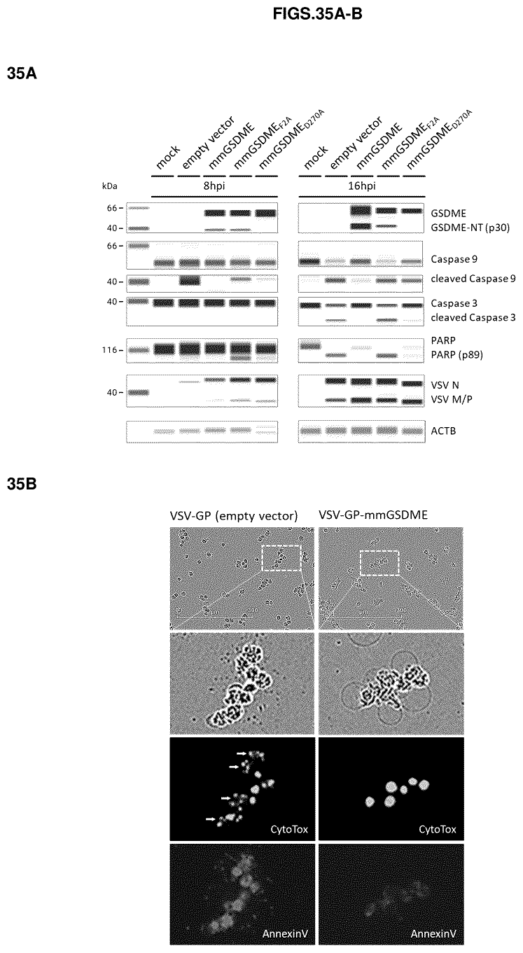

### FIGS. 35A-B

Expression and activation of GSDME by VSV-GP-GSDME involves early loss of cell membrane integrity and cell swelling due to enhanced membrane permeability and water influx into the pyroptotic dying cell. However, GSDME expression by the oncolytic VSV-GP and Caspase3 induction by the virus, may result in GSDME activation without altering apoptotic events up and -downstream of Caspase 3 activation such as Caspase 9 or PARP cleavage. To test if VSV-GP-GSDME influences the induction of the apoptotic cascade, we examined additional up- and downstream events in the apoptotic pathway by western blotting and detailed live cell imaging.

In brief, EMT6 IFNaR^{-/-} GSDME^{-/-} mouse breast cancer cells were infected at an MOI of 3 with either VSV-GP-mmGSDME (GSDME=SEQ ID NO:109), VSV-GP-mmGSDME_{F2A}, VSV-GP-mmGSDME_{D270A} or VSV-GP. Untreated cells served as controls. The experiment was conducted in a 6-well plate with 1×10⁶ cells per well. Cell extracts were prepared after 8 and 16 h post infection using RIPA buffer. 0.8 mg of total protein was loaded onto a JESS capillary simple western system (BioTechne). For protein detection, the following primary antibodies were used: Caspase 3, Cell Signaling (#9662); cleaved Caspase 3, Cell Signaling (#5A1E); Caspase 9, Cell Signaling (#9504), cleaved Caspase 9 (#9509); DFNA5/GSDME, Abcam (#ab215191); PARP, Cell Signaling (#46D11).

In comparison to mock, VSV-GP (empty vector) and mmGSDMEF2A, a loss-of-function variant that is unable to form active GSDME pores at cellular membranes, VSV-GP-mmGSDME infected cells exhibited reduced or even non-detectable signals for cleaved caspase 9, Caspase3 and PARP (FIG. 35A). A non-cleavable GSDME_{D270A} variant that contains a mutation in the Cas3 cleavage signal, showed a comparable decrease in apoptotic signals, indicating that this observation may be cleavage independent of GSDME. This finding is consistent with recent reports that show that under certain conditions gasdermin pore formation may lead to pyroptosis independent from cleavage.

Furthermore, live cell imaging reveals that VSV-GP-mmGSDME (GSDME=SEQ ID NO:109) cells show less nuclear DNA fragmentation than cells that are infected with VSV-GP (FIG. 35B). This finding is consistent with decreased PARP activation, since PARP plays an essential role in maintaining genomic stability by either facilitating DNA repair/replication or triggering DNA fragmentation.

In conclusion, VSV-GP-mmGSDME not only exhibited pyroptotic features directly related to GSDME pore formation and loss of membrane integrity, but also resulted in an overall decrease in apoptotic pathway activation upstream and downstream of Caspase 3 cleavage.

1.-38. (canceled)

39. A recombinant vesicular stomatitis virus encoding in its genome at least one GSDM or a functional variant thereof, preferably a human GSDM, wherein the gene coding for the glycoprotein G of the recombinant vesicular stomatitis virus is replaced by the gene coding for the glycoprotein GP of Lymphocyte choriomeningitis virus (LCMV), and/or the glycoprotein G is replaced by the glycoprotein GP of LCMV.

40. The recombinant vesicular stomatitis virus according to claim 39, wherein the GSDM is selected from the group consisting of: Gasdermin A (GSDMA), Gasdermin B (GSDMB), Gasdermin C (GSDMC), Gasdermin D (GSDMD), Gasdermin E (GSDME or DFNA5) or DFNB59 (Pejvakin).

41. The recombinant vesicular stomatitis virus according to claim 39, wherein the GSDM or functional variant thereof comprises (i) the N-terminal domain (GSDM-NT) of a GSDM, and (ii) the C-terminal domain (GSDM-CT) of a GSDM.

42. The recombinant vesicular stomatitis virus according to claim 41, wherein the GSDM-NT comprises or consists of any one of SEQ ID NOs: 51-55.

43. The recombinant vesicular stomatitis virus according to claim 41, wherein the GSDM-CT comprises or consists of any one of SEQ ID NOs: 56-60.

44. The recombinant vesicular stomatitis virus according to claim 41, wherein the GSDM-NT comprises or consists of any one of SEQ ID NOs: 51-55, and the GSDM-CT comprises or consists of any one of SEQ ID NOs: 56-60.

45. The recombinant vesicular stomatitis virus according to claim 40, wherein the GSDM further comprises a cleavable peptide sequence not naturally occurring in said GSDM.

46. The recombinant vesicular stomatitis virus according to claim 45, wherein the cleavable peptide sequence is protease cleavable.

47. The recombinant vesicular stomatitis virus according to claim 46, wherein the protease cleavable peptide sequence is specifically cleavable by caspases, preferably caspase-3.

48. The recombinant vesicular stomatitis virus according to claim 46, wherein the cleavable peptide sequence comprises the consensus sequence DxxD (SEQ ID NO:62).

49. The recombinant vesicular stomatitis virus according to claim 48, wherein the cleavable peptide sequence comprises the sequence DMPD (SEQ ID NO: 63), DEVD (SEQ ID NO:64) or DLPD (SEQ ID NO:65).

50. The recombinant vesicular stomatitis virus according to claim 39, wherein the GSDM comprises or consists of any one of SEQ ID NOs: 45-50.

51. The recombinant vesicular stomatitis virus according to claim 39, further encoding for at least one cytokine, preferably an interleukin or an interferon.

52. The recombinant vesicular stomatitis virus according to claim 51, wherein the cytokine is interleukin18 (IL18), interleukin12 (IL12), and/or interleukin1 (IL1).

53. The recombinant vesicular stomatitis virus according to claim 51, wherein the interferon is an interferon-type-I (IFN-type-I), preferably IFN-alpha.

54. The recombinant vesicular stomatitis virus according to claim 51, further encoding for (i) IL18 and IL12, (ii) IL18 and IL1, or (iii) IL18 and IL1 and IFN-alpha-2.

55. The recombinant vesicular stomatitis virus according to claim 39, further encoding for an IL12p35 and an IL12p40 subunit of IL12.

56. The recombinant vesicular stomatitis virus according to claim 55, wherein the IL12p35 subunit and the IL12p40 subunit are human.

57. The recombinant vesicular stomatitis virus according to claim 55, wherein the IL12p35 subunit comprises a polypeptide having at least 95% identity to SEQ ID NO:1 and the IL12p40 subunit comprises a polypeptide having at least 95% identity to SEQ ID NO:2.

58. The recombinant vesicular stomatitis virus according to claim 55, wherein the IL12p40 subunit and the IL12p35 subunit are linked in a single-chain having the configuration IL12p40-IL12p35 or IL12p35-IL12p40.

59. The recombinant vesicular stomatitis virus according to claim 58, wherein the IL12p40 subunit and the IL12p35 subunit are linked to each other via a linker that is rich in amino acid residues glycine and serine, preferably having a length of 5 to 20 amino acids and only including the amino acids glycine and serine, more preferably a glycine and serine linker having the amino acid sequence of SEQ ID NO:22.

60. The recombinant vesicular stomatitis virus according to claim 58, wherein the single-chain IL12p40-IL12p35 comprises a polypeptide having at least 95% identity to SEQ ID NO:3 or SEQ ID NO:5; or the single-chain IL12p35-IL12p40 comprises a polypeptide having at least 95% identity to SEQ ID NO:4 or SEQ ID NO:6.

61. The recombinant vesicular stomatitis virus according to claim 55, further comprising a signal peptide sequence linked to the single-chain IL12p40-IL12p35 or IL12p35-IL12p40.

62. The recombinant vesicular stomatitis virus according to claim 61, wherein the signal peptide sequence comprises an amino acid sequence having at least 90% identity to SEQ ID NO: 68.

63. The recombinant vesicular stomatitis virus according to claim 58, wherein the single-chain IL12p40-IL12p35 comprises a polypeptide having at least 95% identity to SEQ ID NO:66 or SEQ ID NO:67.

64. The recombinant vesicular stomatitis virus according to claim 55 further comprising a 2A-peptide, selected from the group consisting of: T2A, P2A, E2A, and F2A peptide.

65. The recombinant vesicular stomatitis virus according to claim 64, wherein the 2A-peptide is located between the GSDM and the IL12 protein.

66. The recombinant vesicular stomatitis virus according to claim 64, wherein the 2A-peptide comprises the consensus sequence DxExNPGP (SEQ ID NO:69).

67. The recombinant vesicular stomatitis virus according to claim 64, wherein the 2A-peptide comprises an amino acid sequence having at least 90% identity to SEQ ID NOs: 70-71 and 73-75.

68. A recombinant vesicular stomatitis virus encoding in its genome at least one GSDM comprising the amino acid of sequence of SEQ ID NO:49, and an IL12p35 and an IL12p40 subunit of IL12 linked in a single-chain having the configuration IL12p40-IL12p35 and comprising the amino acid sequence of SEQ ID NO:5 or SEQ ID NO:66, wherein the gene coding for the glycoprotein G of the recombinant vesicular stomatitis virus is replaced by the gene coding for the glycoprotein GP of Lymphocyte choriomeningitis virus (LCMV), and/or the glycoprotein G is replaced by the glycoprotein GP of LCMV.

69.-121. (canceled)

122. A pharmaceutical composition, characterized in that the composition comprises a a recombinant vesicular stomatitis virus according to claim 39.

123. (canceled)

124. A method for the treatment of solid cancer comprising administering a pharmaceutical composition according to claim 122.

125. The method according to claim 124, wherein the solid cancer is selected from the list consisting of: reproductive cancer, ovarian cancer, testicular cancer, endocrine cancer, gastrointestinal cancer, pancreatic cancer, pancreatic adenocarcinoma, liver cancer, kidney cancer, colon cancer, colorectal cancer, bladder cancer, bladder urothelial carcinoma, muscle invasive bladder cancer (MIBC), non-muscle invasive bladder cancer (NMIBC), prostate cancer or carcinoma, skin cancer, (metastatic) melanoma, respiratory cancer, lung cancer, non-small cell lung cancer, small cell lung cancer, (metastatic) breast cancer or carcinoma, (metastatic) triple negative breast cancer (TNBC), head & neck cancer, head and neck squamous-cell carcinoma (HNSCC), bone cancer, gastric cancer, brain cancer, endometrial cancer, vaginal cancer, anal cancer, oropharyngeal squamous cell carcinoma, gastroesophageal junction adenocarcinoma, esophageal carcinoma, gastro esophageal junction (GEJ) cancer, oesophageal and gastroesophageal junction cancer, adenocarcinoma of the GEJ, hepatocellular carcinoma, cholangiocarcinoma, squamous cell carcinoma, and glioblastoma.

126.-128. (canceled)

129. A composition comprising a recombinant vesicular stomatitis virus according to claim 1 and further a PD-1 pathway inhibitor.

130. The composition according to claim 129, wherein the PD-1 pathway inhibitor is an antagonistic antibody, which is directed against PD-1 or PD-L1.

131. The composition according to claim 1297, wherein the PD-1 pathway inhibitor is an antagonist selected from the group consisting of pembrolizumab, nivolumab, pidilizumab, atezolizumab, avelumab, durvalumab, PDR-001, PD1-1, PD1-2, PD1-3, PD1-4 and PD1-5.

132. A kit of parts comprising:
a) a pharmaceutical composition according to claim 39, and
b) a PD-1 pathway inhibitor.

133.-137. (canceled)

138. A virus producing cell, characterized in that the cell produces a recombinant rhabdovirus or recombinant vesicular stomatitis virus according to any of the preceding claims.

139. (canceled)

140. A recombinant rhabdovirus encoding in its RNA genome at least one GSDME or a functional variant thereof and an IL12p35 and an IL12p40 subunit of IL12 linked in a single-chain having the configuration IL12p40-IL12p35, wherein the RNA genome of the recombinant rhabdovirus comprises or consists of a coding sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 111.


SEQUENCE LISTING (111 sequences)

SEQ ID NO: 1 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 2 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 3 - protein, 503 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 4 - protein, 503 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 5 - protein, 518 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 6 - protein, 518 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 7 - protein, 662 aa, Homo sapiens
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 8 - protein, 862 aa, Homo sapiens
MAHTFRGCSLAFMFIITWLLIKAKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 9 - protein, 4 aa, synthetic construct
GGGS

SEQ ID NO: 10 - protein, 6 aa, synthetic construct
GGSGGS

SEQ ID NO: 11 - protein, 10 aa, synthetic construct
GGGGSGGGGS

SEQ ID NO: 12 - protein, 15 aa, synthetic construct
GGGGSGGGGSGGGGS

SEQ ID NO: 13 - protein, 20 aa, synthetic construct
GGGGSGGGGSGGGGSGGGGS

SEQ ID NO: 14 - protein, 9 aa, synthetic construct
GGGGSGGGG

SEQ ID NO: 15 - protein, 4 aa, synthetic construct
GSGG

SEQ ID NO: 16 - protein, 25 aa, synthetic construct
GGGGSGGGGSGGGGSGGGGSGGGGS

SEQ ID NO: 17 - protein, 5 aa, synthetic construct
GGGGS

SEQ ID NO: 18 - protein, 7 aa, synthetic construct
SGGSGGS

SEQ ID NO: 19 - protein, 8 aa, synthetic construct
GGGSGGGS

SEQ ID NO: 20 - protein, 9 aa, synthetic construct
GGGGSGGGS

SEQ ID NO: 21 - protein, 10 aa, synthetic construct
GGGGSGGGGS

SEQ ID NO: 22 - protein, 15 aa, synthetic construct
GGGGSGGGGSGGGGS

SEQ ID NO: 23 - protein, 18 aa, synthetic construct
GGGGSGGGGSGGGGGGGS

SEQ ID NO: 24 - protein, 20 aa, synthetic construct
GGGGSGGGGSGGGGSGGGGS

SEQ ID NO: 25 - protein, 25 aa, synthetic construct
GGGGSGGGGSGGGGSGGGGSGGGGS

SEQ ID NO: 26 - protein, 30 aa, synthetic construct
GGGGSGGGGSGGGGSGGGGSGGGGSGGGGS

SEQ ID NO: 27 - protein, 35 aa, synthetic construct
GGGGSGGGGSGGGGSGGGGSGGGGSGGGGSGGGGS

SEQ ID NO: 28 - protein, 422 aa, Vesicular stomatitis virus
MSVTVKRIIDNTVVVPKLPANEDPVEYPADYFRKSKEIPLYINTTKSLSDLRGYVYQGLKSGNVSIIHVNSYLYGALKDIRGKLDKDWSSFGINIGKAGDTIGIFDLVSLKALDGVLPDGVSDASRTSADDKWLPLYLLGLYRVGRTQMPEYRKKLMDGLTNQCKMINEQFEPLVPEGRDIFDVWGNDSNYTKIVAAVDMFFHMFKKHECASFRYGTIVSRFKDCAALATFGHLCKITGMSTEDVTTWILNREVADEMVQMMLPGQEIDKADSYMPYLIDFGLSSKSPYSSVKNPAFHFWGQLTALLLRSTRARNARQPDDIEYTSLTTAGLLYAYAVGSSADLAQQFCVGDNKYTPDDSTGGLTTNAPPQGRDVVEWLGWFEDQNRKPTPDMMQYAKRAVMSLQGLREKTIGKYAKSEFDK

SEQ ID NO: 29 - protein, 265 aa, Vesicular stomatitis virus
MDNLTKVREYLKSYSRLDQAVGEIDEIEAQRAEKSNYELFQEDGVEEHTKPSYFQAADDSDTESEPEIEDNQGLYAPDPEAEQVEGFIQGPLDDYADEEVDVVFTSDWKQPELESDEHGKTLRLTSPEGLSGEQKSQWLSTIKAVVQSAKYWNLAECTFEASGEGVIMKERQITPDVYKVTPVMNTHPSQSEAVSDVWSLSKTSMTFQPKKASLQPLTISLDELFSSRGEFISVGGDGRMSHKEAILLGLRYKKLYNQARVKYSL

SEQ ID NO: 30 - protein, 2109 aa, Vesicular stomatitis virus
MEVHDFETDEFNDFNEDDYATREFLNPDERMTYLNHADYNLNSPLISDDIDNLIRKFNSLPIPSMWDSKNWDGVLEMLTSCQANPIPTSQMHKWMGSWLMSDNHDASQGYSFLHEVDKEAEITFDVVETFIRGWGNKPIEYIKKERWTDSFKILAYLCQKFLDLHKLTLILNAVSEVELLNLARTFKGKVRRSSHGTNICRIRVPSLGPTFISEGWAYFKKLDILMDRNFLLMVKDVIIGRMQTVLSMVCRIDNLFSEQDIFSLLNIYRIGDKIVERQGNFSYDLIKMVEPICNLKLMKLARESRPLVPQFPHFENHIKTSVDEGAKIDRGIRFLHDQIMSVKTVDLTLVIYGSFRHWGHPFIDYYTGLEKLHSQVTMKKDIDVSYAKALASDLARIVLFQQFNDHKKWFVNGDLLPHDHPFKSHVKENTWPTAAQVQDFGDKWHELPLIKCFEIPDLLDPSIIYSDKSHSMNRSEVLKHVRMNPNTPIPSKKVLQTMLDTKATNWKEFLKEIDEKGLDDDDLIIGLKGKERELKLAGRFFSLMSWKLREYFVITEYLIKTHFVPMFKGLTMADDLTAVIKKMLDSSSGQGLKSYEAICIANHIDYEKWNNHQRKLSNGPVFRVMGQFLGYPSLIERTHEFFEKSLIYYNGRPDLMRVHNNTLINSTSQRVCWQGQEGGLEGLRQKGWSILNLLVIQREAKIRNTAVKVLAQGDNQVICTQYKTKKSRNVVELQGALNQMVSNNEKIMTAIKIGTGKLGLLINDDETMQSADYLNYGKIPIFRGVIRGLETKRWSRVTCVTNDQIPTCANIMSSVSTNALTVAHFAENPINAMIQYNYFGTFARLLLMMHDPALRQSLYEVQDKIPGLHSSTFKYAMLYLDPSIGGVSGMSLSRFLIRAFPDPVTESLSFWRFIHVHARSEHLKEMSAVFGNPEIAKFRITHIDKLVEDPTSLNIAMGMSPANLLKTEVKKCLIESRQTIRNQVIKDATIYLYHEEDRLRSFLWSINPLFPRFLSEFKSGTFLGVADGLISLFQNSRTIRNSFKKKYHRELDDLIVRSEVSSLTHLGKLHLRRGSCKMWTCSATHADTLRYKSWGRTVIGTTVPHPLEMLGPQHRKETPCAPCNTSGFNYVSVHCPDGIHDVFSSRGPLPAYLGSKTSESTSILQPWERESKVPLIKRATRLRDAISWFVEPDSKLAMTILSNIHSLTGEEWTKRQHGFKRTGSALHRFSTSRMSHGGFASQSTAALTRLMATTDTMRDLGDQNFDFLFQATLLYAQITTTVARDGWITSCTDHYHIACKSCLRPIEEITLDSSMDYTPPDVSHVLKTWRNGEGSWGQEIKQIYPLEGNWKNLAPAEQSYQVGRCIGFLYGDLAYRKSTHAEDSSLFPLSIQGRIRGRGFLKGLLDGLMRASCCQVIHRRSLAHLKRPANAVYGGLIYLIDKLSVSPPFLSLTRSGPIRDELETIPHKIPTSYPTSNRDMGVIVRNYFKYQCRLIEKGKYRSHYSQLWLFSDVLSIDFIGPFSISTTLLQILYKPFLSGKDKNELRELANLSSLLRSGEGWEDIHVKFFTKDILLCPEEIRHACKFGIAKDNNKDMSYPPWGRESRGTITTIPVYYTTTPYPKMLEMPPRIQNPLLSGIRLGQLPTGAHYKIRSILHGMGIHYRDFLSCGDGSGGMTAALLRENVHSRGIFNSLLELSGSVMRGASPEPPSALETLGGDKSRCVNGETCWEYPSDLCDPRTWDYFLRLKAGLGLQIDLIVMDMEVRDSSTSLKIETNVRNYVHRILDEQGVLIYKTYGTYICESEKNAVTILGPMFKTVDLVQTEFSSSQTSEVYMVCKGLKKLIDEPNPDWSSINESWKNLYAFQSSEQEFARAKKVSTYFTLTGIPSQFIPDPFVNIETMLQIFGVPTGVSHAAALKSSDRPADLLTISLFYMAIISYYNINHIRVGPIPPNPPSDGIAQNVGIAITGISFWLSLMEKDIPLYQQCLAVIQQSFPIRWEAVSVKGGYKQKWSTRGDGLPKDTRISDSLAPIGNWIRSLELVRNQVRLNPFNEILFNQLCRTVDNHLKWSNLRRNTGMIEWINRRISKEDRSILMLKSDLHEENSWRD

SEQ ID NO: 31 - protein, 229 aa, Vesicular stomatitis virus
MSSLKKILGLKGKGKKSKKLGIAPPPYEEDTSMEYAPSAPIDKSYFGVDEMDTYDPNQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTIELTMTIYDDESLEAAPMIWDHFNSSKFSDFREKALMFGLIVEKKASGAWVLDSIGHFK

SEQ ID NO: 32 - protein, 498 aa, Lymphocytic choriomeningitis mammarenavirus
MGQIVTMFEALPHIIDEVINIVIIVLIIITSIKAVYNFATCGILALVSFLFLAGRSCGMYGLNGPDIYKGVYQFKSVEFDMSHLNLTMPNACSANNSHHYISMGSSGLELTFTNDSILNHNFCNLTSAFNKKTFDHTLMSIVSSLHLSIRGNSNHKAVSCDFNNGITIQYNLSFSDPQSAISQCRTFRGRVLDMFRTAFGGKYMRSGWGWAGSDGKTTWCSQTSYQYLIIQNRTWENHCRYAGPFGMSRILFAQEKTKFLTRRLAGTFTWTLSDSSGVENPGGYCLTKWMILAAELKCFGNTAVAKCNVNHDEEFCDMLRLIDYNKAALSKFKQDVESALHVFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLISIFLHLVKIPTHRHIKGGSCPKPHRLTNKGICSCGAFKVPGVKTIWKRR

SEQ ID NO: 33 - protein, 498 aa, Dandenong virus
MGQLITMFEALPHIIDEVINIVIIVLVIITSIKAVYNFATCGIIALISFCLLAGRSCGLYGVTGPDIYKGLYQFKSVEFNMSQLNLTMPNACSANNSHHYISMGKSGLELTFTNDSIISHNFCNLTDGFKKKTFDHTLMSIVASLHLSIRGNTNYKAVSCDFNNGITIQYNLSFSDAQSAINQCRTFRGRVLDMFRTAFGGKYMRSGYGWKGSDGKTTWCSQTSYQYLIIQNRTWENHCEYAGPFGLSRVLFAQEKTKFLTRRLAGTFTWTLSDSSGTENPGGYCLTKWMLIAAELKCFGNTAVAKCNINHDEEFCDMLRLIDYNKAALKKFKEDVESALHLFKTTVNSLISDQLLMRNHLRDLMGVPYCNYSKFWYLEHVKTGDTSVPKCWLVSNGSYLNETHFSDQIEQEADNMITEMLRKDYIKRQGSTPLALMDLLMFSTSAYLISVFLHLMKIPTHRHIKGGTCPKPHRLTSKGICSCGAFKVPGVKTVWKRR

SEQ ID NO: 34 - protein, 489 aa, Mopeia mammarenavirus
MGQIVTFFQEVPHILEEVMNIVLMTLSILAILKGIYNVMTCGIIGLITFLFLCGRSCSSIYKDNYEFFSLDLDMSSLNATMPLSCSKNNSHHYIQVGNETGLELTLTNTSIIDHKFCNLSDAHRRNLYDKALMSILTTFHLSIPDFNQYEAMSCDFNGGKISIQYNLSHSNYVDAGNHCGTIANGIMDVFRRMYWSTSLSVASDISGTQCIQTDYKYLIIQNTSWEDHCMFSRPSPMGFLSLLSQRTRNFYISRRLLGLFTWTLSDSEGNDMPGGYCLTRSMLIGLDLKCFGNTAIAKCNQAHDEEFCDMLRLFDFNKQAISKLRSEVQQSINLINKAVNALINDQLVMRNHLRDLMGIPYCNYSKFWYLNDTRTGRTSLPKCWLVTNGSYLNETQFSTEIEQEANNMFTDMLRKEYEKRQSTTPLGLVDLFVFSTSFYLISVFLHLIKIPTHRHIKGKPCPKPHRLNHMAICSCGFYKQPGLPTQWKR

SEQ ID NO: 35 - protein, 446 aa, synthetic construct
EVMLVESGGGLVQPGGSLRLSCTASGFTFSASAMSWVRQAPGKGLEWVAYISGGGGDTYYSSSVKGRFTISRDNAKNSLYLQMNSLRAEDTAVYYCARHSNVNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 36 - protein, 218 aa, synthetic construct
EIVLTQSPATLSLSPGERATMSCRASENIDTSGISFMNWYQQKPGQAPKLLIYVASNQGSGIPARFSGSGSGTDFTLTISRLEPEDFAVYYCQQSKEVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 446 aa, synthetic construct
EVMLVESGGGLVQPGGSLRLSCTASGFTFSASAMSWVRQAPGKGLEWVAYISGGGGDTYYSSSVKGRFTISRDNAKNSLYLQMNSLRAEDTAVYYCARHSNPNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 38 - protein, 218 aa, synthetic construct; identical to SEQ ID NO: 36

SEQ ID NO: 39 - protein, 446 aa, synthetic construct
EVMLVESGGGLVQPGGSLRLSCTASGFTFSKSAMSWVRQAPGKGLEWVAYISGGGGDTYYSSSVKGRFTISRDNAKNSLYLQMNSLRAEDTAVYYCARHSNVNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 40 - protein, 218 aa, synthetic construct
EIVLTQSPATLSLSPGERATMSCRASENIDVSGISFMNWYQQKPGQAPKLLIYVASNQGSGIPARFSGSGSGTDFTLTISRLEPEDFAVYYCQQSKEVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 446 aa, synthetic construct; identical to SEQ ID NO: 39

SEQ ID NO: 42 - protein, 218 aa, synthetic construct; identical to SEQ ID NO: 40

SEQ ID NO: 43 - protein, 446 aa, synthetic construct; identical to SEQ ID NO: 39

SEQ ID NO: 44 - protein, 218 aa, synthetic construct; identical to SEQ ID NO: 40

SEQ ID NO: 45 - protein, 445 aa, Homo sapiens
MTMFENVTRALARQLNPRGDLTPLDSLIDFKRFHPFCLVLRKRKSTLFWGARYVRTDYTLLDVLEPGSSPSDPTDTGNFGFKNMLDTRVEGDVDVPKTVKVKGTAGLSQNSTLEVQTLSVAPKALETVQERKLAADHPFLKEMQDQGENLYVVMEVVETVQEVTLERAGKAEACFSLPFFAPLGLQGSINHKEAVTIPKGCVLAFRVRQLMVKGKDEWDIPHICNDNMQTFPPGEKSGEEKVILIQASDVGDVHEGFRTLKEEVQRETQQVEKLSRVGQSSLLSSLSKLLGKKKELQDLELALEGALDKGHEVTLEALPKDVLLSKEAVGAILYFVGALTELSEAQQKLLVKSMEKKILPVQLKLVESTMEQNFLLDKEGVFPLQPELLSSLGDEELTLTEALVGLSGLEVQRSGPQYMWDPDTLPRLCALYAGLSLLQQLTKAS

SEQ ID NO: 46 - protein, 416 aa, Homo sapiens
MFSVFEEITRIVVKEMDAGGDMIAVRSLVDADRFRCFHLVGEKRTFFGCRHYTTGLTLMDILDTDGDKWLDELDSGLQGQKAEFQILDNVDSTGELIVRLPKEITISGSFQGFHHQKIKISENRISQQYLATLENRKLKRELPFSFRSINTRENLYLVTETLETVKEETLKSDRQYKFWSQISQGHLSYKHKGQREVTIPPNRVLSYRVKQLVFPNKETMNIHFRGKTKSFPEEKDGASSCLGKSLGSEDSRNMKEKLEDMESVLKDLTEEKRKDVLNSLAKCLGKEDIRQDLEQRVSEVLISGELHMEDPDKPLLSSLFNAAGVLVEARAKAILDFLDALLELSEEQQFVAEALEKGTLPLLKDQVKSVMEQNWDELASSPPDMDYDPEARILCALYVVVSILLELAEGPTSVSS

SEQ ID NO: 47 - protein, 508 aa, Homo sapiens
MPSMLERISKNLVKEIGSKDLTPVKYLLSATKLRQFVILRKKKDSRSSFWEQSDYVPVEFSLNDILEPSSSVLETVVTGPFHFSDIMIQKHKADMGVNVGIEVSVSGEASVDHGCSLEFQIVTIPSPNLEDFQKRKLLDPEPSFLKECRRRGDNLYVVTEAVELINNTVLYDSSSVNILGKIALWITYGKGQGQGESLRVKKKALTLQKGMVMAYKRKQLVIKEKAILISDDDEQRTFQDEYEISEMVGYCAARSEGLLPSFHTISPTLFNASSNDMKLKPELFLTQQFLSGHLPKYEQVHILPVGRIEEPFWQNFKHLQEEVFQKIKTLAQLSKDVQDVMFYSILAMLRDRGALQDLMNMLELDSSGHLDGPGGAILKKLQQDSNHAWFNPKDPILYLLEAIMVLSDFQHDLLACSMEKRILLQQQELVRSILEPNFRYPWSIPFTLKPELLAPLQSEGLAITYGLLEECGLRMELDNPRSTWDVEAKMPLSALYGTLSLLQQLAEA

SEQ ID NO: 48 - protein, 484 aa, Homo sapiens
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNFLTDGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

SEQ ID NO: 49 - protein, 496 aa, Homo sapiens
MFAKATRNFLREVDADGDLIAVSNLNDSDKLQLLSLVTKKKRFWCWQRPKYQFLSLTLGDVLIEDQFPSPVVVESDFVKYEGKFANHVSGTLETALGKVKLNLGGSSRVESQSSFGTLRKQEVDLQQLIRDSAERTINLRNPVLQQVLEGRNEVLCVLTQKITTMQKCVISEHMQVEEKCGGIVGIQTKTVQVSATEDGNVTKDSNVVLEIPAATTIAYGVIELYVKLDGQFEFCLLRGKQGGFENKKRIDSVYLDPLVFREFAFIDMPDAAHGISSQDGPLSVLKQATLLLERNFHPFAELPEPQQTALSDIFQAVLFDDELLMVLEPVCDDLVSGLSPTVAVLGELKPRQQQDLVAFLQLVGCSLQGGCPGPEDAGSKQLFMTAYFLVSALAEMPDSAAALLGTCCKLQIIPTLCHLLRALSDDGVSDLEDPTLTPLKDTERFGIVQRLFASADISLERLKSSVKAVILKDSKVFPLLLCITLNGLCALGREHS

SEQ ID NO: 50 - protein, 352 aa, Homo sapiens
MFAAATKSFVKQVGDGGRLVPVPSLSEADKYQPLSLVVKKKRCFLFPRYKFTSTPFTLKDILLGDREISAGISSYQLLNYEDESDVSLYGRRGNHIVNDVGINVAGSDSIAVKASFGIVTKHEVEVSTLLKEITTRKINFDHSLIRQSRSSRKAVLCVVMESIRTTRQCSLSVHAGIRGEAMRFHFMDEQNPKGRDKAIVFPAHTTIAFSVFELFIYLDGAFDLCVTSVSKGGFEREETATFALLYRLRNILFERNRRVMDVISRSQLYLDDLFSDYYDKPLSMTDISLKEGTHIRVNLLNHNIPKGPCILCGMGNFKRETVYGCFQCSVDGQKYVRLHAVPCFDIWHKRMK

SEQ ID NO: 51 - protein, 246 aa, Homo sapiens
MTMFENVTRALARQLNPRGDLTPLDSLIDFKRFHPFCLVLRKRKSTLFWGARYVRTDYTLLDVLEPGSSPSDPTDTGNFGFKNMLDTRVEGDVDVPKTVKVKGTAGLSQNSTLEVQTLSVAPKALETVQERKLAADHPFLKEMQDQGENLYVVMEVVETVQEVTLERAGKAEACFSLPFFAPLGLQGSINHKEAVTIPKGCVLAFRVRQLMVKGKDEWDIPHICNDNMQTFPPGEKSGEEKVILIQ

SEQ ID NO: 52 - protein, 244 aa, Homo sapiens
MFSVFEEITRIVVKEMDAGGDMIAVRSLVDADRFRCFHLVGEKRTFFGCRHYTTGLTLMDILDTDGDKWLDELDSGLQGQKAEFQILDNVDSTGELIVRLPKEITISGSFQGFHHQKIKISENRISQQYLATLENRKLKRELPFSFRSINTRENLYLVTETLETVKEETLKSDRQYKFWSQISQGHLSYKHKGQREVTIPPNRVLSYRVKQLVFPNKETMNIHFRGKTKSFPEEKDGASSCLGK

SEQ ID NO: 53 - protein, 365 aa, Homo sapiens
MPSMLERISKNLVKEIGSKDLTPVKYLLSATKLRQFVILRKKKDSRSSFWEQSDYVPVEFSLNDILEPSSSVLETVVTGPFHFSDIMIQKHKADMGVNVGIEVSVSGEASVDHGCSLEFQIVTIPSPNLEDFQKRKLLDPEPSFLKECRRRGDNLYVVTEAVELINNTVLYDSSSVNILGKIALWITYGKGQGQGESLRVKKKALTLQKGMVMAYKRKQLVIKEKAILISDDDEQRTFQDEYEISEMVGYCAARSEGLLPSFHTISPTLFNASSNDMKLKPELFLTQQFLSGHLPKYEQVHILPVGRIEEPFWQNFKHLQEEVFQKIKTLAQLSKDVQDVMFYSILAMLRDRGALQDLMNMLELD

SEQ ID NO: 54 - protein, 275 aa, Homo sapiens
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNFLTD

SEQ ID NO: 55 - protein, 270 aa, Homo sapiens
MFAKATRNFLREVDADGDLIAVSNLNDSDKLQLLSLVTKKKRFWCWQRPKYQFLSLTLGDVLIEDQFPSPVVVESDFVKYEGKFANHVSGTLETALGKVKLNLGGSSRVESQSSFGTLRKQEVDLQQLIRDSAERTINLRNPVLQQVLEGRNEVLCVLTQKITTMQKCVISEHMQVEEKCGGIVGIQTKTVQVSATEDGNVTKDSNVVLEIPAATTIAYGVIELYVKLDGQFEFCLLRGKQGGFENKKRIDSVYLDPLVFREFAFIDMPD

SEQ ID NO: 56 - protein, 199 aa, Homo sapiens
ASDVGDVHEGFRTLKEEVQRETQQVEKLSRVGQSSLLSSLSKLLGKKKELQDLELALEGALDKGHEVTLEALPKDVLLSKEAVGAILYFVGALTELSEAQQKLLVKSMEKKILPVQLKLVESTMEQNFLLDKEGVFPLQPELLSSLGDEELTLTEALVGLSGLEVQRSGPQYMWDPDTLPRLCALYAGLSLLQQLTKAS

SEQ ID NO: 57 - protein, 172 aa, Homo sapiens
SLGSEDSRNMKEKLEDMESVLKDLTEEKRKDVLNSLAKCLGKEDIRQDLEQRVSEVLISGELHMEDPDKPLLSSLFNAAGVLVEARAKAILDFLDALLELSEEQQFVAEALEKGTLPLLKDQVKSVMEQNWDELASSPPDMDYDPEARILCALYVVVSILLELAEGPTSVSS

SEQ ID NO: 58 - protein, 143 aa, Homo sapiens
SSGHLDGPGGAILKKLQQDSNHAWFNPKDPILYLLEAIMVLSDFQHDLLACSMEKRILLQQQELVRSILEPNFRYPWSIPFTLKPELLAPLQSEGLAITYGLLEECGLRMELDNPRSTWDVEAKMPLSALYGTLSLLQQLAEA

SEQ ID NO: 59 - protein, 209 aa, Homo sapiens
GVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

SEQ ID NO: 60 - protein, 226 aa, Homo sapiens
AAHGISSQDGPLSVLKQATLLLERNFHPFAELPEPQQTALSDIFQAVLFDDELLMVLEPVCDDLVSGLSPTVAVLGELKPRQQQDLVAFLQLVGCSLQGGCPGPEDAGSKQLFMTAYFLVSALAEMPDSAAALLGTCCKLQIIPTLCHLLRALSDDGVSDLEDPTLTPLKDTERFGIVQRLFASADISLERLKSSVKAVILKDSKVFPLLLCITLNGLCALGREHS

SEQ ID NO: 61 - protein, 277 aa, Homo sapiens
MENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETDSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYH

SEQ ID NO: 62 - skipped

SEQ ID NO: 63 - protein, 4 aa, synthetic construct
DMPD

SEQ ID NO: 64 - protein, 4 aa, synthetic construct
DEVD

SEQ ID NO: 65 - protein, 4 aa, synthetic construct
DLPD

SEQ ID NO: 66 - protein, 540 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 67 - protein, 540 aa, synthetic construct
MCHQQLVISWFSLVFLASPLVARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 68 - protein, 22 aa, Homo sapiens
MCHQQLVISWFSLVFLASPLVA

SEQ ID NO: 69 - protein, 8 aa, synthetic construct
DXEXNPGP

SEQ ID NO: 70 - protein, 18 aa, synthetic construct
EGRGSLLTCGDVEENPGP

SEQ ID NO: 71 - protein, 21 aa, synthetic construct
GSGEGRGSLLTCGDVEENPGP

SEQ ID NO: 72 - protein, 1057 aa, synthetic construct
MFAKATRNFLREVDADGDLIAVSNLNDSDKLQLLSLVTKKKRFWCWQRPKYQFLSLTLGDVLIEDQFPSPVVVESDFVKYEGKFANHVSGTLETALGKVKLNLGGSSRVESQSSFGTLRKQEVDLQQLIRDSAERTINLRNPVLQQVLEGRNEVLCVLTQKITTMQKCVISEHMQVEEKCGGIVGIQTKTVQVSATEDGNVTKDSNVVLEIPAATTIAYGVIELYVKLDGQFEFCLLRGKQGGFENKKRIDSVYLDPLVFREFAFIDMPDAAHGISSQDGPLSVLKQATLLLERNFHPFAELPEPQQTALSDIFQAVLFDDELLMVLEPVCDDLVSGLSPTVAVLGELKPRQQQDLVAFLQLVGCSLQGGCPGPEDAGSKQLFMTAYFLVSALAEMPDSAAALLGTCCKLQIIPTLCHLLRALSDDGVSDLEDPTLTPLKDTERFGIVQRLFASADISLERLKSSVKAVILKDSKVFPLLLCITLNGLCALGREHSGSGEGRGSLLTCGDVEENPGPMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 73 - protein, 19 aa, synthetic construct
ATNFSLLKQAGDVEENPGP

SEQ ID NO: 74 - protein, 20 aa, synthetic construct
QCTNYALLKLAGDVESNPGP

SEQ ID NO: 75 - protein, 22 aa, synthetic construct
VKQTLNFDLLKLAGDVESNPGP

SEQ ID NO: 76 - protein, 424 aa, Mus musculus
MFAKATRNFLKEVDAGGDLISVSHLNDSDKLQLLSLVTKKKRYWCWQRPKYQILSATLEDVLTEGHCLSPVVVESDFVKYESKCENHKSGAIGTVVGKVKLNVGGKGVVESHSSFGTLRKQEVDVQQLIQDAVKRTVNMDNLVLQQVLESRNEVLCVLTQKIMTTQKCVISEHVQSEETCGGMVGIQTKTIQVSATEDGTVTTDTNVVLEIPAATTIAYGIMELFVKQDGQFEFCLLQGKHGGFEHERKLDSVYLDPLAYREFAFLDMLDGGQGISSQDGPLRVVKQATLHLERSFHPFAVLPAQQQRALFCVLQKILFDEELLRALEQVCDDVAGGLWSSQAVLAMEELTDSQQQDLTAFLQLVGYRIQGEHPGPQDEVSNQKLFATAYFLVSALAEMPDNATVFLGTCCKLHVISSLCCLGG

SEQ ID NO: 77 - protein, 487 aa, Mus musculus
MPSAFEKVVKNVIKEVSGSRGDLIPVDSLRNSTSFRPYCLLNRKFSSSRFWKPRYSCVNLSIKDILEPSAPEPEPECFGSFKVSDVVDGNIQGRVMLSGMGEGKISGGAAVSDSSSASMNVCILRVTQKTWETMQHERHLQQPENKILQQLRSRGDDLFVVTEVLQTKEEVQITEVHSQEGSGQFTLPGALCLKGEGKGHQSRKKMVTIPAGSILAFRVAQLLIGSKWDILLVSDEKQRTFEPSSGDRKAVGQRHHGLNVLAALCSIGKQLSLLSDGIDEEELIEAADFQGLYAEVKACSSELESLEMELRQQILVNIGKILQDQPSMEALEASLGQGLCSGGQVEPLDGPAGCILECLVLDSGELVPELAAPIFYLLGALAVLSETQQQLLAKALETTVLSKQLELVKHVLEQSTPWQEQSSVSLPTVLLGDCWDEKNPTWVLLEECGLRLQVESPQVHWEPTSLIPTSALYASLFLLSSLGQKPC

SEQ ID NO: 78 - protein, 487 aa, synthetic construct
MPSAFEKVVKNVIKEVSGSRGDLIPVDSLRNSTSFRPYCLLNRKFSSSRFWKPRYSCVNLSIKDILEPSAPEPEPECFGSFKVSDVVDGNIQGRVMLSGMGEGKISGGAAVSDSSSASMNVCILRVTQKTWETMQHERHLQQPENKILQQLRSRGDDLFVVTEVLQTKEEVQITEVHSQEGSGQFTLPGALCLKGEGKGHQSRKKMVTIPAGSILAFRVAQLLIGSKWDILLVSDEKQRTFEPSSGDRKAVGQRHHGLNVLAALCSIGKQLSDEVDGIDEEELIEAADFQGLYAEVKACSSELESLEMELRQQILVNIGKILQDQPSMEALEASLGQGLCSGGQVEPLDGPAGCILECLVLDSGELVPELAAPIFYLLGALAVLSETQQQLLAKALETTVLSKQLELVKHVLEQSTPWQEQSSVSLPTVLLGDCWDEKNPTWVLLEECGLRLQVESPQVHWEPTSLIPTSALYASLFLLSSLGQKPC

SEQ ID NO: 79 - protein, 193 aa, Mus musculus
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 80 - protein, 313 aa, Mus musculus
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 81 - protein, 19 aa, Homo sapiens
MGWSCIILFLVATATGVHS

SEQ ID NO: 82 - protein, 521 aa, synthetic construct
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 83 - protein, 540 aa, synthetic construct
MGWSCIILFLVATATGVHSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 84 - protein, 985 aa, synthetic construct
MFAKATRNFLKEVDAGGDLISVSHLNDSDKLQLLSLVTKKKRYWCWQRPKYQILSATLEDVLTEGHCLSPVVVESDFVKYESKCENHKSGAIGTVVGKVKLNVGGKGVVESHSSFGTLRKQEVDVQQLIQDAVKRTVNMDNLVLQQVLESRNEVLCVLTQKIMTTQKCVISEHVQSEETCGGMVGIQTKTIQVSATEDGTVTTDTNVVLEIPAATTIAYGIMELFVKQDGQFEFCLLQGKHGGFEHERKLDSVYLDPLAYREFAFLDMLDGGQGISSQDGPLRVVKQATLHLERSFHPFAVLPAQQQRALFCVLQKILFDEELLRALEQVCDDVAGGLWSSQAVLAMEELTDSQQQDLTAFLQLVGYRIQGEHPGPQDEVSNQKLFATAYFLVSALAEMPDNATVFLGTCCKLHVISSLCCLGGGSGEGRGSLLTCGDVEENPGPMGWSCIILFLVATATGVHSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 85 - protein, 228 aa, synthetic construct
MSSLKKILGLKGKGKKSKKLGIAPPPYEEDTSMEYAPSAPIDKSYFGVDEDTYDPNQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTIELTMTIYDDESLEAAPMIWDHFNSSKFSDFREKALMFGLIVEKKASGAWVLDSIGHFK

SEQ ID NO: 86 - protein, 662 aa, synthetic construct
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNDEVDGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPHGSGEGRGSLLTCGDVEENPGPHFGRLHCTTAVIRNINDQVLFVDKRQPVFEDMTDIDQSASEPQTRLIIYAYGDSRARGKAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 87 - protein, 1224 aa, synthetic construct
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNDEVDGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPHGSGEGRGSLLTCGDVEENPGPHFGRLHCTTAVIRNINDQVLFVDKRQPVFEDMTDIDQSASEPQTRLIIYAYGDSRARGKAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQSGSGATNFSLLKQAGDVEENPGPMGWSCIILFLVATATGVHSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 88 - protein, 836 aa, synthetic construct
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNDEVDGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPHGSGEGRGSLLTCGDVEENPGPHFGRLHCTTAVIRNINDQVLFVDKRQPVFEDMTDIDQSASEPQTRLIIYAYGDSRARGKAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQSGSGATNFSLLKQAGDVEENPGPVPIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIDFTMESVSS

SEQ ID NO: 89 - protein, 1048 aa, synthetic construct
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNDEVDGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPHGSGATNFSLLKQAGDVEENPGPVPIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIDFTMESVSSGSGEGRGSLLTCGDVEENPGPNFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQSGSGQCTNYALLKLAGDVESNPGPARLCAFLVMLIVMSYWSICSLGCDLPHTYNLRNKRALKVLAQMRRLPFLSCLKDRQDFGFPLEKVDNQQIQKAQAIPVLRDLTQQTLNLFTSKASSAAWNATLLDSFCNDLHQQLNDLQTCLMQQVGVQEPPLTQEDALLAVRKYFHRITVYLREKKHSPCAWEVVRAEVWRALSSSVNLLPRLSEEKE

SEQ ID NO: 90 - protein, 1048 aa, synthetic construct
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNDEVDGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPHGSGEGRGSLLTCGDVEENPGPNFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQSGSGATNFSLLKQAGDVEENPGPVPIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIDFTMESVSSGSGQCTNYALLKLAGDVESNPGPARLCAFLVMLIVMSYWSICSLGCDLPHTYNLRNKRALKVLAQMRRLPFLSCLKDRQDFGFPLEKVDNQQIQKAQAIPVLRDLTQQTLNLFTSKASSAAWNATLLDSFCNDLHQQLNDLQTCLMQQVGVQEPPLTQEDALLAVRKYFHRITVYLREKKHSPCAWEVVRAEVWRALSSSVNLLPRLSEEKE

SEQ ID NO: 91 - protein, 270 aa, Mus musculus
MAKVPDLFEDLKNCYSENEDYSSAIDHLSLNQKSFYDASYGSLHETCTDQFVSLRTSETSKMSNFTFKESRVTVSATSSNGKILKKRRLSFSETFTEDDLQSITHDLEETIQPRSAPYTYQSDLRYKLMKLVRQKFVMNDSLNQTIYQDVDKHYLSTTWLNDLQQEVKFDMYAYSSGGDDSKYPVTLKISDSQLFVSAQGEDQPVLLKELPETPKLITGSETDLIFFWKSINSKNYFTSAAYPELFIATKEQSRVHLARGLPSMTDFQIS

SEQ ID NO: 92 - protein, 156 aa, Mus musculus
SAPYTYQSDLRYKLMKLVRQKFVMNDSLNQTIYQDVDKHYLSTTWLNDLQQEVKFDMYAYSSGGDDSKYPVTLKISDSQLFVSAQGEDQPVLLKELPETPKLITGSETDLIFFWKSINSKNYFTSAAYPELFIATKEQSRVHLARGLPSMTDFQIS

SEQ ID NO: 93 - protein, 269 aa, Mus musculus
MATVPELNCEMPPFDSDENDLFFEVDGPQKMKGCFQTFDLGCPDESIQLQISQQHINKSFRQAVSLIVAVEKLWQLPVSFPWTFQDEDMSTFFSFIFEEEPILCDSWDDDDNLLVCDVPIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIDFTMESVSS

SEQ ID NO: 94 - protein, 151 aa, Mus musculus
VPIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIDFTMESVS

SEQ ID NO: 95 - protein, 192 aa, Mus musculus
MAAMSEDSCVNFKEMMFIDNTLYFIPEENGDLESDNFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 96 - protein, 157 aa, Mus musculus
NFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 97 - protein, 157 aa, Mus musculus
HFGRLHCTTAVIRNINDQVLFVDKRQPVFEDMTDIDQSASEPQTRLIIYAYGDSRARGKAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 98 - protein, 190 aa, Mus musculus
MARLCAFLVMLIVMSYWSICSLGCDLPHTYNLRNKRALKVLAQMRRLPFLSCLKDRQDFGFPLEKVDNQQIQKAQAIPVLRDLTQQTLNLFTSKASSAAWNATLLDSFCNDLHQQLNDLQTCLMQQVGVQEPPLTQEDALLAVRKYFHRITVYLREKKHSPCAWEVVRAEVWRALSSSVNLLPRLSEEKE

SEQ ID NO: 99 - protein, 271 aa, Homo sapiens
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 100 - protein, 159 aa, Homo sapiens
SAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 101 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 102 - protein, 153 aa, Homo sapiens
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 103 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 104 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 105 - protein, 157 aa, Homo sapiens
YFGKLESKLSVIRNLNDQVLFIDQGNRPLFEDMTDSDCRDNAPRTIFIISKYSDSLARGLAVTISVKCEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 106 - protein, 188 aa, Homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 107 - protein, 484 aa, synthetic construct
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNDEVDGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

SEQ ID NO: 108 - protein, 664 aa, synthetic construct
MHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKPGSGATNFSLLKQAGDVEENPGPMHQKRTAMFQDPQERPRKLPQLCTELQTTIHDIILECVYCKQQLLRREVYDFAFRDLCIVYRGGNPYAVCDKCLKFYSKISEYRHYCYSLYGTTLEQQYNKPLCDGLIRCINCQKPLCPEEKQRHLDKKQRFHNIRGRWTGRCMSCCRSSRTRRETQLGSGEGRGSLLTCGDVEENPGPMETLCQRLNVCQDKILTHYENDSTDLRDHIDYWKHMRLECAIYYKAREMGFKHINHQVVPTLAVSKNKALQAIELQLTLETIYNSQYSNEKWTLQDVSLEVYLTAPTGCIKKHGYTVEVQFDGDICNTMHYTNWTHIYICEEASVTVVEGQVDYYGLYYVHEGIRTYFVQFKDDAEKYSKNKVWEVHAGGQVILCPTSVFSSNEVSSPEIIRQHLANHPAATHTKAVALGTEETQTTIQRPRSEPDTGNPCHTTKLLHRDSVDSAPILTAFNSSHKGRINCNSNTTPIVHLKGDANTLKCLRYRFKKHCTLYTAVSSTWHWTGHNVKHKSAIVTLTYDSEWQRDQFLSQVKIPKTITVSTGFMSI

SEQ ID NO: 109 - protein, 512 aa, Mus musculus
MFAKATRNFLKEVDAGGDLISVSHLNDSDKLQLLSLVTKKKRYWCWQRPKYQILSATLEDVLTEGHCLSPVVVESDFVKYESKCENHKSGAIGTVVGKVKLNVGGKGVVESHSSFGTLRKQEVDVQQLIQDAVKRTVNMDNLVLQQVLESRNEVLCVLTQKIMTTQKCVISEHVQSEETCGGMVGIQTKTIQVSATEDGTVTTDTNVVLEIPAATTIAYGIMELFVKQDGQFEFCLLQGKHGGFEHERKLDSVYLDPLAYREFAFLDMLDGGQGISSQDGPLRVVKQATLHLERSFHPFAVLPAQQQRALFCVLQKILFDEELLRALEQVCDDVAGGLWSSQAVLAMEELTDSQQQDLTAFLQLVGYRIQGEHPGPQDEVSNQKLFATAYFLVSALAEMPDNATVFLGTCCKLHVISSLCCLLHALSDDSVCDFHNPTLAPLRDTERFGIVQRLFASADIALERMQFSAKATILKDSCIFPLILHITLSGLSTLSKEHEEELCQSGHATGQD

SEQ ID NO: 110 - protein, 1073 aa, synthetic construct
MFAKATRNFLKEVDAGGDLISVSHLNDSDKLQLLSLVTKKKRYWCWQRPKYQILSATLEDVLTEGHCLSPVVVESDFVKYESKCENHKSGAIGTVVGKVKLNVGGKGVVESHSSFGTLRKQEVDVQQLIQDAVKRTVNMDNLVLQQVLESRNEVLCVLTQKIMTTQKCVISEHVQSEETCGGMVGIQTKTIQVSATEDGTVTTDTNVVLEIPAATTIAYGIMELFVKQDGQFEFCLLQGKHGGFEHERKLDSVYLDPLAYREFAFLDMLDGGQGISSQDGPLRVVKQATLHLERSFHPFAVLPAQQQRALFCVLQKILFDEELLRALEQVCDDVAGGLWSSQAVLAMEELTDSQQQDLTAFLQLVGYRIQGEHPGPQDEVSNQKLFATAYFLVSALAEMPDNATVFLGTCCKLHVISSLCCLLHALSDDSVCDFHNPTLAPLRDTERFGIVQRLFASADIALERMQFSAKATILKDSCIFPLILHITLSGLSTLSKEHEEELCQSGHATGQDGSGEGRGSLLTCGDVEENPGPMGWSCIILFLVATATGVHSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 111 - RNA, 14373 nt, Vesicular stomatitis virus
  source 1..14373: mol_type = genomic RNA
tgcttctgtttgtttggtaataatagtaattttccgagtcctctttgaaattgtcattagttttacagacaatgtcagttctcttagtaactgttgtgtcagcatcaaggttttgaaggacgtttactcctaggtcaccttatgggccgtctaatgaagtcttttagtttcctctaaggagaaatgtagttatgatgtttttcaaacagtctagattctcctatacagatggttccggagtttaggcctttacatagttagtatgtacagttgtcgatgaacatacctcgtaatttcctgtaggccccattcaacctatttctaaccagttcaaagccttatttgtagccctttcgtcccctatgttagccttataaactggaacataggaactttcgggacctgccgcatgaaggtctacctcatagcctacgaaggtcttggtcgcgtctactgtttaccaacggaaacatagatgaaccgaatatgtctcacccgtcttgtgtttacggacttatgtcttttttcgagtacctacccgactgtttagttacgttttactagttacttgtcaaacttggagaacacggtcttccagcactgtaaaaactacacacccctttactgtcattaatgtgtttttaacagcgacgtcacctgtacaagaaggtgtacaagttttttgtacttacacggagcaagtctatgccttgataacaaaggtctaagtttctaacacgacgtaaccgttgtaaacctgtggagacgttttattggccttacagatgtcttctacattgctggacctagaacttggctcttcaacgtctactttaccaggtttactacgaaggtccggttctttaactgttccggctaagtatgtacggaataaactagctgaaacctaacagaagattcagaggtataagaaggcagtttttgggacggaaggtgaagacccccgttaactgtcgagaagacgagtctaggtggtctcgttccttacgggctgtcggactactgtaactcatatgtagagaatgatgtcgtccaaacaacatgcgaatacgtcatcctaggagacggctgaaccgtgttgtcaaaacacaacctctattgtttatgtgaggtctactatcatggcctcctaactgctgattacgtggcggtgttccgtctctacaccagcttaccgagcctaccaaacttctagttttgtcttttggctgaggactatactacgtcatacgcttttctcgtcagtacagtgacgttccggattctctcttctgttaaccgttcatacgattcagtcttaaactgtttactgggatattaagagtctagtggataatatataatacgatgtatactttttttgattgtctatagtacctattagagtgttttcaagcactcatagagttcaggataagagcagacctagtccgccatcctctctatctactctagcttcgtgttgctcgacttttcaggttaatactcaacaaggttctcctacctcaccttctcgtatgattcgggagaataaaagtccgtcgtctactaagactgtgtcttagacttggtctttaacttctgttagttccgaacatacgtggtctaggtcttcgactcgttcaacttccgaaatatgtccccggaaatctactgatacgtctactccttcacctacaacataaatgaagcctgacctttgtcggactcgaacttagactgctcgtacctttctggaatgccaactgtagcggtctcccaaattcacctctcgtctttagggtcaccgaaagctgctaatttcgtcagcacgtttcacggtttatgaccttagaccgtctcacgtgtaaacttcgtagccctcttccccagtaatacttcctcgcggtctattgaggcctacatatattccagtgaggtcactacttgtgtgtaggcagggttagtcttcgtcatagtctacaaaccagagagagtttctgtaggtactgaaaggttgggttctttcgttcagaagtcggagagtggtataggaacctacttaacaagagtagatctcctctcaagtagagacagcctccactgcctgcttacagagtatttctccggtaggacgagccggactctatgtttttcaacatgttagtccgctctcagtttataagagacatctgatactttttttcattgtctatagtgctagattcacaatagggttaggtaagtagtactcaaggaatttcttctaagagccagacttcccctttccattctttagattctttaatccctagcgtggtgggggaatacttctcctgtgatcgtacctcatacgaggctcgcgaggttaactgtttaggataaaacctcaactgctctacctgtggatactaggcttagttaattctatactctttaagaagaaatgtcacttttactgccaatctagattagcaggcaagtcttgtatgagtctacaccgtcggcgacatagggtaaccctagtgtacatgtagccttaccgtccctttgcagggaagatgttttagaaccgaaaaaacccaagaagattagatttccggtgaggtcgccataaccgtctagttccagttggtctcatagtgcgagtgacgcttccgtcccgaataaacggtgtatcctaccccttctggggagggtacgagttacatggtctcgtgaagtcttctggtaagttatatccagaaatgttcccttgctaactcgagtgttactggtagatgctactactcagtgaccttcgtcgaggatactagaccctagtaaagttaagaaggtttaaaagactaaagtctctcttccggaattacaaaccggactaacagctctttttccgtagacctcgcacccaggacctgagatagccggtgaagtttactcgatcagattgaagatcgaagacttgttaggggccaaatgagtcagaggggattaaggtcggagagcttgttgattataggacagaaaagatagggatactttttttgattgtctctagctagacaaatgcgcagtgcctagggggcccgacgtccttaagcggtggtacccggtctagcactggtacaagctccgggacggggtgtagtagctgctccactagttgtagcactagtagcacgagtagtagtagtggtcgtagttccggcacatgttgaagcggtggacgccgtaggaccgggaccactcgaaggacaaggaccggccgtcttcgacgccgtacatgccggacttaccggggctatagatgttcccgcacatggtcaagttctcgcacctcaagctgtactcggtggacttggagtggtacgggttgcggacgtcgcggttgttatcggtggtgatgtagtcgtacccgtcgtcgccggacctcaactggaagtggttgctgtcgtaggacttggtgttgaagacgttggagtggtcgcggaagttgttcttttggaagctggtgtgggagtactcgtagcactcgtcggacgtggactcgtagtctccgttgtcgttggtgttccggcactcgacgctgaagttgttgccgtagtggtaggtcatgttggactcgaagtcgctaggagtctcgcggtagtcggtcacgtcttggaagtctccgtctcacgacctgtacaagtcttggcggaagccgccgttcatgtactcttcgccgaccccgacccggccgtcgctgccgttctggtggaccacgtcggtctggtcgatggtcatggagtagtaggtcttgtcttggaccctcttggtgacgtctatgcggcctggaaagccgtactcgtcttaggacaagcgggtcctcttttggttcaaggagtggtcctctgaccggccgtggaagtggacctgggactcgctgtcgtcgccgcacctcttgggaccgccgatgacggagtggttcacctactaggaccggcggctcgacttcacgaagccgttgtggcggcaccggttcacgttgcacttggtgctgctcctcaagacgctgtacgactctgagtagctgatgttgttccggcgggactcgttcaagttcgtcctgcacctctcgcgggacgtgcacaagttctggtggcacttgtcggagtagtcgctggtcgacgagtactctttggtggactctctggagtacccgcacgggatgacgttgatgtcgttcaagaccatagacctcgtgcggttctggccgctctggtcgcacgggttcacgaccgaccactggttaccgtcgatggacttgctctgggtgaagtcgctggtctagctcgtccttcggctgttgtactagtggctctacgactccttcctgatgtagttctctgtcccgtcgtggggggaccgggagtacctagacgagtacaagtcgtggtcgcggatggagtagtcgtagaaggacgtggaccacttctaggggtgggtgtctgtgtagttcccgccgtcgacggggttcggggtgtctgagtggttgttcccgtagacgtcgacgccgcggaagttccacgggccgcacttttggtagaccttctcctctattcgccggcgatgctggagctgatactttttttgattgtctataggagctgcggtggtacaagcggttccgatgggccttgaaggactctcttcacctgcggctgccgctggactagcggcacaggttagacttgctgtcgctgtttgacgtcgacgactcggaccagtggttctttttcgccaagaccacgaccgtcgccgggttcatggtcaaggacagagactgggacccgctgcacgactagctcctagtcaagggtagaggacaccaccacctttcgctgaagcacttcatgctcccgttcaagcggttggtgcacaggccgtgtgacctttgtcgggacccgtttcacttcgacttagagccgccgtcgtcgtctcacctttcggtcagatcgaagccgtgggacgccttcgttcttcacctagacgtcgtcgactaggccctgtcgcggctctcttggtagttggactctttggggcacgacgttgtccacgaccttccgtctttactccacgacacgcacgactgggtcttttagtggtggtacgtctttacgcactagtcgctcgtgtacgtccagctccttttcacgccgccgtagcacccgtaggtctggttctgtcacgtccaaaggcggtggctcctgccgttgcactggttcctatcgttgcaccacgagctctagggacggcggtgttgttaacggatgccgcactagctcgacatgcacttcgacctgccggtcaagctcaagacggacgactctcctttcgttccgccgaagctcttgttctttgcctagctgaggcacatggacctaggagaccacaagtctctcaagcggaagtagctgtacgggctacggcgagtgccttagtcgagagtcctaccgggagactcgcacgacttcgtccgatgtgacgacgaccttgccttgaaagtggggaagcggctcgacggacttggagtcgtttgtcgagactcgctgtagaaggtccggcacgacaagctgctgctcgacgactaccacgaactcgggcacacactgctagaccacagaccggacagaggatggcaccgacaagaccctctcgacttcggatctgtcgtcgtcctagaccaccggaaggacgtcgaacacccgacaagagacgttccgcctacagggcctggactcctacggcctagattcgtcgacaaatactggcggatgaaggaccacagtcgggaccggctctacggtctatcacgacgacgagacgacccgtgtacgacatttgacgtctagtaggggtgtgacacggtagacgactcccgggacagactactaccgcacaggctagaccttctagggtgtgactggggagacttcctgtggctctctaaaccgtagcacgtcgccgacaaacggtcgcggctatagtcggaccttgccgacttcagatcgcacttccggcactaggacttcctgaggttccacaagggagacgaggacacgtagtgggacttaccggacacacgggacccttctctcgtgtcgccgaggccgcttccgtctcctagagaagactgtacgcctctacagctccttttgggtcccggatacacggtggtcgtcgaccagtagtcgaccaagtcggaccacaaggaccggagaggagaccaccggtagaccctcgacttctttctgcacatgcaccaccttgacctgaccatagggctacgaggaccgctctaccaccacgactggacgctatggggacttctcctgccgtagtggacctgtgacctagtcagatcgctccacgagccgtcgccgttctgggactggtaggttcactttctcaaaccgctgcggccggtcatgtggacagtgtttccgcctcttcacgactcggtgtcggacgacgacgaggtgttctttctcctaccgtaaacctcgtggctgtaggacttcctggtctttctcgggttcttgttctggaaggactctacgctccggttcttgatgtcgccggccaagtgtacaaccaccgactggtggtagtcgtggctggactggaagtcgcacttcaggtcgtctccgtcgtcactaggagtcccgcaatgtacaccgcggcgatgtgacagacggctttctcacgccccgctgttgtttcttatgctcatgtcgcaccttacggttctcctgtcgcggacaggtcggcggcttctcagagacggatagcttcactaccacctgcggcacgtgttcgacttcatgctcttgatgtggaggtcgaaaaagtaggccctgtagtagttcgggctaggaggtttcttggacgtcgacttcggagacttcttgtcgtctgtccaccttcacaggaccctcatggggctgtggaccagatgtggggtgtcgatgaagtcggactggaaaacgcacgttcacgtcccgttcaggttcgcgctctttttcctggcccacaagtggctgttctggtcgcggtggcactagacgtctttcttgcggtcgtagtcgcagtctcgggtcctggccatgatgtcgtcgagaacctcgcttacccggtcgcacggtacaagaccaccgcctcctagaccgcctccaccttcgccgcctccgcctagatctttagacggacaccggtgaggactaggaccgtacaagggaacagacgtggtgtcggtcttggacgactctcggcacaggttgtacgacgtcttccggtctgtctgggaccttaagatggggacgtggtcgctcctttagctggtgctcctgtagtggttcctattctggtcgtggcaccttcggacggacggagaccttgactggttcttgctctcgacggacttgtcggccctttggtcgaagtagtggttgccgagaacggaccggtcgtctttctggaggaagtactaccgggacacggactcgtcgtagatgctcctggacttctacatggtccaccttaagttctggtacttgcggttcgacgactacctggggttcgccgtctagaaggacctggtcttatacgaccggcactagctgctcgactacgtccgggacttgaagttgtcgctctgtcacggggtcttcagatcggaccttcttgggctgaagatgttctggttctagttcgacacgtaggacgacgtgcggaaggcctagtctcggcactggtagctgtctcactactcgatggacttgcggaggactagttgatcggtctaagaagtacaaacctggtttagttgaacactatggtacgagtttctccggagttaatataaactcaaaaattaaaaatactttttttgattgtcgttagtaccttcaggtgctaaaactctggctgctcaagttactaaagttacttctactgatacggtgttctcttaaggacttagggctactcgcgtactgcatgaacttagtacgactaatgttggacttaagaggagattaatcactactataactgttaaattagtcctttaagttaagagaaggttaagggagctacaccctatcattcttgaccctacctcaagaactctacaattgcagtacagttcggttagggtagggttgtagagtctacgtatttacctacccttcaaccaattacagactattagtactacggtcagttcccatatcaaaaaatgtacttcacctgtttctccgtctttattgtaaactgcaccacctctggaagtaggcgccgaccccgttgtttggttaacttatgtagtttttcctttctacctgactgagtaagttttaagagcgaataaacacagttttcaaaaacctgaatgtgttcaactgtaattagaatttacgacagagactccaccttaacgagttgaaccgctcctgaaagtttccgtttcagtcttcttcaagagtaccttgcttgtatacgtcctaatcccaagggtcgaacccaggatgaaaataaagtcttcctacccgaatgaagttctttgaactataagattacctggctttgaaagacaattaccagtttctacactaatatccctcctacgtttgccacgataggtaccatacatcttatctgttggacaagagtctcgttctgtagaagagggaagatttatagatgtcttaacctctattttaacacctctccgtccctttaaaaagaatactgaactaattttaccaccttggctatacgttgaacttcgactactttaatcgttctcttagttccggaaatcagggtgttaagggagtaaaacttttagtatagttctgaagacaactacttccccgtttttaactggctccatattctaaggaggtactagtctattactcacacttttgtcacctagagtgtgaccactaaatacctagcaagtctgtaaccccagtaggaaaatatctaataatgtgacctgatctttttaatgtaagggttcattggtacttctttctataactacacagtatacgttttcgtgaacgttcactaaatcgagcctaacaagataaagttgtcaagttactagtatttttcaccaagcacttacctctgaacgagggagtactagtagggaaattttcagtacaatttcttttatgtaccgggtgtcgacgagttcaagttctaaaacctctatttaccgtacttgaaggcgactaatttacaaaactttatgggctgaatgatctgggtagctattatatgagactgttttcagtaagttacttatccagtctccacaactttgtacaggcttacttaggcttgtgaggatagggatcatttttccacaacgtctgatacaacctgtgtttccgatggttaacctttcttaaagaatttctctaactactcttcccgaatctactactactagattaataaccagaatttcctttcctctcccttgacttcaaccgtccatctaaaaagagggattacagaacctttaacgctcttatgaaacattaatggcttataaactatttctgagtaaagcagggatacaaatttccggactgttaccgcctgctagattgacgtcagtaatttttctacaatctaaggagtaggccggttcctaacttcagtatactccgttaaacgtatcggttagtgtaactaatgctttttaccttattggtggtttccttcaatagtttgccgggtcacaaggctcaatacccggtcaagaatccaataggtaggaattagctctcttgagtacttaaaaaactcttttcagaatatatgatgttaccttctggtctgaactacgcacaagtgttgttgtgtgactagttaagttggagggttgctcaaacaaccgttcctgttctcccacctgaccttccagatgccgtttttcctacctcataggagttagatgaccaataagtttctctccgattttagtctttgtgacgacagtttcagaaccgtgttccactattagttcaataaacgtgtgtcatattttgcttctttagctctttgcaacatcttaatgtcccacgagagttagtttaccaaagattattactcttttaatactgacgttagttttatccctgtcccttcaatcctgaaaactatttactgctactctgatacgttagacgtctaatgaacttaatacctttttatggctaaaaggcacctcactaatctcccaatctctggttctctaccagtgctcactgaacacagtggttactggtttatgggtgaacacgattatattactcgagtcaaaggtgtttacgagagtggcatcgagtaaaacgactcttgggttagttacggtactatgtcatgttaataaaaccctgtaaacgatctgagaacaactactacgtactaggacgagaagcagttagtaacatacttcaagttctattctatggcccgaacgtgtcaagatgaaagtttatgcggtacaacataaacctgggaaggtaacctcctcacagcccgtacagaaacaggtccaaaaactaatctcggaagggtctagggcattgtctttcagagagtaagacctctaagtaggtacatgtacgagcttcactcgtagacttcctctactcacgtcataaacctttggggctctatcggttcaaagcttattgagtgtatctgttcgatcatcttctaggttggagagacttgtagcgatacccttactcaggtcgcttgaacaatttctgactccaattttttacgaattagcttagttctgtttggtagtccttggtccactaattcctacgttggtatataaacatagtacttctcctagccgagtcttcaaagaataccagttatttaggagacaagggatctaaaaattcacttaagtttagtccgtgaaaaaaccctcagcgtctgcccgagtagtcagataaagttttaagagcatgataagccttgaggaaattctttttcatagtatcccttaacctactaaactaacactcctcactccataggagaaactgtgtaaatccctttgaagtaaactcttcccctagtacattttacacctgtacaagtcgatgagtacgactgtgtaattctatgtttaggaccccggcatgtcaataaccctgttgacatggggtaggtaatctttacaacccaggtgttgtagcttttctctgaggaacacgtggtacattgtgtagtcccaagttaatacaaagacacgtaacaggtctgccctaggtactgcagaaatcaagtgcccctggtaacggacgaatagatcccagattttgtagacttagatgtagataaaacgtcggaaccctttccctttcgtttcagggtgactaattttctcgatgtgcagaatctctacgatagagaaccaaacaacttgggctgagatttgatcgttactgatatgaaagattgtaggtgagaaattgtccgcttcttacctggttttccgtcgtacccaagttttcttgtcccagacgggaagtatccaaaagctgtagagcctactcggtaccacccaagcgtagagtctcgtgacgtcgtaactggtccaactaccgttgatgtctgtggtactccctagaccctctagtcttaaagctgaaaaataaggttcgttgcaacgagatacgagtttaatggtggtgacaacgttctctgcctacctagtggtcaacatgtctagtaatagtataacggacattcaggacaaactctgggtatcttctctagtgggacctgagttcatacctgatgtgcgggggtctacatagggtacacgacttctgtacctccttaccccttccaagcacccctgttctctattttgtctagataggaaatcttcccttaaccttcttaaatcgtggacgactcgttaggatagttcagccgtctacatatccaaaagatatacctctgaaccgcatatcttttagatgagtacggctcctgtcaagagataaaggagatagatatgttccagcataatctccagctccaaagaattttcccaacgatctgcctaattactctcgttcaacgacggttcattatgtggcctcttcagaccgagtaaacttctccggccggttgcgtcacatgcctccaaactaaatgaactaactatttaactcacatagtggaggtaaggaaagagaatgatctagtcctggataatctctgcttaatctttgctaaggggtgttctagggttggaggataggctgttcgttggcactatacccccactaacagtctttaatgaagtttatggttacggcagattaacttttcccttttatgtctagtgtaataagtgttaataccaataagagtctacagaataggtatctgaagtaacctggtaagagataaaggtggtgggagaacgtttaggatatgttcggtaaaaatagaccctttctattcttactcaactctctcgaccgtttagaaagaagtaacgattctagtcctctccccacccttctgtatgtacactttaagaagtggttcctgtataataacacaggtctcctttagtctgtacgaacgttcaagccctaacgattcctattattatttctgtactcgatagggggaaccccttcccttaggtctccctgttaatgttgttagggacaaataatatgctggtggggaatgggtttctacgatctctacggaggttcttaggttttaggggacgacaggccttagtccaacccggttaatggttgaccgcgagtaatattttaagcctcatataatgtaccttacccttaggtaatgtccctgaagaactcaacacctctgccgaggcctccctactgacgacgtaatgatgctcttttacacgtatcgtctccttataagttatcagacaatcttaatagtcccagtcagtacgctccgcggagaggactcggggggtcacgggatctttgaaatcctcctctatttagctctacacatttaccactttgtacaacccttataggtagactgaatacactgggttcctgaaccctgataaaggaggctgagtttcgtccgaaccccgaagtttaactaaattaacattacctataccttcaagccctaagaagatgatcggacttttaactctgcttacaatctttaatacacgtggcctaaaacctactcgttcctcaaaattagatgttctgaataccttgtatataaacactctcgcttttcttacgtcattgttaggaaccagggtacaagttctgccagctgaatcaagtttgtcttaaatcatcaagagtttgcagacttcatatataccatacatttccaaacttctttaattagctacttgggttagggctaaccagaaggtagttacttaggacctttttggacatgcgtaaggtcagtagtcttgtccttaaacggtctcgtttcttccaatcatgtatgaaatggaactgtccataagggagggttaagtaaggactaggaaaacatttgtaactctgatacgatgtttataagcctcatgggtgcccacacagagtacgccgacggaattttagtagactatctggacgtctaaataactggtaatcggaaaaaatataccgctaatatagcataatattgtagttagtatagtctcatcctggctatggaggcttggggggtagtctaccttaacgtgttttacacccctagcgatattgaccatattcgaaaaccgactcaaactacctctttctgtaaggtgatatagttgtcacaaatcgtcaataggtcgttagtaagggctaatccaccctccgacaaagtcattttcctcctatgttcgtcttcacctcatgatctccactacccgagggttttctatgggcttaaagtctgaggaaccggggttagcccttgacctagtctagagaccttaaccaggctttggttcaagcagatttaggtaagttactctagaacaagttagtcgatacagcatgtcacctattagtaaactttaccagtttaaacgcttctttgtgtccttactaacttacctagttatctgcttaaagttttcttctggccagatatgactacaacttctcactggatgtgctccttttgagaacctctctaattttttagtactcctctgaggtttgaaattcatactttttttgaaactaggaattctgggagaacaccaaaaataaaaaatagaccaaaacaccagaagca